SEQUENCE LISTING (921 sequences)

SEQ ID NO: 1 - DNA, 1154 bp, synthetic construct
  source 1..1154: mol_type = other DNA; note = Ai14 mouse transgene region 1
taaccatgttcatgccttcttctttttcctacagctcctgggcaacgtgctggttattgtgctgtctcatcattttggcaaagaattgatttgataccgcgggccctaagaagttcctattctctagaaagtataggaacttcgtcgacatttaaatcatttaaatataacttcgtataatgtatgctatacgaagttattcgcgatgaataaatgaaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagttattaggtccctcgacctgcagcccaagctagatcgaattcggccggccttgtacgcgttaagtgcaacacgatcccgccaccatg

SEQ ID NO: 2 - DNA, 34 bp, synthetic construct
  source 1..34: mol_type = other DNA; note = LoxP
ataacttcgtataatgtatgctatacgaagttat

SEQ ID NO: 3 - DNA, 128 bp, synthetic construct
  source 1..128: mol_type = other DNA; note = SV40 terminator
aacttgtttattgcagcttataatggttacaaataaagcaatagcatcacaaatttcacaaataaagcatttttttcactgcattctagttgtggtttgtccaaactcatcaatgtatcttatcatgt

SEQ ID NO: 4-19 - skipped (16 entries)

SEQ ID NO: 20 - DNA, 23 bp, synthetic construct
  source 1..23: mol_type = other DNA; note = Ai14 mouse transgene region 2 (20 nt + PAM)
gtctggatctgcgactctagagg

SEQ ID NO: 21 - DNA, 22 bp, synthetic construct
  source 1..22: mol_type = other DNA; note = Ai14 mouse transgene region 2 (19 nt + PAM)
tctggatctgcgactctagagg

SEQ ID NO: 22 - DNA, 21 bp, synthetic construct
  source 1..21: mol_type = other DNA; note = Ai14 mouse transgene region 2 (18 nt + PAM)
ctggatctgcgactctagagg

SEQ ID NO: 23 - DNA, 20 bp, synthetic construct
  source 1..20: mol_type = other DNA; note = Ai14 mouse transgene region 2 (17 nt + PAM)
tggatctgcgactctagagg

SEQ ID NO: 24-29 - skipped (6 entries)

SEQ ID NO: 30 - DNA, 23 bp, synthetic construct
  source 1..23: mol_type = other DNA; note = Ai14 mouse transgene region 3 (20 nt + PAM)
tcaatgtatcttatcatgtctgg

SEQ ID NO: 31 - DNA, 22 bp, synthetic construct
  source 1..22: mol_type = other DNA; note = Ai14 mouse transgene region 3 (19 nt + PAM)
caatgtatcttatcatgtctgg

SEQ ID NO: 32 - DNA, 21 bp, synthetic construct
  source 1..21: mol_type = other DNA; note = Ai14 mouse transgene region 3 (18 nt + PAM)
aatgtatcttatcatgtctgg

SEQ ID NO: 33 - DNA, 20 bp, synthetic construct
  source 1..20: mol_type = other DNA; note = Ai14 mouse transgene region 3 (17 nt + PAM)
atgtatcttatcatgtctgg

SEQ ID NO: 34-39 - skipped (6 entries)

SEQ ID NO: 40 - DNA, 23 bp, synthetic construct
  source 1..23: mol_type = other DNA; note = Ai14 mouse transgene region 4 (20 nt + PAM)
aaagaattgatttgataccgcgg

SEQ ID NO: 41 - DNA, 22 bp, synthetic construct
  source 1..22: mol_type = other DNA; note = Ai14 mouse transgene region 4 (19 nt + PAM)
aagaattgatttgataccgcgg

SEQ ID NO: 42 - DNA, 21 bp, synthetic construct
  source 1..21: mol_type = other DNA; note = Ai14 mouse transgene region 4 (18 nt + PAM)
agaattgatttgataccgcgg

SEQ ID NO: 43 - DNA, 20 bp, synthetic construct
  source 1..20: mol_type = other DNA; note = Ai14 mouse transgene region 4 (17 nt + PAM)
gaattgatttgataccgcgg

SEQ ID NO: 44-49 - skipped (6 entries)

SEQ ID NO: 50 - DNA, 23 bp, synthetic construct
  source 1..23: mol_type = other DNA; note = Ai14 mouse transgene region 5 (20 nt + PAM)
gtatgctatacgaagttattagg

SEQ ID NO: 51 - DNA, 22 bp, synthetic construct
  source 1..22: mol_type = other DNA; note = Ai14 mouse transgene region 5 (19 nt + PAM)
tatgctatacgaagttattagg

SEQ ID NO: 52 - DNA, 21 bp, synthetic construct
  source 1..21: mol_type = other DNA; note = Ai14 mouse transgene region 5 (18 nt + PAM)
atgctatacgaagttattagg

SEQ ID NO: 53 - DNA, 20 bp, synthetic construct
  source 1..20: mol_type = other DNA; note = Ai14 mouse transgene region 5 (17 nt + PAM)
tgctatacgaagttattagg

SEQ ID NO: 54-59 - skipped (6 entries)

SEQ ID NO: 60 - DNA, 40 bp, synthetic construct
  source 1..40: mol_type = other DNA; note = Ai14 mouse transgene region 6 (region within region 1 for targeting by one PS2 gRNA and one PS3 gRNA)
tcaatgtatcttatcatgtctggatctgcgactctagagg

SEQ ID NO: 61-69 - skipped (9 entries)

SEQ ID NO: 70 - DNA, 531 bp, synthetic construct
  source 1..531: mol_type = other DNA; note = Ai14 mouse transgene region 7 (region within region 1 for targeting by two PS2 gRNAs and three PS3 gRNAs)
tcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 71-79 - skipped (9 entries)

SEQ ID NO: 80 - DNA, 973 bp, synthetic construct
  source 1..973: mol_type = other DNA; note = Ai14 mouse transgene region 8 (region within region 1 between LaRo gRNAcleavage site and LoxP gRNA cleavage site)
ccgcgggccctaagaagttcctattctctagaaagtataggaacttcgtcgacatttaaatcatttaaatataacttcgtataatgtatgctatacgaagttattcgcgatgaataaatgaaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagtt

SEQ ID NO: 81 - DNA, 973 bp, synthetic construct
  source 1..973: mol_type = other DNA; note = Ai14 mouse transgene region 8 (complementary to SEQ ID NO: 80)
aacttcgtatagcatacattatacgaagttatattaagggttccggatcagcttgatggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctgcaagctttcatttattcatcgcgaataacttcgtatagcatacattatacgaagttatatttaaatgatttaaatgtcgacgaagttcctatactttctagagaataggaacttcttagggcccgcgg

SEQ ID NO: 82-89 - skipped (8 entries)

SEQ ID NO: 90 - DNA, 996 bp, synthetic construct
  source 1..996: mol_type = other DNA; note = Ai14 mouse transgene region 9 (region within region 1 for targeting by two PS2 gRNAs, three PS3 gRNAs, one LaRo gRNA, and one LoxP gRNA)
aaagaattgatttgataccgcgggccctaagaagttcctattctctagaaagtataggaacttcgtcgacatttaaatcatttaaatataacttcgtataatgtatgctatacgaagttattcgcgatgaataaatgaaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagttattagg

SEQ ID NO: 91-100 - skipped (10 entries)

SEQ ID NO: 101 - RNA, 12 nt, synthetic construct
  source 1..12: mol_type = other RNA; note = crRNA flagpole sequence (option 1)
gttttagagcta

SEQ ID NO: 102 - RNA, 12 nt, synthetic construct
  source 1..12: mol_type = other RNA; note = crRNA flagpole sequence (option 2)
gtttaagagcta

SEQ ID NO: 103-105 - skipped (3 entries)

SEQ ID NO: 106 - RNA, 13 nt, synthetic construct
  source 1..13: mol_type = other RNA; note = tracrRNA flagpole (option 1)
tagcaagttaaaa

SEQ ID NO: 107 - RNA, 13 nt, synthetic construct
  source 1..13: mol_type = other RNA; note = tracrRNA flagpole (option 2)
tagcaagtttaaa

SEQ ID NO: 108 - RNA, 47 nt, synthetic construct
  source 1..47: mol_type = other RNA; note = tracrRNA nuclease binding domain (may be followed by multiple uracils
taaggctagtccgttatcaacttgaaaaagtggcaccgagtcggtgc

SEQ ID NO: 109-110 - skipped (2 entries)

SEQ ID NO: 111 - RNA, 76 nt, synthetic construct
  source 1..76: mol_type = other RNA; note = sgRNA backbone (3' to targeting sequence) (option 1) (may be followed by multiple uracils)
gttttagagctagaaatagcaagttaaaataaggctagtccgttatcaacttgaaaaagtggcaccgagtcggtgc

SEQ ID NO: 112 - RNA, 76 nt, synthetic construct
  source 1..76: mol_type = other RNA; note = sgRNA backbone (3' to targeting sequence) (option 2) (may be followed by multiple uracils)
gtttaagagctagaaatagcaagtttaaataaggctagtccgttatcaacttgaaaaagtggcaccgagtcggtgc

SEQ ID NO: 113 - RNA, 86 nt, synthetic construct
  source 1..86: mol_type = other RNA; note = sgRNA backbone (3' to targeting sequence) (option 3) (may be followed by multiple uracils)
gttttagagctatgctggaaacagcatagcaagttaaaataaggctagtccgttatcaacttgaaaaagtggcaccgagtcggtgc

SEQ ID NO: 114 - RNA, 86 nt, synthetic construct
  source 1..86: mol_type = other RNA; note = sgRNA backbone (3' to targeting sequence) (option 4) (may be followed by multiple uracils)
gtttaagagctatgctggaaacagcatagcaagtttaaataaggctagtccgttatcaacttgaaaaagtggcaccgagtcggtgc

SEQ ID NO: 115 - RNA, 22 nt, synthetic construct
  source 1..22: mol_type = other RNA; note = dgRNA crRNA backbone (option 1)
gttttagagctatgctgttttg

SEQ ID NO: 116 - RNA, 74 nt, synthetic construct
  source 1..74: mol_type = other RNA; note = dgRNA tracrRNA (option 1)
aacagcatagcaagttaaaataaggctagtccgttatcaacttgaaaaagtggcaccgagtcggtgcttttttt

SEQ ID NO: 117 - RNA, 22 nt, synthetic construct
  source 1..22: mol_type = other RNA; note = dgRNA crRNA backbone (option 2)
gtttaagagctatgctgttttg

SEQ ID NO: 118 - RNA, 74 nt, synthetic construct
  source 1..74: mol_type = other RNA; note = dgRNA tracrRNA (option 2)
aacagcatagcaagtttaaataaggctagtccgttatcaacttgaaaaagtggcaccgagtcggtgcttttttt

SEQ ID NO: 119 - skipped

SEQ ID NO: 120 - RNA, 20 nt, synthetic construct
  source 1..20: mol_type = other RNA; note = PS2 gRNA targeting sequence (20 nt)
gtctggatctgcgactctag

SEQ ID NO: 121 - RNA, 19 nt, synthetic construct
  source 1..19: mol_type = other RNA; note = PS2 gRNA targeting sequence (19 nt)
tctggatctgcgactctag

SEQ ID NO: 122 - RNA, 18 nt, synthetic construct
  source 1..18: mol_type = other RNA; note = PS2 gRNA targeting sequence (18 nt)
ctggatctgcgactctag

SEQ ID NO: 123 - RNA, 17 nt, synthetic construct
  source 1..17: mol_type = other RNA; note = PS2 gRNA targeting sequence (17 nt)
tggatctgcgactctag

SEQ ID NO: 124 - skipped

SEQ ID NO: 125 - RNA, 96 nt, synthetic construct
  source 1..96: mol_type = other RNA; note = PS2 gRNA targeting sequence (20 nt) + backbone (option 1)
gtctggatctgcgactctaggttttagagctagaaatagcaagttaaaataaggctagtccgttatcaacttgaaaaagtggcaccgagtcggtgc

SEQ ID NO: 126 - RNA, 95 nt, synthetic construct
  source 1..95: mol_type = other RNA; note = PS2 gRNA targeting sequence (19 nt) + backbone (option 1)
tctggatctgcgactctaggttttagagctagaaatagcaagttaaaataaggctagtccgttatcaacttgaaaaagtggcaccgagtcggtgc

SEQ ID NO: 127 - RNA, 94 nt, synthetic construct
  source 1..94: mol_type = other RNA; note = PS2 gRNA targeting sequence (18 nt) + backbone (option 1)
ctggatctgcgactctaggttttagagctagaaatagcaagttaaaataaggctagtccgttatcaacttgaaaaagtggcaccgagtcggtgc

SEQ ID NO: 128 - RNA, 93 nt, synthetic construct
  source 1..93: mol_type = other RNA; note = PS2 gRNA targeting sequence (17 nt) + backbone (option 1)
tggatctgcgactctaggttttagagctagaaatagcaagttaaaataaggctagtccgttatcaacttgaaaaagtggcaccgagtcggtgc

SEQ ID NO: 129 - skipped

SEQ ID NO: 130 - RNA, 20 nt, synthetic construct
  source 1..20: mol_type = other RNA; note = PS3 gRNA targeting sequence (20 nt)
tcaatgtatcttatcatgtc

SEQ ID NO: 131 - RNA, 19 nt, synthetic construct
  source 1..19: mol_type = other RNA; note = PS3 gRNA targeting sequence (19 nt)
caatgtatcttatcatgtc

SEQ ID NO: 132 - RNA, 18 nt, synthetic construct
  source 1..18: mol_type = other RNA; note = PS3 gRNA targeting sequence (18 nt)
aatgtatcttatcatgtc

SEQ ID NO: 133 - RNA, 17 nt, synthetic construct
  source 1..17: mol_type = other RNA; note = PS3 gRNA targeting sequence (17 nt)
atgtatcttatcatgtc

SEQ ID NO: 134 - skipped

SEQ ID NO: 135 - RNA, 96 nt, synthetic construct
  source 1..96: mol_type = other RNA; note = PS3 gRNA targeting sequence (20 nt) + backbone (option 1)
tcaatgtatcttatcatgtcgttttagagctagaaatagcaagttaaaataaggctagtccgttatcaacttgaaaaagtggcaccgagtcggtgc

SEQ ID NO: 136 - RNA, 95 nt, synthetic construct
  source 1..95: mol_type = other RNA; note = PS3 gRNA targeting sequence (19 nt) + backbone (option 1)
caatgtatcttatcatgtcgttttagagctagaaatagcaagttaaaataaggctagtccgttatcaacttgaaaaagtggcaccgagtcggtgc

SEQ ID NO: 137 - RNA, 94 nt, synthetic construct
  source 1..94: mol_type = other RNA; note = PS3 gRNA targeting sequence (18 nt) + backbone (option 1)
aatgtatcttatcatgtcgttttagagctagaaatagcaagttaaaataaggctagtccgttatcaacttgaaaaagtggcaccgagtcggtgc

SEQ ID NO: 138 - RNA, 93 nt, synthetic construct
  source 1..93: mol_type = other RNA; note = PS3 gRNA targeting sequence (17 nt) + backbone (option 1)
atgtatcttatcatgtcgttttagagctagaaatagcaagttaaaataaggctagtccgttatcaacttgaaaaagtggcaccgagtcggtgc

SEQ ID NO: 139 - skipped

SEQ ID NO: 140 - RNA, 20 nt, synthetic construct
  source 1..20: mol_type = other RNA; note = LaRo gRNA targeting sequence (20 nt)
aaagaattgatttgataccg

SEQ ID NO: 141 - RNA, 19 nt, synthetic construct
  source 1..19: mol_type = other RNA; note = LaRo gRNA targeting sequence (19 nt)
aagaattgatttgataccg

SEQ ID NO: 142 - RNA, 18 nt, synthetic construct
  source 1..18: mol_type = other RNA; note = LaRo gRNA targeting sequence (18 nt)
agaattgatttgataccg

SEQ ID NO: 143 - RNA, 17 nt, synthetic construct
  source 1..17: mol_type = other RNA; note = LaRo gRNA targeting sequence (17 nt)
gaattgatttgataccg

SEQ ID NO: 144 - skipped

SEQ ID NO: 145 - RNA, 96 nt, synthetic construct
  source 1..96: mol_type = other RNA; note = LaRo gRNA targeting sequence (20 nt) + backbone (option 1)
aaagaattgatttgataccggttttagagctagaaatagcaagttaaaataaggctagtccgttatcaacttgaaaaagtggcaccgagtcggtgc

SEQ ID NO: 146 - RNA, 95 nt, synthetic construct
  source 1..95: mol_type = other RNA; note = LaRo gRNA targeting sequence (19 nt) + backbone (option 1)
aagaattgatttgataccggttttagagctagaaatagcaagttaaaataaggctagtccgttatcaacttgaaaaagtggcaccgagtcggtgc

SEQ ID NO: 147 - RNA, 94 nt, synthetic construct
  source 1..94: mol_type = other RNA; note = LaRo gRNA targeting sequence (18 nt) + backbone (option 1)
agaattgatttgataccggttttagagctagaaatagcaagttaaaataaggctagtccgttatcaacttgaaaaagtggcaccgagtcggtgc

SEQ ID NO: 148 - RNA, 93 nt, synthetic construct
  source 1..93: mol_type = other RNA; note = LaRo gRNA targeting sequence (17 nt) + backbone (option 1)
gaattgatttgataccggttttagagctagaaatagcaagttaaaataaggctagtccgttatcaacttgaaaaagtggcaccgagtcggtgc

SEQ ID NO: 149 - skipped

SEQ ID NO: 150 - RNA, 20 nt, synthetic construct
  source 1..20: mol_type = other RNA; note = LoxP gRNA targeting sequence (20 nt)
gtatgctatacgaagttatt

SEQ ID NO: 151 - RNA, 19 nt, synthetic construct
  source 1..19: mol_type = other RNA; note = LoxP gRNA targeting sequence (19 nt)
tatgctatacgaagttatt

SEQ ID NO: 152 - RNA, 18 nt, synthetic construct
  source 1..18: mol_type = other RNA; note = LoxP gRNA targeting sequence (18 nt)
atgctatacgaagttatt

SEQ ID NO: 153 - RNA, 17 nt, synthetic construct
  source 1..17: mol_type = other RNA; note = LoxP gRNA targeting sequence (17 nt)
tgctatacgaagttatt

SEQ ID NO: 154 - skipped

SEQ ID NO: 155 - RNA, 96 nt, synthetic construct
  source 1..96: mol_type = other RNA; note = LoxP gRNA targeting sequence (20 nt) + backbone (option 1)
gtatgctatacgaagttattgttttagagctagaaatagcaagttaaaataaggctagtccgttatcaacttgaaaaagtggcaccgagtcggtgc

SEQ ID NO: 156 - RNA, 95 nt, synthetic construct
  source 1..95: mol_type = other RNA; note = LoxP gRNA targeting sequence (19 nt) + backbone (option 1)
tatgctatacgaagttattgttttagagctagaaatagcaagttaaaataaggctagtccgttatcaacttgaaaaagtggcaccgagtcggtgc

SEQ ID NO: 157 - RNA, 94 nt, synthetic construct
  source 1..94: mol_type = other RNA; note = LoxP gRNA targeting sequence (18 nt) + backbone (option 1)
atgctatacgaagttattgttttagagctagaaatagcaagttaaaataaggctagtccgttatcaacttgaaaaagtggcaccgagtcggtgc

SEQ ID NO: 158 - RNA, 93 nt, synthetic construct
  source 1..93: mol_type = other RNA; note = LoxP gRNA targeting sequence (17 nt) + backbone (option 1)
tgctatacgaagttattgttttagagctagaaatagcaagttaaaataaggctagtccgttatcaacttgaaaaagtggcaccgagtcggtgc

SEQ ID NO: 159 - skipped

SEQ ID NO: 160 - RNA, 20 nt, synthetic construct
  source 1..20: mol_type = other RNA; note = LTciferase gRNA targeting sequence (20 nt)
cttcgaaatgtccgttgggt

SEQ ID NO: 161-224 - skipped (64 entries)

SEQ ID NO: 225 - RNA, 96 nt, synthetic construct
  source 1..96: mol_type = other RNA; note = PS2 gRNA targeting sequence (20 nt) + backbone (option 2)
gtctggatctgcgactctaggtttaagagctagaaatagcaagtttaaataaggctagtccgttatcaacttgaaaaagtggcaccgagtcggtgc

SEQ ID NO: 226 - RNA, 95 nt, synthetic construct
  source 1..95: mol_type = other RNA; note = PS2 gRNA targeting sequence (19 nt) + backbone (option 2)
tctggatctgcgactctaggtttaagagctagaaatagcaagtttaaataaggctagtccgttatcaacttgaaaaagtggcaccgagtcggtgc

SEQ ID NO: 227 - RNA, 94 nt, synthetic construct
  source 1..94: mol_type = other RNA; note = PS2 gRNA targeting sequence (18 nt) + backbone (option 2)
ctggatctgcgactctaggtttaagagctagaaatagcaagtttaaataaggctagtccgttatcaacttgaaaaagtggcaccgagtcggtgc

SEQ ID NO: 228 - RNA, 93 nt, synthetic construct
  source 1..93: mol_type = other RNA; note = PS2 gRNA targeting sequence (17 nt) + backbone (option 2)
tggatctgcgactctaggtttaagagctagaaatagcaagtttaaataaggctagtccgttatcaacttgaaaaagtggcaccgagtcggtgc

SEQ ID NO: 229-234 - skipped (6 entries)

SEQ ID NO: 235 - RNA, 96 nt, synthetic construct
  source 1..96: mol_type = other RNA; note = PS3 gRNA targeting sequence (20 nt) + backbone (option 2)
tcaatgtatcttatcatgtcgtttaagagctagaaatagcaagtttaaataaggctagtccgttatcaacttgaaaaagtggcaccgagtcggtgc

SEQ ID NO: 236 - RNA, 95 nt, synthetic construct
  source 1..95: mol_type = other RNA; note = PS3 gRNA targeting sequence (19 nt) + backbone (option 2)
caatgtatcttatcatgtcgtttaagagctagaaatagcaagtttaaataaggctagtccgttatcaacttgaaaaagtggcaccgagtcggtgc

SEQ ID NO: 237 - RNA, 94 nt, synthetic construct
  source 1..94: mol_type = other RNA; note = PS3 gRNA targeting sequence (18 nt) + backbone (option 2)
aatgtatcttatcatgtcgtttaagagctagaaatagcaagtttaaataaggctagtccgttatcaacttgaaaaagtggcaccgagtcggtgc

SEQ ID NO: 238 - RNA, 93 nt, synthetic construct
  source 1..93: mol_type = other RNA; note = PS3 gRNA targeting sequence (17 nt) + backbone (option 2)
atgtatcttatcatgtcgtttaagagctagaaatagcaagtttaaataaggctagtccgttatcaacttgaaaaagtggcaccgagtcggtgc

SEQ ID NO: 239-244 - skipped (6 entries)

SEQ ID NO: 245 - RNA, 96 nt, synthetic construct
  source 1..96: mol_type = other RNA; note = LaRo gRNA targeting sequence (20 nt) + backbone (option 2)
aaagaattgatttgataccggtttaagagctagaaatagcaagtttaaataaggctagtccgttatcaacttgaaaaagtggcaccgagtcggtgc

SEQ ID NO: 246 - RNA, 95 nt, synthetic construct
  source 1..95: mol_type = other RNA; note = LaRo gRNA targeting sequence (19 nt) + backbone (option 2)
aagaattgatttgataccggtttaagagctagaaatagcaagtttaaataaggctagtccgttatcaacttgaaaaagtggcaccgagtcggtgc

SEQ ID NO: 247 - RNA, 94 nt, synthetic construct
  source 1..94: mol_type = other RNA; note = LaRo gRNA targeting sequence (18 nt) + backbone (option 2)
agaattgatttgataccggtttaagagctagaaatagcaagtttaaataaggctagtccgttatcaacttgaaaaagtggcaccgagtcggtgc

SEQ ID NO: 248 - RNA, 93 nt, synthetic construct
  source 1..93: mol_type = other RNA; note = LaRo gRNA targeting sequence (17 nt) + backbone (option 2)
gaattgatttgataccggtttaagagctagaaatagcaagtttaaataaggctagtccgttatcaacttgaaaaagtggcaccgagtcggtgc

SEQ ID NO: 249-254 - skipped (6 entries)

SEQ ID NO: 255 - RNA, 96 nt, synthetic construct
  source 1..96: mol_type = other RNA; note = LoxP gRNA targeting sequence (20 nt) + backbone (option 2)
gtatgctatacgaagttattgtttaagagctagaaatagcaagtttaaataaggctagtccgttatcaacttgaaaaagtggcaccgagtcggtgc

SEQ ID NO: 256 - RNA, 95 nt, synthetic construct
  source 1..95: mol_type = other RNA; note = LoxP gRNA targeting sequence (19 nt) + backbone (option 2)
tatgctatacgaagttattgtttaagagctagaaatagcaagtttaaataaggctagtccgttatcaacttgaaaaagtggcaccgagtcggtgc

SEQ ID NO: 257 - RNA, 94 nt, synthetic construct
  source 1..94: mol_type = other RNA; note = LoxP gRNA targeting sequence (18 nt) + backbone (option 2)
atgctatacgaagttattgtttaagagctagaaatagcaagtttaaataaggctagtccgttatcaacttgaaaaagtggcaccgagtcggtgc

SEQ ID NO: 258 - RNA, 93 nt, synthetic construct
  source 1..93: mol_type = other RNA; note = LoxP gRNA targeting sequence (17 nt) + backbone (option 2)
tgctatacgaagttattgtttaagagctagaaatagcaagtttaaataaggctagtccgttatcaacttgaaaaagtggcaccgagtcggtgc

SEQ ID NO: 259-324 - skipped (66 entries)

SEQ ID NO: 325 - RNA, 106 nt, synthetic construct
  source 1..106: mol_type = other RNA; note = PS2 gRNA targeting sequence (20 nt) + backbone (option 3)
gtctggatctgcgactctaggttttagagctatgctggaaacagcatagcaagttaaaataaggctagtccgttatcaacttgaaaaagtggcaccgagtcggtgc

SEQ ID NO: 326 - RNA, 105 nt, synthetic construct
  source 1..105: mol_type = other RNA; note = PS2 gRNA targeting sequence (19 nt) + backbone (option 3)
tctggatctgcgactctaggttttagagctatgctggaaacagcatagcaagttaaaataaggctagtccgttatcaacttgaaaaagtggcaccgagtcggtgc

SEQ ID NO: 327 - RNA, 104 nt, synthetic construct
  source 1..104: mol_type = other RNA; note = PS2 gRNA targeting sequence (18 nt) + backbone (option 3)
ctggatctgcgactctaggttttagagctatgctggaaacagcatagcaagttaaaataaggctagtccgttatcaacttgaaaaagtggcaccgagtcggtgc

SEQ ID NO: 328 - RNA, 103 nt, synthetic construct
  source 1..103: mol_type = other RNA; note = PS2 gRNA targeting sequence (17 nt) + backbone (option 3)
tggatctgcgactctaggttttagagctatgctggaaacagcatagcaagttaaaataaggctagtccgttatcaacttgaaaaagtggcaccgagtcggtgc

SEQ ID NO: 329-334 - skipped (6 entries)

SEQ ID NO: 335 - RNA, 106 nt, synthetic construct
  source 1..106: mol_type = other RNA; note = PS3 gRNA targeting sequence (20 nt) + backbone (option 3)
tcaatgtatcttatcatgtcgttttagagctatgctggaaacagcatagcaagttaaaataaggctagtccgttatcaacttgaaaaagtggcaccgagtcggtgc

SEQ ID NO: 336 - RNA, 105 nt, synthetic construct
  source 1..105: mol_type = other RNA; note = PS3 gRNA targeting sequence (19 nt) + backbone (option 3)
caatgtatcttatcatgtcgttttagagctatgctggaaacagcatagcaagttaaaataaggctagtccgttatcaacttgaaaaagtggcaccgagtcggtgc

SEQ ID NO: 337 - RNA, 104 nt, synthetic construct
  source 1..104: mol_type = other RNA; note = PS3 gRNA targeting sequence (18 nt) + backbone (option 3)
aatgtatcttatcatgtcgttttagagctatgctggaaacagcatagcaagttaaaataaggctagtccgttatcaacttgaaaaagtggcaccgagtcggtgc

SEQ ID NO: 338 - RNA, 103 nt, synthetic construct
  source 1..103: mol_type = other RNA; note = PS3 gRNA targeting sequence (17 nt) + backbone (option 3)
atgtatcttatcatgtcgttttagagctatgctggaaacagcatagcaagttaaaataaggctagtccgttatcaacttgaaaaagtggcaccgagtcggtgc

SEQ ID NO: 339-344 - skipped (6 entries)

SEQ ID NO: 345 - RNA, 106 nt, synthetic construct
  source 1..106: mol_type = other RNA; note = LaRo gRNA targeting sequence (20 nt) + backbone (option 3)
aaagaattgatttgataccggttttagagctatgctggaaacagcatagcaagttaaaataaggctagtccgttatcaacttgaaaaagtggcaccgagtcggtgc

SEQ ID NO: 346 - RNA, 105 nt, synthetic construct
  source 1..105: mol_type = other RNA; note = LaRo gRNA targeting sequence (19 nt) + backbone (option 3)
aagaattgatttgataccggttttagagctatgctggaaacagcatagcaagttaaaataaggctagtccgttatcaacttgaaaaagtggcaccgagtcggtgc

SEQ ID NO: 347 - RNA, 104 nt, synthetic construct
  source 1..104: mol_type = other RNA; note = LaRo gRNA targeting sequence (18 nt) + backbone (option 3)
agaattgatttgataccggttttagagctatgctggaaacagcatagcaagttaaaataaggctagtccgttatcaacttgaaaaagtggcaccgagtcggtgc

SEQ ID NO: 348 - RNA, 103 nt, synthetic construct
  source 1..103: mol_type = other RNA; note = LaRo gRNA targeting sequence (17 nt) + backbone (option 3)
gaattgatttgataccggttttagagctatgctggaaacagcatagcaagttaaaataaggctagtccgttatcaacttgaaaaagtggcaccgagtcggtgc

SEQ ID NO: 349-354 - skipped (6 entries)

SEQ ID NO: 355 - RNA, 106 nt, synthetic construct
  source 1..106: mol_type = other RNA; note = LoxP gRNA targeting sequence (20 nt) + backbone (option 3)
gtatgctatacgaagttattgttttagagctatgctggaaacagcatagcaagttaaaataaggctagtccgttatcaacttgaaaaagtggcaccgagtcggtgc

SEQ ID NO: 356 - RNA, 105 nt, synthetic construct
  source 1..105: mol_type = other RNA; note = LoxP gRNA targeting sequence (19 nt) + backbone (option 3)
tatgctatacgaagttattgttttagagctatgctggaaacagcatagcaagttaaaataaggctagtccgttatcaacttgaaaaagtggcaccgagtcggtgc

SEQ ID NO: 357 - RNA, 104 nt, synthetic construct
  source 1..104: mol_type = other RNA; note = LoxP gRNA targeting sequence (18 nt) + backbone (option 3)
atgctatacgaagttattgttttagagctatgctggaaacagcatagcaagttaaaataaggctagtccgttatcaacttgaaaaagtggcaccgagtcggtgc

SEQ ID NO: 358 - RNA, 103 nt, synthetic construct
  source 1..103: mol_type = other RNA; note = LoxP gRNA targeting sequence (17 nt) + backbone (option 3)
tgctatacgaagttattgttttagagctatgctggaaacagcatagcaagttaaaataaggctagtccgttatcaacttgaaaaagtggcaccgagtcggtgc

SEQ ID NO: 359-424 - skipped (66 entries)

SEQ ID NO: 425 - RNA, 106 nt, synthetic construct
  source 1..106: mol_type = other RNA; note = PS2 gRNA targeting sequence (20 nt) + backbone (option 4)
gtctggatctgcgactctaggtttaagagctatgctggaaacagcatagcaagtttaaataaggctagtccgttatcaacttgaaaaagtggcaccgagtcggtgc

SEQ ID NO: 426 - RNA, 105 nt, synthetic construct
  source 1..105: mol_type = other RNA; note = PS2 gRNA targeting sequence (19 nt) + backbone (option 4)
tctggatctgcgactctaggtttaagagctatgctggaaacagcatagcaagtttaaataaggctagtccgttatcaacttgaaaaagtggcaccgagtcggtgc

SEQ ID NO: 427 - RNA, 104 nt, synthetic construct
  source 1..104: mol_type = other RNA; note = PS2 gRNA targeting sequence (18 nt) + backbone (option 4)
ctggatctgcgactctaggtttaagagctatgctggaaacagcatagcaagtttaaataaggctagtccgttatcaacttgaaaaagtggcaccgagtcggtgc

SEQ ID NO: 428 - RNA, 103 nt, synthetic construct
  source 1..103: mol_type = other RNA; note = PS2 gRNA targeting sequence (17 nt) + backbone (option 4)
tggatctgcgactctaggtttaagagctatgctggaaacagcatagcaagtttaaataaggctagtccgttatcaacttgaaaaagtggcaccgagtcggtgc

SEQ ID NO: 429-434 - skipped (6 entries)

SEQ ID NO: 435 - RNA, 106 nt, synthetic construct
  source 1..106: mol_type = other RNA; note = PS3 gRNA targeting sequence (20 nt) + backbone (option 4)
tcaatgtatcttatcatgtcgtttaagagctatgctggaaacagcatagcaagtttaaataaggctagtccgttatcaacttgaaaaagtggcaccgagtcggtgc

SEQ ID NO: 436 - RNA, 105 nt, synthetic construct
  source 1..105: mol_type = other RNA; note = PS3 gRNA targeting sequence (19 nt) + backbone (option 4)
caatgtatcttatcatgtcgtttaagagctatgctggaaacagcatagcaagtttaaataaggctagtccgttatcaacttgaaaaagtggcaccgagtcggtgc

SEQ ID NO: 437 - RNA, 104 nt, synthetic construct
  source 1..104: mol_type = other RNA; note = PS3 gRNA targeting sequence (18 nt) + backbone (option 4)
aatgtatcttatcatgtcgtttaagagctatgctggaaacagcatagcaagtttaaataaggctagtccgttatcaacttgaaaaagtggcaccgagtcggtgc

SEQ ID NO: 438 - RNA, 103 nt, synthetic construct
  source 1..103: mol_type = other RNA; note = PS3 gRNA targeting sequence (17 nt) + backbone (option 4)
atgtatcttatcatgtcgtttaagagctatgctggaaacagcatagcaagtttaaataaggctagtccgttatcaacttgaaaaagtggcaccgagtcggtgc

SEQ ID NO: 439-444 - skipped (6 entries)

SEQ ID NO: 445 - RNA, 106 nt, synthetic construct
  source 1..106: mol_type = other RNA; note = LaRo gRNA targeting sequence (20 nt) + backbone (option 4)
aaagaattgatttgataccggtttaagagctatgctggaaacagcatagcaagtttaaataaggctagtccgttatcaacttgaaaaagtggcaccgagtcggtgc

SEQ ID NO: 446 - RNA, 105 nt, synthetic construct
  source 1..105: mol_type = other RNA; note = LaRo gRNA targeting sequence (19 nt) + backbone (option 4)
aagaattgatttgataccggtttaagagctatgctggaaacagcatagcaagtttaaataaggctagtccgttatcaacttgaaaaagtggcaccgagtcggtgc

SEQ ID NO: 447 - RNA, 104 nt, synthetic construct
  source 1..104: mol_type = other RNA; note = LaRo gRNA targeting sequence (18 nt) + backbone (option 4)
agaattgatttgataccggtttaagagctatgctggaaacagcatagcaagtttaaataaggctagtccgttatcaacttgaaaaagtggcaccgagtcggtgc

SEQ ID NO: 448 - RNA, 103 nt, synthetic construct
  source 1..103: mol_type = other RNA; note = LaRo gRNA targeting sequence (17 nt) + backbone (option 4)
gaattgatttgataccggtttaagagctatgctggaaacagcatagcaagtttaaataaggctagtccgttatcaacttgaaaaagtggcaccgagtcggtgc

SEQ ID NO: 449-454 - skipped (6 entries)

SEQ ID NO: 455 - RNA, 106 nt, synthetic construct
  source 1..106: mol_type = other RNA; note = LoxP gRNA targeting sequence (20 nt) + backbone (option 4)
gtatgctatacgaagttattgtttaagagctatgctggaaacagcatagcaagtttaaataaggctagtccgttatcaacttgaaaaagtggcaccgagtcggtgc

SEQ ID NO: 456 - RNA, 105 nt, synthetic construct
  source 1..105: mol_type = other RNA; note = LoxP gRNA targeting sequence (19 nt) + backbone (option 4)
tatgctatacgaagttattgtttaagagctatgctggaaacagcatagcaagtttaaataaggctagtccgttatcaacttgaaaaagtggcaccgagtcggtgc

SEQ ID NO: 457 - RNA, 104 nt, synthetic construct
  source 1..104: mol_type = other RNA; note = LoxP gRNA targeting sequence (18 nt) + backbone (option 4)
atgctatacgaagttattgtttaagagctatgctggaaacagcatagcaagtttaaataaggctagtccgttatcaacttgaaaaagtggcaccgagtcggtgc

SEQ ID NO: 458 - RNA, 103 nt, synthetic construct
  source 1..103: mol_type = other RNA; note = LoxP gRNA targeting sequence (17 nt) + backbone (option 4)
tgctatacgaagttattgtttaagagctatgctggaaacagcatagcaagtttaaataaggctagtccgttatcaacttgaaaaagtggcaccgagtcggtgc

SEQ ID NO: 459-509 - skipped (51 entries)

SEQ ID NO: 510 - DNA, 20 bp, synthetic construct
  source 1..20: mol_type = other DNA; note = ssODN 20T (5-prime homology arm (10 nt) + 3-prime homology arm (10 nt))
tgatttgataattaggtccc

SEQ ID NO: 511 - DNA, 10 bp, synthetic construct
  source 1..10: mol_type = other DNA; note = 5-prime homology arm (10 nt)
tgatttgata

SEQ ID NO: 512 - DNA, 10 bp, synthetic construct
  source 1..10: mol_type = other DNA; note = 3-prime homology arm (10 nt)
attaggtccc

SEQ ID NO: 513-519 - skipped (7 entries)

SEQ ID NO: 520 - DNA, 40 bp, synthetic construct
  source 1..40: mol_type = other DNA; note = ssODN 40T (5-prime homology arm (20 nt) + 3-prime homology arm (20 nt))
ggcaaagaattgatttgataattaggtccctcgacctgca

SEQ ID NO: 521 - DNA, 20 bp, synthetic construct
  source 1..20: mol_type = other DNA; note = 5-prime homology arm (20 nt)
ggcaaagaattgatttgata

SEQ ID NO: 522 - DNA, 20 bp, synthetic construct
  source 1..20: mol_type = other DNA; note = 3-prime homology arm (20 nt)
attaggtccctcgacctgca

SEQ ID NO: 523-529 - skipped (7 entries)

SEQ ID NO: 530 - DNA, 60 bp, synthetic construct
  source 1..60: mol_type = other DNA; note = ssODN 60T (5-prime homology arm (30 nt) + 3-prime homology arm (30 nt))
tcatcattttggcaaagaattgatttgataattaggtccctcgacctgcagcccaagcta

SEQ ID NO: 531 - DNA, 30 bp, synthetic construct
  source 1..30: mol_type = other DNA; note = 5-prime homology arm (30 nt)
tcatcattttggcaaagaattgatttgata

SEQ ID NO: 532 - DNA, 30 bp, synthetic construct
  source 1..30: mol_type = other DNA; note = 3-prime homology arm (30 nt)
attaggtccctcgacctgcagcccaagcta

SEQ ID NO: 533-539 - skipped (7 entries)

SEQ ID NO: 540 - DNA, 80 bp, synthetic construct
  source 1..80: mol_type = other DNA; note = ssODN 80T (5-prime homology arm (40 nt) + 3-prime homology arm (40 nt))
ttgtgctgtctcatcattttggcaaagaattgatttgataattaggtccctcgacctgcagcccaagctagatcgaattc

SEQ ID NO: 541 - DNA, 40 bp, synthetic construct
  source 1..40: mol_type = other DNA; note = 5-prime homology arm (40 nt)
ttgtgctgtctcatcattttggcaaagaattgatttgata

SEQ ID NO: 542 - DNA, 40 bp, synthetic construct
  source 1..40: mol_type = other DNA; note = 3-prime homology arm (40 nt)
attaggtccctcgacctgcagcccaagctagatcgaattc

SEQ ID NO: 543-549 - skipped (7 entries)

SEQ ID NO: 550 - DNA, 100 bp, synthetic construct
  source 1..100: mol_type = other DNA; note = ssODN 100T (5-prime homology arm (50 nt) + 3-prime homology arm (50 nt))
gtgctggttattgtgctgtctcatcattttggcaaagaattgatttgataattaggtccctcgacctgcagcccaagctagatcgaattcggccggcctt

SEQ ID NO: 551 - DNA, 50 bp, synthetic construct
  source 1..50: mol_type = other DNA; note = 5-prime homology arm (50 nt)
gtgctggttattgtgctgtctcatcattttggcaaagaattgatttgata

SEQ ID NO: 552 - DNA, 50 bp, synthetic construct
  source 1..50: mol_type = other DNA; note = 3-prime homology arm (50 nt)
attaggtccctcgacctgcagcccaagctagatcgaattcggccggcctt

SEQ ID NO: 553-559 - skipped (7 entries)

SEQ ID NO: 560 - DNA, 120 bp, synthetic construct
  source 1..120: mol_type = other DNA; note = ssODN 120T (5-prime homology arm (60 nt) + 3-prime homology arm (60 nt))
cctgggcaacgtgctggttattgtgctgtctcatcattttggcaaagaattgatttgataattaggtccctcgacctgcagcccaagctagatcgaattcggccggccttgtacgcgtta

SEQ ID NO: 561 - DNA, 60 bp, synthetic construct
  source 1..60: mol_type = other DNA; note = 5-prime homology arm (60 nt)
cctgggcaacgtgctggttattgtgctgtctcatcattttggcaaagaattgatttgata

SEQ ID NO: 562 - DNA, 60 bp, synthetic construct
  source 1..60: mol_type = other DNA; note = 3-prime homology arm (60 nt)
attaggtccctcgacctgcagcccaagctagatcgaattcggccggccttgtacgcgtta

SEQ ID NO: 563-569 - skipped (7 entries)

SEQ ID NO: 570 - DNA, 140 bp, synthetic construct
  source 1..140: mol_type = other DNA; note = ssODN 140T (5-prime homology arm (70 nt) + 3-prime homology arm (70 nt))
tcctacagctcctgggcaacgtgctggttattgtgctgtctcatcattttggcaaagaattgatttgataattaggtccctcgacctgcagcccaagctagatcgaattcggccggccttgtacgcgttaagtgcaacac

SEQ ID NO: 571 - DNA, 70 bp, synthetic construct
  source 1..70: mol_type = other DNA; note = 5-prime homology arm (70 nt)
tcctacagctcctgggcaacgtgctggttattgtgctgtctcatcattttggcaaagaattgatttgata

SEQ ID NO: 572 - DNA, 70 bp, synthetic construct
  source 1..70: mol_type = other DNA; note = 3-prime homology arm (70 nt)
attaggtccctcgacctgcagcccaagctagatcgaattcggccggccttgtacgcgttaagtgcaacac

SEQ ID NO: 573-579 - skipped (7 entries)

SEQ ID NO: 580 - DNA, 160 bp, synthetic construct
  source 1..160: mol_type = other DNA; note = ssODN 160T (5-prime homology arm (80 nt) + 3-prime homology arm (80 nt))
ttcttctttttcctacagctcctgggcaacgtgctggttattgtgctgtctcatcattttggcaaagaattgatttgataattaggtccctcgacctgcagcccaagctagatcgaattcggccggccttgtacgcgttaagtgcaacacgatcccgcca

SEQ ID NO: 581 - DNA, 80 bp, synthetic construct
  source 1..80: mol_type = other DNA; note = 5-prime homology arm (80 nt)
ttcttctttttcctacagctcctgggcaacgtgctggttattgtgctgtctcatcattttggcaaagaattgatttgata

SEQ ID NO: 582 - DNA, 80 bp, synthetic construct
  source 1..80: mol_type = other DNA; note = 3-prime homology arm (80 nt)
attaggtccctcgacctgcagcccaagctagatcgaattcggccggccttgtacgcgttaagtgcaacacgatcccgcca

SEQ ID NO: 583-599 - skipped (17 entries)

SEQ ID NO: 600 - protein, 1368 aa, synthetic construct [sequence omitted]

SEQ ID NO: 601 - protein, 1393 aa, synthetic construct
  source 1..1393: note = SpCas9 Variant 1 (MA sequence-NSL-SpCas9 WT-SRAD linker-NSL)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 602 - protein, 1393 aa, synthetic construct
  source 1..1393: note = SpCas9 Variant 2 (MA sequence-NSL-SpCas9 WT plus 2 substitutions-SRAD linker-NSL-6His)
MAPKKKRKVDKKYSIGLDIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 603 - protein, 1399 aa, synthetic construct
  source 1..1399: note = SpCas9 Variant 3 (MGSS leader sequence-6His-TEV protease cleavage site (ENLYFQGSM)-SpCas9 WT-GGGS linker-NSL)
MGSSHHHHHHHHENLYFQGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 604 - protein, 1396 aa, synthetic construct
  source 1..1396: note = SpCas9 Variant 4 (MA sequence-6His-linker-NSL-SpCas9 WT-SRAD linker-NSL)
MAHHHHHHGGSPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 605 - protein, 1386 aa, synthetic construct
  source 1..1386: note = SpCas9 Variant 5 (MA sequence-NSL-SpCas9 WT-SRAD linker-6His)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADHHHHHH

SEQ ID NO: 606 - protein, 1389 aa, synthetic construct
  source 1..1389: note = SpCas9 Variant 6 (MA sequence-6His-linker-SpCas9 WT-SRAD linker-NSL)
MAHHHHHHGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 607 - protein, 1399 aa, synthetic construct; identical to SEQ ID NO: 603
  source 1..1399: note = SpCas9 Variant 7 (MGSS leader sequence-6His-TEV protease cleavage site (ENLYFQGSM)-SpCas9 WT-GGGS linker-NSL)

SEQ ID NO: 608 - protein, 1393 aa, synthetic construct
  source 1..1393: note = SpCas9 Variant 8 (MA sequence-NSL-SpCas9 WT plus 1 substitution-SRAD linker-NSL-6His)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 609 - protein, 1393 aa, synthetic construct
  source 1..1393: note = SpCas9 Variant 9 (MA sequence-NSL-SpCas9 WT plus 3 substitutions-SRAD linker-NSL-6His)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEALYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 610 - protein, 1393 aa, synthetic construct
  source 1..1393: note = SpCas9 Variant 10 (MA sequence-NSL-SpCas9 WT plus 3 substitutions-SRAD linker-NSL-6His)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 611 - protein, 1393 aa, synthetic construct
  source 1..1393: note = SpCas9 Variant 11 (MA sequence-NSL-SpCas9 WT plus 4 substitutions-SRAD linker-NSL-6His)
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 612-619 - skipped (8 entries)

SEQ ID NO: 620 - protein, 1367 aa, synthetic construct
  source 1..1367: note = Dead SpCas9 (dCas9) (SpCas9 D10A and H840A)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 621 - protein, 1367 aa, synthetic construct
  source 1..1367: note = SpCas9 nickase (nCas9 or Cas9n) (SpCas9 D10A)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 622-630 - skipped (9 entries)

SEQ ID NO: 631 - protein, 1367 aa, synthetic construct
  source 1..1367: note = VQR-SpCas9 nickase
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 632 - protein, 1367 aa, synthetic construct
  source 1..1367: note = EQR-SpCas9 nickase
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 633 - protein, 1367 aa, synthetic construct
  source 1..1367: note = VRER-SpCas9 nickase
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 634 - protein, 1388 aa, synthetic construct
  source 1..1388: note = CP1028-SpCas9
EIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQ

SEQ ID NO: 635 - protein, 1386 aa, synthetic construct
  source 1..1386: note = CP1041-SpCas9
IMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYS

SEQ ID NO: 636 - protein, 1367 aa, synthetic construct
  source 1..1367: note = SpCas9-NG
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 637-639 - skipped (3 entries)

SEQ ID NO: 640 - protein, 1053 aa, synthetic construct
  source 1..1053: note = SaCas9 nickase
GKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 641 - protein, 1053 aa, synthetic construct
  source 1..1053: note = SaCas9-KKH
GKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 642-649 - skipped (8 entries)

SEQ ID NO: 650 - protein, 1225 aa, synthetic construct
  source 1..1225: note = Catalytically dead LbCas12a (dLbCas12a)
KLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 651 - protein, 1307 aa, synthetic construct
  source 1..1307: note = Engineered AsCas12a (enAsCas12a)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 652-680 - skipped (29 entries)

SEQ ID NO: 681 - protein, 16 aa, synthetic construct
  source 1..16: note = XTEN linker
SGSETPGTSESATPES

SEQ ID NO: 682 - protein, 4 aa, synthetic construct
  source 1..4: note = SGGS linker Unit
SGGS

SEQ ID NO: 683 - protein, 32 aa, synthetic construct
  source 1..32: note = (SGGS)2-XTEN-(SGGS)2 linker
SGGSSGGSSGSETPGTSESATPESSGGSSGGS

SEQ ID NO: 684 - protein, 10 aa, synthetic construct
  source 1..10: note = (SGG)3S linker
SGGSGGSGGS

SEQ ID NO: 685 - protein, 4 aa, synthetic construct
  source 1..4: note = GGGS linker Unit
GGGS

SEQ ID NO: 686 - protein, 5 aa, synthetic construct
  source 1..5: note = GGGGS
GGGGS

SEQ ID NO: 687-690 - skipped (4 entries)

SEQ ID NO: 691 - protein, 7 aa, synthetic construct
  source 1..7: note = NLS1 (SV40 NLS)
PKKKRKV

SEQ ID NO: 692 - protein, 17 aa, synthetic construct
  source 1..17: note = NLS2 (bpNLS)
KRTADGSEFEPKKKRKV

SEQ ID NO: 693 - protein, 19 aa, synthetic construct
  source 1..19: note = NLS3 (M+bpNLS)
MKRTADGSEFESPKKKRKV

SEQ ID NO: 694-699 - skipped (6 entries)

SEQ ID NO: 700 - protein, 1710 aa, synthetic construct
  source 1..1710: note = BE3 (rAPOBEC1-XTEN-Cas9n-SGGS-UGI-SGGS-NLS1)
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 701 - protein, 1710 aa, synthetic construct
  source 1..1710: note = YE1-BE3 (BE3 W90Y+R126E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 702 - protein, 1710 aa, synthetic construct
  source 1..1710: note = YE2-BE3 (BE3 W90Y+R132E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPRNRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 703 - protein, 1710 aa, synthetic construct
  source 1..1710: note = EE-BE3 (BE3 R126E+R132E)
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 704 - protein, 1710 aa, synthetic construct
  source 1..1710: note = YEE-BE3 (BE3 W90Y+R126E+R132E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 705 - protein, 1689 aa, synthetic construct
  source 1..1689: note = CDA1-BE3 (BE3 but rAPOBEC1 is replaced with CDA1; CDA1-XTEN-Cas9n-SGGS-UGI-SGGS-NLS1)
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 706 - protein, 1679 aa, synthetic construct
  source 1..1679: note = AID-BE3 (BE3 but rAPOBEC1 is replaced with AID; AID-XTEN-Cas9n-SGGS-UGI-SGGS-NLS1)
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 707 - protein, 1899 aa, synthetic construct
  source 1..1899: note = BE3-Gam (Gam-XTEN-rAPOBEC1-XTEN-Cas9n-SGGS-UGI-SGGS-NLS1)
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGISGSETPGTSESATPESSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 708-709 - skipped (2 entries)

SEQ ID NO: 710 - protein, 1824 aa, synthetic construct
  source 1..1824: note = BE4 (rAPOBEC1-(SGGS)2XTEN(SGGS)2-Cas9n-(SGG)3S-UGI- (SGG)3S-UGI-SGGS-NLS1)
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 711 - protein, 2013 aa, synthetic construct
  source 1..2013: note = BE4-Gam (Gam-XTEN-rAPOBEC1-(SGGS)2XTEN(SGGS)2-Cas9n -(SGG)3S-UGI-(SGG)3S-UGI-SGGS-NLS1)
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGISGSETPGTSESATPESSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 712 - protein, 1853 aa, synthetic construct
  source 1..1853: note = BE4max (NLS3-rAPOBEC1-(SGGS)2XTEN(SGGS)2-Cas9n-(SGG )3S-UGI-(SGG)3S-UGI-SGGS-NLS2)
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 713 - protein, 1853 aa, synthetic construct
  source 1..1853: note = AncBE4max (NLS3-Anc689APOBEC-(SGGS)2XTEN(SGGS)2-Cas 9n-(SGG)3S-UGI-(SGG)3S-UGI-SGGS-NLS2)
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 714-719 - skipped (6 entries)

SEQ ID NO: 720 - protein, 229 aa, synthetic construct [sequence omitted]

SEQ ID NO: 721 - protein, 229 aa, synthetic construct
  source 1..229: note = YE1-rAPOBEC1 (rAPOBEC1 W90Y+R126E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 722 - protein, 229 aa, synthetic construct
  source 1..229: note = YE2-rAPOBEC1 (rAPOBEC1 W90Y+R132E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPRNRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 723 - protein, 229 aa, synthetic construct
  source 1..229: note = EE-rAPOBEC1 (rAPOBEC1 R126E+R132E)
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 724 - protein, 229 aa, synthetic construct
  source 1..229: note = YEE-rAPOBEC1 (rAPOBEC1 W90Y+R126E+R132E)
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 725 - protein, 208 aa, synthetic construct
  source 1..208: note = CDA1
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 726 - protein, 198 aa, synthetic construct [sequence omitted]

SEQ ID NO: 727-730 - skipped (4 entries)

SEQ ID NO: 731 - protein, 228 aa, synthetic construct
  source 1..228: note = Anc689 APOBEC
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 732 - protein, 228 aa, synthetic construct
  source 1..228: note = Anc687 APOBEC
SSETGPVAVDPTLRRRIEPHEFEVFFDPRELRKEACLLYEIKWGTSHKIWRNSGKNTTKHVEVNFIEKFTSERHFCPSISCSITWFLSWSPCWECSKAIREFLSQHPNVTLVIYVARLFQHMDQQNRQGLRDLVNSGVTIQIMTASEYDHCWRNFVNYPPGKEAHWPRYPPLWMKLYALELHAGILGLPPCLNILRRKQPQLTFFTIALQSCHYQRLPPHILWATGLK

SEQ ID NO: 733-740 - skipped (8 entries)

SEQ ID NO: 741 - protein, 167 aa, synthetic construct
  source 1..167: note = TadA-CDa
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRQVFNSQKKAQSSIN

SEQ ID NO: 742 - protein, 167 aa, synthetic construct
  source 1..167: note = TadA-CDb
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRMPRRVFNSQKKAQSSIN

SEQ ID NO: 743 - protein, 167 aa, synthetic construct
  source 1..167: note = TadA-CDc
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRIPRQVFNSQKKAQSSIN

SEQ ID NO: 744 - protein, 167 aa, synthetic construct
  source 1..167: note = TadA-CDd
MSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 745 - protein, 167 aa, synthetic construct
  source 1..167: note = TadA-CDe
MSEVEFSHEYWMRHALTLAKRARDERAGPVGAVLVLNNRVIGEGWNRAIGLHDPTAHAEIMALRQGGLVMQNHRLIDATLYVTFEPCVMCAGAMINSRIGRVVFGVRNSKRGAAGSLMNVLNYPGMNHRVEITEGILADECAALLCDFYRMPRHVFNSQKKAQSSIN

SEQ ID NO: 746-750 - skipped (5 entries)

SEQ ID NO: 751 - protein, 167 aa, synthetic construct
  source 1..167: note = TadA-CDa-V106W
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGWRNSKRGAAGSLMNVLNYPGMNHRVEITEGILADECAALLCDFYRMPRQVFNSQKKAQSSIN

SEQ ID NO: 752 - protein, 167 aa, synthetic construct
  source 1..167: note = TadA-CDb-V106W
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGWRNSKRGAAGSLMNVLNYPGMNHRVEITEGILADECAALLCDFYRMPRRVFNSQKKAQSSIN

SEQ ID NO: 753 - protein, 167 aa, synthetic construct
  source 1..167: note = TadA-CDc-V106W
MSEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGWRNSKRGAAGSLMNVLNYPGMNHRVEITEGILADECAALLCDFYRIPRQVFNSQKKAQSSIN

SEQ ID NO: 754 - protein, 167 aa, synthetic construct
  source 1..167: note = TadA-CDd-V106W
MSEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGWRNSKRGAAGSLMNVLNYPGMNHRVEITEGILADECAALLCDFYRMPRQVFNAQKKAQSSIN

SEQ ID NO: 755 - protein, 167 aa, synthetic construct
  source 1..167: note = TadA-CDe-V106W
MSEVEFSHEYWMRHALTLAKRARDERAGPVGAVLVLNNRVIGEGWNRAIGLHDPTAHAEIMALRQGGLVMQNHRLIDATLYVTFEPCVMCAGAMINSRIGRVVFGWRNSKRGAAGSLMNVLNYPGMNHRVEITEGILADECAALLCDFYRMPRHVFNSQKKAQSSIN

SEQ ID NO: 756-759 - skipped (4 entries)

SEQ ID NO: 760 - protein, 83 aa, synthetic construct
  source 1..83: note = Uracil DNA glycosylase (UDG) inhibitor (UGI)
TNLSDIIEKETGKQLVIQESILMLPEEVEEVIGNKPESDILVHTAYDESTDENVMLLTSDAPEYKPWALVIQDSNGENKIKML

SEQ ID NO: 761 - protein, 174 aa, synthetic construct
  source 1..174: note = Gam (bacteriophage Mu protein)
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGI

SEQ ID NO: 762-799 - skipped (38 entries)

SEQ ID NO: 800 - protein, 1775 aa, synthetic construct
  source 1..1775: note = ABE7.10 (ecTadA-(SGGS)2XTEN(SGGS)2-ecTadA 7.10-(SGGS)2XTEN(SGGS)2- Cas9n-SGGS-NLS1)
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 801 - protein, 1800 aa, synthetic construct
  source 1..1800: note = ABEmax (NLS3-ecTadA-(SGGS)2XTEN(SGGS)2-ecTadA 7.10-(SGGS)2XTEN(SGGS)2-Cas9n-NLS2)
MKRTADGSEFESPKKKRKVMSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRTADGSEFEPKKKRKV

SEQ ID NO: 802-809 - skipped (8 entries)

SEQ ID NO: 810 - protein, 1605 aa, synthetic construct
  source 1..1605: note = ABE8e (NLS3-ecTadA 8e-(SGGS)2XTEN(SGGS)2- Cas9n-SGGS-NLS2)
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 811 - protein, 1803 aa, synthetic construct
  source 1..1803: note = ABE8e dimer (NLS3-ecTadA-(SGGS)2XTEN(SGGS)2-ecTadA 8e-(SGGS)2XTEN(SGGS)2-Cas9n-SGGS-NLS2)
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 812-819 - skipped (8 entries)

SEQ ID NO: 820 - protein, 167 aa, synthetic construct
  source 1..167: note = Wild-type E. coli TadA (ecTadA)
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 821 - protein, 166 aa, synthetic construct
  source 1..166: note = ecTadA 6.3 (ecTadA containing A106V, D108N, D147Y, and E155V; L84F, H123Y, and I157F; H36L, R51L, S146C, and K157N; and P48S)
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 822 - protein, 166 aa, synthetic construct
  source 1..166: note = ecTadA 6.4 (ecTadA containing A106V, D108N, D147Y, and E155V; L84F, H123Y, and I157F; H36L, R51L, S146C, and K157N; and P48S and A142N)
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 823 - protein, 166 aa, synthetic construct
  source 1..166: note = ecTadA 7.8 (ecTadA containing A106V, D108N, D147Y, and E155V; L84F, H123Y, and I157F; H36L, R51L, S146C, and K157N; A142N; and W23L and P48A)
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 824 - protein, 166 aa, synthetic construct
  source 1..166: note = ecTadA 7.9 (ecTadA containing A106V, D108N, D147Y, and E155V; L84F, H123Y, and I157F; H36L, R51L, S146C, and K157N; A142N; and W23L, P48A, and R152P)
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 825 - protein, 166 aa, synthetic construct
  source 1..166: note = ecTadA 7.10 (ecTadA containing A106V, D108N, D147Y, and E155V; L84F, H123Y, and I157F; H36L, R51L, S146C, and K157N; and W23R, P48A, and R152P)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 826 - protein, 166 aa, synthetic construct
  source 1..166: note = ecTadA 8e (ecTadA 7.10 further containing A109S, T111R, D119N, H122N, Y147D, F149Y, T166I, and D167N)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 827 - protein, 166 aa, synthetic construct
  source 1..166: note = ecTadA 8e-V106W
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 828 - protein, 166 aa, synthetic construct
  source 1..166: note = ecTadA 8e-V82G
SEVEFSHEYWMRHALTLAKRARDEREVPVGAVLVLNNRVIGEGWNRAIGLHDPTAHAEIMALRQGGLVMQNYRLIDATLYGTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 829 - protein, 166 aa, synthetic construct
  source 1..166: note = ecTadA 8e-K20A-R21A
SEVEFSHEYWMRHALTLAAAARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 830-899 - skipped (70 entries)

SEQ ID NO: 900 - protein, 1791 aa, synthetic construct
  source 1..1791: note = TadDE (NLS3-TadA Dual-(SGGS)2XTEN(SGGS)2-Cas9n-(SGG )3S-UGI-(SGG)3S-UGI-SGGS-NLS2)
MKRTADGSEFESPKKKRKVSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 901 - protein, 1791 aa, synthetic construct
  source 1..1791: note = TadDE-V106W (NLS3-TadA DualV106W-(SGGS)2XTEN(SGGS)2 -Cas9n-(SGG)3S-UGI-(SGG)3S-UGI-SGGS-NLS2)
MKRTADGSEFESPKKKRKVSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 902-919 - skipped (18 entries)

SEQ ID NO: 920 - protein, 167 aa, synthetic construct
  source 1..167: note = TadA Dual
MSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 921 - protein, 167 aa, synthetic construct
  source 1..167: note = TadA Dual-V106W
MSEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGWRNSKRGAAGSLMNVLNYPGMNHRVEITEGILADECAALLCDFYRMPRQVFNAQKKAQSSIN

(19) United States
(12) Patent Application Publication
(10) Pub. No.: US 2025/0235559 A1
(43) Pub. Date: Jul. 24, 2025

(54) GENE EDITING REPORTER SYSTEM AND GUIDE RNA AND COMPOSITION RELATED THERETO; COMPOSITION AND METHOD FOR KNOCKING OUT DNA WITH MORE THAN TWO GRNAS; GENE EDITING IN THE EYE; AND GENE EDITING USING BASE EDITORS

(71) Applicants: Incisive Genetics Inc., Vancouver (CA); The University of British Columbia, Vancouver (CA)

(72) Inventors: Blair LEAVITT, Vancouver (CA); Austin HILL, Vancouver (CA); Pamela WAGNER, Vancouver (CA); Elizabeth SIMPSON, Vancouver (CA); Zeinab MOHANNA, Vancouver (CA); Bethany ADAIR, Vancouver (CA)

(21) Appl. No.: 18/840,939

(22) PCT Filed: Feb. 24, 2023
(86) PCT No.: PCT/IB2023/051742

§ 371 Date: Aug. 23, 2024

Related U.S. Application Data
(60) Provisional application No. 63/314,044, filed on Feb. 25, 2022.

(51) Int. Cl. A61K 48/00; C12N 9/22; C12N 15/11; C12N 15/90

(52) U.S. Cl. CPC A61K 48/0058; C12N 9/226; C12N 15/11; C12N 15/907; C12N 2310/20

(57) ABSTRACT

The present disclosure provides: reporter systems for CRISPR-mediated gene editing; gRNAs, RNPs, and compositions for the reporter systems; and methods of testing CRISPR-mediated gene editing. The present disclosure further provides compositions and methods for knocking out a DNA segment of interest; and methods of effecting CRISPR-mediated gene editing in the eye. The present disclosure also provides compositions and methods for effecting base editing using base editors.

### RELATED APPLICATIONS

This application is a U.S. National Phase application of Int'l Appl. No. PCT/IB2023/051742, filed Feb. 24, 2023, which claims priority to U.S. Provisional Appl. No. 63/314,044, filed Feb. 25, 2022, each of which are incorporated herein by reference in their entireties.

### SEQUENCE LISTING DISCLOSURE

The contents of the electronic sequence listing (29574150001801.xml; size: 465,987 bytes; and Date of Creation: Aug. 15, 2024) is herein incorporated by reference in its entirety.

### BACKGROUND OF THE INVENTION

Currently, a substantial amount of research utilizes CRISPR-mediated gene editing as a tool and/or aim to develop CRISPR/Cas-mediated gene therapy. Several in vitro and in vivo gene editing reporter systems have been developed for use at a variety of stages in such research. In particular gene editing has been effected using mTomato-derived sequence-containing cassettes (see e.g., Yang et al., Mol Ther Nucleic Acids. 2017 Jun. 16; 7:378-386; Tabebordbar et al., Science. 2016 Jan. 22;351 (6271): 407-411; Staahl et al., Nat Biotechnol. 2017 May; 35 (5): 431-434; Wei et al., Nat Commun. 2020 Jun. 26;11 (1): 3232; Knudsen et al., Front Cell Dev Biol. 2018 May 15; 6:54; Miura et al., Mol Ther Nucleic Acids. 2021 Mar. 5; 24:325-336; and de Jong et al., Nat Commun. 2020 Mar. 31;11 (1): 1701). However, the use of such mTomato-containing cassettes has had some shortcomings, e.g., insufficient sensitivity, high off target effects, and/or poor reporter gene expression in a particular tissue. Accordingly, there is a need for improved reporter systems.

### SUMMARY OF THE INVENTION

Some aspects of the present disclosure relate to reporter systems for CRISPR-mediated gene editing.

In one aspect, the present disclosure provides gRNAs for CRISPR-mediated gene editing, and a gRNA may comprise a crRNA sequence comprising a targeting sequence. Such a gRNA may be used as part of and/or in relation to a reporter system according to the present disclosure.

In some embodiments, a targeting sequence may comprise at least or consist of 17 nucleic acids contained in SEQ ID NO: 80 or 81. In some embodiments, a targeting sequence may comprise at least or consist of 18 nucleic acids. In some embodiments, a targeting sequence may comprise at least or consist of 19 nucleic acids. In some embodiments, a targeting sequence may comprise at least or consist of 20 nucleic acids. In some embodiments, a targeting sequence may comprise at least or consist of 21 nucleic acids. In some embodiments, a targeting sequence may comprise at least or consist of 22 nucleic acids. In some embodiments, a targeting sequence may comprise at least or consist of 23 nucleic acids. In some embodiments, a targeting sequence may comprise at least or consist of 24 nucleic acids. In some embodiments, a targeting sequence may comprise at least or consist of 25 nucleic acids. In some embodiments, a targeting sequence may comprise at least or consist of 26 nucleic acids. In some embodiments, a targeting sequence may comprise at least or consist of 27 nucleic acids. In some embodiments, a targeting sequence may comprise at least or consist of 28 nucleic acids. In some embodiments, a targeting sequence may comprise at least or consist of 29 nucleic acids. In some embodiments, a targeting sequence may comprise at least or consist of 30 nucleic acids.

In some embodiments, such a targeting sequence may comprise a sequence of at least 17 consecutive nucleic acids contained in SEQ ID NO: 80 or 81. In some embodiments, such a targeting sequence may comprise a sequence of at least 18 consequent nucleic acids, 19 consequent nucleic acids, or 20 consequent nucleic acids consequent nucleic acids, contained in SEQ ID NO: 80 or 81. In some embodiments, such a targeting sequence may comprise a sequence of at least 21, 22, 23, 24, 25, 26, 27, 28, 29, or 30 consequent nucleic acids, contained in SEQ ID NO: 80 or 81.

In some embodiments, a targeting sequence may comprise a sequence of at least 17 nucleic acids comprising one or more mutations (such as one, two, three, four, or five mutations). Such one or more mutations may be relative to the sequence of at least 17 consecutive nucleic acids (such as 17, 18, 19, 20, 21, 22, 23, 24, 25, 26, 27, 28, 29, or 30 consequent nucleic acids) contained in SEQ ID NO: 80 or 81. In certain embodiments, such one or more mutations may be at any nucleic acid position(s) of the at least 17 consecutive nucleic acids. In certain embodiments, such one or more mutations may be at position(s) other than the 4th to the 7th nucleic acid positions from the 3′-end of the at least 17 consecutive nucleic acids.

In some embodiments, a targeting sequence may comprise a sequence of at least 17 nucleic acids which comprises at least 85%, at least 86%, at least 87%, at least 88%, at least 89% at least 90%, at least 91%, at least 92%, at least 93%, at least 94%, at least 95%, at least 96%, at least 97%, at least 98%, or at least 99% sequence identity to the sequence of at least 17 consecutive nucleic acids (e.g., 17, 18, 19, 20, 21, 22, 23, 24, 25, 26, 27, 28, 29, or 30 consequent nucleic acids) contained in SEQ ID NO: 80 or 81.

In some embodiments, the at least 17 consecutive nucleic acids may be immediately upstream of or adjacent to a protospacer adjacent motif (PAM) or protospacer flanking site (PFS) of a CRISPR-associated (Cas) endonuclease in SEQ ID NO: 80 or 81. In certain embodiments, the Cas endonuclease may be Cas9. In certain embodiments, the Cas endonuclease may be Streptococcus pyogenes Cas9 (SpCas9). In certain embodiments, the PAM sequence may be 5′-NGG-3′, wherein N represents any nucleotide.

In some embodiments, the at least 17 consecutive nucleic acids may be immediately downstream of or adjacent to the PAM or PFS of a Cas endonuclease in SEQ ID NO: 80 or 81. In certain embodiments, the Cas endonuclease may be Cpf1. In certain embodiments, the PAM sequence may be 5′-TTTN-3′ wherein N represents any nucleotide.

In some embodiments, the sequence of the at least 17 consecutive nucleic acids may comprise or consist of SEQ ID NO: 120, 121, 122, or 123. In some embodiments, the sequence of the at least 17 consecutive nucleic acids may comprise or consist of SEQ ID NO: 130, 131, 132, or 133.

In some embodiments, the targeting sequence may be 17, 18, 19, 20, 21, 22, 23, 24, 25, 26, 27, 28, 29, or 30 nucleotides in length.

In some embodiments, the gRNA may be a single guide RNA (sgRNA). In some embodiments, such a sgRNA may comprise (i) a crRNA sequence comprising the targeting sequence and a crRNA backbone sequence and (ii) a trans-activating CRISPR RNA (tracrRNA) sequence in a single strand. In certain embodiments, the crRNA sequence and the tracrRNA sequence may be linked via a linker. In particular embodiments, the linker may comprise the nucleic acid sequence of GAAA. In particular embodiments, the gRNA may comprise the targeting sequence followed by a sgRNA backbone sequence. In particular embodiments, the sgRNA backbone sequence may comprise or consist of any of SEQ ID NOS: 111-114. In particular embodiments, the sgRNA backbone sequence may comprise any of SEQ ID NOS: 111-114 followed by one or more uracils, such as 1-10 uracils. In particular embodiments, the one or more uracils may be four consecutive uracils (i.e., 5′-UUUU-3′).

In some embodiments, the gRNA may be a dual guide RNA (dgRNA). In some embodiments, such a dgRNA may be formed by hybridization between (i) a crRNA sequence comprising the targeting sequence and a crRNA backbone sequence and (ii) a tracrRNA. In certain embodiments, the crRNA backbone sequence and the tracrRNA may comprise SEQ ID NOS: 115 and 116, respectively. In certain embodiments, the crRNA backbone sequence and the tracrRNA may comprise SEQ ID NOS: 117 and 118, respectively.

In particular embodiments, the sequence of the gRNA may comprise or consist of any of SEQ ID NOS: 125-128, 225-228, 325-328, and 425-428. In particular embodiments, the sequence of the gRNA may comprise or consist of any of SEQ ID NOS: 135-138, 235-238, 335-338, and 435-438. In certain embodiments, such gRNA may comprise one or more uracils at the 3′ end, such as 1-10 uracils. In particular embodiments, the one or more uracils may be four consecutive uracils (i.e., 5′-UUUU-3′).

In some embodiments, a gRNA as described herein may be synthetic or recombinant.

In some embodiments, a gRNA as described herein may comprise at least one modification, such as a chemical modification, which may be any of the RNA modifications described herein. In particular embodiments, the at least one modification may be (i) 2′-O-methylation, which is optionally at first three and last three bases and/or (ii) one or more 3′ phosphorothioate bonds, optionally between first three and last two bases.

In one aspect, the present disclosure provides polynucleotides encoding one or more gRNAs according to the present disclosure.

In some embodiments, such a polynucleotide may be a DNA. In some embodiments, such a polynucleotide may be a RNA.

In one aspect, the present disclosure provides vectors comprising a polynucleotide encoding one or more gRNAs according to the present disclosure.

In some embodiments, such a vector may comprise a polynucleotide encoding one or more gRNAs according to the present disclosure, which optionally may be operably linked to one or more regulatory sequences such as but not limited to a promoter and/or terminator.

In some embodiments, such a vector may be any of the vectors described herein, including but not limited to plasmids and viral vectors such as adenoviral, adeno-associated virus, lentivirus, and/or retrovirus vectors.

In one aspect, the present disclosure provides RNPs comprising a gRNA according to the present disclosure and a Cas endonuclease. Such a RNP may be used as part of and/or in relation to a reporter system according to the present disclosure.

In some embodiments, a RNP may comprise any of the isolated gRNAs described above complexed with a Cas endonuclease.

In some embodiments, the Cas endonuclease may be selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2. In some embodiments, a Cas endonuclease may be a class 2 Cas endonuclease. In some embodiments, a Cas endonuclease may be a type II, type V, or type VI Cas nuclease. In some embodiments, a Cas endonuclease may be Cas9 of Streptococcus pyogenes (SpCas9), Staphylococcus aureus (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of SpCas9 (e.g., CP1012-SpCas9, CP1028-SpCas9, CP1041-SpCaS9, CP1249-SpCas9, and CP1300-SpCas9; see e.g., Oakes et al., Cell. 2019 Jan. 10;176 (1-2): 254-267.e16), eSpCas9, HypaCas9, FokI-Fused dCas9, xCas9, SpRY (variant of SpCas9), and SpG (variant of SpCas9). In particular embodiments, a Cas endonuclease may be a WT SpCas9, optionally comprising SEQ ID NO: 600. In particular embodiments, a Cas endonuclease may be a variant SpCas9, optionally comprising any one of SEQ ID NOS: 601-611. In some embodiments, a Cas endonuclease may be Cas12a of Lachnospiraceae bacterium ND2006 (LbCas12a), Cas12a of Acidaminococcus sp. BV3L6 (AsCas12a), or Cas12a of Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a.

In some embodiments, a RNP may be formed by mixing a solution comprising the gRNA and a solution comprising the Cas endonuclease at an approximately equimolar ratio. In particular embodiments, the mixing may be for about 5 minutes. In particular embodiments, the solution comprising the gRNA may have a pH of about 6 to 8, about 6.5 to 7.5, or about 7. In particular embodiments, the solution comprising the Cas endonuclease may have a pH of about 6 to 8, about 6.5 to 7.5, or about 7. In particular embodiments, the resulting RNP solution may have a pH of about 6 to 8, about 6.5 to 7.5, or about 7.

In one aspect, the present disclosure provides compositions. Such compositions may be for CRISPR-mediated gene editing and may be used as part of and/or in relation to a reporter system according to the present disclosure.

In some embodiments, a composition may comprise (A) a pharmaceutically acceptable carrier; and (B) one or more RNPs as described above; and (C) optionally a template DNA.

In certain embodiments, the one or more RNPs may comprise (I) a first RNP comprising a first isolated gRNA and a first Cas endonuclease and/or (II) a second RNA comprising a second isolated gRNA and a second Cas endonuclease.

In certain embodiments, the first isolated gRNA may comprise a first crRNA sequence comprising a first targeting sequence. In particular embodiments, the first targeting sequence may comprise or consists of SEQ ID NO: 120, 121, 122, or 123. In particular embodiments, the first targeting sequence may comprise or consists of a sequence of at least 17 nucleic acids (which may comprise or consist of a sequence of at least 17 nucleic acids) comprising one or more mutations (e.g., one, two, three, four, or five mutations) relative to SEQ ID NO: 120, 121, 122, or 123. In particular embodiments, the one or more mutations may be at any nucleic acid position(s) or are at position(s) other than the 4th to the 7th nucleic acid positions from the 3′-end of SEQ ID NO: 120, 121, 122, or 123. In yet particular embodiments, the sequence of the first isolated gRNA may comprise or consist of any of SEQ ID NOS: 125-128, 225-228, 325-328 and 425-428. Such a gRNA may comprise multiple uracils at the 3′ end, such as 1-10 uracils, e.g., 5′-UUUU-3′.

In certain embodiments, the second isolated gRNA may comprise a second crRNA sequence comprising a second targeting sequence. In particular embodiments, the second targeting sequence may comprise or consists of SEQ ID NO: 130, 131, 132, or 133. In particular embodiments, the second targeting sequence may comprise or consists of a sequence of at least 17 nucleic acids (which may comprise or consist of a sequence of at least 17 nucleic acids) comprising one or more mutations (e.g., one, two, three, four, or five mutations) relative to SEQ ID NO: 130, 131, 132, or 133. In particular embodiments, the one or more mutations may be at any nucleic acid position(s) or are at position(s) other than the 4th to the 7th nucleic acid positions from the 3′-end of SEQ ID NO: 130, 131, 132, or 133. In yet particular embodiments, the sequence of the second isolated gRNA may comprise or consist of any of SEQ ID NOS: 135-138, 235-238, 335-338, and 435-438. Such a gRNA may comprise multiple uracils at the 3′ end, such as 1-10 uracils, e.g., 5′-UUUU-3′.

In some embodiments, a composition may further comprise a third RNP and/or a fourth RNP.

In certain embodiments, the third RNPs may comprise a third isolated gRNA and a third Cas endonuclease, wherein the third isolated gRNA comprises a third crRNA sequence comprising a third targeting sequence. In particular embodiments, the third targeting sequence may comprise or consists of SEQ ID NO: 140, 141, 142, or 143. In particular embodiments, the third targeting sequence may comprise or consists of a sequence of at least 17 nucleic acids (which may comprise or consist of a sequence of at least 17 nucleic acids) comprising one or more mutations (e.g., one, two, three, four, or five mutations) relative to SEQ ID NO: 140, 141, 142, or 143. In particular embodiments, the one or more mutations may be at any nucleic acid position(s) or are at position(s) other than the 4th to the 7th nucleic acid positions from the 3′-end of SEQ ID NO: 140, 141, 142, or 143. In yet particular embodiments, the sequence of the second isolated gRNA may comprise or consist of any of SEQ ID NOS: 145-148, 245-248, 345-348, and 445-448. Such a gRNA may comprise multiple uracils at the 3′ end, such as 1-10 uracils, e.g., 5′-UUUU-3′.

In certain embodiments, the fourth RNPs may comprise a fourth isolated gRNA and a fourth Cas endonuclease, wherein the fourth isolated gRNA comprises a fourth crRNA sequence comprising a fourth targeting sequence. In particular embodiments, the fourth targeting sequence may comprise or consists of SEQ ID NO: 150, 151, 152, or 153. In particular embodiments, the fourth targeting sequence may comprise or consists of a sequence of at least 17 nucleic acids (which may comprise or consist of a sequence of at least 17 nucleic acids) comprising one or more mutations (e.g., one, two, three, four, or five mutations) relative to SEQ ID NO: 150, 151, 152, or 153. In particular embodiments, the one or more mutations may be at any nucleic acid position(s) or are at position(s) other than the 4th to the 7th nucleic acid positions from the 3′-end of SEQ ID NO: 150, 151, 152, or 153. In yet particular embodiments, the sequence of the fourth isolated gRNA may comprise or consist of any of SEQ ID NOS: 155-158, 255-258, 355-358, and 455-458. Such a gRNA may comprise multiple uracils at the 3′ end, such as 1-10 uracils, e.g., 5′-UUUU-3′.

In certain embodiments, the targeting sequence of the first, second, third, and/or fourth gRNA is 17, 18, 19, 20, 21, 22, 23, 24, 25, 26, 27, 28, 29, or 30 nucleotides in length.

In certain embodiments, the first, second, third, and/or fourth gRNA may be a sgRNA. Such a sgRNA may comprise (i) a crRNA sequence comprising the targeting sequence as described above and a crRNA backbone sequence and (ii) a tracrRNA sequence in a single strand. In some cases, the crRNA sequence and the tracrRNA sequence are linked via a linker, which optionally may comprise the nucleic acid sequence of GAAA. In particular embodiments, the gRNA may comprise the targeting sequence followed by a sgRNA backbone sequence of any of SEQ ID NOS: 111-114. In yet particular embodiments, the sgRNA backbone sequence may be followed by one or more uracils, such as 1-10 uracils.

In certain embodiments, the first, second, third, and/or fourth gRNA may be a dgRNA. Such a dgRNA may be formed by hybridization between (i) a crRNA comprising the targeting sequence as described above and a crRNA backbone sequence and (ii) a tracrRNA. In yet particular embodiments, the crRNA backbone sequence and the tracrRNA comprise SEQ ID NOS: 115 and 116, respectively. In yet particular embodiments, the crRNA backbone sequence and the tracrRNA comprise SEQ ID NOS: 117 and 118.

In certain embodiments, the first, second, third, and/or fourth gRNA may be synthetic or recombinant. In certain embodiments, the first, second, third, and/or fourth gRNA may be a synthetic sgRNA and may comprise at least one chemical modification, such as any of the RNA modifications described herein. In particular embodiments, the first, second, third, and/or fourth gRNA may comprise 2′-O-methylation optionally at first three and last three bases and/or one or more 3′ phosphorothioate bonds, optionally between first three and last two bases.

In certain embodiments, the pharmaceutically acceptable carrier may comprise a lipid-based transfection competent vesicle (TCV), which may be any of the lipid-based TCVs described herein, In particular embodiments, such a lipid-based TCV may comprise at least one ionizable cationic lipid such as DODOMA. In particular embodiments, such a lipid-based TCV may comprise DODMA, DOPE, DSPC, and cholesterol, optionally at an about 20:30:10:40 molar ratio. In particular embodiments, such a lipid-based TCV may comprise DODMA, DOPE, DSPC, cholesterol, and PEG-lipid (such as PEG-DMG), optionally at an about 20:30:10:39:1 molar ratio.

In certain embodiments, the template DNA, if present, and/or the one or more RNPs may be encapsulated in the TCV. In particular embodiments, if a template DNA is present, the template DNA and the one or more RNPs may be co-encapsulated in the TCV. In yet particular embodiments, if a template DNA is present, the template DNA and the one or more RNPs may be separately encapsulated in the TCV, i.e., the template DNA and the RNPs do not exist within a same TCV.

In certain embodiments, the encapsulation may be achieved by (i) providing an aqueous solution comprising the TCV and (ii) mixing the template DNA, if present, and/or one or more of the one or more RNPs with the aqueous solution.

In particular embodiments, the aqueous solution may have the pH of about 3 to about 8, about 4 to about 7.5, 3.5 to 4.5, or about 4. In particular embodiments, the aqueous solution comprises an acetate buffer. In particular embodiments, the aqueous solution may be substantially, essentially, or entirely free of ethanol, methanol, isopropanol, tetrahydrofuran (THF), dimethyl sulfoxide (DMSO), dimethyl formamide (DMF), and acetonitrile (ACN), and/or substantially, essentially, or entirely free of sodium dodecyl sulfate (SDS). In yet particular embodiments, the aqueous solution may be substantially, essentially, or entirely free of organic solvents and detergents. In yet particular embodiments, the aqueous solution may be substantially, essentially, or entirely free of destabilizing agents.

In particular embodiments, the mixing may comprise gentle mixing (optionally repeated manual reciprocation of the TCV-generating fluid in a pipette), micromixing, mixing using a staggered herringbone micromixer (SHM), T-junction mixing, or extrusion. In particular embodiments, the mixing time may be about 0.1 second to about 20 minutes. In particular embodiments, the mixing may be performed substantially, essentially, or entirely free of ethanol, methanol, isopropanol, tetrahydrofuran (THF), dimethyl sulfoxide (DMSO), dimethyl formamide (DMF), and acetonitrile (ACN), and/or substantially, essentially, or entirely free of sodium dodecyl sulfate (SDS). In particular embodiments, the mixing may be performed substantially, essentially, or entirely free of organic solvents and/or detergents. In particular embodiments, the mixing may be performed substantially, essentially, or entirely free of destabilizing agents.

In particular embodiments, when more than one RNPs are encapsulated in the TCV, the mixing may comprise mixing an equimolar ratio of the more than one RNPs with the aqueous solution.

In particular embodiments, when a template DNA is present, the template DNA may be co-encapsulated with or separately encapsulated from one or more of the one or more RNPs.

In some embodiments, a composition may comprise (A) a pharmaceutically acceptable carrier; and (B) (a) one or more isolated gRNAs as described above or one or more polynucleotides encoding such one or more isolated gRNAs, and (b) a Cas endonuclease or a polynucleotide encoding a Cas endonuclease; and (C) optionally a template DNA or a polynucleotide encoding a template DNA,

In certain embodiments, the one or more isolated gRNAs may comprise a first isolated gRNA and/or a second isolated gRNA. In particular embodiments, the first isolated gRNA may be any of the first isolated gRNAs as described above. In particular embodiments, the second isolated gRNA may be any of the second isolated gRNAs as described above. In certain embodiments, the one or more isolated gRNAs may further comprise a third isolated gRNA and/or a fourth isolated gRNA. In particular embodiments, the third isolated gRNA may be any of the third isolated gRNAs as described above. In particular embodiments, the fourth isolated gRNA may be any of the fourth isolated gRNAs as described above. In certain embodiments, the Cas endonuclease may any of those described above.

In some cases, the components in (B) of the composition may be (a) said one or more isolated gRNAs, and (b) a vector comprising said polynucleotide encoding a Cas endonuclease. In some cases, the components in (B) of the composition may be (a) one or more vectors comprising said one or more polynucleotides encoding the one or more isolated gRNAs, and (b) said Cas endonuclease. In some cases, the components in (B) of the composition may be (a) one or more vectors comprising said one or more polynucleotides encoding the one or more isolated gRNAs, and (b) a vector comprising said polynucleotide encoding a Cas endonuclease. In some cases, the components in (B) of the composition may be (a) said one or more isolated gRNAs, and (b) said Cas endonuclease.

In certain cases, said vector or said one or more vectors may be individually selected from plasmids, RNA replicons, virus-like particles (VLPs), and viral vectors, optionally retroviral, lentiviral, or adenoviral vectors. In certain cases, when said one or more isolated gRNAs are more than one isolated gRNAs, said more than one gRNAs may be encoded by one or more nucleic acids comprised in a single vector or comprised in separate vectors. In certain cases, said one or more isolated gRNAs and said Cas endonuclease may be encoded by one or more nucleic acids comprised in a single vector or comprised in separate vectors.

As noted above, any of the compositions described above may comprise a template DNA. In some embodiments, the template DNA may comprise a single-strand oligo DNA nucleotide molecule (ssODN) comprising or consisting of a 5′ homology arm, an optional central region (e.g., 0-100 nt in length), and a 3′ homology arm.

In certain embodiments, said 5′ homology arm may comprise or consist of a sequence corresponding to the first nucleotide to at least the 10th nucleotide counting from the 3′-end of SEQ ID NO: 581 or a sequence selected from any of SEQ ID NOS: 511, 521, 531, 541, 551, 561, 571, and 581. In further embodiments, the 5′ homology arm may comprise or consist of a sequence comprising at least one (such as one, two, three, four, five, six, seven, eight, nine, or ten) silent mutation(s) relative to any of such 5′ homology arm sequences.

In certain embodiments, said optional central region may be 1-100 nucleotides (nt) in length.

In certain embodiments, said 3′ homology arm may comprise or consist of a sequence corresponding to the first nucleotide to at least the 10th nucleotide counting from the 5′-end of SEQ ID NO: 582 or a sequence selected from any of SEQ ID NOS: 512, 522, 532, 542, 552, 562, 572, and 582. In further embodiments, the 3′ homology arm may comprise or consist of a sequence comprising at least one (such as one, two, three, four, five, six, seven, eight, nine, or ten) silent mutation(s) relative to any of such 3′ homology arm sequences.

In particular embodiments, the ssODN may comprises or consists of a sequence selected from any of SEQ ID NOs: 510, 520, 530, 540, 550, 560, 570, and 580.

In particular embodiments, the ssODN may be fully complementary to the sequence any of the ssODNs described above.

In some embodiments, the template DNA may comprise a double-strand DNA molecule, which comprises a first strand comprising or consisting of any of the ssODN sequences described above and a second strand complementary thereto.

Any of the compositions described above may comprise a TCV such as a lipid-based TCV.

In some embodiments, said TCV may comprise at least one cationic or ionizable cationic lipid. In certain embodiments, said at least one cationic or ionizable cationic lipid may comprise, essentially consist of, or consist of a lipid selected from the group consisting of N,N-dimethyl-2,3-dioleyloxy) propylamine (DODMA), 1,2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino) butanoate (MC3), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxyl) propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAR.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl) propyl-N,N-N-triethylammonium chloride (“DOTMA”); 1,2-Dioleyloxy-3-trimethylaminopropane chloride salt (“DOTAP.C1”); 3.beta.-(N-(N′,N′-dimethylaminoethane)-carbamoyl) cholesterol (“DC-Chol”), N-(1-(2,3-dioleyloxyl) propyl)-N-2-(sperminecarboxamido)ethyl)-N,N-dimethyl-ammonium trifluoracetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof.

In particular embodiments, the TCV may comprise at least one ionizable cationic lipid. In particular embodiments, the at least one ionizable cationic lipid may comprise DODMA.

In some embodiments, said TCV may further comprise at least one helper lipid. In certain embodiments, the at least one helper lipid may comprise, essentially consist of, or consist of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof. In particular embodiments, the at least one helper lipid may comprise DOPE and/or DSPC.

In some embodiments, said TCV may further comprise at least one phospholipid, optionally wherein the at least one phospholipid may comprise, essentially consist of, or consist of a lipid selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof. In certain embodiments, the at least one phospholipid may comprise DSPC and/or DOPE.

In some embodiments, said TCV may further comprise at least one cholesterol or cholesterol derivative, optionally wherein the at least one cholesterol or cholesterol derivative may comprise, essentially consist of, or consist of a cholesterol or cholesterol derivative selected from the group consisting of cholesterol, N,N-dimethyl-N-ethylcarboxamidocholesterol (DC-Chol), 1,4-bis(3-N-oleylamino-propyl) piperazine, imidazole cholesterol ester (ICE), and any combinations thereof. In certain embodiments, the at least one cholesterol or cholesterol derivative may comprise cholesterol.

In some embodiments, said TCV may further comprise at least one PEG or PEG-lipid, optionally wherein the at least one PEG-lipid may comprise, essentially consist of, or consist of a PEG-lipid selected from the group consisting of PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combinations thereof. In certain embodiments, the at least one PEG or PEG-lipid may comprise PEG-DMG. In certain embodiments, the total amount of said at least one PEG or PEG-lipid in said TCV may be at most 2 mol %, 1.5 mol %, 1.0 mol %, 0.5 mol %, or 0.1 to 0.5 mol %. In certain embodiments, said TCV further does not comprise any PEG or PEG-lipid.

In certain embodiments, said TCV may be substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; and further optionally substantially, essentially, or entirely free of destabilizing agents. In certain embodiments, said TCV may be substantially, essentially, or entirely free of any permanently cationic lipids and/or any permanently anionic lipids.

The TCV in any of the compositions according to the present invention may comprise any of and any combinations of the features of a TCV described above.

In some embodiments, the TCV may be formed by: (a) providing a first solution comprising all components of the TCV, optionally at about 20-35 mM, in ethanol; (b) providing a second solution, which is aqueous and may contain acetate and/or citrate, optionally sodium acetate and/or sodium citrate, optionally at about 25 mM, optionally wherein the pH of the solution may be about 3.5 to 4.5 or is about 4; (c) combining the first and second solutions by gentle mixing, optionally by one or more of repeated manual reciprocation of the TCV-generating fluid in a pipette, micromixing, mixing using a staggered herringbone micromixer (SHM), T-junction mixing, or extrusion; and (d) removing ethanol, optionally by dialysis or evaporation.

In certain embodiments, the size of said TCV before encapsulation may range from about 9 nm to about 80 nm, optionally about 10-40 nm, further optionally about 20-35 nm at pH of about 3.5 to 4.5 or at pH of about 4. In certain embodiments, the size of said TCV after encapsulation of the at least one cargo may range from about 80 nm to about 1500 nm. In particular embodiments, the TCV after encapsulation at pH of about 3-5 (such as about 3.5-4.5 or about 4) may be in a range of about 800 nm to about 1400 or about 1000 nm to about 1200 nm. In alternative embodiments, said TCV after encapsulation at pH of about 3-5 (such as about 3.5-4.5 or about 4) may range from about 80 nm to about 300 nm or about 100 nm to about 250 nm. In certain embodiments, said TCV after encapsulation may be further comprised in a matrix vesicle, which may optionally be for gradual release of the TCV. In certain embodiments, the final ethanol concentration of the composition may be 5% (v/v) or below, preferably 0.5% (v/v) or below.

In certain embodiments, when at least one cationic or ionizable cationic lipid is present in a TCV, the amount of said at least one cationic or ionizable cationic lipid relative to the total components of the TCV may be: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %

In certain embodiments, when at least one helper lipid is present in a TCV, the amount of said at least one helper lipid relative to the total components of the TCV is about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %.

In certain embodiments, when at least one phospholipid is present in a TCV, the amount of said at least one phospholipid relative to the total components of the TCV may be about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %.

In certain embodiments, when at least one cholesterol or cholesterol derivative is present in a TCV, the amount of said at least one cholesterol or cholesterol derivative relative to the total components of the TCV may be about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%.

In certain embodiments, when at least one PEG or PEG-lipid is present in a TCV, the amount of said at least one PEG or PEG-lipid relative to the total components of the TCV may be about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %.

In certain embodiments, said TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; and (iv) at least one cholesterol or cholesterol derivative. In particular embodiments, the amounts of said at least one ionizable cationic lipid, said at least one helper lipid, said at least one phospholipid, and said at least one cholesterol or cholesterol derivative, relative to the total components of the TCV, may be about 20 mol %, about 30 mol %, about 10 mol %, and about 40 mol %, respectively.

In certain embodiments, the TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; (iv) at least one cholesterol or cholesterol derivative; and (v) at least one PEG or PEG-lipid, which is optionally PEG-DMG. In particular embodiments, the amounts of said at least one ionizable cationic lipid, said at least one helper lipid, said at least one phospholipid, said at least one cholesterol or cholesterol derivative, and said at least one PEG or PEG-lipid, relative to the total components of the TCV, may be about 20 mol %, about 30 mol %, about 10 mol %, about 39 mol %, and about 1 mol %, respectively.

In particular embodiments, the TCV may be substantially, essentially, or entirely free of ethanol, methanol, isopropanol, tetrahydrofuran (THF), dimethyl sulfoxide (DMSO), dimethyl formamide (DMF), and acetonitrile (ACN). In particular embodiments, the TCV may be substantially, essentially, or entirely free of organic solvents and detergents. In particular embodiments, the TCV may be substantially, essentially, or entirely free of destabilizing agents. In particular embodiments, the TCV may be substantially, essentially, or entirely free of any permanently cationic lipids and/or any permanently anionic lipids. In particular embodiments, the TCV may be stable for prolonged periods of time at about 1 to about 40° C., about 5 to about 35° C., about 10 to about 30° C., or about 15 to about 25° C.

In particular embodiments, said TCV or said composition may further comprise and/or may be stored in the presence of at least one cryoprotectant. In some cases, the cryoprotectant may comprise a sugar-based molecule, which is optionally sucrose, trehalose, or a combination thereof. In some cases, the concentration of the cryoprotectant may be about 1% to about 40%, about 3% to about 30%, about 5% to about 30%, about 10% to about 20%, or about 15%. In some cases, said TCV may be stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months.

Any of the TCVs or the compositions according to the present invention may comprise any of or any combinations of the features of a TCV or a composition described herein.

In one aspect, the present disclosure provides CRISPR-mediated gene editing reporter systems. A CRISPR-mediated gene editing reporter system may comprise: (A) a cell, a tissue comprising a cell, or a transgenic animal; and (B) CRISPR-mediated gene editing agents.

In some embodiments, a cell in (A) may comprise a DNA molecule comprising (i) at least one first segment comprising or consisting of SEQ ID NO: 20, 21, 22, or 23 or comprising or consisting of a sequence comprising one or more mutations, optionally one, two, three, four, or five mutations, relative to SEQ ID NO: 20, 21, 22, or 23; and/or (ii) at least one second segment comprising or consisting of SEQ ID NO: 30, 31, 32, or 33 or comprising or consisting of a sequence comprising one or more mutations, optionally one, two, three, four, or five mutations, relative to SEQ ID NO: 30, 31, 32, or 33, wherein the DNA span is/are flanked by: (iii) a third segment comprising or consisting of SEQ ID NO: 40, 41, 42, or 43 or comprising or consisting of a sequence comprising one or more mutations, optionally one, two, three, four, or five mutations, relative to SEQ ID NO: 40, 41, 42, or 43; and (iv) a fourth segment comprising or consisting of SEQ ID NO: 50, 51, 52, or 53 or comprising or consisting of a sequence comprising one or more mutations, optionally one, two, three, four, or five mutations, relative to SEQ ID NO: 50, 51, 52, or 53. In certain embodiments, the third and fourth segments may be upstream and downstream, respectively, of the DNA span, or the third and fourth segments may be downstream and upstream, respectively, of the DNA span. In certain embodiments, at least one terminator sequence may be contained within said DNA span. In certain embodiments, the DNA span may further comprise a reporter gene sequence located downstream of the third and fourth segments. In particular embodiments, the DNA molecule may comprise any of SEQ ID NOS: 1, 60, 70, 80, 81, and 90. In certain embodiments, the cell may be a cell line or a primary cell. In certain embodiments, the cell may be a cell of a tissue or organ of interest. In certain embodiments, the reporter gene may encode a fluorescent marker, optionally monomeric cherry (mCherry), tandem dimer Tomato (tdTomato), red fluorescent protein (RFP), DsRed1, DsRed S197Y, green fluorescent protein (GFP), enhanced FP (EGFP), yellow fluorescent protein (YFP), enhanced yellow fluorescent protein (EYFP), cyan fluorescent protein (CFP), or enhanced cyan (ECFP).

In some embodiments, a tissue in (A) may comprise any of the cell described above.

In some embodiments, a transgenic animal in (A) may comprise any of the cell described above and/or a tissue comprising such a cell. In certain embodiments, the transgenic animal may be a non-human animal or optionally, without limitation, a rodent (mouse, rat, guinea pig, hamster), rabbit, cat, dog, pig, goat, sheep, horse, or monkey, further optionally a mouse or a rat. In certain embodiments, the transgenic animal may comprise the above-mentioned DNA span flanked by said third and fourth segments at the Rosa26 locus. In certain embodiments, the transgenic animal may be the Ai9 or Ai14 mouse. In particular embodiments, the transgenic animal may be B6;129S6-Gt(ROSA)26Sor^{tm14(CAG-tdTomato)Hze}/J or its congenic version B6.Cg-Gt(ROSA)26Sor^{tm14(CAG-tdTomato)Hze}/J, such as Strain #007908 or 007914, respectively, from the Jackson Laboratory, commonly referred to as Ai14, Ai14D, or Ai14(RCL-tdT)-D mouse. In certain embodiments, the transgenic animal may be B6.Cg-Gt(ROSA)26Sor^{tm9(CAG-tdTomato)Hze}/J, such as Strain #007909 from the Jackson Laboratory, commonly referred to as Ai9 or Ai9 (RCL-tdT) mouse.

In some embodiments, CRISPR-mediated gene editing agents in (B) may comprise multiple isolated gRNAs or one or more polynucleotides encoding the multiple isolated gRNAs. In certain embodiments, the multiple isolated gRNAs may comprise a first isolated gRNA and/or a second isolated gRNA and may further comprise a third isolated gRNA and a fourth isolated gRNA. The first, second, third, and/or fourth isolated gRNAs may be according to any of the first, second, third, and/or fourth isolated gRNAs, respectively, as already described above.

In some embodiments, CRISPR-mediated gene editing agents in (B) may further comprise a Cas endonuclease or a polynucleotide encoding a Cas endonuclease, and the Cas endonuclease may be according to any of the Cas endonucleases described herein. In some embodiments, CRISPR-mediated gene editing agents in (B) may further comprise a template DNA or a polynucleotide encoding a template DNA, and the template DNA may be according to any of the template DNAs described herein.

In certain embodiments, (1) the multiple isolated gRNAs or the one or more polynucleotides encoding the multiple isolated gRNAs and (2) the Cas endonuclease or the polynucleotide encoding a Cas endonuclease, and (3) optionally the template DNA or the polynucleotide encoding a template DNA, of a CRISPR-mediated gene editing reporter system described herein may be comprised in a composition according to any of the compositions described above.

In one aspect, the present disclosure provides methods of testing in vitro the level of CRISPR-mediated gene editing events in a cell, which method may use any of the CRISPR-mediated gene editing reporter systems described above.

In some embodiments, the method may comprise (a) applying in vitro to one or more cells CRISPR-mediated gene editing agents, wherein said CRISPR-mediated gene editing agents comprise or are comprised in a CRISPR-mediated gene editing reporter system according to the present disclosure and said one or more cells are according to a cell of a CRISPR-mediated gene editing reporter system according to the present disclosure; and (b) analyzing the level of CRISPR-mediated gene editing events in said one or more cells.

In certain embodiments, the analyzing may comprise (i) quantifying the reporter gene expression in said one or more cells, optionally via flow cytometry, fluorescent microscopy, or qPCR; and/or (ii) determining the presence or absence or level of (ii-1) the DNA sequence flanked by the sites cleavable by the third and fourth isolated gRNAs or (ii-2) the transcript thereof in said one or more cells, optionally via PCR or qPCR, respectively.

In particular embodiments, said CRISPR-mediated gene editing agents may be contained in a composition comprising a pharmaceutically acceptable carrier of interest and the method may be for testing the effect of the carrier of interest on CRISPR-mediated gene editing events. In particular embodiments, said cell may be of a cell type of interest, and the method is for testing whether the cell type of interest is compatible with CRISPR-mediated gene editing. In particular embodiments, the cell may be of a cell type of interest, the CRISPR-mediated gene editing agents are contained in a composition comprising a pharmaceutically acceptable carrier of interest, and the method may be for testing whether the carrier of interest may be compatible with the cell type of interest.

In one aspect, the present disclosure provides methods of testing ex vivo the level of CRISPR-mediated gene editing events in a tissue, which method may use any of the CRISPR-mediated gene editing reporter systems described above.

In some embodiments, the method may comprise (a) applying ex vivo to one or more tissues CRISPR-mediated gene editing agents, wherein said CRISPR-mediated gene editing agents comprise or are comprised in a CRISPR-mediated gene editing reporter system according to the present disclosure, and said one or more tissues are according to a tissue of a CRISPR-mediated gene editing reporter system according to the present disclosure; and (b) analyzing the level of CRISPR-mediated gene editing events in said one or more tissues (or cells contained therein).

In certain embodiments, the analyzing may comprise (i) quantifying the reporter gene expression in said one or more tissues (or cells contained therein), optionally via flow cytometry, fluorescent microscopy, or qPCR; and/or (ii) determining the presence or absence or level of (ii-1) the DNA sequence flanked by the sites cleavable by the third and fourth isolated gRNAs or (ii-2) the transcript thereof in said one or more tissues (or cells contained therein), optionally via PCR or qPCR, respectively.

In particular embodiments, the CRISPR-mediated gene editing agents may be contained in a composition comprising a pharmaceutically acceptable carrier of interest and the method may be for testing the effect of the carrier of interest on CRISPR-mediated gene editing events. In particular embodiments, the tissue may be of a tissue type of interest, and the method is for testing whether the tissue type of interest is compatible with CRISPR-mediated gene editing. In particular embodiments, the cell or tissue may be of a tissue of interest, the CRISPR-mediated gene editing agents are contained in a composition comprising a pharmaceutically acceptable carrier of interest, and the method may be for testing whether the carrier of interest may be compatible with the tissue type of interest.

In one aspect, the present disclosure provides methods of testing in vivo the level of CRISPR-mediated gene editing events in an animal, which method may use any of the CRISPR-mediated gene editing reporter systems described above.

In some embodiments, the method may comprise (a) applying to one or more transgenic animals CRISPR-mediated gene editing agents, wherein said CRISPR-mediated gene editing agents comprise or are comprised in a CRISPR-mediated gene editing reporter system described herein and said one or more transgenic animals are according to a transgenic animal of a CRISPR-mediated gene editing reporter system described herein; and (b) analyzing the level of CRISPR-mediated gene editing events in said one or more transgenic animals.

In certain embodiments, the analyzing may comprise (i) quantifying the reporter gene expression in the one or more transgenic animals or tissues or cells derived therefrom, optionally via flow cytometry, fluorescent microscopy, or qPCR; and/or (ii) determining the presence or absence or level of (ii-1) the DNA sequence flanked by the sites cleavable by the third and fourth isolated gRNAs or (ii-2) the transcript thereof in the one or more transgenic animals or tissues or cells derived therefrom, optionally via PCR or qPCR, respectively.

In particular embodiments, the CRISPR-mediated gene editing agents may be contained in a composition comprising a pharmaceutically acceptable carrier of interest and the method may be for testing the effect of the carrier of interest on CRISPR-mediated gene editing events in vivo.

In particular embodiments, the transgenic animal may be of a species of interest, and the method may be for testing whether the species of interest is compatible with CRISPR-mediated gene editing.

In particular embodiments, the transgenic animal may be of a species of interest, the CRISPR-mediated gene editing agents may be contained in a composition comprising a pharmaceutically acceptable carrier of interest, and the method may be for testing whether the carrier of interest is compatible with the transgenic animal is of a species of interest.

In particular embodiments, the method may be for evaluating the level of side effects and/or adverse events.

In particular embodiments, the applying of step (a) may comprise administrating via an administration route of interest, and the method may be for testing whether the administration route of interest is suited for effecting CRISPR-mediated gene editing, optionally based on the level of gene editing evens and/or the level of side effects and/or adverse events.

In particular embodiments, the applying of step (a) may comprise administrating the CRISPR-mediated gene editing agents at a dose or a dose range of interest, and the method may be for determining an approximate dose or dose rage suited for effecting CRISPR-mediated gene editing, optionally based on the level of gene editing evens and/or the level of side effects and/or adverse events.

Some aspects of the present disclosure relate to knocking out a gene segment of interest, for example by using more than two gRNAs.

In one aspect, the present disclosure provides method of knocking out a DNA segment of interest in a cell, tissue, or subject, wherein: (i) said DNA segment of interest may be comprised in an intervening sequence flanked by a 5′ first site cleavable by CRISPR-mediated gene editing via a first gRNA and a 3′ second site cleavable by CRISPR-mediated gene editing via a second gRNA and (ii) said intervening sequence may comprise at least one third site cleavable by CRISPR-mediated gene editing via a third gRNA.

In some embodiments, such a method may comprise applying CRISPR-mediated gene editing agents to the cell, tissue, or subject, and the CRISPR-mediated gene editing agents may comprise: (a) said first gRNA, said second gRNA, and said third gRNA (which are optionally comprised at equimolar ratios) or one or more polynucleotides encoding said first gRNA, said second gRNA, and said third gRNA; (b) a Cas endonuclease or a polynucleotide encoding a Cas endonuclease; and (c) optionally a template DNA or a polynucleotide encoding a template DNA.

In certain embodiments, the template DNA may comprise (i) a 5′ homology arm homologous or complementary to the DNA sequence immediately upstream of the first site and (ii) a 3′ homology arm homologous or complementary to the DNA sequence immediately downstream of the second site.

In certain embodiments, the subject may be a human or a non-human subject, further optionally a non-human primate. In certain embodiments, the subject may selected from a rodent (mouse, rat, guinea pig, hamster), rabbit, cat, dog, pig, goat, sheep, horse, or monkey. In certain embodiments, the subject may be a mouse or a rat.

In certain embodiments, the intervening sequence may comprise two or more third sites cleavable by CRISPR-mediated gene editing via the third gRNA. In certain embodiments, the intervening sequence may be about 10-10000 nucleotides in length, about 20-5000 nucleotides in length, about 50-2500 nucleotides in length, about 100-2000 nucleotides in length, about 500-2000 nucleotides in length, or about 500-1500 nucleotides in length.

In certain embodiments, the intervening sequence may further comprise at least one fourth site cleavable by CRISPR-mediated gene editing via a fourth gRNA. In some cases, the fourth gRNA may comprise a different target specificity relative to the third gRNA, and the CRISPR-mediated gene editing agents may further comprise the fourth gRNA. In particular cases, the intervening sequence may comprise two or more fourth sites cleavable by CRISPR-mediated gene editing via the fourth gRNA.

In another aspect, the present disclosure provides compositions for knocking out a DNA segment of interest in a cell, tissue, or subject, wherein: (i) said DNA segment of interest may be comprised in an intervening sequence flanked by a 5′ first site cleavable by CRISPR-mediated gene editing via a first gRNA and a 3′ second site cleavable by CRISPR-mediated gene editing via a second gRNA and (ii) said intervening sequence may comprise at least one third site cleavable by CRISPR-mediated gene editing via a third gRNA.

In some embodiments, such a composition may comprise (a) said first gRNA, said second gRNA, and said third gRNA (optionally at an equimolar ratio) or one or more polynucleotides encoding said first gRNA, said second gRNA, and said third gRNA; (b) a Cas endonuclease or a polynucleotide encoding a Cas endonuclease; and (c) optionally a template DNA or a polynucleotide encoding a template DNA. In certain embodiments, the template DNA may comprise (i) a 5′ homology arm homologous or complementary to (i.e., fully complementary or comprising one or more mismatches) the DNA sequence immediately upstream of the first site and (ii) a 3′ homology arm homologous or complementary to (i.e., fully complementary or comprising one or more mismatches) the DNA sequence immediately downstream of the second site.

In certain embodiments, the presence of said third gRNA may increase the efficiency or probability of knocking out the DNA segment of interest. In certain embodiments, said intervening sequence comprises two or more third sites cleavable by CRISPR-mediated gene editing via said third gRNA. In certain embodiments, the intervening sequence may be about 10-10000 nucleotides in length, about 20-5000 nucleotides in length, about 50-2500 nucleotides in length, about 100-2000 nucleotides in length, about 500-2000 nucleotides in length, or about 500-1500 nucleotides in length.

In some embodiments, the intervening sequence may further comprise at least one fourth site cleavable by CRISPR-mediated gene editing via a fourth gRNA. In some cases, the fourth gRNA may comprise a different target specificity relative to said third gRNA, and the CRISPR-mediated gene editing agents may further comprise the fourth gRNA. In certain cases, the intervening sequence comprises two or more fourth sites cleavable by CRISPR-mediated gene editing via the fourth gRNA.

In certain embodiments, the presence of the fourth gRNA may further increase the efficiency or probability of knocking out the DNA segment of interest.

Some aspects of the present disclosure relate to effecting CRISPR-mediated gene editing in the eye, optionally in the cornea, iris, retina, or subretinal tissue.

In some embodiments, the method may comprise administering CRISPR-mediated gene editing agents directly into the eye of a subject, optionally into the cornea (e.g., the epithelial, stromal, and/or endothelial tissue of the cornea), iris, retina, or subretinal tissue. In some embodiments, the method may comprise administering CRISPR-mediated gene editing agents intravitreally or via ocular drops.

In certain embodiments, the CRISPR-mediated gene editing agents may comprise: (a) one or more gRNAs or one or more polynucleotides encoding the one or more gRNAs; (b) a Cas endonuclease or a polynucleotide encoding a Cas endonuclease; and (c) optionally a template DNA or a polynucleotide encoding a template DNA. In some cases, any one or more of (a)-(c) are encapsulated in a TCV. In particular cases, such a TCV may comprise any one or more of the TCV features described herein. In certain embodiments, the subject may a human, non-human, optionally non-human primate, a rodent (optionally mouse, rat, guinea pig, hamster), rabbit, cat, dog, pig, goat, sheep, horse, or monkey.

In particular embodiments, the method may be for knocking out a DNA segment of interest in the eye or a cell thereof of the subject, and (i) said DNA segment of interest may be comprised in an intervening sequence flanked by a 5′ first site cleavable by CRISPR-mediated gene editing via a first gRNA and a 3′ second site cleavable by CRISPR-mediated gene editing via a second gRNA; and (ii) said intervening sequence may comprise at least one third site cleavable by CRISPR-mediated gene editing via a third gRNA. In some instances, the one or more gRNAs of the CRISPR-mediated gene editing agents may comprise or consist of the first gRNA, the second gRNA, and the third gRNA (optionally at an equimolar ratio).

In some instances, the one or more polynucleotides encoding the one or more gRNAs of the CRISPR-mediated gene editing agents may comprise or consist of one or more polynucleotides encoding the first gRNA, the second gRNA, and the third gRNA. In some instances, if a template DNA is present in the CRISPR-mediated gene editing agents, the template DNA may optionally comprise (c-1) a 5′ homology arm homologous or complementary to the DNA sequence immediately upstream of the first site and (c-2) a 3′ homology arm homologous or complementary to the DNA sequence immediately downstream of the second site. In some instances, if a polynucleotide encoding a template DNA is present, the polynucleotide may optionally encode (c-1) a 5′ homology arm homologous or complementary to the DNA sequence immediately upstream of the first site and (c-2) a 3′ homology arm homologous or complementary to the DNA sequence immediately downstream of the second site. In particular instances, the intervening sequence may comprise two or more third sites cleavable by CRISPR-mediated gene editing via the third gRNA. In particular instances, the intervening sequence may be about 10-10000 nucleotides in length, about 20-5000 nucleotides in length, about 50-2500 nucleotides in length, about 100-2000 nucleotides in length, about 500-2000 nucleotides in length, or about 500-1500 nucleotides in length.

In further embodiments, the intervening sequence may further comprise at least one fourth site cleavable by CRISPR-mediated gene editing via a fourth gRNA which comprises a different target specificity relative to the third gRNA. In such an embodiment, the CRISPR-mediated gene editing agents may further comprise the fourth gRNA. In particular instances, the intervening sequence comprises two or more fourth sites cleavable by CRISPR-mediated gene editing via the fourth gRNA.

In certain embodiments, such a method of effecting may be for treating or preventing a genetic disease or disorder of the eye.

In one aspect, the present disclosure provides compositions for base editing.

Such a composition may comprise: (A) a pharmaceutically acceptable carrier, which is or comprises a lipid-based TCV; and (B) a RNP, which is or comprises a base editor complexed with a gRNA, wherein the RNP is encapsulated in the lipid based TCV.

In some embodiments, the based editor may be an adenine base editor (ABE), a cytidine base editor (CBE), or a dual editor (DE).

In some embodiments, the base editor may comprise a Cas-derived platform protein linked to a deaminase.

In certain embodiments, the Cas-derived platform protein may be, may comprise, or may be derived from a Cas-derived nickase (nCas) or a catalytically dead Cas (dCas).

In certain embodiments, the deaminase may be: (i) an adenine deaminase; (ii) a cytidine deaminase; or (iii) an adenine cytidine deaminase (dual deaminase).

In certain embodiments, the gRNA may be designed to effect base editing in the presence of a target DNA and the Cas-derived platform protein. Optionally, the base editing may be or may comprise: (i) transversion of a target adenine to a guanine; (ii) transversion of a target cytidine to a thymidine; or (iii) transversion of a target adenine to a guanine and transversion of a target cytidine to a thymidine.

In some embodiments, said gRNA may comprises a targeting sequence of 17, 18, 19, 20, 21, 22, 23, 24, 25, 26, 27, 28, 29, or 30 nucleotides in length.

In some embodiments, said gRNA may be a sgRNA comprising (i) a crRNA sequence comprising the targeting sequence and a crRNA backbone sequence and (ii) a tracrRNA sequence in a single strand. In certain embodiments, the crRNA sequence and the tracrRNA sequence may be linked via a linker optionally comprising the nucleic acid sequence of GAAA. In certain embodiments, the gRNA may comprise the targeting sequence followed by a sgRNA backbone sequence of any of SEQ ID NOS: 111-114. Optionally said sgRNA backbone sequence is followed by one or more uracils, further optionally 1-10 uracils.

In some embodiments, said gRNA may be a dgRNA formed by hybridization between (i) a crRNA comprising the targeting sequence and a crRNA backbone sequence and (ii) a tracrRNA. In certain embodiments, the crRNA backbone sequence and the tracrRNA may comprise SEQ ID NOS: 115 and 116, respectively, or SEQ ID NOS: 117 and 118.

In some embodiments, said gRNA may be synthetic or recombinant.

In some embodiments, said gRNA may comprise at least one chemical modification. In certain embodiments, said gRNA may comprise at least one chemical modification such as 2′-O-methylation optionally at first three and last three bases and/or one or more 3′ phosphorothioate bonds, optionally between first three and last two bases.

In certain embodiments, the Cas-derived platform protein may be or may comprise a Cas-derived nickase (nCas) or a catalytically dead Cas (dCas).

In certain embodiments, the Cas-derived platform protein may be derived from a Cas endonuclease selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2. In certain embodiments, the Cas-derived platform protein may be a class 2 Cas endonuclease, optionally a type II, type V, or type VI Cas nuclease. In certain embodiments, the Cas-derived platform protein may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of SpCas9 (e.g., CP1012-SpCas9, CP1028-SpCas9, CP1041-SpCaS9, CP1249-SpCas9, and CP1300-SpCas9), eSpCas9, HypaCas9, FokI-Fused dCas9, xCas9, SpRY (variant of SpCas9), and SpG (variant of SpCas9). In certain embodiments, the Cas-derived platform protein may be Cas9, optionally comprising any one of SEQ ID NOS: 600-611. In certain embodiments, the Cas-derived platform protein may be Cas12a of Lachnospiraceae bacterium ND2006 (LbCas12a), Acidaminococcus sp. BV3L6 (AsCas12a), or Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a.

In certain embodiments, the Cas-derived platform protein may be or may comprise any of the following or a variant thereof: (1) a SpCas9 nickase, optionally comprising the sequence of SEQ ID NO: 621 or a Cas9 variant (optionally SpCas9 variant) comprising the D10A substitution of SpCas9; (2) a dead SpCas9, (dCas9) optionally comprising the sequence of SEQ ID NO: 620 or a Cas9 variant, optionally SpCas9 variant further optionally one comprising the D10A and H840A substitutions of dCas9; (3) a VQR-SpCas9 nickase, optionally comprising the sequence of SEQ ID NO: 631; (4) a EQR-SpCas9 nickase, optionally comprising the sequence of SEQ ID NO: 632; (5) a VRER-SpCas9 nickase, optionally comprising the sequence of SEQ ID NO: 633; (6) a CP1028-SpCas9, optionally comprising the sequence of SEQ ID NO: 634; (7) a CP1041-SpCas9, optionally comprising the sequence of SEQ ID NO: 635; (8) a SpCas9-NG, optionally comprising the sequence of SEQ ID NO: 636; (9) SaCas9 nickase (SEQ ID NO: 640); (10) SaCas9-KKH (SEQ ID NO: 641); (11) a catalytically dead LbCas12a (dLbCas12a), optionally comprising the sequence of SEQ ID NO: 650; or (12) an engineered AsCas12a (enAsCas12a), optionally comprising the sequence of SEQ ID NO: 660.

In some embodiments, the deaminase may be an adenine deaminase derived from a TadA, optionally from TadA of E. coli (ecTadA), optionally comprising the sequence of SEQ ID NO: 820.

In certain embodiments, the deaminase may be or may comprises any of the following: (1) ecTadA*8e (SEQ ID NO: 826) or a TadA variant (optionally ecTadA variant or ecTadA*7.10 variant) comprising at least 90%, at least 95%, at least 96%, at least 97%, at least 98%, at least 99% sequence identity to SEQ ID NO: 826 and/or comprising one or more of the substitutions A109S, T111R, D119N, H122N, Y147D, F149Y, T166I, and D167N of ecTadA*8e; (2) ecTadA*8e-V106W (SEQ ID NO: 827) or a TadA variant (optionally ecTadA variant or ecTadA*8e variant) comprising at least 90%, at least 95%, at least 96%, at least 97%, at least 98%, at least 99% sequence identity to SEQ ID NO: 827 and/or 106W of ecTadA*8e-V106W; (3) ecTadA*8e-V82G (SEQ ID NO: 828) or a TadA variant (optionally ecTadA variant or ecTadA*8e variant) comprising at least 90%, at least 95%, at least 96%, at least 97%, at least 98%, at least 99% sequence identity to SEQ ID NO: 828 and/or the V82G substitution of ecTadA*8e-V82G; (4) ecTadA*8e-K20A-R21A (SEQ ID NO: 829) or a TadA variant (optionally ecTadA variant or ecTadA*8e variant) comprising at least 90%, at least 95%, at least 96%, at least 97%, at least 98%, at least 99% sequence identity to SEQ ID NO: 829 and/or comprising the K20A and/or R21A substitutions of ecTadA*8e-K20A-R21A; (5) ecTadA*6.3 (SEQ ID NO: 821) or a TadA variant (optionally ecTadA variant) comprising at least 90%, at least 95%, at least 96%, at least 97%, at least 98%, at least 99% sequence identity to SEQ ID NO: 821 and/or comprising one or more of the substitutions A106V, D108N, D147Y, and E155V; L84F, H123Y, and I157F; H36L, R51L, S146C, and K157N; and P48S of ecTadA*6.3; (6) ecTadA*6.4 (SEQ ID NO: 822) or a TadA variant (optionally ecTadA variant) comprising at least 90%, at least 95%, at least 96%, at least 97%, at least 98%, at least 99% sequence identity to SEQ ID NO: 822 and/or comprising one or more of the substitutions A106V, D108N, D147Y, and E155V; L84F, H123Y, and I157F; H36L, R51L, S146C, and K157N; and P48S and A142N of ecTadA*6.4; (7) ecTadA*7.8 (SEQ ID NO: 823) or a TadA variant (optionally ecTadA variant) comprising at least 90%, at least 95%, at least 96%, at least 97%, at least 98%, at least 99% sequence identity to SEQ ID NO: 823 and/or comprising one or more of the substitutions A106V, D108N, D147Y, and E155V; L84F, H123Y, and I157F; H36L, R51L, S146C, and K157N; A142N; and W23L and P48A of ecTadA*7.8; (8) ecTadA*7.9 (SEQ ID NO: 824) or a TadA variant (optionally ecTadA variant) comprising at least 90%, at least 95%, at least 96%, at least 97%, at least 98%, at least 99% sequence identity to SEQ ID NO: 824 and/or comprising one or more of the substitutions A106V, D108N, D147Y, and E155V; L84F, H123Y, and I157F; H36L, R51L, S146C, and K157N; A142N; and W23L, P48A, and R152P of ecTadA*7.9; or (9) ecTadA*7.10 (SEQ ID NO: 825) or a TadA variant (optionally ecTadA variant) comprising at least 90%, at least 95%, at least 96%, at least 97%, at least 98%, at least 99% sequence identity to SEQ ID NO: 825 and/or comprising one or more of the substitutions A106V, D108N, D147Y, and E155V; L84F, H123Y, and I157F; H36L, R51L, S146C, and K157N; and W23R, P48A, and R152P of ecTadA*7.10.

In some embodiments, the deaminase may be a cytidine deaminase derived from APOBEC, optionally from APOBEC1, further optionally from APOBEC1 of rat (rAPOBEC1) (SEQ ID NO: 720) or of human.

In certain embodiments, the deaminase may be a cytidine deaminase which may be or may comprise any of the following: (1) rAOPBEC1 (SEQ ID NO: 720) or a AOPBEC1 variant (optionally rAOPBEC1 variant) comprising at least 90%, at least 95%, at least 96%, at least 97%, at least 98%, at least 99% sequence identity to SEQ ID NO: 720; (2) YE1-rAPOBEC1 (SEQ ID NO: 721) or a AOPBEC1 variant (optionally rAOPBEC1 variant or YE1-rAPOBEC1 variant) comprising at least 90%, at least 95%, at least 96%, at least 97%, at least 98%, at least 99% sequence identity to SEQ ID NO: 721 and/or comprising the W90Y and/or R126E substitutions of YE1-rAPOBEC1; (3) YE2-rAPOBEC1 (SEQ ID NO: 722) or a AOPBEC1 variant (optionally rAOPBEC1 variant or YE2-rAPOBEC1 variant) comprising at least 90%, at least 95%, at least 96%, at least 97%, at least 98%, at least 99% sequence identity to SEQ ID NO: 722 and/or comprising the W90Y and/or R132E substitutions of YE2-rAPOBEC1; (4) EE-rAPOBEC1 (SEQ ID NO: 723) or a AOPBEC1 variant (optionally rAOPBEC1 variant or EE-rAPOBEC1 variant) comprising at least 90%, at least 95%, at least 96%, at least 97%, at least 98%, at least 99% sequence identity to SEQ ID NO: 723 and/or comprising the R126E and/or R132E substitutions of EE-rAPOBEC1; (5) YEE-rAPOBEC1 (SEQ ID NO: 724) or a AOPBEC1 variant (optionally rAOPBEC1 variant or YEE-rAPOBEC1 variant) comprising at least 90%, at least 95%, at least 96%, at least 97%, at least 98%, at least 99% sequence identity to SEQ ID NO: 724 and/or comprising one or more of the substitutions W90Y, R126E, and R132E of YEE-rAPOBEC1; (6) Anc689 APOBEC (SEQ ID NO: 731) or a AOPBEC variant (optionally Anc689 APOBEC variant) comprising at least 90%, at least 95%, at least 96%, at least 97%, at least 98%, at least 99% sequence identity to SEQ ID NO: 731; or (7) Anc687 APOBEC (SEQ ID NO: 732) or a AOPBEC variant (optionally Anc687 APOBEC variant) comprising at least 90%, at least 95%, at least 96%, at least 97%, at least 98%, at least 99% sequence identity to SEQ ID NO: 732.

In some embodiments, the deaminase may be a cytidine deaminase derived from CDA1 or from AID.

In certain embodiments, the deaminase may be a cytidine deaminase which may be or may comprise any of the following or a variant thereof comprising at least 90%, at least 95%, at least 96%, at least 97%, at least 98%, at least 99% sequence identity to thereto: (1) CDA1 (SEQ ID NO: 725); or (2) AID (SEQ ID NO: 726).

In some embodiments, the deaminase may be a cytidine deaminase derived from a TadA deaminase, optionally from TadA of E. coli (ecTadA), optionally comprising the sequence of SEQ ID NO: 820.

In certain embodiments, the deaminase may be a cytidine deaminase which may be or may comprise any of the following or a variant thereof comprising at least 90%, at least 95%, at least 96%, at least 97%, at least 98%, at least 99% sequence identity to thereto: (1) TadA-CDa (SEQ ID NO: 741); (2) TadA-CDb (SEQ ID NO: 742); (3) TadA-CDc (SEQ ID NO: 743); (4) TadA-CDd (SEQ ID NO: 744); (5) TadA-CDe (SEQ ID NO: 745); (6) TadA-CDa-V106W (SEQ ID NO: 751); (7) TadA-CDb-V106W (SEQ ID NO: 752); (8) TadA-CDc-V106W (SEQ ID NO: 753); (9) TadA-CDd-V106W (SEQ ID NO: 754); or (10) TadA-CDe-V106W (SEQ ID NO: 755).

In some embodiments, the deaminase may be an adenine and cytidine deaminase (dual deaminase) derived from TadA, optionally from TadA of E. coli (ecTadA) (SEQ ID NO: 820).

In certain embodiments, the deaminase may be a dual deaminase which may be or may comprise any of the following or a variant thereof comprising at least 90%, at least 95%, at least 96%, at least 97%, at least 98%, at least 99% sequence identity to thereto: (1) TadA*Dual (SEQ ID NO: 920); or (2) TadA*Dual-V106W (SEQ ID NO: 921).

In some embodiments, the base editor may be an ABE, which comprises: (a) optionally a nuclear localization signal (NLS); (b) an/the adenine deaminase; (c) optionally a linker; (d) an/the adenine deaminase; (e) optionally a linker; (f) a/the Cas-derived platform protein; and (g) optionally a NLS, wherein each of (a) to (g) if present, are optionally comprised in the ABE in the recited order in the direction from the N-terminus to the C-terminus.

In some embodiments, the base editor may be a CBE, which comprises: (a) optionally a NLS; (b) optionally Gam; (c) optionally a linker; (d) an/the cytidine deaminase; (e) optionally a linker; (f) a/the Cas-derived platform protein; and (g) optionally a linker; (h) optionally a uracil DNA glycosylase inhibitor (UGI); (i) optionally a linker; (j) optionally a UGI; (k) optionally a linker; and (l) optionally a NLS, wherein each of (a) to (l) if present, are optionally comprised in the CBE in the recited order in the direction from the N-terminus to the C-terminus.

In some embodiments, the base editor may be a DE, which comprises: (a) optionally a NLS; (b) optionally Gam; (c) optionally a linker; (d) an/the dual deaminase; (e) optionally a linker; (f) a/the Cas-derived platform protein; and (g) optionally a linker; (h) optionally a UGI; (i) optionally a linker; (j) optionally a UGI; (k) optionally a linker; and (l) optionally a NLS, wherein each of (a) to (l) if present, are optionally comprised in the DE in the recited order in the direction from the N-terminus to the C-terminus.

In certain embodiments, the NLS may be or may comprise any of the following or a variant thereof comprising at least 90%, at least 95%, at least 96%, at least 97%, at least 98%, at least 99% sequence identity to thereto; NLS1 (SEQ ID NO: 691); NLS2 (SEQ ID NO: 692); and/or NLS3 (SEQ ID NO: 693);

In certain embodiments, the linker individually may: (1) comprise one or more amino acids, optionally one, two, three, four, five, six, seven, eight, nine, ten, eleven, or twelve amino acids; (2) comprise or consist of G, S, and/or A; (3) comprise or consist of or comprise or consist of multiple repeats of an amino acid sequence selected from the group consisting of G, GG, GGG, GS, SG, GGS, GSG, SGG, GSS, SGS, SSG, SEQ ID NO: 682, SEQ ID NO: 685, and SEQ ID NO: 686; and/or (4) comprise or consist of the amino acid sequence of SEQ ID NO: 681, 683, or 684.

In certain embodiments, the UGI may comprise the amino acid sequence of SEQ ID NO: 760 or an amino acid sequence comprising at least 90%, at least 95%, at least 96%, at least 97%, at least 98%, at least 99% sequence identity to thereto.

In certain embodiments, the Gam may comprise the amino acid sequence of SEQ ID NO: 770 or an amino acid sequence comprising at least 90%, at least 95%, at least 96%, at least 97%, at least 98%, at least 99% sequence identity to thereto.

In some embodiments the base editor may be an ABE which is or comprises any of the following or a variant thereof comprising at least 90%, at least 95%, at least 96%, at least 97%, at least 98%, at least 99% sequence identity to thereto: (i) ABE8e (SEQ ID NO: 810); (ii) ABE8e dimer (SEQ ID NO: 811); (iii) ABEmax (SEQ ID NO: 801); and/or (iv) ABE7.10 (SEQ ID NO: 800).

In some embodiments the base editor may be a CBE which is or comprises any of the following or a variant thereof comprising at least 90%, at least 95%, at least 96%, at least 97%, at least 98%, at least 99% sequence identity to thereto: (i) BE4max (SEQ ID NO: 712); (ii) AncBE4max (SEQ ID NO: 713); (iii) BE4 (SEQ ID NO: 710); (iv) BE4-Gam (SEQ ID NO: 711); (v) BE3 (SEQ ID NO: 700); (vi) YE1-BE3 (SEQ ID NO: 701); (vii) YE2-BE3 (SEQ ID NO: 702); (viii) EE-BE3 (SEQ ID NO: 703); (ix) YEE-BE3 (SEQ ID NO: 704); (x) CDA1-BE3 (SEQ ID NO: 705); (xi) AID-BE3 (SEQ ID NO: 706); and/or (xii) BE3-Gam (SEQ ID NO: 707).

In some embodiments the base editor may be a DE which is or comprises any of the following or a variant thereof comprising at least 90%, at least 95%, at least 96%, at least 97%, at least 98%, at least 99% sequence identity to thereto: (i) TadDE (SEQ ID NO: 900); and/or (ii) TadDE-V106W (SEQ ID NO: 901).

In some embodiments, the RNP which is or comprises a base editor complexed with a gRNA may be encapsulated in the TCV by any of the methods for encapsulating a RNP in a TCV described herein. Such a method may comprise (i) providing an aqueous solution comprising the TCV and (ii) mixing the RNP with the aqueous solution as described herein.

In some embodiments, the TCV may be any of the TCVs described herein. Such a TCV may comprises at least one cationic or ionizable cationic lipid as described herein. In certain embodiments, the TCV may comprise essentially consists of, or consists of: (i) at least one ionizable cationic lipid, which is optionally DODMA; (ii) at least one helper lipid, which is optionally DOPE; (iii) at least one phospholipid, which is optionally DSPC; and (iv) at least one cholesterol or cholesterol derivative, optionally wherein the amounts of the at least one ionizable cationic lipid, the at least one helper lipid, the at least one phospholipid, and the at least one cholesterol or cholesterol derivative, relative to the total components of the TCV, is about 20 mol %, about 30 mol %, about 10 mol %, and about 40 mol %, respectively. In certain embodiments, the TCV may comprise essentially consists of, or consists of: (i) at least one ionizable cationic lipid, which is optionally DODMA; (ii) at least one helper lipid, which is optionally DOPE; (iii) at least one phospholipid, which is optionally DSPC; (iv) at least one cholesterol or cholesterol derivative; and (v) at least one PEG or PEG-lipid, which is optionally PEG-DMG, optionally wherein the amounts of the at least one ionizable cationic lipid, the at least one helper lipid, the at least one phospholipid, the at least one cholesterol or cholesterol derivative, and the at least one PEG or PEG-lipid, relative to the total components of the TCV, is about 20 mol %, about 30 mol %, about 10 mol %, about 39 mol %, and about 1 mol %, respectively.

In another aspect, the present disclosure provides methods of effecting base editing in one or more target cells. In some embodiments, such a method may comprise applying an effective amount of a composition for base editing according to the present disclosure to the one or more target cells. In certain embodiments, the applying may occur in vitro. In certain embodiments, the applying may occur ex vivo. In certain embodiments, the applying may occur in vivo.

In another aspect, the present disclosure provides methods of treating a subject. In some embodiments, such a method may comprise administering an effective amount of a composition for base editing according to the present disclosure to the subject. In certain embodiments, the subject may have or have a risk of contracting with a genetic disease associated with one or more genetic mutations and the composition may be designed to reverse or alter the genetic mutation(s).

In another aspect, the present disclosure provides methods of treating a disease, a disorder, or a condition in a subject. In some embodiments, such a method may comprise administering an effective amount of a composition for base editing according to the present disclosure to the subject. In certain embodiments, the disease a disorder, or a condition may be associated with one or more genetic mutations and the composition may be designed to reverse or alter the genetic mutation(s).

In some embodiments, in any of the methods of treating described above, the administering may be (i) to one or more of the subject's eyes, optionally (i-1) intravitreally or via ocular drops; (i-2) into the cornea, optionally the epithelial, stromal, and/or endothelial cells of the cornea; (i-3) into the iris, (i-4) into the retina; or (i-5) into the subretinal tissue; (ii) locally administering, optionally to the eye, ear, nose (optionally intranasally), skin (optionally transdermally or epicutaneously), mucosa, skin, or vagina, or by inhalation; (iii) parenterally administering, optionally by injection (optionally intravenous, intramuscular, subcutaneous, intradermal, intrathecal, intra-arterial, intraarticular, intraosseous, or intraperitoneal administration) or by inhalation; or (iv) enterally administering, optionally orally, sublingually, buccally, or rectally. In some embodiments, any of the methods of treating described above may be for effecting base editing in: the cornea, optionally an epithelial, stromal, and/or endothelial cell of the cornea; the iris; the retina, optionally a photoreceptor cell, a bipolar cell, a retinal ganglion cell, a muller glial cell, a horizontal cell, and/or a amacrine cell of the retina; or the subretinal tissue. In some embodiments, the subject may be a human, non-human, non-human primate, a rodent (mouse, rat, guinea pig, hamster), rabbit, cat, dog, pig, goat, sheep, horse, or monkey.

In another aspect, the present disclosure provides methods of preparing a composition for base editing, e.g., any of the compositions for gene editing described herein. In some embodiments, the method may comprise the same steps used for encapsulating a RNP with a lipid-based TCV described herein. In some embodiments, the method may comprise (i) providing an aqueous solution comprising the TCV, optionally wherein the aqueous solution and (ii) mixing the RNP (which is or comprises a base editor complexed with a gRNA) with the aqueous solution.

### BRIEF DESCRIPTION OF THE DRAWING

FIG. 1A is a schematic of the transgene construct of Ai14 mice and approximate locations of gRNA target sites. Ai14 mice harbor a modification at the Rosa26 locus with a ubiquitous CAG promoter followed by a floxed-stop cassette (three repeats of the SV40 polyadenylation (poly A) sequence) that prevents expression of the tdTomato fluorescent marker protein. The Ai14 strain was originally designed and generated by Madisen et al. (Madisen et al., Nat Neurosci. 2010 January; 13 (1): 133-140), along with several other reporter mouse strains having the same stop cassette, for Cre-mediated recombination at the loxP sites leading to excision of the stop cassette and tdTomato expression.

FIG. 1B shows the DNA sequence of an Ai14 mice transgene segment starting with the 3′end portion of the CAG promoter, encompassing two loxP sites (underlined, having SEQ ID NO: 2), which sandwich three SV40 terminator sequences (gray highlight, having SEQ ID NO: 3; within the gray highlight, AATAAA in bold encodes to the polyadenylation (polyA) motif (AAUAAA) and GTTTGT in bold italics encodes to the GU-rich region of the corresponding mRNA), and ending with the start codon (ATG) for the tdTomato gene. Sequences corresponding to the LaRo gRNA (light blue highlight), LoxP gRNA (blue), PS2 gRNA (red), and PS3 gRNA (arrowed) are also indicated. PAM sequences (NGG) are shown in bold.

FIGS. 2A-2B provide exemplary results from Example 2. FIG. 2A shows primary cortical neurons (from Ai14 mice) ex vivo subjected to CRISPR-mediated gene editing by LoxP gRNA (Single Guide) or a mixture of LaRo gRNA, PS2 gRNA, PS3 gRNA, and LoxP gRNA (Multiple Guides D), analyzed by fluorescent microscopy. FIG. 2B shows levels of cells with red fluorescence in Multiple Guide groups (subjected to CRISPR-mediated gene editing by different combinations of multiple gRNAs) relative to Single Guide group.

FIGS. 3A-3B provide exemplary results from Example 3. FIG. 3A provides exemplary fluorescent microscopy of corneal sections from different treatment groups. FIG. 3B. provides exemplary fluorescent microscopy of a corneal section from Multiple Guide group with an arrow indicating red fluorescence observed in the iris.

FIG. 4 provides exemplary results from Example 4. % cells with red fluorescence were compared between groups subjected to CRISPR-mediated gene editing by a mixture of LaRo gRNA, PS2 gRNA, PS3 gRNA, and LoxP gRNA, in the presence or absence of a ssODN having homology arms of varied lengths.

FIG. 5 provides exemplary results from Example 5. % cells with red fluorescence were compared between groups subjected once on DIV6 or twice on DIV3 and DIV6 to CRISPR-mediated gene editing by a mixture of LaRo gRNA, PS2 gRNA, PS3 gRNA, and LoxP gRNA, in the presence or absence of a ssODN having homology arms of varied lengths.

FIGS. 6A-6B provide exemplary results from Example 6. FIG. 6A provides representative immunocytochemistry observed in different treatment groups. Images taken at 20× magnification. Scale bar=50 μM. FIG. 6B provides exemplary comparison of % FLAG+ cells among PAX6+ cells (left) and % cells containing the intended gene alternation (right).

### DETAILED DESCRIPTION OF THE INVENTION

### Definitions

All references cited herein, including patent documents and non-patent documents, are hereby incorporated by reference in their entirety. Nothing herein is to be construed as an admission that the present invention is not entitled to antedate such disclosure by virtue of prior invention.

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

It should be understood that, unless clearly indicated otherwise, in any methods disclosed or claimed herein that comprise more than one step, the order of the steps to be performed is not restricted by the order of the steps specifically cited.

Unless defined otherwise, all technical and scientific terms used herein have the same meaning as commonly understood by one of ordinary skill in the art to which this disclosure belongs.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

The term “base editor” as used herein refers to an agent comprising a polypeptide that is capable of converting one base to another within a nucleic acid sequence (e.g., DNA or RNA). In some embodiments, the base editor is capable of deaminating a base within a nucleic acid. In some embodiments, the base editor is capable of deaminating a base within a DNA molecule.

In some embodiments, the base editor is capable of deaminating an adenine (A) in DNA, in which case the base editor is referred to adenine base editor (ABE). In certain embodiments, an ABE (e.g., when in a cell) may deaminate A bases within a target DNA span to result in inosine bases, which are then converted to G bases during DNA replication, thereby converting target A: T base pairs to G: C base pairs. In some embodiments, the base editor is capable of deaminating a cytosine (C) in DNA, in which case the base editor is referred to cytosine base editor (CBE). In certain embodiments, a CBE (e.g., when in a cell) may deaminate C bases within a target DNA span to result in U bases, which are then converted to T bases during DNA replication, thereby converting target C: G base pairs to T:A base pairs. In some embodiments, the base editor is capable of deaminating both A and C bases in DNA, in which case the base editor is referred to dual base editor (DE). In certain embodiments, a DE (e.g., when in a cell) may deaminate A and C bases within a target DNA span to result in inosine and U bases, respectively, which are then converted to G and T bases, respectively, during DNA replication, thereby converting target A: T base pairs to G: C base pairs and target C: G base pairs to T:A base pairs.

In some embodiments, the base editor is or comprises a protein (e.g., a fusion protein) comprising a Cas-derived platform protein linked to a deaminase. In certain embodiments, the Cas-derived platform protein is capable of forming a complex with a gRNA and binding to a target DNA span via the gRNA. In particular embodiments, the Cas-derived platform protein is or comprises a Cas nickase. In certain embodiments, the deaminase may be an adenine deaminase (in case of ABE), a cytidine deaminase (in case of CBE), or a dual deaminase (in case of DE). In certain embodiments, the base editor, e.g., if capable of converting a base to a U base, may further comprise an inhibitor of a uracil DNA glycosylase (UDG), also referred to as a uracil DNA glycosylase inhibitor (UGI). In particular embodiments, an ABE may comprise an adenine deaminase and a Cas nickase, In particular embodiments, a CBE may comprise a cytidine deaminase, a Cas nickase, and one or more UGIs. In particular embodiments, a DE may comprise a dual deaminase, a Cas nickase, and one or more UGIs.

The term “cargo” or “cargo molecule” as used herein is one or more materials carried by and/or encapsulated by/in a TCV according to the present disclosure. In some embodiments, the combination of materials carried by a TCV may be collectively referred to as a “cargo”. In some embodiments, a TCV may carry an endonuclease protein such as Cas9 as a cargo. In some embodiments, a TCV may carry one or more gRNAs as a cargo. In some embodiments, a TCV may carry template DNA as a cargo. In some embodiments, a TCV may carry a combination of an endonuclease protein and a guide RNA as a cargo, optionally in a form of a RNP formed by the gRNA and the endonuclease. In some embodiments, a TCV may carry a mixture of different RNPs comprising different gRNAs. In some embodiments, a TCV may carry a polynucleotide encoding an endonuclease protein such as Cas9 as a cargo. In some embodiments, a TCV may carry a polynucleotide encoding one or more gRNAs as a cargo. In some embodiments, a TCV may carry (i) a polynucleotide encoding an endonuclease protein such as Cas9 and (ii) a polynucleotide encoding one or more gRNAs as a cargo. In some embodiments, a TCV may carry (i) a polynucleotide encoding an endonuclease protein such as Cas9 and (ii) one or more gRNAs as a cargo. In some embodiments, a TCV may carry (i) an endonuclease protein such as Cas9 and (ii) a polynucleotide encoding one or more gRNAs as a cargo. In some embodiments, a TCV may carry a polynucleotide encoding both an endonuclease protein such as Cas9 and one or more gRNAs as a cargo. Optionally, any of such TCVs may further carry a template DNA and/or a polynucleotide encoding a template RNA.

The term “cholesterol derivative” as used herein, in its broadest sense, encompasses any derivatives of cholesterol. Non-limiting examples of cholesterol derivatives include: DC-Chol (N,N-dimethyl-N-ethylcarboxamidocholesterol), 1,4-bis(3-N-oleylamino-propyl) piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE) (US20210220273A1).

Clustered regularly interspaced short palindromic repeats (CRISPR)/CRISPR-associated (Cas) systems are a class of genome-editing tools that target desired genomic sites in mammalian cells. A CRISPR/Cas system involves at least one Cas endonuclease and a gRNA. Typically, the Cas endonuclease may recognize a protospacer adjacent motif (PAM) sequence specific to the Cas endonuclease (with certain Cas types, a protospacer flanking site (PFS) instead of a PAM) in the target gene (sense or antisense strand) and if the gRNA is able to hybridize with a target sequence in the target gene proximate to the PAM or PFS site, the Cas endonuclease may mediate cleavage of the target gene at about 2-6 nucleotides upstream of the PAM site or a particular position relative to the PAM or PFS site. Class 2 Type II CRISPR/Cas systems use Cas9 endonuclease, and for example the PAM sequence for Streptococcus pyogenes Cas9 (SpCas9) is 5′-NGG-3′. SpCas9 is targeted to a genomic site by complexing with a guide RNA that hybridizes to an approximately 17-24-nucleotide DNA sequence immediately preceding an 5′-NGG-3′ motif (where “N” can be any nucleotide) recognized by SpCas9 (e.g., a (N)₁₇₋₂₄NGG target DNA sequence; “N” represents any nucleotide). This results in a double-strand break (DSB) between the third and fourth nucleotides upstream of the NGG motif. The DSB instigates either non-homologous end-joining (NHEJ), which typically leads to the introduction of one or more nucleotide insertions or deletions resulting in frameshift mutations that knock out gene alleles, or homology-directed repair (HDR), which can be exploited with the use of an exogenously introduced double-strand or single-strand template DNA to knock in or correct a mutation in the genome.

Any appropriate Cas endonucleases may mediate CRISPR-mediated gene editing. In some embodiments, a Cas endonuclease (as a protein) or a Cas endonuclease-encoding polynucleotide (e.g., DNA or RNA) may be used. In some embodiments, the Cas endonuclease may be a Cas of Class 1 CRISPR/Cas system (Type I, III, or IV) or Class 2 CRISPR/Cas system (Type II, V, or VI). In some embodiments, the Cas endonuclease may be Cas 9, Cas3, Cas8a2, Cas8b, Cas8c, Cas10, Cas11, Cas12, Cas12a or Cpf1, Cas13, Cas13a, C2c1, C2c3, or C2c2. In some embodiments, the Cas endonuclease may be a class 2 Cas endonuclease. In some embodiments, the Cas endonuclease may be a type II, V, or VI Cas endonuclease. In certain embodiments, the Cas endonuclease is Cas9. In certain embodiments, the Cas9 may be Cas9 of Streptococcus pyogenes (SpCas9), Staphylococcus aureus (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of spCas9 (e.g., CP1012-SpCas9, CP1028-SpCas9, CP1041-SpCaS9, CP1249-SpCas9, and CP1300-SpCas9; see e.g., Oakes et al., Cell. 2019 Jan. 10;176 (1-2): 254-267.e16), eSpCas9, HypaCas9, FokI-Fused dCas9, xCas9, SpRY (variant of SpCas9), or SpG (variant of SpCas9), which are collectively referred to as Cas9 herein. In some embodiments, the Cas12a may be Cas12a of Lachnospiraceae bacterium ND2006 (LbCas12a), Cas12a of Acidaminococcus sp. BV3L6 (AsCas12a), or Cas12a of Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a.

Cas endonucleases of different bacterial origins often recognize different PAM sequences and/or different cleavage accuracy or specificity. In some cases, the type of Cas endonuclease to use may be selected based on the presence or absence or a certain PAM sequence in the target gene.

In some embodiments, the Cas endonuclease may be a wild-type (WT) SpCas9. WT SpCas9 may comprise the amino acid sequence of SEQ ID NO: 600. In some embodiments, the Cas endonuclease may be a variant SpCas9. A variant SpCas9 may comprise one or more amino acid modifications relative to SEQ ID NO: 600.

In embodiments, the Cas9 variant may comprise a substitution at position 80 of SEQ ID NO: 600, e.g., includes a leucine at position 80 of SEQ ID NO: 600 (i.e., comprises, e.g., consists of, SEQ ID NO: 600 with a C80L substitution). In embodiments, the Cas9 variant may comprise a substitution at position 574 of SEQ ID NO: 600, e.g., includes a glutamic acid at position 574 of SEQ ID NO: 600 (i.e., comprises, e.g., consists of, SEQ ID NO: 600 with a C574E substitution). In embodiments, the Cas9 variant may comprise a substitution at position 80 and a substitution at position 574 of SEQ ID NO: 600, e.g., includes a leucine at position 80 of SEQ ID NO: 600, and a glutamic acid at position 574 of SEQ ID NO: 600 (i.e., comprises, e.g., consists of, SEQ ID NO: 600 with a C80L substitution and a C574E substitution). Without being bound by theory, it is believed that such substitutions improve the solution properties of Cas9.

In embodiments, the Cas9 variant may comprise a substitution at position 147 of SEQ ID NO: 600, e.g., includes a tyrosine at position 147 of SEQ ID NO: 600 (i.e., comprises, e.g., consists of, SEQ ID NO: 600 with a D147Y substitution). In embodiments, the Cas9 variant may comprise a substitution at position 411 of SEQ ID NO: 600, e.g., includes a threonine at position 411 of SEQ ID NO: 600 (i.e., comprises, e.g., consists of, SEQ ID NO: 600 with a P411T substitution). In embodiments, the Cas9 variant may comprise a substitution at position 147 and a substitution at position 411 of SEQ ID NO: 600, e.g., includes a tyrosine at position 147 of SEQ ID NO: 600, and a threonine at position 411 of SEQ ID NO: 600 (i.e., comprises, e.g., consists of, SEQ ID NO: 600 with a D147Y substitution and a P411T substitution). Without being bound by theory, it is believed that such substitutions improve the targeting efficiency of Cas9, e.g., in yeast.

In embodiments, the Cas9 variant may comprise a substitution at position 1135 of SEQ ID NO: 600, e.g., includes a glutamic acid at position 1135 of SEQ ID NO: 600 (i.e., comprises, e.g., consists of, SEQ ID NO: 600 with a D1135E substitution). Without being bound by theory, it is believed that such substitutions improve the selectivity of Cas9 for the NGG PAM sequence versus the NAG PAM sequence.

In embodiments, Cas9 may be a variant SpCas9 that includes one or more substitutions relative to SEQ ID NO: 600 that introduce an uncharged or nonpolar amino acid, e.g., alanine, at certain positions. In embodiments, Cas9 may be a variant SpCas9, which, relative to SEQ ID NO: 600, includes a substitution at position 497, a substitution at position 661, a substitution at position 695 and/or a substitution at position 926 of SEQ ID NO: 600, for example a substitution to alanine at position 497, position 661, position 695 and/or position 926 of SEQ ID NO: 600. In embodiments, Cas9 has a substitution only at position 497, position 661, position 695, and position 926 of SEQ ID NO: 600, relative to SEQ ID NO: 600, e.g., where each substitution is to an uncharged amino acid, for example, alanine. Without being bound by theory, it is believed that such substitutions reduce the cutting by Cas9 at off-target sites.

It will be understood that the substitutions described herein to Cas9 may be combined, and may be combined with any of the fusions or other modifications described herein. In certain embodiments, Cas9 may comprise or consist of any of the amino acid sequences of SEQ ID NOS: 600-611. In particular embodiments, Cas9 may comprise or consist of the amino acid sequence of SEQ ID NO: 600. Some of the Cas endonucleases and variants thereof that may be used in the present invention further include but are not limited to those described in, e.g., WO2017115268A1 or SEQ ID NO: 1-612 of U.S. Pat. No. 11,118,177.

The term “destabilizing agent” as used herein encompasses any agents that destabilizes the cargo of a TCV according to the present disclosure. In some embodiments, a destabilizing agent may destabilize or degrade a nucleic acid cargo such as a gRNA, a protein cargo such as a Cas endonuclease, and/or a RNP. Exemplary destabilizing agents include but are not limited to: organic solvents such as ethanol and detergents such as sodium dodecyl sulfate. In some embodiments, a TCV or a composition according to the present disclosure may be substantially free of destabilizing agents. In some embodiments, a TCV or a composition according to the present disclosure may be substantially free of organic solvents and detergents. In some embodiments, a TCV or a composition according to the present disclosure may be substantially free of organic solvents. In some embodiments, a TCV or a composition according to the present disclosure may be substantially free of detergents. In some embodiments, such a TCV or a composition according to the present disclosure may be substantially, essentially, or entirely free of ethanol, methanol, isopropanol, tetrahydrofuran (THF), dimethyl sulfoxide (DMSO), dimethyl formamide (DMF), and acetonitrile (ACN). In some embodiments, a TCV or a composition according to the present disclosure may have a final ethanol concentration of 5% (v/v) or below, preferably 0.5% (v/v).

“Guide RNA” or “gRNA”, as used herein in relation to the CRISPR/Cas gene editing (also referred to as “CRISPR-mediated gene editing) or base editing, refers to a RNA fragment (e.g., single guide RNA (“sgRNA”)) or a hybrid of two RNA fragments (e.g., dual guide RNA (“dgRNA”)) that binds to a target DNA sequence and guide a Cas endonuclease protein or a variant thereof a base editor, to the specific site of a DNA (e.g., in a genome) to allow for Cas-mediated cleavage of a DNA molecule. In some embodiments, gRNA may be dgRNA comprising: (I) a crispr RNA (crRNA), which comprises (i) a targeting sequence of about 15-75 nucleotides that is complementary to (or comprising some mismatches relative to) the target DNA sequence and (ii) a crRNA flagpole sequence; and (II) a trans-activating crispr RNA (tracrRNA), which comprises (i) a tracrRNA flagpole sequence and (ii) tracrRNA endonuclease binding domain, which serves as a binding scaffold for the Cas endonuclease, wherein the crRNA and tracrRNA hybridize with each other via the flagpole sequences. In some embodiments, a gRNA may be sgRNA comprising (I) a crRNA sequence linked to (II) a trarRNA sequence as a single polynucleotide. In certain embodiments, such a dgRNA or sgRNA may be used with a Cas9 endonuclease or a variant thereof or a base editor comprising such. In some embodiments, a gRNA may be a sgRNA comprising a crispr RNA (crRNA), which comprises (i) a direct repeat (5′ handle) sequence of about 10-30 nt (e.g., 20 nt) and (ii) a targeting sequence of about 15-30 (e.g., 20-25, such as 23) nucleotides that is complementary to (or comprising some mismatches relative to) the target DNA sequence. In certain embodiments, such a dgRNA or sgRNA may be used with a Cas12a endonuclease or a variant thereof or a base editor comprising such.

In some embodiments, the dgRNA and sgRNA may have the following formats:

dgRNA

crRNA (polynucleotide 1 having a crRNA sequence):
[targeting sequence]-[crRNA flagpole sequence]-[(optional) crRNA first flagpole extension]-[(optional) crRNA second flagpole extension]
the sequence of [crRNA flagpole sequence]-[(optional) crRNA first flagpole extension]-[(optional) crRNA second flagpole extension] may be referred to herein as “crRNA backbone sequence”. tracrRNA (polynucleotide 2 having a tracrRNA):
[(optional) tracrRNA first extension]-[tracrRNA flagpole sequence]-[tracrRNA endonuclease binding domain]
   sgRNA (having a crRNA sequence linked to a tracrRNA sequence)
[targeting sequence]-[crRNA flagpole sequence]-[(optional) crRNA first flagpole extension]-[(optional) linker]-[(optional) tracrRNA first extension]-[tracrRNA flagpole sequence]-[tracrRNA endonuclease binding domain]
the sequence of [crRNA flagpole sequence]-[(optional) crRNA first flagpole extension]-[(optional) linker]-[(optional) tracrRNA first extension]-[tracrRNA flagpole sequence]-[tracrRNA endonuclease binding domain] may be referred to herein as “sgRNA backbone sequence”.

In some embodiments, the crRNA flagpole sequence may comprise SEQ ID NO: 101 or 102. In some embodiments, the optional crRNA first flagpole extension may comprise the nucleic acid sequence, UGCUG. In some embodiments, the optional crRNA second flagpole extension may comprise the nucleic acid sequence, UUUUG. In some embodiments, the optional tracrRNA first extension may comprise the nucleic acid sequence, CAGCA. In some embodiments, the tracrRNA flagpole sequence may comprise SEQ ID NO: 106 or 107. In some embodiments, the tracrRNA endonuclease binding domain may comprise SEQ ID NO: 108. In some embodiments, the tracrRNA endonuclease binding domain may further comprise or may be followed by one or more uracil based, e.g., 5′-U-3′, 5′-UU-3′, 5′-UUU-3′, 5′-UUUU-3′, 5′-UUUUU-3′, 5′-UUUUUU-3′, 5′-UUUUUUU-3′, or 5′-UUUUUUUU-3′.

In certain embodiments, the crRNA flagpole sequence may comprise SEQ ID NO: 101 and the tracrRNA flagpole sequence may comprise SEQ ID NO: 106. In certain embodiments, the crRNA flagpole sequence may comprise SEQ ID NO: 102 and the tracrRNA flagpole sequence may comprise SEQ ID NO: 107. In some embodiments, the optional linker which links a crRNA and tracrRNA in a sgRNA may comprise or consist of the nucleic acid sequence of GAAA.

In some embodiments, a sgRNA may comprise a sgRNA backbone sequence (the sequence which is placed 3′ to a targeting sequence in a sgRNA) of any of SEQ ID NOS: 111-114. In certain embodiments, the sgRNA backbone sequence may be followed by one or more uracils. In particular embodiments, the sgRNA backbone sequence may be followed by 1-10 uracils, such as 3 uracils, 4 uracils, 5 uracils, 6 uracils, 7 uracils, or 8 uracils.

In some embodiments, a dgRNA may comprise (I) a crRNA sequence comprising a crRNA backbone sequence (the sequence which is placed 3′ to a targeting sequence in a crRNA) comprising SEQ ID NO: 115 and (II) a tracrRNA sequence comprising SEQ ID NO: 116. In some embodiments, a dgRNA may comprise (I) a crRNA sequence comprising a sgRNA backbone sequence (the sequence which is placed 3′ to a targeting sequence in a crRNA) comprising SEQ ID NO: 117 and (II) a tracrRNA sequence comprising SEQ ID NO: 118.

When Cas9 is used, in some embodiments, the targeting sequence may comprise a GC content in the range of 40-80%, and in some embodiments, and the targeting sequence may have a length of 17-24 nucleotides.

In some embodiments, a gRNA according to the present disclosure may comprise one or more modifications. In some embodiments, the modification may be selected from the group consisting of: 2′-O—C1-4alkyl such as 2′-O-methyl (2′-OMe), 2′-deoxy (2′-H), 2′-O—C1-3alkyl-O-C1-3alkyl such as 2′-methoxyethyl (2′-MOE), 2′-fluoro (2′-F), 2′-amino (2′—NH2), 2′-arabinosyl (2′-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in 1 form (1-sugar), and 4′-thioribosyl nucleotide. In some embodiments, the modification is an internucleotide linkage modification selected from the group consisting of: phosphorothioate, phosphonocarboxylate, thiophosphonocarboxy late, alkylphosphonate, and phosphorodithioate. In some embodiments, the modification is selected from the group consisting of: 2-thiouracil (2-thioU), 2-thiocytosine (2-thioC), 4-thiouracil (4-thioU), 6-thioguanine (6-thioG), 2-aminoadenine (2-aminoA), 2-aminopurine, pseudouracil, hypoxanthine, 7-deazaguanine, 7-deaza-8-azaguanine, 7-deazaadenine, 7-deaza-8-azaadenine, 5-methylcytosine (5-methylC), 5-methyluracil (5-methylU), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (5-allylU), 5-allylcytosine (5-allyIC), 5-aminoallyluracil (5-aminoallylU), 5-aminoallyl-cytosine (5-aminoallylC), an abasic nucleotide, Z base, P base, Unstructured Nucleic Acid (UNA), isoguanine (isoG), isocytosine (isoC), and 5-methyl-2-pyrimidine. In particular embodiments, a gRNA may comprise (i-1) 2′-O-methylation further optionally at first three and last three bases and/or (i-2) one or more 3′ phosphorothioate bonds, further optionally between first three and last two bases.

A targeting sequence of a gRNA may be any appropriate length. The most frequently used targeting sequence length is 20 nt. In some embodiments, a gRNA longer than 20 nt may be used. For example, Ran et al. demonstrated that longer gRNAs are commonly cleaved to a shorter length so that the targeting sequence is e.g., 20 nt and thus the complementarity in the segment in excess of 20 nt may not be important, i.e., may or may not be complementary to a target sequence (Ran et al., Cell. 2013 Sep. 12;154 (6): 1380-9). In some embodiments, a gRNA shorter than 20 nt may be used. For example, Fu et al. demonstrated that truncated (i.e., <20 nt) gRNAs, which is as short as 17, 18, or 19 nt, may also target the same target as a corresponding 20 nt-long gRNA and perhaps even may have decreased off-target effects (Fu et al. Nat Biotechnol. 2014 March; 32 (3): 279-284).

A targeting sequence of a gRNA may or may not comprise a mismatch relative to the target sequence. In some cases, a mismatch at a particular position may reduce gRNA specificity to the target sequence. For example, in the context of SpCas9, Cong et al demonstrated that complementarity at up to 11 nt from the 3′-end of a targeting sequence is more important than that at a more upstream region (Cong et al., Science. 2013 Feb. 15; 339 (6121): 819-823). Again in the context of SpCas9, Zheng et al demonstrated that the core sequence which is from the 4^{th} to the 7^{th} nt from the 3′-end is more sensitive to target mismatch compared to the rest of the targeting sequence (Zheng et al., Sci Rep. 2017 Jan. 18; 7:40638). Therefore, in some embodiments, a gRNA targeting sequence may comprise a mismatch relative to its target sequence outside of such a core sequence.

The term “helper lipid” or “structural lipid” as used herein refers to a type of lipid that may be comprised in a TCV in addition to an ionizable cationic lipid. In some embodiments, a helper lipid may be a non-cationic lipid and may be neutral, zwitterionic, or anionic lipid. In some embodiments, a helper lipid may be a lipid that carries a net negative charge at a selected pH, such as physiological pH. Without wishing to be bound by theory, helper lipids in TCVs in general are used to provide particle stability and/or biocompatibility and/or to enhance cargo delivery efficiency. Non-limiting helper lipids include, but are not limited to dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. In some embodiments, a helper lipid is dioleoylphosphatidylethanolamine (DOPE).

The term “ionizable cationic lipid” as used herein, refers to any lipid that carries a net neutral charge at about physiological pH but is capable of becoming positively charged at a lower pH, e.g., pH below about 7, below about 6.5, below about 6, below about 5.5, below about 5, below about 4.5, below about 4, below about 3.5, or below about 3, typically at pH below about 6.5 or below about 6.5-7. Without wishing to be bound by theory, a net neutral charge helps toxicity, and positive charges under a low pH may be useful in forming a complex with a negatively charged cargo such as a nucleic acid molecule and/or protein. Becoming positive charges under as the pH decreases may also help release of the cargo from an endosome once in a cell (endosomal escape), e.g., by taking protons in an endosome thereby destabilizing and bursting the endosome. For example, N-dimethyl-2,3-dioleyloxy) propylamine (DODMA), 1,2-dioleoyl-3-dimethylammonium propane (“DODAP”), 1,2-Dilinoleoyl-3-dimethylaminopropane (DLinDAP), 1,2-DiLinoleyloxy-N,N-dimethylaminopropane (DLinDMA), N,N-dimethyl-2,2-di-(9Z,12Z)-9,12-octadecadien-1-yl-1,3-dioxolane-4-ethanamine (KC2), and (6Z,9Z,28Z,31Z)-heptatriaconta-6,9,28,31-tetraen-19-yl 4-(dimethylamino) butanoate (MC3) are ionizable cationic lipids.

The term “permanently cationic lipids” as used herein, refers to any lipid that carries a net positive charge without pKa or pKa>8. For example, N-(1-(2,3-dioleyloxyl) propyl)-N,N,N-trimethylammonium chloride (DOTMA), N-(1-(2,3-dioleyloxyl) propyl)-N-2-(sperminecarboxamido)ethyl)-N,N-dimethyl-ammonium trifluoracetate (DOSPA) (which is a lipid component of Lipofectamine®), and N-(1-(2,3-dioleoyloxy) propyl)-N,N,N-trimethylammonium chloride (DOTAP) are permanently cationic lipids.

Examples of cationic lipids (including ionizable cationic lipids) may include, for example, N,N-dimethyl-2,3-dioleyloxy) propylamine (DODMA), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxyl) propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAR.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino) butanoate (MC3), or a mixture thereof.

Additional examples of cationic lipids (including ionizable cationic lipids) include, but are not limited to, N-(2,3-dioleyloxyl) propyl-N,N-N-triethylammonium chloride (“DOTMA”); 1,2-Dioleyloxy-3-trimethylaminopropane chloride salt (“DOTAP.C1”); 3.beta.-(N-(N′,N′-dimethylaminoethane)-carbamoyl)cholesterol (“DC-Chol”), N-(1-(2,3-dioleyloxyl) propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”), and mixtures thereof. Additionally, a number of commercial preparations of cationic lipids can be used, such as, e.g., LIPOFECTIN® (available from GIBCO/BRL), and LIPOFECTAMINE® (available from GIBCO/BRL).

The term “complementary” or “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

The term “mutation” or “point mutation” as used herein in relation to nucleic acid or nucleotide sequence means a change in a nucleotide in a DNA or RNA molecule. A mutation may be a change from a nucleotide to another nucleotide or deletion of a nucleotide or an insertion of a nucleotide. When a mutation causes replacement of a nucleotide with another nucleotide in an open reading frame, the mutation may cause an amino acid substitution (“missense mutation”) or appearance of an early stop codon (“nonsense mutation”) leading to a shorter protein product or may not cause any changes in the protein product (“silent mutation”). When a mutation causes insertion or deletion of a nucleotide in an open reading frame, unless the number of insertion or deletion is divisible by three, the mutation changes the grouping of the codons to be read (“frame shift mutation”), causing dramatic changes in the protein sequence.

“Lipid-based TCVs” as used in are TCVs that comprise at least one lipid and encompass lipid nanoparticles. In some embodiments, a lipid-based TCV may comprise at least one ionizable cationic lipid. In some embodiments, a lipid-based TCV may comprise at least one helper lipid. In some embodiments, a lipid-based TCV may comprise at least one phospholipid. In some embodiments, a lipid-based TCV may comprise at least one cholesterol (or cholesterol derivative). In some embodiments, a lipid-based TCV may comprise, essentially consist of, or consist of at least one ionizable cationic lipid, at least one helper lipid, at least one phospholipid, and at least one cholesterol (or cholesterol derivative), and optionally polyethylene glycol (PEG) or PEG-lipid. Exemplary TCVs include but not are limited to those described in Applicant's WO2020077007A1. In some embodiments, a lipid-based TCV may comprise, essentially consist of, or consist of an ionizable cationic lipid, one or more phospholipids, and cholesterol, the ratio of which are about 20:30:10:40 in mol %. In some embodiments, a lipid-based TCV may comprise, essentially consist of, or consist of an ionizable cationic lipid, one or more phospholipids, cholesterol, and PEG-lipid, the ratio of which are about 20:30:10:39:1 in mol %. TCVs may be generated using gentle mixing such as repeated manual reciprocation of the TCV-generating fluid in a pipette, staggered herringbone micromixer (SHM), T-junction mixing or extrusion methods, or other TCV-mixing methods as desired.

In some embodiments, a lipid-based TCV and/or a composition according to the present disclosure may substantially, essentially, or entirely lack organic solvents and/or detergents, which may help improve the stability and/or integrity of the TCV and/or its cargo. In some embodiments, the manufacturing method of a TCV according to the present disclosure may contribute to such a characteristic.

In some embodiments, a TCV and/or a composition may be stored at a freezing temperature. In some embodiments, when a TCV and/or a composition may be prepared, a cryoprotectant may be added. In some embodiments, a cryoprotectant may comprise a sugar-based molecule. Non-limiting examples of cryoprotectants include sucrose, trehalose, and a combination thereof. In certain embodiments, at least one cryoprotectant may be or may comprise a sugar-based molecule, e.g., a sugar molecule or a derivative thereof. In particular embodiments, the at least one cryoprotectant may be sucrose, trehalose, or a combination thereof. In a particular embodiment, the at least one cryoprotectant may be sucrose. In some embodiments, the concentration of the at least one cryoprotectant contained in the TCV or composition may be about 1% to about 40%, about 3% to about 30%, about 5% to about 30%, about 10% to about 20%, or about 15%.

In some embodiments, a TCV and/or a composition according to the present disclosure, which may comprise at least one cryoprotectant, may be stable at a freezing temperature, optionally at about −20° C. or about −80° C., optionally for at least about one week, at least about two weeks, at least about three weeks, at least about a month, at least about two months, at least about four months, at least about five months, at least about 6 months, at least about 9 months, at least about a year, or at least about two year, or longer, or about one week to about two year, about two weeks to about a year, about three weeks to about nine month, about one to about six months, about one to five months, about one to four months, about one to three months, or about one to two months.

A TCV according to the present disclosure may be prepared by any appropriate methods. In some embodiments, a TCV may be prepared by (a) generating a first solution by dissolving all components of the TCV in ethanol; (b) providing a second solution, which is aqueous; (c) combining the first and second solutions; and (d) removing ethanol, optionally by dialysis or evaporation. In some embodiments, the first solution in step (a) may contain the TCV lipid components at about 20-35 mM. In some embodiments, the second solution in step (b) may be an acidic buffer and optionally may contain acetate and/or citrate (e.g., sodium acetate and/or sodium citrate), which optionally may be at about 25 mM. In some embodiments, the pH of the second solution in step (b) may be about 3-8, about 4-7, about 3.5-4.5, or about 4. In some embodiments, the combining in step (c) may be by gentle mixing (optionally repeated manual reciprocation of the TCV-generating fluid in a pipette), mixing using a staggered herringbone micromixer (SHM), T-junction mixing, or extrusion. In a particular embodiment, the removing in step (d) is by dialysis. In a particular embodiment, the suspension resulting from step (c) may be dialyzed against an acidic buffer. In yet a particular embodiment, the acidic buffer may have a pH of about 3-5, about 3.5-4.5, or about 4. In some instances, the acidic buffer may contain acetate and/or citrate, such as sodium acetate and/sodium citrate. In a particular embodiment, the dialysis is performed against a 1000-fold volume of 25 mM sodium acetate (approximately pH 4) buffer.

Encapsulation of a cargo by a TCV may be performed by any appropriate methods. In some embodiments, wherein the TCV comprise a RNP as a cargo, the RNP encapsulation by TCVs may be performed by any appropriate methods. In some embodiments, the encapsulation may be performed by (i) providing an aqueous solution comprising the TCV, optionally in an acidic buffer (e.g., pH of about 3-5, about 3.5-4.5, or about 4); and (ii) mixing a RNP solution containing one or more RNPs with the aqueous solution. Mixing may be effected under conditions suitable for the at least one RNP to be encapsulate within the TCV. In some embodiments, the aqueous solution in step.

The term “nuclease” as used herein refers to an enzyme capable of catalyzing the cleavage of phosphodiester bonds between nucleotides of nucleic acids. An “endonuclease” cleaves phosphodiester bonds to separating nucleotides in a polynucleotide other than the two end nucleotides. In the CRISPR/Cas system, which involves a gRNA and a CRISPR-associated (Cas) endonuclease, the Cas endonuclease recognizes a PAM sequence in the target gene (sense or antisense) and if the gRNA is able to hybridize with a target sequence of the target gene proximate to the PAM sequence, the Cas endonuclease may mediate cleavage of the target gene at about 2-6 nucleotides upstream of the PAM. The PAM sequence is specific to the Cas endonuclease. Any appropriate Cas endonucleases may be used in the invention disclosed herein. Appropriate Cas endonucleases include but are not limited to Cas9 of different bacterial species such as Streptococcus pyogenes (SpCas9, which recognizes the PAM sequence of 5′-NGG-3′), Staphylococcus aureus (SaCas9, which recognizes the PAM sequence of 5′-NNGRRT-3′), Streptococcus thermophilus (StCas9, which recognizes the PAM sequence of 5′-NGGNG-3′), Neisseria meningitidis (NmCas9, which recognizes the PAM sequence of 5′-NNNNGATT-3′), Francisella novicida (FnCas9, which recognizes the PAM sequence of 5′-NG-3′), Campylobacter jejuni (CjCas9, which recognizes the PAM sequence of 5′-NNNNACA-3′), Streptococcus canis (ScCas9, which recognizes the PAM sequence of 5′-NNGG-3′), Staphylococcus auricularis (SauriCas9, which recognizes the PAM sequence of 5′-NNG-3′), or any engineered variants thereof, including but not limited to SaCas9-HF, SpCas9-HF1, SpCas9-NG (which recognizes the PAM sequence of NGN or NG), KKHSaCas9 or SaCas9-KKH (which recognizes the PAM sequence of NNNRRT), circular permutants of spCas9 (e.g., CP1012-SpCas9, CP1028-SpCas9, CP1041-SpCaS9, CP1249-SpCas9, and CP1300-SpCas9; see e.g., Oakes et al., Cell. 2019 Jan. 10;176 (1-2): 254-267.e16), SpCas9-VQR/SpCas9-VRQR (which recognizes the PAM sequence of NGA), SpCas9-VRER (which recognizes the PAM sequence of NGCG), SpCas9-EQR (which recognizes the PAM sequence of NGAG), eSpCas9, HypaCas9, FokI-Fused dCas9, xCas9 (which recognizes the PAM sequence of NGN), SpRY (variant of SpCas9), SpG (variant of SpCas9). Other Cas endonuclease examples include Cas3, Cas8a2, Cas8b, Cas8c, Cas10, Cas11, Cas12, Cas12a or Cpf1, Cas13, Cas13a, C2c1, C2c3, and C2c2. In some embodiments, Cas9 may be a wild-type SpCas, e.g., comprising SEQ ID NO: 600 or its variant, e.g., comprising any of SEQ ID NOS: 601-611. Additional examples of appropriate Cas endonucleases include but are not limited to Cas12a of different bacterial species, such as Lachnospiraceae bacterium ND2006 (LbCas12a, which recognizes the PAM sequence of 5′-TTTV-3′), Acidaminococcus sp. BV3L6 (AsCas12a, which recognizes the PAM sequence of 5′-TTTV-3′, or engineered AsAcas12a (“enAsCas12a”) or high-fidelity enAsCas12a (“enAsCas12a-HF1”) (Kleinstiver et al., Nat Biotechnol. 2019 March;37 (3): 276-282)), and Francisella tularensis subsp. novicidain U112 (FnCas12a, which recognizes the PAM sequence of 5′-TTN-3′). Further examples of Cas12a endonucleases include but are not limited to BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, and SsCas12a.

The terms “nucleic acid”, “nucleic acid molecule”, and “polynucleotide” are used interchangeably herein and encompass any compounds that comprise a polymer of nucleotides linked via a phosphodiester bond. Exemplary nucleic acids include but are not limited to RNA and DNA molecules, including molecules comprising cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Other non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

The term “phospholipid” as used herein refers to any lipid comprising a phosphate group. Non-limiting examples of suitable phospholipids include: distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. In one embodiment, the phospholipid is distearoylphosphatidylcholine (DSPC).

The term “polyethylene glycol-lipid” or “PEG-lipid” as used herein refers to any lipid modified or conjugated to one or more polyethylene glycol (PEG) molecules. Without wishing to be bound by theory, containing PEG or a PEG-lipid in a TCV may help maintain TCV particle size (keep a TCV from getting too big) and/or help maintain particle stability in vivo. Some examples of PEG-lipids that are useful in the present invention may have a variety of “anchoring” lipid portions to secure the PEG to the surface of the lipid-based TCVs. Non-limiting examples of suitable PEG-lipids include PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20) which are described in U.S. Pat. No. 5,820,873, incorporated herein by reference, PEG-modified dialkylamines, and PEG-modified 1,2-diacyloxypropan-3-amines. Particularly examples include PEG-modified diacylglycerols and dialkylglycerols.

The phrase “pharmaceutically acceptable” refers to molecular entities and compositions that are physiologically tolerable and do not typically produce an unintended and intolerable response such as an allergic response, when administered to a human. In certain embodiments, the term “pharmaceutically acceptable”, as used herein, means approved by a regulatory agency of the Federal or a state government or listed in the U.S. Pharmacopeia or other generally recognized pharmacopeia for use in animals, and more particularly in humans. The phrase “pharmaceutically acceptable carrier” refers to substances or collections of substances capable of being combined with an active ingredient that is suitable for use in contact with the cells or tissues of mammals for purposes of a therapeutic treatment in the mammals under anticipated exposure conditions.

The term “ribonucleoprotein”, “RNP”, or “RNP complex” as used herein refers to a complex of one or more RNA molecules and an RNA-binding protein. In the context of the CRISPR/Cas system, an RNP may be a complex of a gRNA and a Cas endonuclease. The gRNA may be for example a sgRNA or a dgRNA. Such a RNP may be generated by any appropriate methods. In some embodiments, the RNP may be formed by mixing Cas9 and gRNA at an approximately equimolar ratio, optionally for about 5 minutes.

“Single-strand oligo DNA nucleotides” or “ssODN” as used herein refers to a short DNA fragment of a single strand comprising a particular polynucleotide sequence that may be useful for some of the embodiments disclosed herein. In one aspect, ssODN may be used as part of CRISPR/Cas-mediated gene editing disclosed herein and may function as a DNA template (may also referred to as a DNA repair template) to mediate a knock-in of a sequence of interest through the Cas9-mediated double-strand break site. Such a knock-in may be via homology-directed repair (HDR). In some embodiments, a ssODN may have homology to the strand that initiates repair in the direction of a desired modification. In some embodiments, a ssODN may comprise (i) a 5′ homology arm and (ii) a 3′ homology arm, and optionally (iii) a central region comprising one or more desired nucleic acids, sandwiched by the 5′ homology arm and the 3′ homology arm. Such a homology arm may comprise approximately 10-2500 nucleotides (nt). 5′ and 3′ homology arms often have the same or similar nucleotide lengths (e.g., 0 or 1 to 10 nt difference), but 5′ and 3′ homology arms that significantly differ in length may also be used as long as the ssODN mediate and/or assist an intended gene repair. 5′ and/or 3′ homology arms may be 100% complementary to the corresponding sequence in the original DNA sequence before gene editing or may have one or more (a few) mutations (e.g., silent mutation) relative to the corresponding sequence in the original DNA sequence before gene editing. In some embodiments, ssODN may have one or more mutations at the PAM sequence (or its reverse (or antisense) sequence of to the PAM sequence, i.e., the opposite strand) and/or at one or more of the 5′-neighbouring bases of the PAM (or the 3′-neighbouring bases of the reverse (or antisense) sequence corresponding to the PAM). In some cases, such a mutation(s) helps prevent or reduce Cas-mediated cleavage of the ssODN itself or of a gene-edited DNA molecule. In some embodiments, a ssODN may comprise complementarity to the gRNA strand. In some embodiments, a ssODN may comprise a total length of approximately 40-5000 nucleotides (nt). As a template DNA, a double-stranded DNA template may also be used instead. In such a case, one of the strands of the template DNA may comprise the same sequence as a desired ssODN and the other strand has a sequence complementary thereto.

A “subject” as used herein, which may be interchangeably referred to as “patient”, “individual”, or “animal”, refers to a vertebrate including members of the mammalian species, such as canine, feline, lupine, mustela, rodent (racine, murine, etc.), equine, bovine, ovine, caprine, porcine species, and primates including humans. In specific embodiments, the subject is a human. In some embodiments, a subject may have or have a risk of developing a target disease. In specific embodiments, a subject may have or have a risk of developing SCD.

The term “target cell” or “host cell” as used herein refers to a cell in which the cargo of a TCV according to the present disclosure is intended to function. A TCV according to the present disclosure may be engineered to specifically carry its cargo in a target cell, for example by comprising one or more targeting moiety on the surface.

The term “target disease”, as used herein, which may be used interchangeably with “target disorder” or “target condition”, refers to a disease, disease, or condition that a TCV containing a cargo or a composition containing such a TCV according to the present disclosure is intended to treat, prevent, or ameliorate. A TCV according to the present disclosure may carry its cargo into a target cell, thereby altering a target gene or target gene expression and thus prevent, treat, or ameliorate a target disease.

The term “target gene” or “target gene of interest” as used herein is a gene (including the gene itself and in some cases a polynucleotide region that regulates the expression of the gene such as a promoter and/or an enhancer of the gene) whose sequence is to be altered (e.g., disrupted, partially or entirely removed, or partially or entirely replaced with an intended sequence, for example by a endonuclease (such as Cas9) and a guide RNA) by a cargo of a TCV according to the present disclosure. In general, “target gene” may be any gene of interest in a target cell. The sequence of “target gene” may be the sense strand sequence or the antisense strand sequence of the gene.

The term “target sequence” or “target polynucleotide sequence” as used herein is the sequence of a polynucleotide that a targeting sequence of a gRNA according to the present disclosure may interact with in a cell. A target sequence may be fully complementary with a targeting sequence or there may be one or more mismatches. In some embodiments, there may be one, two, three, four, or five mismatches. In some embodiments, a target sequence and a targeting sequence may share 100% sequence identity, about 99% sequence identity, about 98% sequence identity, about 97% sequence identity, about 96% sequence identity, about 95% sequence identity, about 94% sequence identity, about 93% sequence identity, about 92% sequence identity, about 91% sequence identity, about 90% identity, about 89% sequence identity, about 87% sequence identity, about 86% sequence identity, about 85% sequence identity, about 84% sequence identity, about 83% sequence identity, about 82% sequence identity, about 81% sequence identity, about 80% identity, about 79% sequence identity, about 78% sequence identity, about 77% sequence identity, about 76% sequence identity, or about 75% sequence identity.

The term “therapeutically effective amount/dose” refers to the quantity of a TCV or a pharmaceutical composition comprising such a TCV or its cargo that is sufficient to provide a therapeutic effect (which may be based on, e.g., the number or percentage of target cells in which the intended target gene alteration occurred, the overall change in the target gene expression, the amelioration of one or more symptom, the number or percentage of target cells exhibiting an intended phenotype such as morphology, etc) upon administration to a subject.

The term “transfection competent vesicle” or “TCV” as used herein, in its broadest sense, encompasses any materials capable of carrying one or more cargoes, such as but not limited to a nucleic acid molecule (e.g., a DNA or a RNA) and/or a nucleic acid molecule complexed with a protein or peptide, into a cell. Examples of TCVs include but are not limited to: compounds, such as calcium phosphate, polycations, cationic lipids, phospholipids, organic and nonorganic polymers, dendrimers, organic and nonorganic nanoparticles and nanobeads, and any combinations thereof; lipid-based compositions capable of carrying a nucleic acid molecule, such as liposomes and lipid nanoparticles (LNPs); plasmids; virus-like particles (VLPs); and viral vectors, such as retroviral, lentiviral, and adenoviral vectors. In some embodiments, a TCV may comprise a targeting moiety (e.g., antibody or antibody fragment such as a Fab fragment), which allows the TCV to carry its cargo preferentially into a target cell.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. In specific embodiments, the effect of the “treatment” may be evaluated by the amelioration of at least one measurable physical parameter of a disease, resulting from the administration of one or more therapies. The parameter may be, for example, gene expression profiles, the number of disease-affected cells, the percentage or frequency of disease-affected cells among the cells of the same lineage, disease-associated marker levels, and/or the presence or absence or levels of certain cytokines or chemokines or other disease-associated molecules and may not necessarily discernible by the patient. In some embodiments “treat”, “treatment,” or “treating” may result in and/or be evaluated based on the inhibition of the progression of a disease, either physically by, e.g., stabilization of a discernible symptom, physiologically by, e.g., stabilization of a physical parameter, or both. In some embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

A “vector” is a compound or a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, viruses, and virus-like particles (VLPs). Thus, the term “vector” includes an autonomously replicating plasmid, a self-replicating RNA, or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The present disclosure provides, among other things, reporter systems for CRISPR-mediated gene editing and gRNAs, RNPs, and compositions for the reporter systems. The present disclosure also provides methods of testing CRISPR-mediated gene editing. The present disclosure further provides compositions and methods for knocking out a DNA segment of interest. The present disclosure further provides compositions and methods of effecting CRISPR-mediated gene editing in the eye.

### Reporter System for CRISPR-Mediated Gene Editing

Applicant discovered that, in a reporter system in which a terminator sequence (and/or a stop codon) is followed by a reporter gene and the terminator sequence (and/or a stop codon) is flanked by two sites cleavable by CRISPR-mediated gene editing, additionally targeting the intervening sequence (the sequence between the two cleavable site) by CRISPR-mediated gene editing surprisingly enhances gene editing which is confirmed by reporter gene expression.

For example, in Example 2, targeting multiple sites using PS2 gRNA (targeting sequence of SEQ ID NO: 120; targeting sequence+PAM of SEQ ID NO: SEQ ID NO: 20) and PS3 gRNA (targeting sequence of SEQ ID NO: 130, targeting sequence+PAM of SEQ ID NO: SEQ ID NO: 30) designed by Applicant within an intervening DNA sequence (the DNA segment of SEQ ID NO: 80 and its complementary SEQ ID NO: 81) flanked by two cleavage sites targetable by LoRA gRNA (targeting sequence of SEQ ID NO: 140, targeting sequence+PAM of SEQ ID NO: SEQ ID NO: 40) and LoxP gRNA (targeting sequence of SEQ ID NO: 150, targeting sequence+PAM of SEQ ID NO: SEQ ID NO: 50), in addition to targeting using LoRA gRNA and LoxP gRNA, enhanced gene editing in Ai9 cells comprising a transgene segment comprising SEQ ID NO: 90.

Without wishing to be bound by theory, when excision of an intervening DNA flanked by two cleavage sites is intended, adding an additional cleavage(s) within the intervening DNA breaks the intervening DNA in smaller pieces, which may reduce the chance that the additionally cleaved intervening DNA participate in DNA repair activities of the host and thus may increase the relative likelihood of the intended joining of the two cleavage sites.

### Gene Editing

gRNAs

In one aspect, the present disclosure provides gRNAs, which may be for a CRISPR-mediated gene editing reporter system, polynucleotides encoding such a gRNA, and vectors comprising such a polynucleotide. In some embodiments, a gRNA may be a sgRNA or dgRNA.

While 20 nt is the most commonly used length for a targeting sequence of a gRNA in the field, truncated gRNAs (e.g., 19, 18, or 17 nt in length) are also known to provide similar or equivalent, or in some cases better gene editing effects (Fu et al. Nat Biotechnol. 2014 March; 32 (3): 279-284). Therefore, in some embodiments, a gRNA may have a crRNA sequence comprising a targeting sequence comprising at least 17 nucleotides. It was further shown that longer gRNAs (e.g., targeting sequences up to 30 nt in length) may be also used because longer gRNAs may be cleaved before participating in gene editing activities, so that the targeting sequence may be e.g., 20 nt in length (Ran et al., Cell. 2013 Sep. 12;154 (6): 1380-9). Therefore, in some embodiments, the length of a targeting sequence may be 17, 18, 19, 20, 21, 22, 23, 24, 25, 26, 27, 28, 29, or 30 nt.

In some embodiments, the targeting sequence may target any portion of SEQ ID NO: 80 or SEQ ID NO: 81. Therefore, in some embodiments, a targeting sequence may comprise (i) a sequence of at least 17 consecutive nucleic acids (optionally 17, 18, 19, 20, 21, 22, 23, 24, 25, 26, 27, 28, 29, or 30 consecutive nucleic acids) contained in SEQ ID NO: 80 or 81, (ii) a sequence comprising one or more mutations (optionally one, two, three, four, or five mutations) relative to the sequence of (i), or (iii) a sequence of at least 17 nucleic acids (optionally 17, 18, 19, 20, 21, 22, 23, 24, 25, 26, 27, 28, 29, or 30 consecutive nucleic acids) comprising at least 85, 90, 95, 96, 97, 98 or 99% sequence identity to the sequence of (i). Such a gRNA may be used in combination with any Cas endonucleases. Therefore, in some cases, the at least 17 consecutive nucleic acids may be immediately upstream or downstream of or adjacent to a PAM or PFS of a Cas endonuclease (depends on the type of Cas endonuclease used; in case of SpCas9 immediately upstream of 5′-NGG-3′; in case of Cpf1, immediately downstream of 5′-TTTN-3′) within SEQ ID NO: 80 or 81. A desired targeting sequence may be selected from all possible sequences, for example based on, the proximity to the desired editing position, the G-C content (e.g., for example in the range of about 40-80%), self-complementarity, the potential editing efficiency, and/or the potential off-target effects.

In particular embodiments, the at least 17 consecutive nucleic acids may comprise or consist of SEQ ID NO: 120, 121, 122, or 123 or SEQ ID NO: 130, 131, 132, or 133. In particular embodiments, the targeting sequence length may be 17, 18, 19, 20, 21, 22, 23, 24, 25, 26, 27, 28, 29, or 30 nucleotides. In yet particular embodiments, the targeting sequence may comprise or consist of SEQ ID NO: 120, 121, 122, or 123 or SEQ ID NO: 130, 131, 132, or 133.

In particular embodiments, the gRNA may comprise any of SEQ ID NOS: 125-128, 225-228, 325-328, and 425-428 any of SEQ ID NOS: 135-138, 235-238, 335-338, and 435-438.

A gRNA according to the present disclosure may comprise any RNA modifications as appropriate. In some embodiments, such a modification may increase gRNA stability and/or targeting efficiency and/or reduce off-target binding. In particular embodiments, the gRNA may comprise (i-1) 2′-O-methylation further optionally at first three and last three bases and/or (i-2) one or more 3′ phosphorothioate bonds, further optionally between first three and last two bases. In specific embodiments, the gRNA may comprise (i-1) 2′-O-methylation at first three and last three bases and (i-2) one or more 3′ phosphorothioate bonds between first three and last two bases.

### Target Sequences

When excision of a particular DNA segment flanked by two sites cleavable using one or more gRNAs results in expression of a reporter gene, any portion within the DNA segment (sense or antisense strand) between the two cleavable sites may also be additionally targeted by another gRNA. In some embodiments, that target sequence targeted by a gRNA according to the present disclosure may be contained in a DNA segment comprising SEQ ID NO: 80 or SEQ ID NO: 81. In certain embodiments, a target sequence may be contained in a DNA comprising any of SEQ ID NO: 20-23 and 30-33 and the sequences complementary to any of SEQ ID NO: 20-23 and 30-33. In certain embodiments, a target sequence may be any of the sequences complementary to any of SEQ ID NO: 120-123 and 130-133. Considering that some mismatches between a targeting sequence and a target sequence may be tolerated, a target sequence targeted by a gRNA according to the present disclosure may target a DNA segment comprising one or more mutations, optionally one, two, three, four, or five mutations, relative to any of the above-described target sequences. In some embodiments, a target sequence may comprise one or more mutations, optionally one, two, three, four, or five mutations, relative to be any of the sequences complementary to any of SEQ ID NO: 120-123 and 130-133.

### Polynucleotides and Vectors

Polynucleotides encoding a gRNA according to the present disclosure are also provided. Such a polynucleotide may encode one or more gRNAs. When a gRNA is a dgRNA (i.e., comprising a crRNA and a tracrRNA), a crRNA and a tracrRNA may be encoded by separate polynucleotides, i.e., two polynucleotides (one encoding a crRNA and one encoding a tracrRNA) may together encode one gRNA.

Vectors comprising such a polynucleotide encoding such a gRNA according to the present disclosure are further provided. Such a vector may comprise one or more polynucleotides each encoding one or more gRNAs. When a gRNA is a dgRNA, a crRNA and a tracrRNA may be encoded in a single vector (one vector comprising both a polynucleotide encoding a crRNA and a polynucleotide encoding a tracrRNA) or separate vectors (one comprising a polynucleotide encoding a crRNA and one comprising a polynucleotide encoding a tracrRNA).

### RNPs

In one aspect, the present disclosure provides RNPs comprising one or more gRNAs and a Cas endonuclease. Such an RNP may be used as part of a CRISPR-mediated gene editing reporter system.

In some embodiments, a gRNA comprised in a RNP may be any of the gRNAs described above. In certain embodiments, a gRNA comprised in a RNP may have a targeting sequence of SEQ ID NO: 120, 121, 122, or 123 or SEQ ID NO: 130, 131, 132, or 133. In particular embodiments, a gRNA comprised in a RNP may comprise any of SEQ ID NOS: 125-128, 225-228, 325-328, and 425-428 or SEQ ID NOS: 135-, 136, 137, or 138, 235-238, 335-338, and 435-438.

In particular embodiment, the one or more gRNAs may comprise two or more gRNAs. In some cases, the one or more gRNAs may comprise (i) a gRNA comprising a targeting sequence of SEQ ID NO: 120, 121, 122, or 123 or a targeting sequence comprising one or more mutations relative to SEQ ID NO: 120, 121, 122, or 123 and (ii) a gRNA comprising a targeting sequence of SEQ ID NO: 130, 131, 132, or 133 or a targeting sequence comprising one or more mutations relative to SEQ ID NO: 130, 131, 132, or 133. In some cases, the RNP may further comprise one or more additional gRNAs. In certain cases, the one or more additional gRNAs may comprise (iii) a gRNA comprising a targeting sequence of SEQ ID NO: 140, 141, 142, or 143 or a targeting sequence comprising one or more mutations relative to SEQ ID NO: 140, 141, 142, or 143 and (iv) a gRNA comprising a targeting sequence of SEQ ID NO: 150, 151, 152, or 153 or a targeting sequence comprising one or more mutations relative to SEQ ID NO: 150, 151, 152, or 153.

In some embodiments, a Cas endonuclease comprised in a RNP may be any of the Cas endonucleases described herein. In certain embodiments, a Cas endonuclease may be Cas9. In particular embodiments, a Cas endonuclease may be SpCas9, optionally having SEQ ID NO: 600, or a variant SpCas9, optionally comprising any of SEQ ID NOS: 601-611.

In some embodiments, a RNP may be formed by mixing a solution comprising a gRNA and a solution comprising a Cas endonuclease at an approximately equimolar ratio. In certain embodiments, the mixing may be for about 5 minutes. In certain embodiments, the solution comprising a gRNA may have a pH of about 6 to 8, about 6.5 to 7.5, or optionally about 7. In certain embodiments, the solution comprising a Cas endonuclease may have a pH of about 6 to 8, about 6.5 to 7.5, or optionally about 7 In certain embodiments, the resulting solution comprising a RNP may comprise a pH of about 6 to 8, about 6.5 to 7.5, or optionally about 7.

### Compositions

In one aspect, the present disclosure provides compositions relating to one or more gRNAs described above. Such a composition may be for a CRISPR-mediated gene editing reporter system, in which excision of a particular DNA segment flanked by two sites cleavable using one or more gRNAs (a 5′ cleavage site and 3′ cleavage site) in a target DNA results in expression of a reporter gene. Applicant discovered that additional targeting within the intervening DNA between the 5′ and 3′ cleavage sites enhances excision of the particular DNA segment.

Therefore, in some embodiments, a composition according to the present disclosure may comprise (A) a pharmaceutically acceptable carrier, (B) (a) one or more isolated gRNAs as described above or one or more polynucleotides encoding the one or more isolated gRNAs, and (b) a Cas endonuclease or a polynucleotide encoding a Cas endonuclease; and (C) optionally a template DNA or a polynucleotide encoding a template DNA.

In some embodiments, when the (B) (a) one or more isolated gRNAs as described above and (b) a Cas endonuclease may be present as one or more RNPs as described above. In such an embodiment, the one or more RNPs may comprise (i) a first RNP comprising a first isolated gRNA and a first Cas endonuclease and/or (ii) a second RNP comprising a second isolated gRNA and a second Cas endonuclease. Such a composition may further comprise (iii) a third RNP comprising a third isolated gRNA and a third Cas endonuclease and (iv) a fourth RNP comprising a fourth isolated gRNA and a fourth Cas endonuclease.

In some embodiments, a composition may comprise (B) (a) one or more isolated gRNAs as described above and (b) a polynucleotide encoding a Cas endonuclease. In some embodiments, a composition may comprise (B) (a) one or more polynucleotides encoding the one or more isolated gRNAs and (b) a Cas endonuclease. In some embodiments, a composition may comprise (B) (a) one or more polynucleotides encoding the one or more isolated gRNAs and (b) a polynucleotide encoding a Cas endonuclease. In any of such embodiments, the composition may comprise (i) (a) a first isolated gRNA or a polynucleotide encoding the first isolated gRNA and (b) a first Cas endonuclease or a polynucleotide encoding the first Cas endonuclease and/or (ii) (a) a second isolated gRNA or a polynucleotide encoding the second isolated gRNA and (b) a second Cas endonuclease or a polynucleotide encoding the second endonuclease. Such a composition may further comprise: (iii) (a) a third isolated gRNA or a polynucleotide encoding the third isolated gRNA and (b) a third Cas endonuclease or a polynucleotide encoding the Cas endonuclease and (iv) (a) a fourth isolated gRNA or a polynucleotide encoding the forth isolated gRNA and (b) a fourth Cas endonuclease or a polynucleotide encoding the fourth Cas endonuclease.

In any of the compositions mentioned above, in some embodiments, the first isolated gRNA may have a first targeting sequence which may comprise or consist of: (i) SEQ ID NO: 120, 121, 122, or 123; or (ii) a sequence of at least 17 nucleic acids comprising one or more mutations, optionally one, two, three, four, or five mutations, relative to SEQ ID NO: 120, 121, 122, or 123, optionally wherein the mutation(s) may be at any nucleic acid position(s) or are at position(s) other than the 4th to the 7th nucleic acid positions from the 3′-end of SEQ ID NO: 120, 121, 122, or 123. In particular embodiments, the first isolated gRNA may have a first targeting sequence which may comprise or consist of any of SEQ ID NOS: 125-128, 225-228, 325-328 and 425-428.

In any of the compositions mentioned above, in some embodiments, the second isolated gRNA may have a second targeting sequence which may comprise or consist of: (i) SEQ ID NO: 130, 131, 132, or 133; or (ii) a sequence of at least 17 nucleic acids comprising one or more mutations, optionally one, two, three, four, or five mutations, relative to SEQ ID NO: 130, 131, 132, or 133, optionally wherein the mutation(s) may be at any nucleic acid position(s) or are at position(s) other than the 4th to the 7th nucleic acid positions from the 3′-end of SEQ ID NO: 120, 121, 122, or 123. In particular embodiments, the second isolated gRNA may have a second targeting sequence which may comprise or consist of any of SEQ ID NOS: 135-138, 235-238, 335-338 and 435-438.

In any of the compositions mentioned above, in some embodiments, the third isolated gRNA may have a third targeting sequence which may comprise or consist of: (i) SEQ ID NO: 140, 141, 142, or 143; or (ii) a sequence of at least 17 nucleic acids comprising one or more mutations, optionally one, two, three, four, or five mutations, relative to SEQ ID NO: 140, 141, 142, or 143, optionally wherein the mutation(s) may be at any nucleic acid position(s) or are at position(s) other than the 4th to the 7th nucleic acid positions from the 3′-end of SEQ ID NO: 140, 141, 142, or 143. In particular embodiments, the third isolated gRNA may have a third targeting sequence which may comprise or consist of any of SEQ ID NOS: 145-148, 245-248, 345-348 and 445-448.

In any of the compositions mentioned above, in some embodiments, the fourth isolated gRNA may have a fourth targeting sequence which may comprise or consist of: (i) SEQ ID NO: 150, 151, 152, or 153; or (ii) a sequence of at least 17 nucleic acids comprising one or more mutations, optionally one, two, three, four, or five mutations, relative to SEQ ID NO: 150, 151, 152, or 153, optionally wherein the mutation(s) may be at any nucleic acid position(s) or are at position(s) other than the 4th to the 7th nucleic acid positions from the 3′-end of SEQ ID NO: 150, 151, 152, or 153. In particular embodiments, the fourth isolated gRNA may have a fourth targeting sequence which may comprise or consist of any of SEQ ID NOS: 155-158, 255-258, 355-358 and 455-458.

In some embodiments, any one or more of the first, second, third, and/or fourth gRNAs may comprise a targeting sequence of 17, 18, 19, 20, 21, 22, 23, 24, 25, 26, 27, 28, 29, or 30 nucleotides in length. In some embodiments, any one or more of the first, second, third, and/or fourth gRNAs may be a sgRNA having a sgRNA backbone comprising any appropriate sgRNA backbone sequences such as but not limited to SEQ ID NO: 111-114. In some embodiments, any one or more of the first, second, third, and/or fourth gRNAs may be a dgRNA formed by a crRNA comprising a crRNA backbone sequence and a tracr RNA. The crRNA backbone sequence and a tracr RNA may comprise any appropriate crRNA backbone and tracrRNA sequence combinations such as but not limited to the combination of SEQ ID NOS: 115 and 116 or SEQ ID NOS: 117 and 118.

Any of the polynucleotides encoding one or more gRNAs and/or one or more Cas endonucleases described above may be comprised in one or more vectors, which may be any of the vectors described herein, such as a plasmid or a viral vector (e.g., an adenoviral vector, an adeno-associated virus vector, a lentiviral vector, or a retroviral vector). In some embodiments, a vector may encode both one or more gRNAs and/or one or more Cas endonucleases. In some embodiments, the gRNA(s) and the Cas endonuclease(s) may be encoded in separate vectors.

In some embodiments, any one or more of the first, second, third, and/or fourth gRNAs may be synthetic or recombinant and may optionally comprise at least one chemical modification. In certain embodiments, the at least one chemical modification may be (i) 2′-O-methylation optionally at first three and last three bases and/or (ii) one or more 3′ phosphorothioate bonds, optionally between first three and last two bases.

In any of the compositions described above, in some embodiments, the Cas endonuclease may be any of Cas9, Cas3, Cas8a2, Cas8b, Cas8c, Cas10, Cas11, Cas12, Cas12a or Cpf1, Cas13, Cas13a, C2c1, C2c3, and C2c2. In some embodiments, the Cas endonuclease may be a class 2 Cas endonuclease, optionally a type II, type V, or type VI Cas nuclease. In certain embodiments, the Cas endonuclease may be Cas9, optionally Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of spCas9 (e.g., CP1012-SpCas9, CP1028-SpCas9, CP1041-SpCaS9, CP1249-SpCas9, and CP1300-SpCas9; see e.g., Oakes et al., Cell. 2019 Jan. 10;176 (1-2): 254-267.e16), eSpCas9, HypaCas9, FokI-Fused dCas9, xCas9, SpRY (variant of SpCas9), and SpG (variant of SpCas9). In particular embodiments, the Cas endonuclease may be Cas9, optionally comprising SEQ ID NO: 600 or any of SEQ ID NOS: 601-611. In certain embodiments, the Cas endonuclease may be Cas12a, optionally Cas12a of Lachnospiraceae bacterium ND2006 (LbCas12a), Acidaminococcus sp. BV3L6 (AsCas12a), or Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a.

In any of the compositions described above, in some embodiments, the pharmaceutically acceptable carrier may comprise a lipid-based TCV. In some embodiments, the TCV may any TCV described herein. In particular embodiments, the TCV may comprise DODMA, DOPE, DSPC, and cholesterol approximately at a 20:30:10:40 ratio (in mol %). In further embodiments, the TCV may comprise DODMA, DOPE, DSPC, cholesterol, and PEG-lipid approximately at a 20:30:10:39:1 ratio (in mol %). In particular embodiments, the TCV does not contain a permanently cationic lipid. In particular embodiments, the TCV does not contain a permanently anionic lipid. In particular embodiments, the ethanol concentration of the composition may be 5% (v/v) or below, preferably 0.5% (v/v) or below.

In particular embodiments, the composition may be substantially, essentially, or entirely free of ethanol.

In some embodiments, the TCV may encapsulate any one or more components of the composition according to the present disclosure. In certain embodiments, the TCV may encapsulate one or more RNPs (e.g., a mixture of different RNPs that are different by the gRNA comprised therein). In certain embodiments, the TCV may encapsulate one or more RNPs and a polynucleotide encoding a Cas endonuclease. In certain embodiments, the TCV may encapsulate one or more polynucleotides encoding one or more RNPs and a Cas endonuclease. In certain embodiments, the TCV may encapsulate one or more polynucleotides encoding one or more RNPs and a polynucleotide encoding a Cas endonuclease. In any of the compositions mentioned above, in some embodiments, the TCV may further encapsulate a template DNA.

### Template DNAs

When excision of a particular DNA segment flanked by two sites cleavable using one or more gRNAs (a 5′ cleavage site and 3′ cleavage site) in a target DNA results in expression of a reporter gene, DNA repair between the two sites may occur in the presence of a template DNA via homology-directed repair (HDR) or in the absence of a template DNA via non-homologous end-joining (NHEJ) and may, regardless of the repair type, result in the reporter gene expression, as also shown in Examples 2-5.

In some embodiments, a composition according to the present disclosure for CRISPR-mediated gene editing may comprise a template DNA, in addition to a gRNA (or a polynucleotide encoding a gRNA) and a Cas endonuclease (or a polynucleotide encoding a Cas endonuclease). In some embodiments, the template DNA may be designed to excise the DNA segment flanked by two sites cleavable using one or more gRNAs (a 5′ cleavage site and 3′ cleavage site) by joining the 5′ cleavage site and the 3′ cleavage site. When the DNA segment contain a particular sequence that should be functionally removed (e.g., terminator signal, poly A motif, etc), the DNA template may be designed so that the post-repair resulting sequence would not fully contain such a particular sequence.

In some embodiments, a repair template comprises or consists of a 5′ homology arm and a 3′ homology arm. In some embodiments, a template DNA may comprise a optional central region between the 5′ homology arm and the 3′ homology arm. In some embodiments, a template DNA may be approximately centered with respect to the 5′ cleavage site and 3′ cleavage site positions to be joined.

In some embodiments, a template DNA may comprise a total length of approximately 20-5000 nt. In some embodiments, the total length may be about 40-2000 nt, about 40-1000 nt, about 40-500 nt, about 60-200 nt, or about 80-160 nt, or about 80, about 100, about 120, about 140, or about 160.

Any appropriate size of a homology arm may be used. In some embodiments, the 5′ and 3′ homology arms may have the same or similar nucleotide lengths (e.g., 1, 2, 3, 4, 5, 6, 7, 8, 9, or 10 nt difference). In some embodiments, the 5′ and 3′ homology arms may significantly differ in length. In some embodiments, the size of a homology arm may be approximately 20-2500 nucleotides (nt), about 20-1000 nt, about 20-500 nt, or about 20-100 nt. In particular embodiments, the size of a homology arm may be about 40-80 nt. In particular embodiments, the size of each homology arm may be or about 40 nt, about 50 nt, about 60 nt, about 70 nt, or about 80 nt.

In some embodiments, a template DNA may be a single-stranded (ssODN) or double-stranded.

In some embodiments, a ssODN may comprise: (i) a 5′ homology arm complementary or nearly complementary (i.e., fully complementary or further comprising one or more mutation) to the sequence of the target DNA (of the strand with the PAM sequence) immediately upstream of the 5′ cleavage site; and (ii) a 3′ homology arm complementary or nearly complementary (i.e., fully complementary or further comprising one or more mutation) to the sequence of the target DNA (of the strand with the PAM sequence) immediately downstream of the 3′ cleavage site. In some embodiments, a ssODN may comprise: (i) a 5′ homology arm complementary or nearly complementary (i.e., fully complementary or further comprising one or more mutation) to the sequence of the target DNA (of the strand opposite to the strand with the PAM sequence) immediately upstream of the 3′ cleavage site; and (ii) a 3′ homology arm complementary or nearly complementary (i.e., fully complementary or further comprising one or more mutation) to the sequence of the target DNA (of the strand opposite to the strand with the PAM sequence) immediately downstream of the 5′ cleavage site.

Therefore, in some embodiments, a ssODN may comprise a 5′ homology arm comprising the first to at least the 10th nucleotides counting from the 3′-end of SEQ ID NO: 581, and a 3′ homology arm comprising the first to at least the 10th nucleotides counting from the 5′-end of SEQ ID NO: 582. In certain embodiments, the template DNA may comprise a 5′ homology arm comprising any of SEQ ID NOS: 511, 521, 531, 541, 551, 561, 571, and 581, and a 3′ homology arm comprising any of SEQ ID NOS: 512, 522, 532, 542, 552, 562, 572, and 582. In some embodiments, such 5′ and/or 3′ homology arms may further comprise at least one (such as one, two, three, four, five, six, seven, eight, nine, or ten) mutation(s) relative to the respective, above-mentioned sequences. In particular embodiments, a ssODN may comprise or consist of the sequence of any of SEQ ID NOs: 510, 520, 530, 540, 550, 560, 570, and 580. In further embodiments, a ssODN may be fully complementary to the sequence any of the ssODNs sequences described above.

When a template DNA is a double-stranded DNA, one of the strands of the template may comprise the same sequence as any of the ssODN sequences described above and the other strand may have a sequence complementary thereto.

In some embodiments, a template DNA may have one or more mutations at one or more of the PAM positions, if applicable. In some embodiments, such a mutation(s) helps prevent or reduce Cas-mediated cleavage of the template DNA itself or of the DNA molecule post repair. In case of a ssODN, such a mutation may be within the PAM bases or the reverse (or antisense) bases, i.e., the opposite strand) and/or at one or more of the 5′-neighbouring bases of the PAM (or the 3′-neighbouring bases of the reverse (or antisense) sequences corresponding to the PAM). In some embodiments, a ssODN may comprise a sequence complementarity to the gRNA strand.

### Reporter Cells, Tissues, and Animals

In one aspect, the present disclosure provides a cell, a tissue (comprising such a cell), or an animal such as a transgenic animal (comprising such a cell and/or such a tissue), which may be used in combination with any of the gRNA(s) or any of the RNPs or any of the compositions described above, for example as part of any of the CRISPR-mediated gene editing reporter systems described herein.

In some embodiments, such a cell may comprise a DNA molecule which may be targeted by multiple gRNAs, such as three or more gRNAs. In some embodiments, a cell may have a DNA molecule comprising a target sequence which may be targeted by PS2 gRNA and/or a target sequence which may be targeted by PS3 gRNA, followed by a reporter gene sequence. In some embodiments, a cell may have a DNA molecule comprising a target sequence which may be targeted by LaRo gRNA and a target sequence which may be targeted by LoxP gRNA, followed by a reporter gene sequence.

In certain embodiments, a cell may have a DNA molecule comprising a DNA span comprising one or more first segments comprising a target sequence which may be targeted by PS2 gRNA and/or one or more second segments comprising a target sequence which may be targeted by PS3 gRNA and the DNA span may be flanked by a third segment comprising a target sequence which may be targeted by LaRo gRNA and a fourth segment comprising a target sequence which may be targeted by LoxP gRNA, wherein the DNA span may be followed by a reporter gene sequence.

In certain embodiments, the first segment may comprise SEQ ID NO: 20, 21, 22, or 23. Considering that some mismatches are tolerated, in certain embodiments, the first segment may be targeted by PS2 gRNA may comprise at least one first segment comprising a sequence comprising one or more mutations, optionally one, two, three, four, or five mutations, relative to SEQ ID NO: 20, 21, 22, or 23. In certain embodiments, the second segment may comprise SEQ ID NO: 30, 31, 32, or 33. Considering that some mismatches are tolerated, in certain embodiments, the second segment may comprise a sequence comprising one or more mutations, optionally one, two, three, four, or five mutations, relative to SEQ ID NO: 30, 31, 32, or 33. In certain embodiments, the third segment may comprise SEQ ID NO: 40, 41, 42, or 43. Considering that some mismatches are tolerated, in certain embodiments, the third segment may comprise a sequence comprising one or more mutations, optionally one, two, three, four, or five mutations, relative to SEQ ID NO: 40, 41, 42, or 43. In certain embodiments, the fourth segment may comprise SEQ ID NO: 50, 51, 52, or 53. Considering that some mismatches are tolerated, in certain embodiments, the fourth segment may comprise a sequence comprising one or more mutations, optionally one, two, three, four, or five mutations, relative to SEQ ID NO: 50, 51, 52, or 53. In some cases, the third and fourth segments may be upstream and downstream, respectively, of the DNA span. In some cases, the third and fourth segments may be downstream and upstream, respectively, of the DNA span. In some cases, at least one terminator sequence is contained within said DNA span. In particular embodiments, the DNA molecule comprises any of SEQ ID NOS: 1, 60, 70, 80, 81, and 90. In some embodiments, the DNA span may be flanked by the third and fourth segments at the Rosa26 locus.

In some embodiments, the cell may be a cell line or a primary cell. In some embodiments, the cell is a cell of a tissue or organ of interest. In some embodiments, the reporter gene encodes a fluorescent marker, optionally monomeric cherry (mCherry), tandem dimer Tomato (tdTomato), red fluorescent protein (RFP), DsRed1, DsRed S197Y, green fluorescent protein (GFP), enhanced FP (EGFP), yellow fluorescent protein (YFP), enhanced yellow fluorescent protein (EYFP), cyan fluorescent protein (CFP), or enhanced cyan (ECFP), or any variants thereof.

Tissues comprising any of the above-mentioned cells and animals comprising such a cell and/or such a tissue are also provided herein. In some embodiments, the tissue is a tissue of interest. In some embodiments, the animal is a rodent, further optionally a mouse or a rat. In particular embodiments, the animal is the Ai9 or Ai14 mouse, and a cell and/or a tissue is a cell and/or tissue derived therefrom. In certain embodiments, the animal may be B6; 129S6-Gt(ROSA)26Sor^{tm14(CAG-tdTomato)Hze}/J or its congenic version B6.Cg-Gt(ROSA)26Sor^{tm14(CAG-tdTomato)Hze}/J, such as Strain #007908 or 007914, respectively, from the Jackson Laboratory, commonly referred to as Ai14, Ai14D, or Ai14(RCL-tdT)-D mouse, and a cell and/or a tissue may be a cell and/or tissue derived therefrom. In certain embodiments, the transgenic animal may be B6.Cg-Gt(ROSA)26Sor^{tm9(CAG-tdTomato)Hze}/J such as Strain #007909 from the Jackson Laboratory, commonly referred to as Ai9 or Ai9 (RCL-tdT) mouse, and a cell and/or a tissue may be a cell and/or tissue derived therefrom.

### CRISPR-Mediated Gene Editing Reporter System

In one aspect, the present disclosure provides a reporter system. In such a reporter system, use of any of the gRNAs described above participates in gene editing may result in the expression of a reporter gene.

In some embodiments, such a reporter system may comprise any of the cells, tissues (comprising such a cell), and/or the animals described above.

In some embodiments, such a reporter system may comprise CRISPR-mediated gene editing agents, which comprise: (1) multiple isolated gRNAs or one or more polynucleotides encoding the multiple isolated gRNAs and (2) a Cas endonuclease or a polynucleotide encoding a Cas endonuclease; and (3) optionally a template DNA or a polynucleotide encoding a template DNA. In certain embodiments, the multiple isolated gRNAs may comprise (I) any of the first isolated gRNAs as described above and/or (II) any of the second isolated gRNAs as described above and may further comprise (I) any of the third isolated gRNAs as described above and (II) any of the third isolated gRNAs as described above. In some embodiments, such CRISPR-mediated gene editing agents may be comprised in any of the above-described compositions.

### Methods of Testing CRISPR-Mediated Gene Editing

Any of the CRISPR-mediated gene editing reporter systems may be used for testing gene editing, such as effects of a given vehicle or carrier, administration route, and/or dose on gene editing events.

In one aspect, the present disclosure provides methods of testing the level of CRISPR-mediated gene editing events. Such a method may be a method of testing in vitro the level of CRISPR-mediated gene editing events in a cell and may comprise (a) applying CRISPR-mediated gene editing agents of the CRISPR-mediated gene editing reporter system as described above to one or more cells of the CRISPR-mediated gene editing reporter system as described above; and (b) analyzing the level of CRISPR-mediated gene editing events in the one or more cells. Such a method may be a method of testing ex vivo the level of CRISPR-mediated gene editing events in a cell or tissue and may comprise (a) applying CRISPR-mediated gene editing agents of the CRISPR-mediated gene editing reporter system as described above to one or more cells or tissues of the CRISPR-mediated gene editing reporter system as described above; and (b) analyzing the level of CRISPR-mediated gene editing events in the one or more cells or tissues. Such a method may be a method of testing in vivo the level of CRISPR-mediated gene editing events in a cell or tissue and may comprise (a) applying CRISPR-mediated gene editing agents of the CRISPR-mediated gene editing reporter system as described above to one or more animals of the CRISPR-mediated gene editing reporter system as described above; and (b) analyzing the level of CRISPR-mediated gene editing events in the one or more animals. In any of such embodiments, the analyzing in step (b) may be performed, e.g., by (i) quantifying the reporter gene expression in the cells, tissues (or cells derived therefrom), or the transgenic animals (or tissues or cells derived therefrom), optionally via flow cytometry, fluorescent microscopy, or qPCR; and/or (ii) determining the presence or absence or level of (ii-1) the DNA sequence flanked by the sites cleavable by the third and fourth isolated gRNAs or (ii-2) the transcript thereof in the one or more transgenic animals or tissues or cells derived therefrom, optionally via PCR or qPCR, respectively.

In some embodiments, such methods may test effects of a vehicle or a carrier in a composition of gene editing events, compatibility of a specific cell type, tissue type, and/or animal species to a given vehicle or carrier or a CRISPR-mediated gene editing agent or composition, or test a given dose or dose range, dosing regimen, or administration route.

Knocking Out DNA Using More than Two Guide RNAs

As demonstrated in Examples, Applicant discovered that when a sequence desired to be excised (i.e., knocking out) is flanked by two sites cleavable by CRISPR-mediated gene editing, additionally targeting the intervening sequence (the sequence between the two cleavable site) by CRISPR-mediated gene editing surprisingly enhances gene editing.

This finding is surprising because, if joining between the two sites are desired, one would expect that the desired editing will be determined by whether a cleavage occurs at both of the two sites and not by what happens within the intervening sequence. One may even believe that, if the intervening sequence is cleaved before a cleavage occurs at the two site, the intended cleavages at the two sites may not occur as efficiently. However, the opposite was observed. Without wishing to be bound by theory, the additional cleavage within the intervening sequence which results in more of smaller polynucleotide pieces may increase the chance of the intended gene editing (excision of the intervening sequence) to occur by reducing the chance of the cleaved intervening sequence to be repaired by the host repair system back to the original location, i.e., between the two cleavable sites flanking the intervening sequence.

Therefore, in one aspect, the present disclosure provides methods and compositions for knocking out a DNA segment of interest in a cell, tissue, or subject, wherein (i) the DNA segment of interest is flanked by a 5′ first site cleavable by CRISPR-mediated gene editing via a first gRNA and a 3′ second site cleavable by CRISPR-mediated gene editing via a second gRNA; and (ii) the intervening sequence flanked by the first site and the second site comprises at least one third site cleavable by CRISPR-mediated gene editing via a third gRNA.

In some embodiments, a composition for knocking out a DNA segment of interest in a cell, tissue, or subject may comprise (a) the first gRNA, the second gRNA, and the third gRNA (optionally at an equimolar ratio) or one or more polynucleotides encoding the first gRNA, the second gRNA, and the third gRNA (optionally at an equimolar ratio); (b) a Cas endonuclease or a polynucleotide encoding a Cas endonuclease; and (c) optionally a template DNA or a polynucleotide encoding a template DNA.

In certain embodiments, the template DNA may comprise a ssODN comprising (c-1) a 5′ homology arm complementary or nearly complementary (i.e., fully complementary or comprising on or more mutations) to the DNA sequence (on the strand with the PAM sequence for the first gRNA) immediately upstream of the first site and (c-2) a 3′ homology arm complementary or nearly complementary (i.e., fully complementary or comprising on or more mutations) to the DNA sequence (on the strand with the PAM sequence for the first gRNA) immediately downstream of the second site. In certain embodiments, the template DNA may comprise a ssODN comprising (c-1) a 5′ homology arm complementary or nearly complementary (i.e., fully complementary or comprising on or more mutations) to the DNA sequence (on the strand opposite to the strand with the PAM sequence for the first gRNA) immediately upstream of the second site and (c-2) a 3′ homology arm complementary or nearly complementary (i.e., fully complementary or comprising on or more mutations) to the DNA sequence (on the strand opposite to the strand with the PAM sequence for the first gRNA) immediately downstream of the first site. In certain embodiments, the template DNA may comprise a double-stranded DNA comprising a first strand comprising any of the above-mentioned ssODN sequence and a second strand complementary thereto.

In certain embodiments, the presence of the third gRNA may increase the efficiency or probability of knocking out the DNA segment of interest. In particular embodiments, the intervening sequence comprises two or more third sites cleavable by CRISPR-mediated gene editing via the third gRNA. Without wishing to be bound by theory, having more than one third cleavable sites in the intervening sequence may allow for cleavage of the intervening sequence into smaller pieces compared to when there is only one third site. This may further reduce the chance of the cleaved intervening sequence to be repaired by the host repair system back to the original location, i.e., between the two cleavable sites flanking the intervening sequence, increasing the chance of the intended gene editing to occur.

In certain embodiments, the intervening sequence may further comprise at least one fourth site cleavable by CRISPR-mediated gene editing via a fourth gRNA which comprises a different target specificity relative to the third gRNA. In such an embodiment, the CRISPR-mediated gene editing agents may further comprise the fourth gRNA. In particular embodiments, the presence of the fourth gRNA may increase the efficiency or probability of knocking out the DNA segment of interest. In particular embodiments, the intervening sequence may comprise two or more fourth sites cleavable by CRISPR-mediated gene editing via the fourth gRNA. Without wishing to be bound by theory, having another cleavable site different from the third site(s) in the intervening sequence may allow for cleavage of the intervening sequence into further smaller pieces compared to when only third site(s) is/are present. This may further reduce the chance of the cleaved intervening sequence to be repaired back to the original location, further increasing the chance of the intended gene editing to occur.

In some embodiments, a method of knocking out a DNA segment of interest in a cell, tissue, or subject may comprise applying any of the compositions for knocking out a DNA segment of interest described above to the cell, tissue, or subject. Similarly, without wishing to be bound by theory, having more than one fourth cleavable sites in the intervening sequence may allow for cleavage of the intervening sequence into yet further smaller pieces compared to when there is only one fourth site. This may further reduce the chance of the cleaved intervening sequence to be repaired back to the original location, further increasing the chance of the intended gene editing to occur.

In certain embodiments, applying may comprise culturing a cell (e.g., cell line, primary cells) with the CRISPR-mediated gene editing agents. In certain embodiments, applying may comprise culturing a tissue (e.g., primary tissue, or an artificial tissue such as a 3D tissue culture) with the CRISPR-mediated gene editing agents. In certain embodiments, applying may comprise administering the CRISPR-mediated gene editing agents to a subject. In certain embodiments, the subject may be a human. In certain embodiments, the subject may be a non-human subject, optionally a non-human primate. In certain embodiments, the subject may be, without limitation, a rodent (mouse, rat, guinea pig, hamster), rabbit, cat, dog, pig, goat, sheep, horse, or monkey, further optionally a mouse or a rat.

### Method of Effecting Gene Editing in the Eye

As demonstrated in Examples, using the gRNAs and the reporter system as described above, Applicant discovered that gene editing may be efficiently effected by directly administering CRISPR-mediated gene editing agents encapsulated in one or more TCVs into the eye.

Therefore, in one aspect, the present disclosure provides methods for effecting CRISPR-mediated gene editing in the eye.

In some embodiments, such a method may comprise administering CRISPR-mediated gene editing agents directly into the eye of a subject. In certain embodiments, the administration may be into the cornea, retina, or subretinal tissue. In some embodiments, the CRISPR-mediated gene editing agents comprise: (a) one or more gRNAs or one or more polynucleotides encoding the one or more gRNAs; (b) a Cas endonuclease or a polynucleotide encoding a Cas endonuclease; and (c) optionally a template DNA or a polynucleotide encoding a template DNA. Any one or more of (a)-(c) may be co-encapsulated in a TCV or separately encapsulated in a TCV.

The TCV used in a methods for effecting CRISPR-mediated gene editing in the eye may be according to any of the TCVs described herein. In particular embodiments, the TCV may comprise DODMA, DOPE, DSCP, and cholesterol at approximately an 20:30:10:40 ratio (in mol %). In particular embodiments, the TCV may comprise DODMA, DOPE, DSCP, cholesterol, and PEG-lipid at approximately an 20:30:10:39:1 ratio (in mol %).

In some embodiments, the subject may be a human, non-human, non-human primate, a rodent (mouse, rat, guinea pig, hamster), rabbit, cat, dog, pig, goat, sheep, horse, or monkey.

In some embodiments, the method may be for knocking out a DNA segment of interest in the eye or a cell thereof of the subject. In certain embodiments, (i) the DNA segment of interest may be flanked by a 5′ first site cleavable by CRISPR-mediated gene editing via a first gRNA and a 3′ second site cleavable by CRISPR-mediated gene editing via a second gRNA; and (ii) the intervening sequence flanked by the first site and the second site may comprise at least one third site cleavable by CRISPR-mediated gene editing via a third gRNA. In such an embodiment, the CRISPR-mediated gene editing agents to be administered may comprise three or more gRNAs or three or more polynucleotides encoding three or more gRNAs. In particular embodiments, the three or more gRNAs may comprise or consist of the first gRNA, the second gRNA, and the third gRNA, optionally at an equimolar ratio. When the CRISPR-mediated gene editing agents comprise a template DNA or a polynucleotide encoding a template DNA, the template DNA may optionally comprise (c-1) a 5′ homology arm homologous or complementary to the DNA sequence immediately upstream of the first site and (c-2) a 3′ homology arm homologous or complementary to the DNA sequence immediately downstream of the second site.

In particular embodiments, the intervening sequence may comprise two or more third sites cleavable by CRISPR-mediated gene editing via the third gRNA. In certain instances, the presence of two or more third sites cleavable via the third gRNA may increase the intended gene editing efficiency. Without wishing to be bound by theory, cleaving the intervening sequence into more pieces may enhance the intended gene editing, for example by reducing the chance of the cleaved pieces (derived from the intervening sequence) being repaired back to into the DNA segment between the 5′ first site and the 3′ second site.

In further embodiments, the intervening sequence may further comprise at least one fourth site cleavable by CRISPR-mediated gene editing via a fourth gRNA which comprises a different target specificity relative to the third gRNA, and wherein the CRISPR-mediated gene editing agents further comprise the fourth gRNA

In some embodiments, a method of knocking out a DNA segment of interest in a cell, tissue, or subject may be for treating a genetic disease or disorder of the eye.

Injection into the Eye

In a method of effecting CRISPR-mediated gene editing in the eye according to the present disclosure, the CRISPR-mediated gene editing agents may be formulated in any formulation appropriate for direct eye injection. The pH, osmolarity, and/or viscosity of a formulation may be adjusted based on the needs. Such formulations include but are not limited to solutions, suspensions, gels, ointments, and solid inserts. Examples of gels include methylcellulose and polyvinyl alcohol. Examples of ointments include anhydrous lanolin in a mineral oil and petrolatum base. The pH, osmolarity, and/or viscosity of a formulation may be adjusted based on the needs. The pH of the vehicle may be adjusted based on the effect on chemical and physical stability of the drug in the formulation. Optimal pH may be as close as possible to the drug's dissociation constant (pKa) to maximize the ratio of ionized and non-ionized drug. Low pH and hypo-or hyperosmolality may cause increased discomfort, tearing, and excessive washout of the drug. More viscous formulations may have a longer residence time, which in turn may affects the rate and extent of absorption. Low viscosity may allow for faster drainage from the conjunctival sac, resulting in less corneal and conjunctival contact time.

In a method of effecting CRISPR-mediated gene editing in the eye according to the present disclosure, the CRISPR-mediated gene editing agents may be administered at any appropriate doses. Such doses may vary, and for example about 25-50 μL may be used. An exemplary maximum volume may be about 230 μL.

In a method of effecting CRISPR-mediated gene editing in the eye according to the present disclosure, the CRISPR-mediated gene editing agents may be administered via any appropriate route. Such routes include but are not limited to subcutaneous, subconjunctival, subTenon, intracameral, intravitreal, or retrobulbar injection.

### Dosing Regimen

The pharmaceutical composition for an ocular injection may comprise any amounts of gene-editing agents sufficient for effecting sufficient gene editing. In some embodiments, the pharmaceutical composition may comprise per mL about 300 pmol to about 30000 pmol, optionally about 500 to about 10000 pmol, about 1000 to about 5000 pmol, about 2000 to about 4000 pmol, about 2500 to about 3000 pmol, or about 2700 pmol of the RNP or the nucleic acid molecule. In particular embodiments, the pharmaceutical composition may comprise about 2700 pmol of the RNP or the nucleic acid molecule per mL.

The ocular injection may be given at any appropriate volume and/or speed suited for effecting sufficient gene editing. In some embodiments, injection on may be via a drop of about 25-50 μL per eye.

In some embodiments, the TCVs encapsulating at least gene-editing agent may be comprised in or loaded in a matrix or material that may be injected or implanted in the eye. Any appropriate matrices or materials may be used. In some embodiments, use of such a matrix or material may lead to improved safety, for example by allowing a smaller thus safer dose to provide efficacy and/or lead to improved feasibility, for example by allowing gradual release of the TCVs, thereby offering less frequent need of injections or shorter injection duration.

### Compositions and Methods for Base Editing

Base editors, which typically comprise at least a deaminase and a Cas-derived platform protein (e.g., Cas nickase), are generally larger than Cas proteins. Therefore, it was unknown whether TCVs capable of encapsulating and delivering Cas proteins to cells would be also capable of encapsulating and delivering base editors to cells. As demonstrated in Example 6, Applicant discovered that the TCV described herein is capable of encapsulating a RNP which comprises a base editor complexed with a gRNA and deliver such a RNP to a cell to provide intended base editing.

Therefore, in one aspect, the present disclosure provides compositions for base editing.

### Compositions

A composition for effecting base editing may comprise a lipid-based TCV (e.g., any of those described herein) as a pharmaceutically acceptable carrier which encapsulates an RNP comprising (i) a base editor which is complexed with (ii) a gRNA. The gRNA may be appropriately designed to provide base editing of interest in a cell in the presence of the base editor with which the gRNA is complexed.

The base editor may comprise a comprises a Cas-derived platform protein linked to a deaminase.

### Cas-Derived Platform Protein

In some embodiments, the Cas-derived platform protein may be derived from any appropriate Cas proteins (e.g., Cas9, Cas12a, etc) of any appropriate species including but not limited to any of the Cas and variants thereof described herein. In some embodiments, the Cas-derived platform protein may be or may comprise: a Cas nickase, a Cas-derived protein capable of creating a single-strand break at a target DNA site rather than a double-strand break; or a catalytically dead Cas protein, a Cas-derived protein incapable of creating a single-strand or double-strand break at a target DNA site.

In certain embodiments, a Cas nickase may be a variant of a Cas protein, in which one of the nuclease domains of the parent domain is catalytically disabled/dead, e.g., an amino acid alteration. For example, in case of SpCas9 (wild-type sequence comprises SEQ ID NO: 600), D10A and H840A substitutions inactivates both of the nuclease domains of SpCas9, resulting in a catalytically dead SpCas9 and either D10A or H840A substitution inactivates one of the nuclease domains of SpCas9, resulting in a SpCas9 nickase. In particular embodiments, a catalytically dead SpCas9, “dCas9” may comprise SEQ ID NO: 620. In particular embodiments, a SpCas9 nickase, “Cas9n” or “nCas9” may comprise SEQ ID NO: 621. The N-terminal “M” residue is not included in SEQ ID NO: 620 or 621, so D10A and/or H840A appear at positions 9 and/or 839, respectively, in SEQ ID NOS: 620 and 621). SpCas9 endonucleases of another species origin may be altered similarly to produce a catalytically dead Cas9 and a Cas9 nickase by incorporating the corresponding substitution(s). In certain embodiments, any other Cas9 nickases, including VQR-SpCas9 nickase (SEQ ID NO: 631), EQR-SpCas9 nickase (SEQ ID NO: 632), VRER-SpCas9 nickase (SEQ ID NO: 633), CP1028-SpCas9 (SEQ ID NO: 634), CP1041-SpCas9 (SEQ ID NO: 635), SpCas9-NG (SEQ ID NO: 636), SaCas9 nickase (SEQ ID NO: 640), or SaCas9-KKH (SEQ ID NO: 641), or a variant thereof, such as a Cas9 of another species origin but comprising one or more or all of the corresponding substitutions contained in the aforementioned Cas nickases, may also be used.

Without wishing to be bound by theory, using a Cas nickase may allow cleavage of the DNA strand complementary to the DNA strand which is to be or has been edited (i.e., deaminated), promoting the use of the edited strand rather than the unedited strand as a template for DNA replication, leading to higher editing efficiencies.

### Deaminase

In some embodiments, the deaminase may be capable of deaminating a base within a target DNA site when the target DNA site is in a single-stranded form. In some embodiments, the deaminase may specifically deaminate adenine (i.e. adenine deaminase), cytidine (i.e., cytidine deaminase), or both (i.e., dual deaminase). When an adenine deaminase is used, the base editor is an ABE and capable of mediating A-to-inosine conversion, and inosine may then be converted to G during DNA replication in a cell, thereby converting target A: T base pairs to G: C base pairs. When a cytidine deaminase is used, the base editor is a CBE and capable of mediating C-to-U conversion, and U may then be converted to T during DNA replication in a cell, thereby converting target C: G base pairs to T: A base pairs. When a dual deaminase is used, the base editor is a DE and capable of mediating both A-to-inosine and C-to-U conversion, and inosine and U may then be converted to G and T, respectively, during DNA replication in a cell, thereby converting target A: T base pairs to G: C base pairs and target C: G base pairs to T:A base pairs.

In certain embodiments, the adenine deaminase may be or be derived from TadA, such as E coli. TadA (SEQ ID NO: 820). Various TadA variants have been reported as a ABE component to date, including but not limited to: ecTadA*8e (SEQ ID NO: 826); ecTadA*8e-V106W (SEQ ID NO: 827); ecTadA*8e-V82G (SEQ ID NO: 828);ecTadA*8e-K20A-R21A (SEQ ID NO: 829); ecTadA*6.3 (SEQ ID NO: 821); ecTadA*6.4 (SEQ ID NO: 822); ecTadA*7.8 (SEQ ID NO: 823); ecTadA*7.9 (SEQ ID NO: 824); orecTadA*7.10 (SEQ ID NO: 825). In particular embodiments, the adenine deaminase may be any of such adenine deaminases such as ecTadA*7.10 (SEQ ID NO: 825) or ecTadA*8e (SEQ ID NO: 826) or a TadA variant comprising one or more or all of the corresponding substitutions contained in any of the aforementioned TadA variants.

In certain embodiments, the cytidine deaminase may be or be derived from APOBEC, such as rat APOBEC1 (SEQ ID NO: 720), or TadA, such as E coli. TadA (SEQ ID NO: 820). Various cytidine deaminases have been reported as a CBE component to date, including but not limited to: rAOPBEC1 (SEQ ID NO: 720); YE1-rAPOBEC1 (SEQ ID NO: 721); YE2-rAPOBEC1 (SEQ ID NO: 722); EE-rAPOBEC1 (SEQ ID NO: 723); YEE-rAPOBEC1 (SEQ ID NO: 724); Anc689 APOBEC (SEQ ID NO: 731); Anc687 APOBEC (SEQ ID NO: 732); CDA1 (SEQ ID NO: 725); AID (SEQ ID NO: 726); TadA-CDa (SEQ ID NO: 741); TadA-CDb (SEQ ID NO: 742); TadA-CDc (SEQ ID NO: 743); TadA-CDd (SEQ ID NO: 744); TadA-CDe (SEQ ID NO: 745); TadA-CDa-V106W (SEQ ID NO: 751); TadA-CDb-V106W (SEQ ID NO: 752); TadA-CDc-V106W (SEQ ID NO: 753); TadA-CDd-V106W (SEQ ID NO: 754); and TadA-CDe-V106W (SEQ ID NO: 755). In particular embodiments, the cytidine deaminase may be any of such cytidine deaminases such as rAOPBEC1 (SEQ ID NO: 720), or an APOBEC variant comprising one or more or all of the corresponding substitutions contained in any of the aforementioned APOBEC variants, or a TadA variant comprising one or more or all of the corresponding substitutions contained in any of the aforementioned TadA variants.

In certain embodiments, the dual deaminase may be or be derived from TadA, such as E coli. TadA (SEQ ID NO: 820). Various dual deaminases have been reported as a DE component to date, including but not limited to: TadA*Dual (SEQ ID NO: 920); and TadA*Dual-V106W (SEQ ID NO: 921). In particular embodiments, the dual deaminase may be any of such dual deaminases, or a variant thereof comprising one or more or all of the corresponding substitutions contained in any of the aforementioned dual deaminases.

In some embodiments, the deaminase may have been evolved from a known adenine deaminase, e.g., via directed evolution and/or genetic pressure, to achieve a desired function, editing efficiency, or selectivity, such as substrate base preference (e.g., which base to deaminate, preference of DNA bases than RNA bases, etc) or target sequence preference (i.e., the nucleic acid sequence/context immediately around the target base to be edited). Any methods to induce such genetic evolution may be used, e.g., those described in Gaudelli et al., Nature. 2017 Nov. 23; 551 (7681): 464-471, Richter et al., Nat Biotechnol. 2020 July;38 (7): 883-891, and Neugebauer et al., Nat Biotechnol. 2022 Nov. 10.

In some embodiments, the deaminase may be place N-terminal to the Cas-derived platform protein.

### Other Base Editor Components

In some embodiments, a base editor may further comprise one or more nuclear localization signal (NLS). In certain embodiments, a NSL may be placed at the N-terminus of the base editor. In certain embodiments, a NSL may be placed at the C-terminus of the base editor. In certain embodiments, a NSL may be placed at the N-terminus of the base editor and another NSL may be placed at the C-terminus of the base editor, and the two NSL may have the same or different sequences. In certain embodiments, the NSL(s) may individually selected from NLS1 (SEQ ID NO: 691); NLS2 (SEQ ID NO: 692); and/or NLS3 (SEQ ID NO: 693).

In some embodiments, one or more linkers may be used to link different components within a base editor. Any appropriate linker may be used as long as the base editor achieves an intended function. In certain cases, the linker(s) in a base editor may individually comprise or consist of or comprise or consist of multiple repeats of an amino acid sequence selected from the group consisting of G, GG, GGG, GS, SG, GGS, GSG, SGG, GSS, SGS, SSG, SGGS (SEQ ID NO: 682), GGGS (SEQ ID NO: 685), and GGGGS (SEQ ID NO: 686). In certain cases, the linker(s) in a base editor may individually be or comprise the XTEN linker (SEQ ID NO: 681), the (SGGS)₂-XTEN-(SGGS) 2 linker (SEQ ID NO: 683), and/or the (SGG) 3S linker (SEQ ID NO: 684).

In some embodiments, a base editor (e.g., CBE or DE) may further comprise a uracil DNA glycosylase inhibitor (UGI) (e.g., SEQ ID NO: 760) or a variant thereof. Without wishing to be bound by theory, Uracil DNA glycosylase (UDG) catalyzes removal of U from DNA in cells and initiates base-excision repair, with reversion of the U:G pair to a C:G pair as the most common outcome and therefore including a UGI may help prevent such base-excision repair, thereby providing higher editing efficiency.

In some embodiments, a base editor (e.g., CBE or DE) may further comprise Gam (e.g., SEQ ID NO: 770), a bacteriophage Mu protein that binds double-stranded breaks, or a variant thereof. Without wishing to be bound by theory, including a Gam may greatly reduce indel formation, thereby providing higher editing efficiency.

### Exemplary Base Editor Sequences

In some embodiments, an ABE may comprise an adenine deaminase and a Cas-derived platform protein, optionally in the direction from the N-terminus to the C-terminus. In certain embodiments, an ABE may comprise a NLS, an adenine deaminase, a Cas-derived platform protein, and a NLS, optionally in the direction from the N-terminus to the C-terminus and optionally comprising a linker between different components. In certain embodiments, an ABE may further comprise an additional adenine deaminase, allowing the two deaminase to form a dimer. In particular embodiments, an ABE may be or may comprise (i) ABE8e (SEQ ID NO: 810), (ii) ABE8e dimer (SEQ ID NO: 811), (iii) ABEmax (SEQ ID NO: 801), and/or (iv) ABE7.10 (SEQ ID NO: 800), or a variant thereof.

In some embodiments, a CBE may comprise a cytidine deaminase and a Cas-derived platform protein, optionally in the direction from the N-terminus to the C-terminus. In certain embodiments, a CBE may comprise a NLS, a cytidine deaminase, a Cas-derived platform protein, and a NLS, optionally in the direction from the N-terminus to the C-terminus and optionally comprising a linker between different components. In certain embodiments, a CBE may further comprise one or more UGI. In particular embodiments, a CBE may be or may comprise BE4max (SEQ ID NO: 712); AncBE4max (SEQ ID NO: 713); BE4 (SEQ ID NO: 710); BE4-Gam (SEQ ID NO: 711); BE3 (SEQ ID NO: 700); YE1-BE3 (SEQ ID NO: 701); (vii) YE2-BE3 (SEQ ID NO: 702); (viii) EE-BE3 (SEQ ID NO: 703); (ix) YEE-BE3 (SEQ ID NO: 704); (x) CDA1-BE3 (SEQ ID NO: 705); (xi) AID-BE3 (SEQ ID NO: 706); and/or (xii) BE3-Gam (SEQ ID NO: 707), or a variant thereof.

In some embodiments, a DE may comprise a dual deaminase and a Cas-derived platform protein, optionally in the direction from the N-terminus to the C-terminus. In certain embodiments, a DE may comprise a NLS, a dual deaminase, a Cas-derived platform protein, and a NLS, optionally in the direction from the N-terminus to the C-terminus and optionally comprising a linker between different components. In certain embodiments, a DE may further comprise one or more UGI. In particular embodiments, a DE may be or may comprise TadDE (SEQ ID NO: 900); and/or TadDE-V106W (SEQ ID NO: 901), or a variant thereof.

Various ABEs, CBEs, and DEs have been reported for effecting gene editing to date. See. e.g., Gaudelli et al., Nature. 2017 Nov. 23;551 (7681): 464-471; Richter et al., Nat Biotechnol. 2020 July;38 (7): 883-891; Neugebauer et al., Nat Biotechnol. 2022 Nov. 10; Koblan et al., Nat Biotechnol. 2018 October;36 (9): 843-846; Komor et al., Nature. 2016 May 19;533 (7603): 420-4; Nishida et al., Science. 2016 Sep. 16;353 (6305): aaf8729; Kim et al., Nat Biotechnol. 2017 April;35 (4): 371-376; Komor et al., Sci Adv. 2017 Aug. 30;3 (8): eaao4774; and Koblan et al., Nat Biotechnol. 2018 October; 36 (9): 843-846.

### Methods (In Vitro, Ex Vivo, or In Vivo Treatment)

A method of effecting base editing according to the present disclosure may comprise applying an effective amount of any of the compositions described herein for base editing to the target cell. In some embodiments, the applying occurs in vitro. In some embodiments, the applying occurs ex vivo. In some embodiments, the applying occurs or in vivo.

A method of treating a subject or preventing or treating a disease, a disorder, or a condition in a subject may comprise administering an effective amount of any of the compositions described herein for base editing to the subject.

In any of the above methods, in some embodiments, the subject may have or have a risk of contracting with a genetic disease associated with one or more genetic mutations, or the target cell may have such one or more genetic mutations. In such as case, the composition may be designed to reverse or alter the genetic mutation(s). For example, the type of a deaminase (e.g., whether to use an adenine deaminase, a cytidine deaminase, or a dual deaminase), which may define the base type to be edited and/or the preference of the sequence context the base to be edited is in, and/or the type of Cas-derived platform protein (e.g., whether to use Cas9 or Cas12a and/or Cas of which species of origin), which may define the PAM sequence, (and such combination may further define the editing window, e.g., the span of editable DNA nt length) may be determined based on the nucleic acid base to be edited and the proximal sequence context in which the target base is in (e.g., proximity to a PAM sequence which may be used).

In certain embodiments, the intended alteration may be reversion to a wild-type base. In certain embodiments, the intended alteration may be alteration to a non-wild-type base. In some cases, the non-wild-type base may provide a silent mutation. In some cases, the non-wild-type base may provide a missense mutation, optionally at least partially restoring wild-type phenotype of the gene.

In some embodiments, when the composition is administered or applied in vivo, the composition may be administered via any appropriate route, which may optionally be selected appropriately based on the target disease and the affected site.

Methods (in vitro, ex vivo, or in vivo treatment)

A method of preparing the composition described herein for base editing may comprise providing an aqueous solution comprising the TCV and mixing a RNP which comprises a base editor complexed to a gRNA with the aqueous solution. In some embodiments, the method may be performed in the same or similar manner as methods of preparing compositions for CRISPR-mediated gene editing described herein.

### Specificity

When a base editor is designed as part of a composition or to be used in a method according to the present disclosure, a combination of an appropriate Cas-derived platform protein, an appropriate deaminase, and an appropriate gRNA may be designed and/or selected based on the desired base change and the sequence context around the desired base change position.

In some embodiments, when an A-to-G conversion is desired at a given nucleic acid position (or given nucleic acid positions that are in proximity (e.g., within 1, 2, 3, 4, 5, 6, 7, or 8 nucleic acid apart from each other)), an adenine deaminase may be selected as a deaminase, a Cas-derived platform protein may be selected based on the potential PAM sequences that are available near said nucleic acid position(s), and a gRNA may be designed to recognize the PAM sequence and to hybridize with a target DNA in a way to create a single-stranded DNA span containing the said given nucleic acid position(s) at which A-to-G conversion is desired.

In some embodiments, when an C-to-T conversion is desired at a given nucleic acid position (or given nucleic acid positions that are in proximity (e.g., within 1, 2, 3, 4, 5, 6, 7, or 8 nucleic acid apart from each other)), an adenine deaminase may be selected as a deaminase, a Cas-derived platform protein may be selected based on the potential PAM sequences that are available near said nucleic acid position(s), and a gRNA may be designed to recognize the PAM sequence and to hybridize with a target DNA in a way to create a single-stranded DNA span containing the said given nucleic acid position(s) at which C-to-T conversion is desired.

In some embodiments, when an A-to-G conversion and an C-to-T conversion are desired within a given nucleic acid span (e.g., a span of 2, 3, 4, 5, 6, 7, 8, 9, or 10 nucleic acids), an dual deaminase may be selected as a deaminase, a Cas-derived platform protein may be selected based on the potential PAM sequences that are available near said nucleic acid span, and a gRNA may be designed to recognize the PAM sequence and to hybridize with a target DNA in a way to create a single-stranded DNA span containing the said given nucleic acid span within which A-to-G conversion and C-to-T conversion are desired.

In certain embodiments, a particular deaminase may be selected which may prefer to provide deamination within the sequence context around said nucleic acid position(s) (e.g., nucleic acid residues up to +1, +2, or +3 nucleic acid position and/or up to −1, −2, or −3 nucleic acid position) or within the sequence context in said given nucleic acid span. Without wishing to be bound by theory, such a selection may increase editing efficacy. In certain embodiments, a particular deaminase (or a combination of a particular deaminase and a particular Cas-derived platform protein) may be selected having a preferred editing window (e.g., starting at a first nucleic acid position having a first relative distance from the PAM site ending at a second nucleic acid position having a second relative distance from the PAM site) which encompasses the nucleic acid position or all of the nucleic acid positions at which nucleic acid conversion is desired. In particular embodiments, a particular deaminase (or a combination of a particular deaminase and a particular Cas-derived platform protein) may be selected having a preferred editing window which contains a minimal number of nucleic acid positions at which conversion is not desired. Without wishing to be bound by theory, such a selection may increase editing specificity.

In certain embodiments, a deaminase may be evolved (e.g., by applying an evolutionary, genetic pressure) to prefer a particular sequence context of interest around a nucleic acid position(s) at which deamination is desired. In certain embodiments, a deaminase may be evolved (e.g., by applying an evolutionary, genetic pressure) to have a preferred editing window. Methods for inducing such an evolution is known in the field and described in some of the publications cited herein.

### Delivery Vehicles

This section further describes variations related to delivery vehicles, particularly TCVs, applicable to any of the aspects and embodiments described herein.

### Vehicle Type

Any components that may be used for effecting gene editing as described herein may be carried into as a cargo (or cargoes) into a cell by a delivery vehicle. In some embodiments, such components for effecting gene editing may be comprised in a composition which comprises a pharmaceutically acceptable carrier. Such a carrier may be a delivery vehicle. Such a delivery vehicle may be a TCV.

### Lipid-Based TCVs

TCVs particularly used in the present disclosure include lipid-based TCVs. Compared to non-lipid-based TCVs such as viral vectors, lipid-based TCVs may have several advantages, e.g., less immunogenicity if needed, no random integration into the target cell genome.

### Cationic Lipid

In some embodiments, a lipid-based TCV may comprise at least one cationic lipid. In some embodiments, the at least one cationic lipid may comprise DODMA, DODAP, DLinDAP, KC2, MC3, DODAC, DDAB, DOTAP, DOTMA, DLinDMA, DLenDMA, DLin-C-DAP, DLin-DAC, DLin-MA, DLin-S-DMA, DLin-2-DMAP, DLin-TMA.Cl, DLin-TAR.C1, DLin-MPZ, DLinAP, DOAP, DLin-EG-DMA, DLin-K-DMA, DLin-K-DMA or analogs thereof, ALNY-100, DOTMA, DOTAP.C1, DC-Chol, DOSPA, DOGS″, DMRIE, or any combinations thereof. In particular embodiments, the at least one ionizable cationic lipid may comprise or consist of DODMA. In some embodiments, the at least one cationic lipid may be an ionizable cationic lipid.

In some embodiments, a lipid-based TCV may comprise at least one ionizable cationic lipid. Examples of ionizable cationic lipids include but are not limited to: N-dimethyl-2,3-dioleyloxy) propylamine (DODMA), 1,2-dioleoyl-3-dimethylammonium propane (“DODAP”), 1,2-Dilinoleoyl-3-dimethylaminopropane (DLinDAP), 1,2-DiLinoleyloxy-N,N-dimethylaminopropane (DLinDMA), N,N-dimethyl-2,2-di-(9Z,12Z)-9,12-octadecadien-1-yl-1,3-dioxolane-4-ethanamine (KC2), and (6Z,9Z,28Z,31Z)-heptatriaconta-6,9,28,31-tetraen-19-yl 4-(dimethylamino) butanoate (MC3).

In some embodiments, a lipid-based TCV may be free of permanently cationic lipid. For example, N-(1-(2,3-dioleyloxyl) propyl)-N,N,N-trimethylammonium chloride (DOTMA), N-(1-(2,3-dioleyloxyl) propyl)-N-2-(sperminecarboxamido)ethyl)-N,N-dimethyl-ammonium trifluoracetate (DOSPA) (which is a lipid component of Lipofectamine®), and N-(1-(2,3-dioleoyloxy) propyl)-N,N,N-trimethylammonium chloride (DOTAP) are permanently cationic lipids.

The amount of the at least one ionizable cationic lipid may be determined as appropriate. In some cases, the amount of the at least one ionizable cationic lipid to be used may be determined based on the type of cargo.

In some embodiments, the amount of ionizable cationic lipid(s) relative to the total amount of TCV components may be about 10 mol % to about 70 mol %. In some embodiments, the total amount of TCV components may be about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %. In particular embodiments, for example when the cargo comprises a nucleic acid and a protein (or a RNP), the total amount of ionizable cationic lipid(s) relative to the total amount of TCV components may be about 20 mol %.

In a preferred embodiment, a lipid-based TCV according to the present disclosure may comprise DODMA at 20 mol % relative to the total amount of TCV components.

In some embodiments, the amount of ionizable cationic lipid(s) relative to the total amount of TCV components may be about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %. In particular embodiments, for example when the cargo comprises a nucleic acid such as a siRNA, sihRNA or miRNA or a RNA or DNA vector, the total amount of ionizable cationic lipid(s) relative to the total amount of TCV components may be about 50 mol %.

In a preferred embodiment, a lipid-based TCV according to the present disclosure comprises DODMA at 50 mol % relative to the total amount of TCV components.

### Helper Lipid

In some embodiments, a lipid-based TCV may comprise at least one helper lipid in addition to the at least one ionizable cationic lipid. In some embodiments, the at least one helper lipid may comprise DOPE, DSPC, DOPC, DPPC, DOPG, DPPG, POPC, POPE, DOPE-mal, DPPE, DMPE, DSPE, 16-O-monomethyl PE, 16-O-dimethyl PE, 18-1-trans PE, SOPE, or any combinations thereof. In particular embodiments, the at least one helper lipid may comprise or consist of DOPE. In some cases, the at least one helper lipid to be used may be determined based on the stability of the TCV.

The amount of the at least one helper lipid may be determined as appropriate.

In some embodiments, the total amount of helper lipid(s) relative to the total amount of TCV components may be about 10 mol % to about 60 mol %. In some embodiments, the total amount of helper lipid(s) relative to the total amount of TCV components may be about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %. In particular embodiments, the total amount of helper lipid(s) relative to the total amount of TCV components may be about 30 mol %.

In some embodiments, the total amount of helper lipid(s) relative to the total amount of TCV components may be about 20 mol % to about 60 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of helper lipid(s) relative to the total amount of TCV components may be about 40 mol %.

In a preferred embodiment, a lipid-based TCV according to the present disclosure comprises DOPE at 30 mol %. Such a TCV may be used, for example when the cargo comprises a nucleic acid and a protein (or a RNP).

### Phospholipid

In some embodiments, a lipid-based TCV may comprise at least one phospholipid in addition to the at least one ionizable cationic lipid. In some embodiments, the at least one phospholipid may comprise DSPC, DOPE, DPPC, DOPC, DMPC, PLPC, DAPC, PE, EPC, DLPC, DMPC, MPPC, PMPC, PSPC, DBPC, SPPC, DEPC, POPC, lysophosphatidyl choline, DSPE, DMPE, DPPE, POPE, lysophosphatidylethanolamine, or any combinations thereof. In particular embodiments, the at least one helper lipid may comprise or consist of DSPC.

In some embodiments, the amount of phospholipid(s) relative to the total amount of TCV components may be about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %. In particular embodiments, the total amount of phospholipid(s) relative to the total amount of TCV components may be about 10 mol %.

In some embodiments, the total amount of phospholipid(s) relative to the total amount of TCV components may be about 5 mol % to about 65 mol %, about 15 mol % to about 65 mol %, about 25 mol % to about 55 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of phospholipid(s) relative to the total amount of TCV components may be about 40 mol %.

In a preferred embodiment, a lipid-based TCV according to the present disclosure comprises DSPC at 10 mol % relative to the total amount of TCV components. Such a TCV may be used, for example when the cargo comprises a nucleic acid molecule or nucleic acid and a protein (or a RNP complex).

### Cholesterol or Cholesterol Derivative

In some embodiments, a lipid-based TCV may comprise at least one cholesterol or cholesterol derivative in addition to the at least one ionizable cationic lipid. In some embodiments, the at least one cholesterol or cholesterol derivative may comprise cholesterol, DC-Chol, 1,4-bis(3-N-oleylamino-propyl) piperazine, ICE, or any combinations thereof. In particular embodiments, the at least one cholesterol or cholesterol derivative may comprise or consist of cholesterol.

In some embodiments, the amount of cholesterol and/or cholesterol derivative(s) relative to the total amount of TCV components may be about 20 mol % to about 60 mol %. some embodiments, the amount of cholesterol and/or cholesterol derivative(s) relative to the total amount of TCV components may be about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%. In particular embodiments, the total amount of cholesterol and/or cholesterol derivative(s) relative to the total amount of TCV components may be about 40 mol % or about 39 mol %.

In a preferred embodiment, a lipid-based TCV according to the present disclosure comprises cholesterol at 40 mol % or 39 mol % relative to the total amount of TCV components. Such a TCV may be used, for example when the cargo comprises a nucleic acid molecule or a nucleic acid and a protein (or a RNP complex).

### PEG-Lipid

In some embodiments, a lipid-based TCV may comprise at least one PEG-lipid in addition to the at least one ionizable cationic lipid. In some embodiments, the at least one PEG-lipid may comprise PEG-DMG (e.g., (Avanti® Polar Lipids (Birmingham, AL)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, or any combinations thereof. In particular embodiments, the at least one PEG-lipid may comprise or consist of PEG-DMG.

In some embodiments, the amount of PEG and/or PEG-lipid(s) relative to the total amount of TCV components may be about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %. In particular embodiments, the total amount of PEG-lipid(s) relative to the total amount of TCV components may be about 1 mol %.

In a preferred embodiment, a lipid-based TCV according to the present disclosure comprises PEG-DMG at 1 mol % relative to the total amount of TCV components.

In a preferred embodiment, a lipid-based TCV according to the present disclosure comprises DODMA at 20 mol %, DOPE at 30 mol %, DSPC at 10 mol %, and cholesterol at 40 mol % relative to the total amount of TCV components. Such a TCV may be used, for example when the cargo comprises a nucleic acid and a protein (or a RNP complex).

In another preferred embodiment, a lipid-based TCV according to the present disclosure comprises DODMA at 50 mol %, DSPC at 10 mol %, cholesterol at 39 mol %, PEG-DMG at 1 mol % relative to the total amount of TCV components. Such a TCV may be used, for example when the cargo comprises a nucleic acid molecule.

### TCV Size

In some embodiments, the size of TCVs may be determined by any appropriate techniques. Non-limiting examples of measurement methods include dynamic light chattering, binding assays, surface plasmon resonance (SPR), static image analysis, and dynamic image analysis. An appropriate measurement technique may be selected based on the accuracy and the approximate size range the technique is optimal for.

In some embodiments, the size of the TCV before encapsulation of the at least one cargo may be in a range of about 3 nm to about 240 nm, about 6 nm to about 160 nm, about 9 nm to about 80 nm, optionally about 10-40 nm, further optionally about 20 nm to about 40 nm or about 20-35 nm, at pH of about 4. In particular embodiments, the size of the TCV before encapsulation of the at least one cargo may be in a range of about 9 nm to about 80 nm at pH of about 4.

### Organic Solvents and Detergents

In some embodiments, one characteristic of a pharmaceutical composition is that the composition is substantially, essentially, or entirely free of destabilizing agents, and/or contains significantly lower amounts of destabilizing agents compared to other pharmaceutical compositions comprising a similar type of TCVs.

In some embodiments, one characteristic of a pharmaceutical composition is that the composition is substantially, essentially, or entirely free of organic solvents and detergents, and/or contains significantly lower amounts of organic solvents and detergents compared to other pharmaceutical compositions comprising a similar type of TCVs.

In some embodiments, one characteristic of a pharmaceutical composition is that the composition is substantially, essentially, or entirely free of ethanol, methanol, isopropanol, tetrahydrofuran (THF), dimethyl sulfoxide (DMSO), dimethyl formamide (DMF), and acetonitrile (ACN), and/or contains significantly lower amounts of ethanol, methanol, isopropanol, THF, DMSO, DMF, and ACN, compared to other pharmaceutical compositions comprising a similar type of TCVs.

In particular embodiments, the pharmaceutical composition may be entirely free of methanol, isopropanol, THF, DMSO, DMF, and ACN.

With regard to ethanol, in some embodiments, the pharmaceutical composition may be substantially free of ethanol, which may mean that the ethanol concentration is 5% (v/v) or below. In particular embodiments, the pharmaceutical composition may be essentially free of ethanol, which may mean that the ethanol concentration is 0.5% (v/v) or below.

In a particular embodiment, the pharmaceutical composition may be entirely free of ethanol, methanol, isopropanol, THF, DMSO, DMF, and ACN.

While in the above set forth preferred construction, specific elements have been recited in order to adequately illustrate the principles of this invention, it will be apparent to those skilled in the art that alterations and modifications in the construction and arrangement of the system may be made without thereby departing from the spirit of said invention. Changes of form, of details of construction and materials may be made without thereby departing from the spirit of invention set forth, which shall be limited only by the scope of the appended claims/Examples are provided below to illustrate the present invention. These examples are not meant to constrain the present invention to any particular application or theory of operation.

### EXAMPLES

### Material Preparation

### Preparation of Transfection Competent Vesicles (TCVs):

### Materials

1,2-Dioleyloxy-3-dimethylamino-propane (DODMA) was purchased from Cayman Chemical (Ann Arbor, MI). 1,2-dioleoyl-sn-glycero-3-phosphoethanolamine (DOPE) and 1,2-distearoyl-sn-glycero-3-phosphocholine (DSPC) were purchased from Avanti Polar Lipids (Alabaster, AL). Cholesterol was purchased from Sigma Aldrich (St. Louis, MO). All lipids were maintained as ethanol stocks.

### TCV Formation

Lipid components (ionizable cationic lipid, helper lipid, phospholipid, and cholesterol) were dissolved in ethanol at appropriate ratios to achieve a final concentration of about 20-35 mM total lipid. Unless otherwise specified, the lipid component ratio of DODMA: DOPE: DSCP: cholesterol=20:30:10:40 (in mol %) was used. An aqueous phase was prepared containing about 25 mM sodium acetate (approximately pH 4) buffer. The two solutions were combined via rapid-mixing. Specifically, the organic phase containing lipids was mixed with the aqueous phase through a T-junction mixer fabricated to meet the specifications of the PEEK Low Pressure Tee Assembly (1/16, 0.02 in thru hole, Part #P-712) at a final flow rate of about 20 mL/min with about 1:3 organic: aqueous (v/v) ratio (Jeffs, Palmer, et al. Pharm Res. 2005;22 (3): 362-372; Kulkarni et al. Nanoscale. 2017 Sep. 21;9 (36): 13600-13609; Kulkarni et al. ACS Nano. 2018 May 22;12 (5): 4787-4795). The resulting suspension was dialyzed against 1000-fold volume of 25 mM sodium acetate (approximately pH 4) buffer to remove ethanol.

### Analysis of TCVs

Lipid concentrations were determined by assaying for the cholesterol content using a T-Cholesterol Assay Kit (Wako Chemicals, Mountain View, CA) and extrapolating total lipid concentration as described elsewhere (Chen et al. J Control Release. 2014 Dec. 28; 196:106-12). Nucleic acid entrapment was determined using the RiboGreen Assay as previously described (Chen et al. J Control Release. 2014 Dec. 28; 196:106-12; Leung et al. J Phys Chem B. 2015 Jul. 16;119 (28): 8698-706).

### Preparation of RNP:

RNP Complex of Cas9 and gRNA

Recombinant Cas9 endonuclease protein was obtained from IDT (San Jose, CA). gRNAs, such as “LaRo gRNA” and “LoxP gRNA”, having the targeting sequence of SEQ ID NOS: 140 and 150, respectively, tdTomato-targeting gRNAs designed by Applicant, e.g., “PS2 gRNA” and “PS3 gRNA”, having the targeting sequence of SEQ ID NOS: 120 and 130, respectively, a gRNA targeting human patient-derived PAX6 gene mutation of c.580G>T (designed to be used with SpCas9) designed by Applicant, and a luciferase-targeting control gRNA, having the targeting sequence of SEQ ID NO: 160 were obtained from Synthego (Redwood City, CA) with chemical modifications of 2′-O-methyl groups to the first and last three bases and 3′-phosphorothioate (PS) bonds between first three and last two bases. RNP formation was performed for example by combining 5 μL of a 10 μM gRNA solution (the gRNA solution may contain one gRNA at 10 μM or an equimolar mixture (but 10 μM in total) of different gRNAs) with 5 μL of a 10 μM Cas9 solution and allowing to stand at room temperature for about 5 minutes prior to encapsulation of the RNP in TCVs. Unless otherwise specified, the same ratio was used for a different scale preparation.

RNP Complex of ABE and gRNA

ABE8e (SEQ ID NO: 810, Richter et al., Nat Biotechnol. 2020 July;38 (7): 883-891) was produced by overexpression of the pABE8e-protein plasmid (#161788, Addgene, Watertown, MA, a gift from Dr. David Liu) followed by protein purification (Huang et al., Nat Protoc. 2021 February;16 (2): 1089-1128).

A gRNA targeting human patient-derived PAX6 gene mutation of c.580G>T (designed to be used with ABE, targets the same region as the gRNA designed to be used with SpCas9 mentioned above) was obtained from Synthego (Redwood City, CA) with chemical modifications of 2′-O-methyl groups to the first and last three bases and 3′-phosphorothioate (PS) bonds between first three and last two bases.

RNP formation was performed for example by combining 5 μL of a 10 μM gRNA solution with 5 μL of a 10 μM ABE8e solution and allowing to stand at room temperature for about 5 minutes prior to encapsulation of the RNP in TCVs. Unless otherwise specified, the same ratio was used for a different scale preparation.

### Tcv Encapsulation:

### Encapsulation of RNP (for Production of RNP-TCV)

For example, 8.33 μL (except for “ABE 100 nM” condition in Example 6) or 4.16 μL (for “ABE 100 nM” condition in Example 6) of a 10 mM TCV solution (molar concentration is the concentration of total lipid components of the TCV) (about pH 4) and 10 μL of about 5 μM RNP (about pH 7) were combined and allowed to incubate at room temperature for 5 minutes. Unless otherwise specified, the same ratio was used for a different scale preparation.

Encapsulation of RNP and ssODN

For example, 8.33 μL (except for Example 6) or 3.33 μL (for Example 6) of a 10 mM TCV solution (molar of total lipid components) (about pH 4), 10 μL of a mixture containing about 5 μM RNP (about pH 7), and 5 μL of a 10 μM (except for Example 6) or 5 μM (for Example 6) solution of ssODN (about pH 7) were combined and allowed to incubate at room temperature for 5 minutes. Unless otherwise specified, the same ratio was used for a different scale preparation.

### Size Distribution and Polydispersity Index (PDI)

The size distribution and polydispersity of the encapsulation products were measured using a Malvern Zetasizer Nano S instrument (Worcestershire, UK) (He-Ne lase, λ=632 nm, detection angle =173°).

### Example 1: gRNA Designing

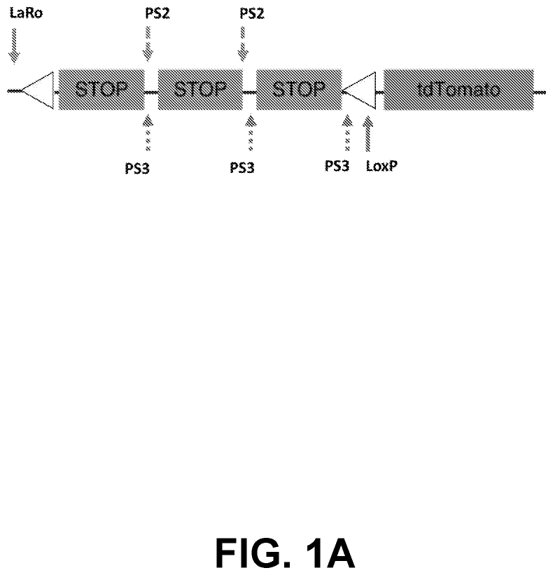

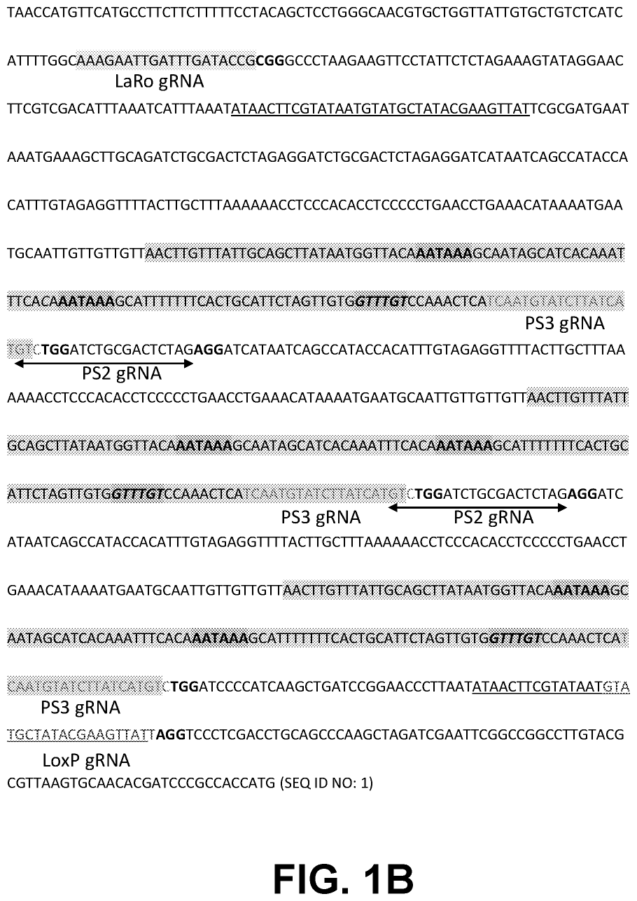

Previously, using Ai9 mice, Tabebordbar et al. (Tabebordbar et al. Science. 2016 Jan. 22;351 (6271): 407-411) showed that CRISPR/Cas-mediated gene editing on the floxed STOP cassette shown in FIGS. 1A and 1B via paired gRNAs targeting near the two loxP sites (“LaRo gRNA” and “LoxP gRNA” in FIG. 1B, having the targeting sequence of SEQ ID NOS: 140 and 150, respectively) result in excision of the intervening DNA segment and expression of tdTomato.

In an effort to search for a gRNA and/or a combination of gRNAs which provide improved gene editing and thus an improved gene editing reporter system, Applicant selected different target sites in the floxed STOP cassette and designed gRNAs. The PAM sites for some of the target sites selected are shown in bold in FIG. 1B. The newly designed gRNA sequences of 20 nt in length include SEQ ID NO: 120 (which is shown as “PS2 gRNA” and targets two sites corresponding to the arrowed segments in FIG. 1B) and SEQ ID NO: 130 (which is shown as “PS3 gRNA” and targets three sites corresponding to the segments in red in FIG. 1B).

### Example 2: Ex vivo gene editing-comparison of single and multiple gRNAs

### Method

First, primary cells from Ai14 mice were used to test the gene editing efficiency of different combinations of gRNAs targeting different sites of the Ai14 transgene upstream of the TdTomato reporter gene (arrowed in FIG. 1).

Cortical neurons were harvested from Ai14 mice and a primary culture was established. On day in vitro 6 (DIV6), 150,000 cells in 1 mL per well were plated on a 24-well culture dish and were cultured with TCVs (at 83.3 μM; molar in terms of the lipid components) encapsulating RNPs (final culture concentration of 50 nM) containing different combinations of gRNAs as shown in Table 1 for 2 hours. After the 2-hour incubation, the medium was changed.

TABLE 1
gRNA combinations and concentrations in culture.

| | | gRNA | | total |
|---|---|---|---|---|
| Group | gRNA(s) | concentration | | gRNA |
| Single Guide | LoxP | 50 | nM | 50 nM |
| Multiple Guides A | LoxP + LaRo | 25 | nM each | 50 nM |
| Multiple Guides B | LoxP + LaRo + | 16.67 | nM each | 50 nM |
| | PS2 | | | |
| Multiple Guides C | LoxP + LaRo + | 16.67 | nM each | 50 nM |
| | PS3 | | | |
| Multiple Guides D | LoxP + LaRo + | 12.5 | nM each | 50 nM |
| | PS2 + PS3 | | | |

The targeting sequences were: SEQ ID NOs: 120, 130, 140, 150 for PS2 gRNA, PS3 gRNA, LaRo gRNA, LoxP gRNA: SEQ ID NO: 150, respectively.

On day in vitro 9 (DIV9) (i.e., 3 days post RNP-TCV treatment), cells were harvested, washed, and fixed with 4% paraformaldehyde. Red fluorescence of tdTomato was analyzed by fluorescent microscopy. Hoechst staining (blue) was used to stain for nuclei.

### Results

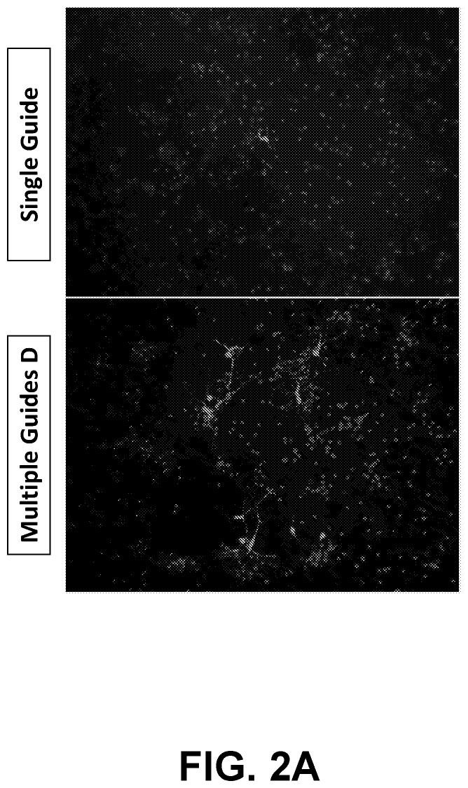

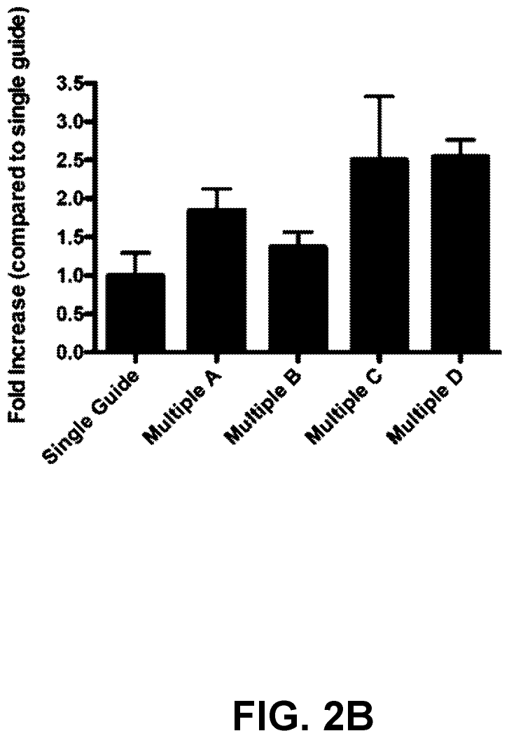

Exemplary microscopic results for Single Guide and Multiple Guides D groups are provided in FIG. 2A and increases in cells with red fluorescence in each group relative to Single Guide are summarized in FIG. 2B. p=0.013 by t-test for Single guide vs Multiple D.

### Example 3: In Vivo Gene Editing-Comparison of Single and Multiple gRNAs

### Method

Next, Applicant tested whether use of multiple gRNAs results in increased gene editing in vivo as well.

2 μL of RNP-TCV containing a total of 4.5 μmol of TCVs (mol in terms of the lipid components) which encapsulate one or more gRNAs and optionally ssODN in as shown in Table 2 was injected directly into the stromal (middle) layer of the cornea of Ai14 mice. 21 days post injection, the corneas were collected, sectioned, and stained with Hoechst. Red fluorescence of tdTomato was analyzed by fluorescent microscopy.

TABLE 2
gRNA and ssODN encapsulated in 4.5 μmol of TCVs.

| | | RNP | ssODN |
|---|---|---|---|
| Group | gRNA(s) | concentration | concentration |
| Control | Off-target gRNA | 2.73 pmol/μL | |
| | (against luciferase | | |
| | gene) | | |
| Single | LoxP | 2.73 pmol/μL | |
| Guide | | | |
| Multiple | LoxP + LaRo + PS2 + | 2.14 pmol/μL (0.535 | 1.07 pmol/μL |
| Guides | PS3 | pmol RNP/μL for | |
| | | each gRNA) | |

The targeting sequences were: SEQ ID NOs: 120, 130, 140, 150 for PS2 gRNA, PS3 gRNA, LaRo gRNA, LoxP gRNA, respectively, and SEQ ID NO: 160 for luciferase gRNA. ssODN sequence was SEQ ID NO: 540.

### Results

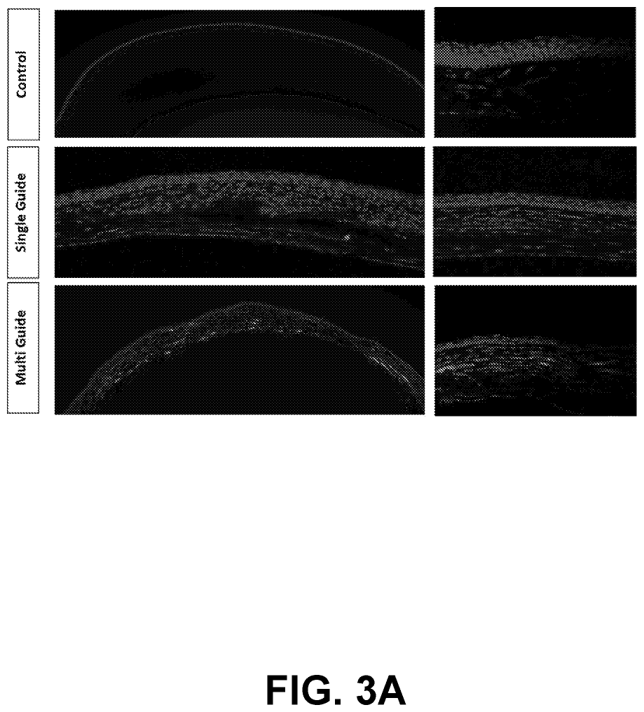

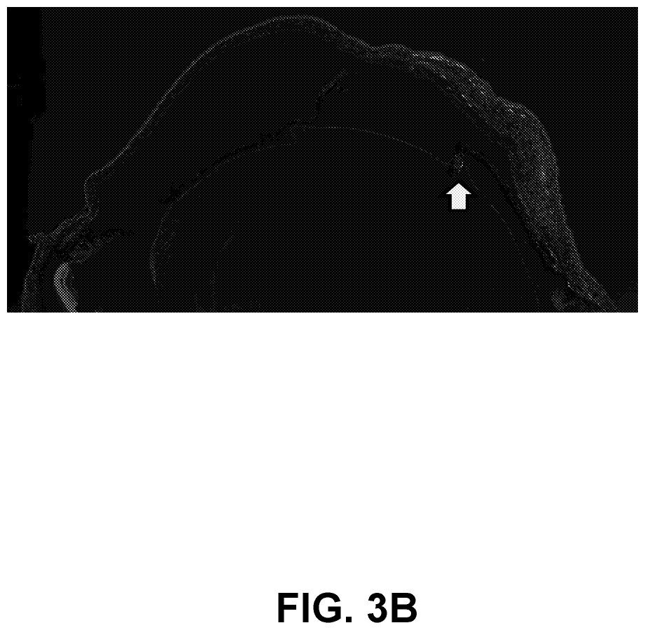

Exemplary microscopic results are provided in FIGS. 3A and 3B. As shown in FIG. 3A, while Control group and Single Guide group respectively had zero or only a small number of cells with red fluorescence, Multi Guide group had significant numbers of cells with red fluorescence. Interestingly, red fluorescence was also observed in the iris in Multiple Guide group, as shown in FIG. 3B.

### Example 4: Ex Vivo Gene Editing-Comparison of Different ssODN Lengths

### Method

Primary cells from Ai14 mice were used to test the gene editing efficiency when a ssODN of different lengths was used.

Cortical neurons were harvested from Ai14 mice and a primary culture was established. On day in vitro 6 (DIV6), 150,000 cells in 1 mL per well were plated on a 24-well culture dish and were cultured with TCV (at 83.3 μM; molar in terms of the lipid components) encapsulating RNPs (final culture concentration of 50 nM) containing the combination of four gRNAs (equimolar ratio of LoxP, LaRo, PS2, and PS3 (at 50 nM in total) as used in Example 2) and a ssODN of 80 nt or 100 nt (referred to as 80T and 100T, respectively) as shown in Table 3 for 2 hours. After the 2-hour incubation, the medium was changed.

TABLE 3
gRNA and ssODN type and concentrations in culture.

| | | gRNA | | ssODN |
|---|---|---|---|---|
| | | concen- | | concen- |
| Group | gRNAs | tration | ssODN | tration |
| All mix | LoxP + LaRo + | 12.5 | None | 0 nM |
| | PS2 + PS3 | nM each | | |
| All mix + | LoxP + LaRo + | 12.5 | 80T (40 nt | 50 nM |
| 80T | PS2 + PS3 | nM each | homology arms) | |
| All mix + | LoxP + LaRo + | 12.5 | 100T (50 nt | 50 nM |
| 100T | PS2 + PS3 | nM each | homology arms) | |

The ssODN sequences were: SEQ ID NOs: 540 and 550 for 80T and 100TssODN, respectively.

On day in vitro 9 (DIV9) (i.e., 3 days post RNP-TCV treatment), cells were harvested, washed, and fixed with 4% paraformaldehyde. Cells were analyzed by fluorescent microscopy. Cells with red fluorescence was counted using a grid of 3000×3000 μm with a probe of 300×300 μm, and % cells with red fluorescence were calculated.

### Results

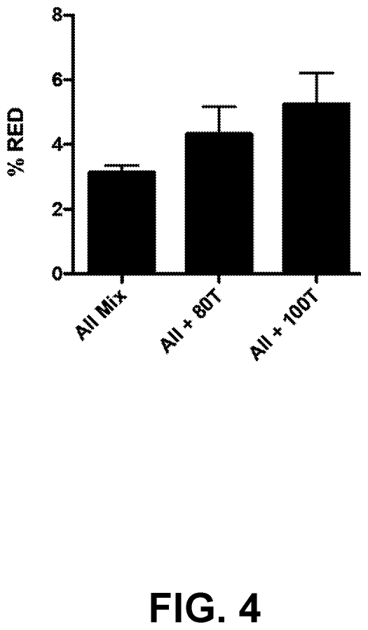

% cells with red fluorescence are summarized in FIG. 4. Both groups using a ssODN (80T or 100T) showed successful gene editing. Editing efficacies in these two groups were significantly higher than a negative control which used a gRNA targeting luciferase (two-way ANOVA with post-hoc Tukey test).

### Example 5: Ex Vivo Gene Editing-Comparison of Single and Multiple Treatments

### Method

Primary cells from Ai14 mice were used to test whether multiple treatments with RNP-TCVs provide higher gene editing.

Cortical neurons were harvested from Ai14 mice and a primary culture was established. On days in vitro 3 and/or 6 (DIV 3 and/or DIV6), 150,000 cells in 1 mL per well were plated on a 24-well culture dish and were cultured with TCV (83.3 μM; molar in terms of the lipid components) encapsulating RNP (at final culture concentration of 50 nM) containing the combination of four gRNAs (equimolar ratio of LoxP, LaRo, PS2, and PS3 (at 50 nM in total) as used in Examples 2 and 5) and a ssODN of 80 nt or 100nt (at 50 nM in total) as shown in Table 4 for 2 hours. After the 2-hour incubation, the medium was changed.

TABLE 4
Treatment timing and gRNA and ssODN type.

| Group | Treatment timing | gRNAs | ssODN |
|---|---|---|---|
| All + 80T | DIV6 | LoxP + LaRo + PS2 + PS3 | 80T |
| All + 80T × 2 | DIV3 and DIV6 | LoxP + LaRo + PS2 + PS3 | 80T |
| All + 100T | DIV6 | LoxP + LaRo + PS2 + PS3 | 100T |

On day in vitro 9 (DIV9) (i.e., 3 days post RNP-TCV treatment), cells were harvested, washed, and fixed with 4% paraformaldehyde. Cells were analyzed by fluorescent microscopy. Cells with red fluorescence was counted using a grid of 3000×3000 μm with a probe of 300×300 μm, and % cells with red fluorescence were calculated.

### Results

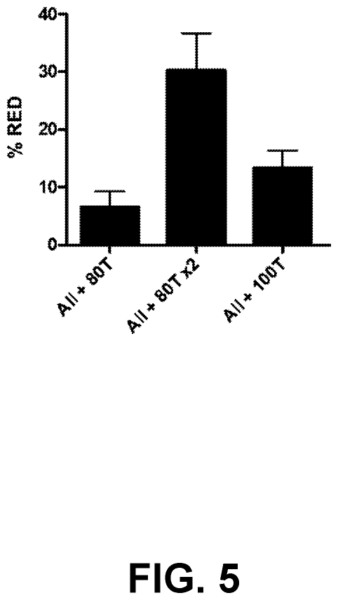

% cells with red fluorescence are summarized in FIG. 5. ANOVA p=0.012. p<0.05 via Tukey post hoc analysis for All+80T vs All+80T x2 and All+100T vs All+80T x2.

### Example 6: Ex Vivo Gene Editing at Human Disease Mutation Site Using Base Editor

Base editors are comprised of a Cas endonuclease variant (typically Cas nickase) linked or fused to a deaminase and thus are generally larger than Cas endonucleases. Therefore, it was unknown whether a TCV such as those described herein would effectively encapsulate base editor-gRNA complexes and/or deliver such complexes to cells to effect intended base editing.

### Method

Fey mice (also called 3xFLAG-tagged Pax6 Sey mice, an aniridia mouse model harboring one Fey allele (3xFLAG-tagged, mutant Pax6 containing the human patient PAX6 gene mutation of c.580G>T, which results in PAX6 deficiency) and one wildtype Pax6 allele (thus also referred to as Fey/+)) (Mohanna et al., Mol Ther Methods Clin Dev. 2020 Mar. 14; 17:478-490), were bred as previously described (Mohanna et al., J Control Release. 2022 October;350:401-413). In Fey mice, restoration of PAX6 expression from the Fey allele can be detected by FLAG expression.

A primary culture of embryonic cortical neurons of Fey mice were established as previously described (Mohanna et al., J Control Release. 2022 October;350:401-413). On ex vitro day 6 (DEV 6), cells were treated under five different conditions as shown in Table 5 for 2 hours. The SpCas9 HDR treatment was designed to correct the 580T mutation back to G, and the ABE treatment was designed to convert the 580T mutation to C (the resulting C provides a missense mutation). The treatment media were then replaced with fresh media and cells were incubated for 72 hours. On ex vitro day 9 (DEV 9), cells were harvested and stained with Hoechst and anti-FLAG and anti-PAX6 antibodies. Stained cells were analyzed by immunocytochemistry as previously described (Mohanna et al., J Control Release. 2022 October;350:401-413) and % FLAG+ cells among all PAX6+ cells were quantified (embryonic cortical neurons generally express PAX6). % cells containing the intended gene alternation (missense mutation) were also quantified by Sanger sequencing.

TABLE 5
Treatment conditions

| Group | Treatment | RNP | gRNA target | ssODN |
|---|---|---|---|---|
| Untreated | Medium only | | | |
| Negative | TCV encapsulating | Cas9-gRNA, | luciferase | |
| control | RNP | at 100 nM | | |
| SpCas9 HDR | TCV encapsulating | Cas9-gRNA, | PAX6 gene mutation | ssODN to correct |
| | RNP and ssODN | at 100 nM | c.580G > T designed | PAX6 gene mutation |
| | | | for SpCas9 | c.580G > T, at 50 nM |
| ABE 50 nM | TCV encapsulating | ABE-gRNA, | PAX6 gene mutation | |
| | RNP | at 50 nM | c.580G > T designed | |
| | | | for ABE | |
| ABE 100 nM | TCV encapsulating | ABE-gRNA, | PAX6 gene mutation | |
| | RNP | at 100 nM | c.580G > T, designed | |
| | | | for ABE | |

### Results

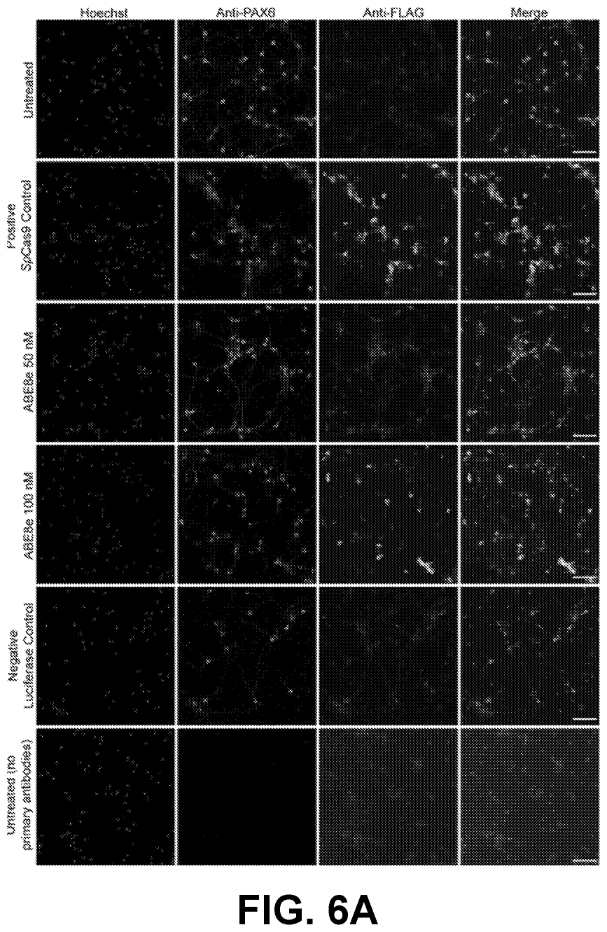

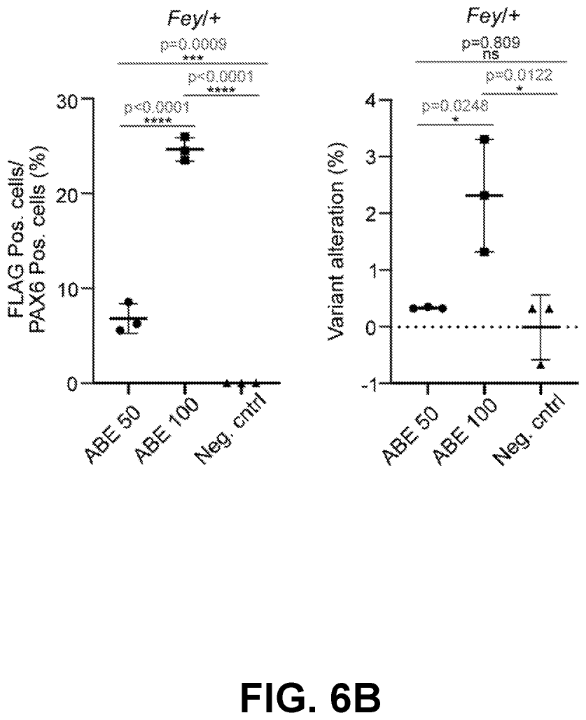

Results are provided in FIGS. 6A-6B. Representative immunocytochemistry results are provided in FIG. 6A. FIG. 6B provides quantification of % FLAG+ cells/PAX6+ cells (left; p<0.0001, one-way ANOVA with Tukey's multiple comparisons tests) and % cells containing the intended gene alternation (right; p=0.0105, one-way ANOVA with Tukey's multiple comparisons tests). The results overall indicate that the TCV is capable of encapsulating and delivering ABE-gRNA complexes to effect intended base editing.

### Example 7: In Vivo Gene Editing in the Retina

### Method

2 μL of RNP-TCV containing a total of 4.5 μmol of TCVs (mol in terms of the lipid components) which encapsulate one or more gRNAs and optionally ssODN in as shown in Table 2 was injected directly into the vitreous humor of Ai14 mice. 21 days post injection, the eyes were collected, sectioned, and stained with Hoechst. Red fluorescence of tdTomato was analyzed by fluorescent microscopy.

### Appendix: Amino Acid and Nucleic Acid Sequences

Transgene segments:
SEQ ID NO: 1
Nucleic acid sequence
Ai14 mouse transgene region 1
TAACCATGTTCATGCCTTCTTCTTTTTCCTACAGCTCCTGGGCAACGTGCTGGTTATTGTGCTGTCTCATCATTTT

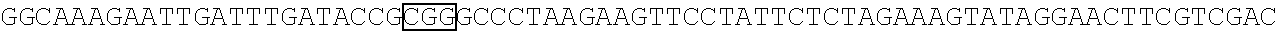

ATTTAAATCATTTAAATATAACTTCGTATAATGTATGCTATACGAAGTTATTCGCGATGAATAAATGAAAGCTT

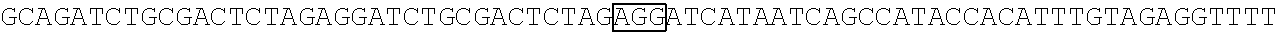

ACTTGCTTTAAAAAACCTCCCACACCTCCCCCTGAACCTGAAACATAAAATGAATGCAATTGTTGTTGTTAACTT
GTTTATTGCAGCTTATAATGGTTACAAATAAAGCAATAGCATCACAAATTTCACAAATAAAGCATTTTTTTCAC

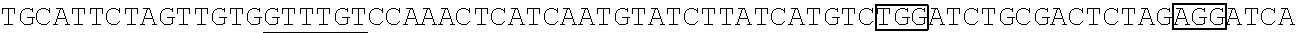

TAATCAGCCATACCACATTTGTAGAGGTTTTACTTGCTTTAAAAAACCTCCCACACCTCCCCCTGAACCTGAAAC
ATAAAATGAATGCAATTGTTGTTGTTAACTTGTTTATTGCAGCTTATAATGGTTACAAATAAAGCAATAGCATC
ACAAATTTCACAAATAAAGCATTTTTTTCACTGCATTCTAGTTGTGGTTTGTCCAAACTCATCAATGTATCTTAT

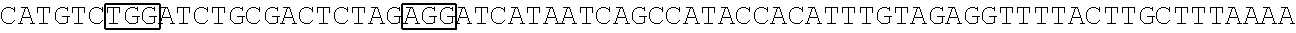

AACCTCCCACACCTCCCCCTGAACCTGAAACATAAAATGAATGCAATTGTTGTTGTTAACTTGTTTATTGCAGCT
TATAATGGTTACAAATAAAGCAATAGCATCACAAATTTCACAAATAAAGCATTTTTTTCACTGCATTCTAGTTG

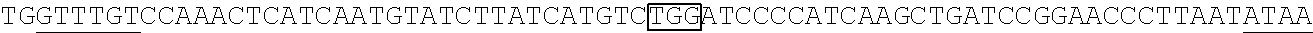

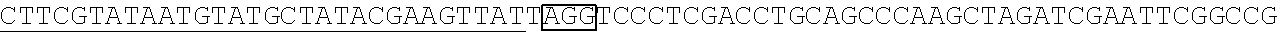

GCCTTGTACGCGTTAAGTGCAACACGATCCCGCCACCATG
SEQ ID NO: 2
Nucleic acid sequence
LoxP
ATAACTTCGTATAATGTATGCTATACGAAGTTAT
SEQ ID NO: 3
Nucleic acid sequence
SV40 terminator
AACTTGTTTATTGCAGCTTATAATGGTTACAAATAAAGCAATAGCATCACAAATTTCACAAATAAAGCATTTTT
TTCACTGCATTCTAGTTGTGGTTTGTCCAAACTCATCAATGTATCTTATCATGT
SEQ ID NO: 20
Nucleic acid sequence
Ai14 mouse transgene region 2 (20 nt + PAM)

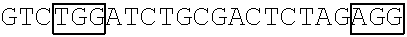

SEQ ID NO: 21
Nucleic acid sequence
Ai14 mouse transgene region 2 (19 nt + PAM)

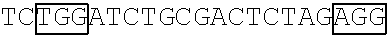

SEQ ID NO: 22
Nucleic acid sequence
Ai14 mouse transgene region 2 (18 nt + PAM)

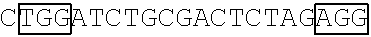

SEQ ID NO: 23
Nucleic acid sequence
Ai14 mouse transgene region 2 (17 nt + PAM)

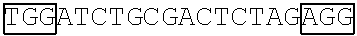

SEQ ID NO: 30
Nucleic acid sequence
Ai14 mouse transgene region 3 (20 nt + PAM)

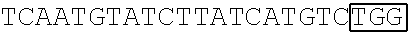

SEQ ID NO: 31
Nucleic acid sequence
Ai14 mouse transgene region 3 (19 nt + PAM)

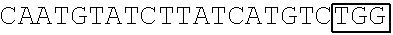

SEQ ID NO: 32
Nucleic acid sequence
Ai14 mouse transgene region 3 (18 nt + PAM)

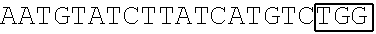

SEQ ID NO: 33
Nucleic acid sequence
Ai14 mouse transgene region 3 (17 nt + PAM)

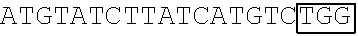

SEQ ID NO: 40
Nucleic acid sequence
Ai14 mouse transgene region 4 (20 nt + PAM)

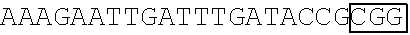

SEQ ID NO: 41
Nucleic acid sequence
Ai14 mouse transgene region 4 (19 nt + PAM)

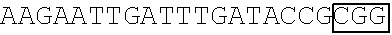

SEQ ID NO: 42
Nucleic acid sequence
Ai14 mouse transgene region 4 (18 nt + PAM)

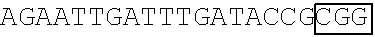

SEQ ID NO: 43
Nucleic acid sequence
Ai14 mouse transgene region 4 (17 nt + PAM)

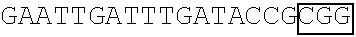

SEQ ID NO: 50
Nucleic acid sequence
Ai14 mouse transgene region 5 (20 nt + PAM)

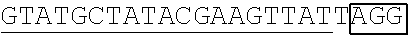

SEQ ID NO: 51
Nucleic acid sequence
Ai14 mouse transgene region 5 (19 nt + PAM)

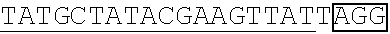

SEQ ID NO: 52
Nucleic acid sequence
Ai14 mouse transgene region 5 (18 nt + PAM)

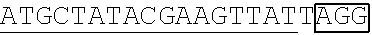

SEQ ID NO: 53
Nucleic acid sequence
Ai14 mouse transgene region 5 (17 nt + PAM)

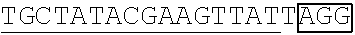

SEQ ID NO: 60
Nucleic acid sequence
Ai14 mouse transgene region 6 (region within region 1 for targeting by one PS2 gRNA and one PS3 gRNA)

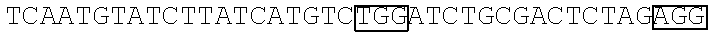

SEQ ID NO: 70
Nucleic acid sequence
Ai14 mouse transgene region 7 (region within region 1 for targeting by two PS2 gRNAs and three PS3 gRNAs)

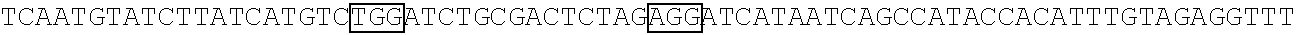

TACTTGCTTTAAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCATTTTTTTCA

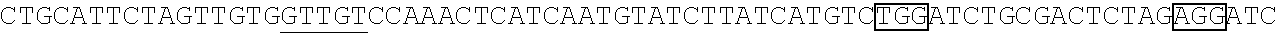

ATAATCAGCCATACCACATTTGTAGAGGTTTTACTTGCTTTAAAAAACCTCCCACACCTCCCCCTGAACCTGAAA
CATAAAATGAATGCAATTGTTGTTGTTAACTTGTTTATTGCAGCTTATAATGGTTACAAATAAAGCAATAGCAT
CACAAATTTCACAAATAAAGCATTTTTTTCACTGCATTCTAGTTGTGGTTTGTCCAAACTCATCAATGTATCTTA

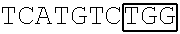

SEQ ID NO: 80
Nucleic acid sequence
Ai14 mouse transgene region 8 (region within region 1 between LaRo gRNAcleavage site and LoxP
gRNA cleavage site)

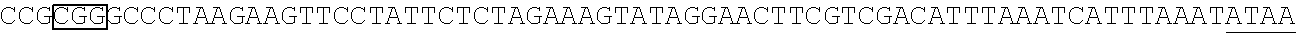

CTTCGTATAATGTATGCTATACGAAGTTATTCGCGATGAATAAATGAAAGCTTGCAGATCTGCGACTCTAGAG

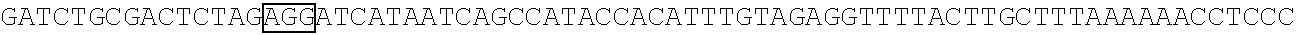

ACACCTCCCCCTGAACCTGAAACATAAAATGAATGCAATTGTTGTTGTTAACTTGTTTATTGCAGCTTATAATG
GTTACAAATAAAGCAATAGCATCACAAATTTCACAAATAAAGCATTTTTTTCACTGCATTCTAGTTGTGGTTTG

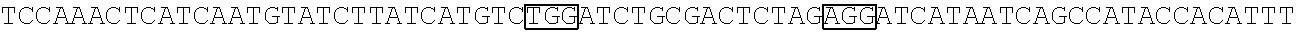

GTAGAGGTTTTACTTGCTTTAAAAAACCTCCCACACCTCCCCCTGAACCTGAAACATAAAATGAATGCAATTGT
TGTTGTTAACTTGTTTATTGCAGCTTATAATGGTTACAAATAAAGCAATAGCATCACAAATTTCACAAATAAAG

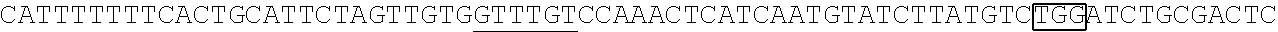

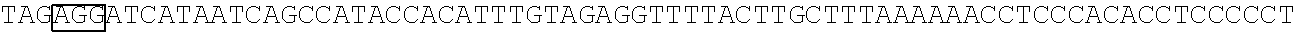

GAACCTGAAACATAAAATGAATGCAATTGTTGTTGTTAACTTGTTTATTGCAGCTTATAATGGTTACAAATAAA
GCAATAGCATCACAAATTTCACAAATAAAGCATTTTTTTCACTGCATTCTAGTTGTGGTTTGTCCAAACTCATCA

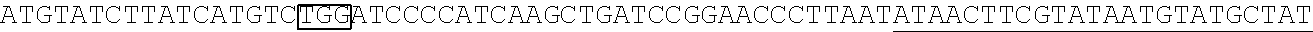

ACGAAGTT
SEQ ID NO: 81
Nucleic acid sequence
Ai14 mouse transgene region 8 (complementary to SEQ ID NO: 80)
AACTTCGTATAGCATACATTATACGAAGTTATATTAAGGGTTCCGGATCAGCTTGATGGGGATCCAGACATGA
TAAGATACATTGATGAGTTTGGACAAACCACAACTAGAATGCAGTGAAAAAAATGCTTTATTTGTGAAATTTG
TGATGCTATTGCTTTATTTGTAACCATTATAAGCTGCAATAAACAAGTTAACAACAACAATTGCATTCATTTTAT
GTTTCAGGTTCAGGGGGAGGTGTGGGAGGTTTTTTAAAGCAAGTAAAACCTCTACAAATGTGGTATGGCTGA
TTATGATCCTCTAGAGTCGCAGATCCAGACATGATAAGATACATTGATGAGTTTGGACAAACCACAACTAGAA
TGCAGTGAAAAAAATGCTTTATTTGTGAAATTTGTGATGCTATTGCTTTATTTGTAACCATTATAAGCTGCAATA
AACAAGTTAACAACAACAATTGCATTCATTTTATGTTTCAGGTTCAGGGGGAGGTGTGGGAGGTTTTTTAAAG
CAAGTAAAACCTCTACAAATGTGGTATGGCTGATTATGATCCTCTAGAGTCGCAGATCCAGACATGATAAGAT
ACATTGATGAGTTTGGACAAACCACAACTAGAATGCAGTGAAAAAAATGCTTTATTTGTGAAATTTGTGATGC
TATTGCTTTATTTGTAACCATTATAAGCTGCAATAAACAAGTTAACAACAACAATTGCATTCATTTTATGTTTCA
GGTTCAGGGGGAGGTGTGGGAGGTTTTTTAAAGCAAGTAAAACCTCTACAAATGTGGTATGGCTGATTATGA
TCCTCTAGAGTCGCAGATCCTCTAGAGTCGCAGATCTGCAAGCTTTCATTTATTCATCGCGAATAACTTCGTATA
GCATACATTATACGAAGTTATATTTAAATGATTTAAATGTCGACGAAGTTCCTATACTTTCTAGAGAATAGGAA
CTTCTTAGGGCCCGCGG
SEQ ID NO: 90
Nucleic acid sequence
Ai14 mouse transgene region 9 (region within region 1 for targeting by two PS2 gRNAs, three PS3
gRNAs, one LaRo gRNA, and one LoxP gRNA)

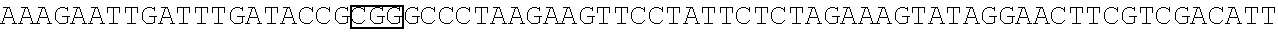

TAAATCATTTAAATATAACTTCGTATAATGTATGCTATACGAAGTTATTCGCGATGAATAAATGAAAGCTTGCA

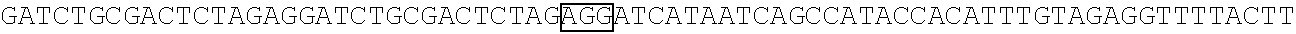

GCTTTAAAAAACCTCCCACACCTCCCCCTGAACCTGAAACATAAAATGAATGCAATTGTTGTTGTTAACTTGTTT
ATTGCAGCTTATAATGGTTACAAATAAAGCAATAGCATCACAAATTTCACAAATAAAGCATTTTTTTCACTGCA

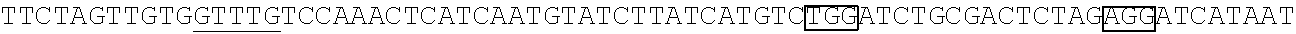

CAGCCATACCACATTTGTAGAGGTTTTACTTGCTTTAAAAAACCTCCCACACCTCCCCCTGAACCTGAAACATAA
AATGAATGCAATTGTTGTTGTTAACTTGTTTATTGCAGCTTATAATGGTTACAAATAAAGCAATAGCATCACAA
ATTTCACAAATAAAGCATTTTTTTCACTGCATTCTAGTTGTGGTTTGTCCAAACTCATCAATGTATCTTATCATG

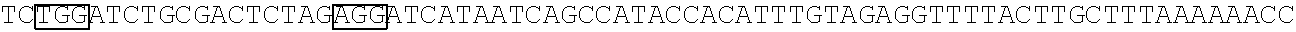

TCCCACACCTCCCCCTGAACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCACTGCATTCTAGTTGTGGT

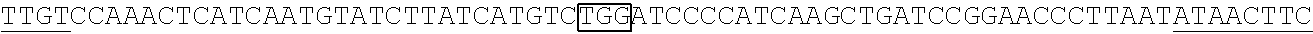

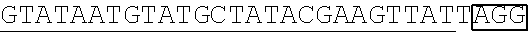

gRNA subparts:
Nucleic acid sequence
crRNA flagpole sequence (option 1)
SEQ ID NO: 101
GUUUUAGAGCUA
Nucleic acid sequence
crRNA flagpole sequence (option 2)
SEQ ID NO: 102
GUUUAAGAGCUA
Nucleic acid sequence
tracrRNA flagpole (option 1)
SEQ ID NO: 106
UAGCAAGUUAAAA
Nucleic acid sequence
tracrRNA flagpole (option 2)
SEQ ID NO: 107
UAGCAAGUUUAAA
Nucleic acid sequence
tracrRNA nuclease binding domain (may be
followed by multiple uracils)
SEQ ID NO: 108
UAAGGCUAGUCCGUUAUCAACUUGAAAAAGUGGCACCGAGUCGGUGC
Nucleic acid sequence
sgRNA backbone (3′ to targeting sequence)
(option 1) (may be followed by multiple uracils)
SEQ ID NO: 111
GUUUUAGAGCUAGAAAUAGCAAGUUAAAAUAAGGCUAGUCCGUUAU
CAACUUGAAAAAGUGGCACCGAGUCGGUGC
Nucleic acid sequence
sgRNA backbone (3′ to targeting sequence)
(option 2) (may be followed by multiple uracils)
SEQ ID NO: 112
GUUUAAGAGCUAGAAAUAGCAAGUUUAAAUAAGGCUAGUCCGUUAUC
AACUUGAAAAAGUGGCACCGAGUCGGUGC
Nucleic acid sequence
sgRNA backbone (3′ to targeting sequence)
(option 3) (may be followed by multiple uracils)
SEQ ID NO: 113
GUUUUAGAGCUAUGCUGGAAACAGCAUAGCAAGUUAAAAUAAGGCUA
GUCCGUUAUCAACUUGAAAAAGUGGCACCGAGUCGGUGC
Nucleic acid sequence
sgRNA backbone (3′ to targeting sequence)
(option 4) (may be followed by multiple uracils)
SEQ ID NO: 114
GUUUAAGAGCUAUGCUGGAAACAGCAUAGCAAGUUUAAAUAAGGCUA
GUCCGUUAUCAACUUGAAAAAGUGGCACCGAGUCGGUGC
Nucleic acid sequence
dgRNA crRNA backbone (option 1)
SEQ ID NO: 115
GUUUUAGAGCUAUGCUGUUUUG
Nucleic acid sequence
dgRNA tracrRNA (option 1)
SEQ ID NO: 116
AACAGCAUAGCAAGUUAAAAUAAGGCUAGUCCGUUAUCAACUUGAAA
AAGUGGCACCGAGUCGGUGCUUUUUUU
Nucleic acid sequence
dgRNA crRNA backbone (option 2)
SEQ ID NO: 117
GUUUAAGAGCUAUGCUGUUUUG
Nucleic acid sequence
dgRNA tracrRNA (option 2)
SEQ ID NO: 118
AACAGCAUAGCAAGUUUAAAUAAGGCUAGUCCGUUAUCAACUUGAAA
AAGUGGCACCGAGUCGGUGCUUUUUUU

gRNA targeting sequence:
SEQ ID NO: 120
Nucleic acid sequence
PS2 gRNA targeting sequence (20 nt)

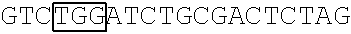

SEQ ID NO: 121
Nucleic acid sequence
PS2 gRNA targeting sequence (19 nt)

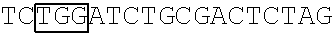

SEQ ID NO: 122
Nucleic acid sequence
PS2 gRNA targeting sequence (18 nt)

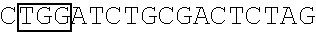

SEQ ID NO: 123
Nucleic acid sequence
PS2 gRNA targeting sequence (17 nt)

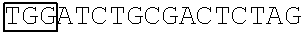

SEQ ID NO: 130
Nucleic acid sequence
PS3 gRNA targeting sequence (20 nt)
TCAATGTATCTTATCATGTC
SEQ ID NO: 131
Nucleic acid sequence
PS3 gRNA targeting sequence (19 nt)
CAATGTATCTTATCATGTC
SEQ ID NO: 132
Nucleic acid sequence
PS3 gRNA targeting sequence (18 nt)
AATGTATCTTATCATGTC
SEQ ID NO: 133
Nucleic acid sequence
PS3 gRNA targeting sequence (17 nt)
ATGTATCTTATCATGTC
SEQ ID NO: 140
Nucleic acid sequence
LaRo gRNA targeting sequence (20 nt)
AAAGAATTGATTTGATACCG
SEQ ID NO: 141
Nucleic acid sequence
LaRo gRNA targeting sequence (19 nt)
AAGAATTGATTTGATACCG
SEQ ID NO: 142
Nucleic acid sequence
LaRo gRNA targeting sequence (18 nt)
AGAATTGATTTGATACCG
SEQ ID NO: 143
Nucleic acid sequence
LaRo gRNA targeting sequence (17 nt)
GAATTGATTTGATACCG
SEQ ID NO: 150
Nucleic acid sequence
LoxP gRNA targeting sequence (20 nt)
GTATGCTATACGAAGTTATT
SEQ ID NO: 151
Nucleic acid sequence
LoxP gRNA targeting sequence (19 nt)
TATGCTATACGAAGTTATT
SEQ ID NO: 152
Nucleic acid sequence
LoxP gRNA targeting sequence (18 nt)
ATGCTATACGAAGTTATT
SEQ ID NO: 153
Nucleic acid sequence
LoxP gRNA targeting sequence (17 nt)
TGCTATACGAAGTTATT
SEQ ID NO: 160
Nucleic acid sequence
Luciferase gRNA targeting sequence (20 nt)
CTTCGAAATGTCCGTTGGGT

sgRNA (targeting sequence + backbone (option 1)):
SEQ ID NO: 125
Nucleic acid sequence
PS2 gRNA targeting sequence (20 nt) + backbone (option 1)

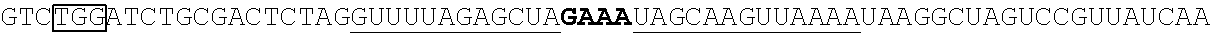

CUUGAAAAAGUGGCACCGAGUCGGUGC
SEQ ID NO: 126
Nucleic acid sequence
PS2 gRNA targeting sequence (19 nt) + backbone (option 1)

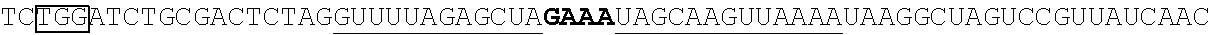

UUGAAAAAGUGGCACCGAGUCGGUGC
SEQ ID NO: 127
Nucleic acid sequence
PS2 gRNA targeting sequence (18 nt) + backbone (option 1)

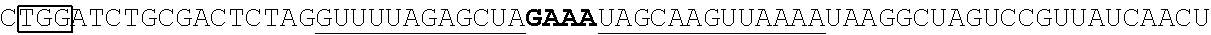

UGAAAAAGUGGCACCGAGUCGGUGC
SEQ ID NO: 128
Nucleic acid sequence
PS2 gRNA targeting sequence (17 nt) + backbone (option 1)

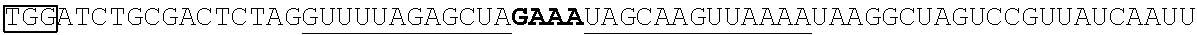

GAAAAAGUGGCACCGAGUCGGUGC
SEQ ID NO: 135
Nucleic acid sequence
PS3 gRNA targeting sequence (20 nt) + backbone (option 1)
TCAATGTATCTTATCATGTCGUUUUAGAGCUAGAAAUAGCAAGUUAAAAUAAGGCUAGUCCGUUAUCAAC
UUGAAAAAGUGGCACCGAGUCGGUGC
SEQ ID NO: 136
Nucleic acid sequence
PS3 gRNA targeting sequence (19 nt) + backbone (option 1)
CAATGTATCTTATCATGTCGUUUUAGAGCUAGAAAUAGCAAGUUAAAAUAAGGCUAGUCCGUUAUCAACU
UGAAAAAGUGGCACCGAGUCGGUGC
SEQ ID NO: 137
Nucleic acid sequence
PS3 gRNA targeting sequence (18 nt) + backbone (option 1)
AATGTATCTTATCATGTCGUUUUAGAGCUAGAAAUAGCAAGUUAAAAUAAGGCUAGUCCGUUAUCAACU
UGAAAAAGUGGCACCGAGUCGGUGC
SEQ ID NO: 138
Nucleic acid sequence
PS3 gRNA targeting sequence (17 nt) + backbone (option 1)
ATGTATCTTATCATGTCGUUUUAGAGCUAGAAAUAGCAAGUUAAAAUAAGGCUAGUCCGUUAUCAACUU
GAAAAAGUGGCACCGAGUCGGUGC
SEQ ID NO: 145
Nucleic acid sequence
LaRo gRNA targeting sequence (20 nt) + backbone (option 1)
AAAGAATTGATTTGATACCGGUUUUAGAGCUAGAAAUAGCAAGUUAAAAUAAGGCUAGUCCGUUAUCAA
CUUGAAAAAGUGGCACCGAGUCGGUGC
SEQ ID NO: 146
Nucleic acid sequence
LaRo gRNA targeting sequence (19 nt) + backbone (option 1)
AAGAATTGATTTGATACCGGUUUUAGAGCUAGAAAUAGCAAGUUAAAAUAAGGCUAGUCCGUUAUCAAC
UUGAAAAAGUGGCACCGAGUCGGUGC
SEQ ID NO: 147
Nucleic acid sequence
LaRo gRNA targeting sequence (18 nt) + backbone (option 1)
AGAATTGATTTGATACCGGUUUUAGAGCUAGAAAUAGCAAGUUAAAAUAAGGCUAGUCCGUUAUCAACU
UGAAAAAGUGGCACCGAGUCGGUGC
SEQ ID NO: 148
Nucleic acid sequence
LaRo gRNA targeting sequence (17 nt) + backbone (option 1)
GAATTGATTTGATACCGGUUUUAGAGCUAGAAAUAGCAAGUUAAAAUAAGGCUAGUCCGUUAUCAACUU
GAAAAAGUGGCACCGAGUCGGUGC
SEQ ID NO: 155
Nucleic acid sequence
LoxP gRNA targeting sequence (20 nt) + backbone (option 1)
GTATGCTATACGAAGTTATTGUUUUAGAGCUAGAAAUAGCAAGUUAAAAUAAGGCUAGUCCGUUAUCAA
CUUGAAAAAGUGGCACCGAGUCGGUGC
SEQ ID NO: 156
Nucleic acid sequence
LoxP gRNA targeting sequence (19 nt) + backbone (option 1)
TATGCTATACGAAGTTATTGUUUUAGAGCUAGAAAUAGCAAGUUAAAAUAAGGCUAGUCCGUUAUCAAC
UUGAAAAAGUGGCACCGAGUCGGUGC
SEQ ID NO: 157
Nucleic acid sequence
LoxP gRNA targeting sequence (18 nt) + backbone (option 1)
ATGCTATACGAAGTTATTGUUUUAGAGCUAGAAAUAGCAAGUUAAAAUAAGGCUAGUCCGUUAUCAACU
UGAAAAAGUGGCACCGAGUCGGUGC
SEQ ID NO: 158
Nucleic acid sequence
LoxP gRNA targeting sequence (17 nt) + backbone (option 1)
TGCTATACGAAGTTATTGUUUUAGAGCUAGAAAUAGCAAGUUAAAAUAAGGCUAGUCCGUUAUCAACUU
GAAAAAGUGGCACCGAGUCGGUGC
sgRNA (targeting sequence + backbone (option 2)):
SEQ ID NO: 225
Nucleic acid sequence
PS2 gRNA targeting sequence (20 nt) + backbone (option 2)

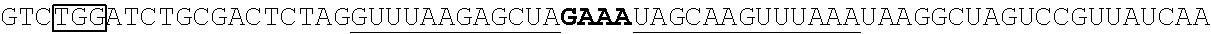

CUUGAAAAAGUGGCACCGAGUCGGUGC
SEQ ID NO: 226
Nucleic acid sequence
PS2 gRNA targeting sequence (19 nt) + backbone (option 2)

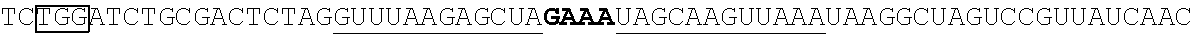

UUGAAAAAGUGGCACCGAGUCGGUGC
SEQ ID NO: 227
Nucleic acid sequence
PS2 gRNA targeting sequence (18 nt) + backbone (option 2)

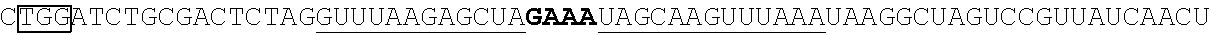

UGAAAAAGUGGCACCGAGUCGGUGC
SEQ ID NO: 228
Nucleic acid sequence
PS2 gRNA targeting sequence (17 nt) + backbone (option 2)

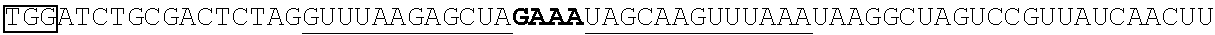

GAAAAAGUGGCACCGAGUCGGUGC
SEQ ID NO: 235
Nucleic acid sequence
PS3 gRNA targeting sequence (20 nt) + backbone (option 2)
TCAATGTATCTTATCATGTCGUUUAAGAGCUAGAAAUAGCAAGUUUAAAUAAGGCUAGUCCGUUAUCAAC
UUGAAAAAGUGGCACCGAGUCGGUGC
SEQ ID NO: 236
Nucleic acid sequence
PS3 gRNA targeting sequence (19 nt) + backbone (option 2)
CAATGTATCTTATCATGTCGUUUAAGAGCUAGAAAUAGCAAGUUUAAAUAAGGCUAGUCCGUUAUCAACU
UGAAAAAGUGGCACCGAGUCGGUGC
SEQ ID NO: 237
Nucleic acid sequence
PS3 gRNA targeting sequence (18 nt) + backbone (option 2)
AATGTATCTTATCATGTCGUUUAAGAGCUAGAAAUAGCAAGUUUAAAUAAGGCUAGUCCGUUAUCAACU
UGAAAAAGUGGCACCGAGUCGGUGC
SEQ ID NO: 238
Nucleic acid sequence
PS3 gRNA targeting sequence (17 nt) + backbone (option 2)
ATGTATCTTATCATGTCGUUUAAGAGCUAGAAAUAGCAAGUUUAAAUAAGGCUAGUCCGUUAUCAACUU
GAAAAAGUGGCACCGAGUCGGUGC
SEQ ID NO: 245
Nucleic acid sequence
LaRo gRNA targeting sequence (20 nt) + backbone (option 2)
AAAGAATTGATTTGATACCGGUUUAAGAGCUAGAAAUAGCAAGUUUAAAUAAGGCUAGUCCGUUAUCAA
CUUGAAAAAGUGGCACCGAGUCGGUGC
SEQ ID NO: 246
Nucleic acid sequence
LaRo gRNA targeting sequence (19 nt) + backbone (option 2)
AAGAATTGATTTGATACCGGUUUAAGAGCUAGAAAUAGCAAGUUUAAAUAAGGCUAGUCCGUUAUCAAC
UUGAAAAAGUGGCACCGAGUCGGUGC
SEQ ID NO: 247
Nucleic acid sequence
LaRo gRNA targeting sequence (18 nt) + backbone (option 2)
AGAATTGATTTGATACCGGUUUAAGAGCUAGAAAUAGCAAGUUUAAAUAAGGCUAGUCCGUUAUCAACU
UGAAAAAGUGGCACCGAGUCGGUGC
SEQ ID NO: 248
Nucleic acid sequence
LaRo gRNA targeting sequence (17 nt) + backbone (option 2)
GAATTGATTTGATACCGGUUUAAGAGCUAGAAAUAGCAAGUUUAAAUAAGGCUAGUCCGUUAUCAACUU
GAAAAAGUGGCACCGAGUCGGUGC
SEQ ID NO: 255
Nucleic acid sequence
LoxP gRNA targeting sequence (20 nt) + backbone (option 2)
GTATGCTATACGAAGTTATTGUUUAAGAGCUAGAAAUAGCAAGUUUAAAUAAGGCUAGUCCGUUAUCAA
CUUGAAAAAGUGGCACCGAGUCGGUGC
SEQ ID NO: 256
Nucleic acid sequence
LoxP gRNA targeting sequence (19 nt) + backbone (option 2)
TATGCTATACGAAGTTATTGUUUAAGAGCUAGAAAUAGCAAGUUUAAAUAAGGCUAGUCCGUUAUCAAC
UUGAAAAAGUGGCACCGAGUCGGUGC
SEQ ID NO: 257
Nucleic acid sequence
LoxP gRNA targeting sequence (18 nt) + backbone (option 2)
ATGCTATACGAAGTTATTGUUUAAGAGCUAGAAAUAGCAAGUUUAAAUAAGGCUAGUCCGUUAUCAACU
UGAAAAAGUGGCACCGAGUCGGUGC
SEQ ID NO: 258
Nucleic acid sequence
LoxP gRNA targeting sequence (17 nt) + backbone (option 2)
TGCTATACGAAGTTATTGUUUAAGAGCUAGAAAUAGCAAGUUUAAAUAAGGCUAGUCCGUUAUCAACUU
GAAAAAGUGGCACCGAGUCGGUGC
sgRNA (targeting sequence + backbone (option 3)):
SEQ ID NO: 325
Nucleic acid sequence
PS2 gRNA targeting sequence (20 nt) + backbone (option 3)

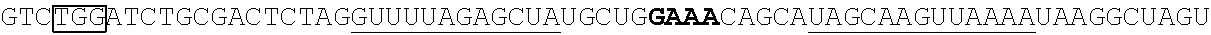

CCGUUAUCAACUUGAAAAAGUGGCACCGAGUCGGUGC
SEQ ID NO: 326
Nucleic acid sequence
PS2 gRNA targeting sequence (19 nt) + backbone (option 3)

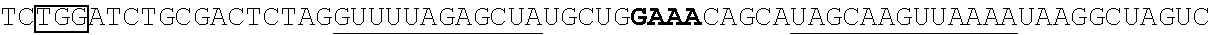

CGUUAUCAACUUGAAAAAGUGGCACCGAGUCGGUGC
SEQ ID NO: 327
Nucleic acid sequence
PS2 gRNA targeting sequence (18 nt) + backbone (option 3)

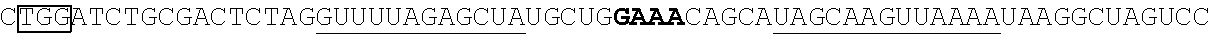

GUUAUCAACUUGAAAAAGUGGCACCGAGUCGGUGC
SEQ ID NO: 328
Nucleic acid sequence
PS2 gRNA targeting sequence (17 nt) + backbone (option 3)

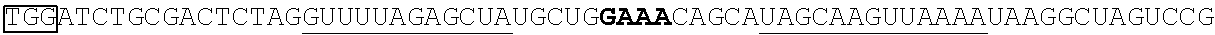

UUAUCAACUUGAAAAAGUGGCACCGAGUCGGUGC
SEQ ID NO: 335
Nucleic acid sequence
PS3 gRNA targeting sequence (20 nt) + backbone (option 3)
TCAATGTATCTTATCATGTCGUUUUAGAGCUAUGCUGGAAACAGCAUAGCAAGUUAAAAUAAGGCUAGUC
CGUUAUCAACUUGAAAAAGUGGCACCGAGUCGGUGC
SEQ ID NO: 336
Nucleic acid sequence
PS3 gRNA targeting sequence (19 nt) + backbone (option 3)
CAATGTATCTTATCATGTCGUUUUAGAGCUAUGCUGGAAACAGCAUAGCAAGUUAAAAUAAGGCUAGUCC
GUUAUCAACUUGAAAAAGUGGCACCGAGUCGGUGC
SEQ ID NO: 337
Nucleic acid sequence
PS3 gRNA targeting sequence (18 nt) + backbone (option 3)
AATGTATCTTATCATGTCGUUUUAGAGCUAUGCUGGAAACAGCAUAGCAAGUUAAAAUAAGGCUAGUCCG
UUAUCAACUUGAAAAAGUGGCACCGAGUCGGUGC
SEQ ID NO: 338
Nucleic acid sequence
PS3 gRNA targeting sequence (17 nt) + backbone (option 3)
ATGTATCTTATCATGTCGUUUUAGAGCUAUGCUGGAAACAGCAUAGCAAGUUAAAAUAAGGCUAGUCCGU
UAUCAACUUGAAAAAGUGGCACCGAGUCGGUGC
SEQ ID NO: 345
Nucleic acid sequence
LaRo gRNA targeting sequence (20 nt) + backbone (option 3)
AAAGAATTGATTTGATACCGGUUUUAGAGCUAUGCUGGAAACAGCAUAGCAAGUUAAAAUAAGGCUAGU
CCGUUAUCAACUUGAAAAAGUGGCACCGAGUCGGUGC
SEQ ID NO: 346
Nucleic acid sequence
LaRo gRNA targeting sequence (19 nt) + backbone (option 3)
AAGAATTGATTTGATACCGGUUUUAGAGCUAUGCUGGAAACAGCAUAGCAAGUUAAAAUAAGGCUAGUC
CGUUAUCAACUUGAAAAAGUGGCACCGAGUCGGUGC
SEQ ID NO: 347
Nucleic acid sequence
LaRo gRNA targeting sequence (18 nt) + backbone (option 3)
AGAATTGATTTGATACCGGUUUUAGAGCUAUGCUGGAAACAGCAUAGCAAGUUAAAAUAAGGCUAGUCC
GUUAUCAACUUGAAAAAGUGGCACCGAGUCGGUGC
SEQ ID NO: 348
Nucleic acid sequence
LaRo gRNA targeting sequence (17 nt) + backbone (option 3)
GAATTGATTTGATACCGGUUUUAGAGCUAUGCUGGAAACAGCAUAGCAAGUUAAAAUAAGGCUAGUCCG
UUAUCAACUUGAAAAAGUGGCACCGAGUCGGUGC
SEQ ID NO: 355
Nucleic acid sequence
LoxP gRNA targeting sequence (20 nt) + backbone (option 3)
GTATGCTATACGAAGTTATTGUUUUAGAGCUAUGCUGGAAACAGCAUAGCAAGUUAAAAUAAGGCUAGU
CCGUUAUCAACUUGAAAAAGUGGCACCGAGUCGGUGC
SEQ ID NO: 356
Nucleic acid sequence
LoxP gRNA targeting sequence (19 nt) + backbone (option 3)
TATGCTATACGAAGTTATTGUUUUAGAGCUAUGCUGGAAACAGCAUAGCAAGUUAAAAUAAGGCUAGUCC
GUUAUCAACUUGAAAAAGUGGCACCGAGUCGGUGC
SEQ ID NO: 357
Nucleic acid sequence
LoxP gRNA targeting sequence (18 nt) + backbone (option 3)
ATGCTATACGAAGTTATTGUUUUAGAGCUAUGCUGGAAACAGCAUAGCAAGUUAAAAUAAGGCUAGUCC
GUUAUCAACUUGAAAAAGUGGCACCGAGUCGGUGC
SEQ ID NO: 358
Nucleic acid sequence
LoxP gRNA targeting sequence (17 nt) + backbone (option 3)
TGCTATACGAAGTTATTGUUUUAGAGCUAUGCUGGAAACAGCAUAGCAAGUUAAAAUAAGGCUAGUCCG
UUAUCAACUUGAAAAAGUGGCACCGAGUCGGUGC
sgRNA (targeting sequence + backbone (option 4)):
SEQ ID NO: 425
Nucleic acid sequence
PS2 gRNA targeting sequence (20 nt) + backbone (option 4)

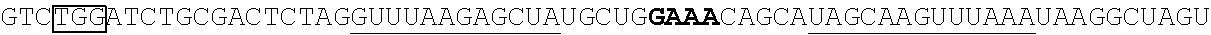

CCGUUAUCAACUUGAAAAAGUGGCACCGAGUCGGUGC
SEQ ID NO: 426
Nucleic acid sequence
PS2 gRNA targeting sequence (19 nt) + backbone (option 4)

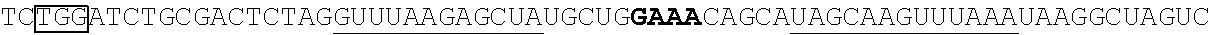

CGUUAUCAACUUGAAAAAGUGGCACCGAGUCGGUGC
SEQ ID NO: 427
Nucleic acid sequence
PS2 gRNA targeting sequence (18 nt) + backbone (option 4)

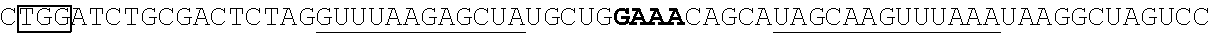

GUUAUCAACUUGAAAAAGUGGCACCGAGUCGGUGC
SEQ ID NO: 428
Nucleic acid sequence
PS2 gRNA targeting sequence (17 nt) + backbone (option 4)

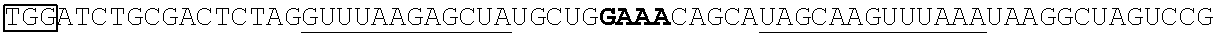

UUAUCAACUUGAAAAAGUGGCACCGAGUCGGUGC
SEQ ID NO: 435
Nucleic acid sequence
PS3 gRNA targeting sequence (20 nt) + backbone (option 4)
TCAATGTATCTTATCATGTCGUUUAAGAGCUAUGCUGGAAACAGCAUAGCAAGUUUAAAUAAGGCUAGUC
CGUUAUCAACUUGAAAAAGUGGCACCGAGUCGGUGC
SEQ ID NO: 436
Nucleic acid sequence
PS3 gRNA targeting sequence (19 nt) + backbone (option 4)
CAATGTATCTTATCATGTCGUUUAAGAGCUAUGCUGGAAACAGCAUAGCAAGUUUAAAUAAGGCUAGUCC
GUUAUCAACUUGAAAAAGUGGCACCGAGUCGGUGC
SEQ ID NO: 437
Nucleic acid sequence
PS3 gRNA targeting sequence (18 nt) + backbone (option 4)
AATGTATCTTATCATGTCGUUUAAGAGCUAUGCUGGAAACAGCAUAGCAAGUUUAAAUAAGGCUAGUCCG
UUAUCAACUUGAAAAAGUGGCACCGAGUCGGUGC
SEQ ID NO: 438
Nucleic acid sequence
PS3 gRNA targeting sequence (17 nt) + backbone (option 4)
ATGTATCTTATCATGTCGUUUAAGAGCUAUGCUGGAAACAGCAUAGCAAGUUUAAAUAAGGCUAGUCCGU
UAUCAACUUGAAAAAGUGGCACCGAGUCGGUGC
SEQ ID NO: 445
Nucleic acid sequence
LaRo gRNA targeting sequence (20 nt) + backbone (option 4)
AAAGAATTGATTTGATACCGGUUUAAGAGCUAUGCUGGAAACAGCAUAGCAAGUUUAAAUAAGGCUAGU
CCGUUAUCAACUUGAAAAAGUGGCACCGAGUCGGUGC
SEQ ID NO: 446
Nucleic acid sequence
LaRo gRNA targeting sequence (19 nt) + backbone (option 4)
AAGAATTGATTTGATACCGGUUUAAGAGCUAUGCUGGAAACAGCAUAGCAAGUUUAAAUAAGGCUAGUC
CGUUAUCAACUUGAAAAAGUGGCACCGAGUCGGUGC
SEQ ID NO: 447
Nucleic acid sequence
LaRo gRNA targeting sequence (18 nt) + backbone (option 4)
AGAATTGATTTGATACCGGUUUAAGAGCUAUGCUGGAAACAGCAUAGCAAGUUUAAAUAAGGCUAGUCC
GUUAUCAACUUGAAAAAGUGGCACCGAGUCGGUGC
SEQ ID NO: 448
Nucleic acid sequence
LaRo gRNA targeting sequence (17 nt) + backbone (option 4)
GAATTGATTTGATACCGGUUUAAGAGCUAUGCUGGAAACAGCAUAGCAAGUUUAAAUAAGGCUAGUCCG
UUAUCAACUUGAAAAAGUGGCACCGAGUCGGUGC
SEQ ID NO: 455
Nucleic acid sequence
LoxP gRNA targeting sequence (20 nt) + backbone (option 4)
GTATGCTATACGAAGTTATTGUUUAAGAGCUAUGCUGGAAACAGCAUAGCAAGUUUAAAUAAGGCUAGU
CCGUUAUCAACUUGAAAAAGUGGCACCGAGUCGGUGC
SEQ ID NO: 456
Nucleic acid sequence
LoxP gRNA targeting sequence (19 nt) + backbone (option 4)
TATGCTATACGAAGTTATTGUUUAAGAGCUAUGCUGGAAACAGCAUAGCAAGUUUAAAUAAGGCUAGUCC
GUUAUCAACUUGAAAAAGUGGCACCGAGUCGGUGC
SEQ ID NO: 457
Nucleic acid sequence
LoxP gRNA targeting sequence (18 nt) + backbone (option 4)
ATGCTATACGAAGTTATTGUUUAAGAGCUAUGCUGGAAACAGCAUAGCAAGUUUAAAUAAGGCUAGUCC
GUUAUCAACUUGAAAAAGUGGCACCGAGUCGGUGC
SEQ ID NO: 458
Nucleic acid sequence
LoxP gRNA targeting sequence (17 nt) + backbone (option 4)
TGCTATACGAAGTTATTGUUUAAGAGCUAUGCUGGAAACAGCAUAGCAAGUUUAAAUAAGGCUAGUCCG
UUAUCAACUUGAAAAAGUGGCACCGAGUCGGUGC

SSODN:
Nucleic acid sequence
SEQ ID NO: 510
ssODN 20T (5′ homology arm (10 nt) + 3′ homology arm (10 nt))
TGATTTGATAATTAGGTCCC
Nucleic acid sequence
SEQ ID NO: 520
ssODN 40T (5′ homology arm (20 nt) + 3′ homology arm (20 nt))
GGCAAAGAATTGATTTGATAATTAGGTCCCTCGACCTGCA
Nucleic acid sequence
SEQ ID NO: 530
ssODN 60T (5′ homology arm (30 nt) + 3′ homology arm (30 nt))
TCATCATTTTGGCAAAGAATTGATTTGATAATTAGGTCCCTCGACCTGCAGCCCAAGCTA
Nucleic acid sequence
ssODN 80T (5′ homology arm (40 nt) + 3′ homology arm (40 nt))
SEQ ID NO: 540
TTGTGCTGTCTCATCATTTTGGCAAAGAATTGATTTGATAATTAGGTCCCTCGACCTGCAGCCCAAGC
TAGATCGAATTC
Nucleic acid sequence
sSODN 100T (5′ homology arm (50 nt) + 3′ homology arm (50 nt))
SEQ ID NO: 550
GTGCTGGTTATTGTGCTGTCTCATCATTTTGGCAAAGAATTGATTTGATAATTAGGTCCCTCGACCTG
CAGCCCAAGCTAGATCGAATTCGGCCGGCCTT
Nucleic acid sequence
ssODN 120T (5′ homology arm (60 nt) + 3′ homology arm (60 nt))
SEQ ID NO: 560
CCTGGGCAACGTGCTGGTTATTGTGCTGTCTCATCATTTTGGCAAAGAATTGATTTGATAATTAGGTC
CCTCGACCTGCAGCCCAAGCTAGATCGAATTCGGCCGGCCTTGTACGCGTTA
Nucleic acid sequence
ssODN 140T (5′ homology arm (70 nt) + 3′ homology arm (70 nt))
SEQ ID NO: 570
TCCTACAGCTCCTGGGCAACGTGCTGGTTATTGTGCTGTCTCATCATTTTGGCAAAGAATTGATTTGA
TAATTAGGTCCCTCGACCTGCAGCCCAAGCTAGATCGAATTCGGCCGGCCTTGTACGCGTTAAGTGCA
ACAC
Nucleic acid sequence
SSODN 160T (5′ homology arm (80 nt) + 3′ homology arm (80 nt))
SEQ ID NO: 580
TTCTTCTTTTTCCTACAGCTCCTGGGCAACGTGCTGGTTATTGTGCTGTCTCATCATTTTGGCAAAGAA
TTGATTTGATAATTAGGTCCCTCGACCTGCAGCCCAAGCTAGATCGAATTCGGCCGGCCTTGTACGCG
TTAAGTGCAACACGATCCCGCCA
ssODN subparts:
Nucleic acid sequence
5′ homology arm (10 nt)
SEQ ID NO: 511
TGATTTGATA
Nucleic acid sequence
3′ homology arm (10 nt)
SEQ ID NO: 512
ATTAGGTCCC
Nucleic acid sequence
5′ homology arm (20 nt)
SEQ ID NO: 521
GGCAAAGAATTGATTTGATA
Nucleic acid sequence
SEQ ID NO: 522
3′ homology arm (20 nt)
ATTAGGTCCCTCGACCTGCA
Nucleic acid sequence
SEQ ID NO: 531
5′ homology arm (30 nt)
TCATCATTTTGGCAAAGAATTGATTTGATA
Nucleic acid sequence
SEQ ID NO: 532
3′ homology arm (30 nt)
ATTAGGTCCCTCGACCTGCAGCCCAAGCTA
Nucleic acid sequence
SEQ ID NO: 541
5′ homology arm (40 nt)
TTGTGCTGTCTCATCATTTTGGCAAAGAATTGATTTGATA
Nucleic acid sequence
SEQ ID NO: 542
3′ homology arm (40 nt)
ATTAGGTCCCTCGACCTGCAGCCCAAGCTAGATCGAATTC
Nucleic acid sequence
SEQ ID NO: 551
5′ homology arm (50 nt)
GTGCTGGTTATTGTGCTGTCTCATCATTTTGGCAAAGAATTGATTTGATA
Nucleic acid sequence
3′ homology arm (50 nt)
SEQ ID NO: 552
ATTAGGTCCCTCGACCTGCAGCCCAAGCTAGATCGAATTCGGCCGGCCTT
Nucleic acid sequence
5′ homology arm (60 nt)
SEQ ID NO: 561
CCTGGGCAACGTGCTGGTTATTGTGCTGTCTCATCATTTTGGCAAAGAATTGATTTGATA
Nucleic acid sequence
3′ homology arm (60 nt)
SEQ ID NO: 562
ATTAGGTCCCTCGACCTGCAGCCCAAGCTAGATCGAATTCGGCCGGCCTTGTACGCGTTA
Nucleic acid sequence
5′ homology arm (70 nt)
SEQ ID NO: 571
TCCTACAGCTCCTGGGCAACGTGCTGGTTATTGTGCTGTCTCATCATTTTGGCAAAGAAT
TGATTTGATA
Nucleic acid sequence
3′ homology arm (70 nt)
SEQ ID NO: 572
ATTAGGTCCCTCGACCTGCAGCCCAAGCTAGATCGAATTCGGCCGGCCTTGTACGCGTT
AAGTGCAACAC
Nucleic acid sequence
5′ homology arm (80 nt)
SEQ ID NO: 581
TTCTTCTTTTTCCTACAGCTCCTGGGCAACGTGCTGGTTATTGTGCTGTCTCATCATT
TTGGCAAAGAATTGATTTGATA
Nucleic acid sequence
3′ homology arm (80 nt)
SEQ ID NO: 582
ATTAGGTCCCTCGACCTGCAGCCCAAGCTAGATCGAATTCGGCCGGCCTTGTACGCGT
TAAGTGCAACACGATCCCGCCA
SpCas9 and Cas9 variants:
Protein sequence
SpCas9 WT
SEQ ID NO: 600
MDKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMK
NYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHY
LDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Protein sequence
SpCas9 Variant 1 (MA sequence-NSL-SpCas9 WT-SRAD linker-NSL)
SEQ ID NO: 601
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Protein sequence
SpCas9 Variant 2 (MA sequence-NSL-SpCas9 WT plus
2 substitutions-SRAD linker-NSL-6His)
SEQ ID NO: 602
MAPKKKRKVDKKYSIGLDIGTNSVGWAVITDEYKVPSKKFKVLGNTDRHSIKKNL
IGALLFDSGETAEATRLKRTARRRYTRRKNRILYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIEEFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Protein sequence
SpCas9 Variant 3 (MGSS leader sequence-6His-TEV
protease cleavage site (ENLYFQGSM)-SpCas9 WT-
GGGS linker-NSL)
SEQ ID NO: 603
MGSSHHHHHHHHENLYFQGSMDKKYSIGLDIGTNSVGWAVITDEYKVPSKKFKVL
GNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGL
YETRIDLSQLGGDGGGSPKKKRKV
Protein sequence
SpCas9 Variant 4 (MA sequence-6His-linker-NSL-SpCas9
WT-SRAD linker-NSL)
SEQ ID NO: 604
MAHHHHHHGGSPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINA
SGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEIT
KAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQN
EKLYLYYLQNGRDMYVDQELDINRLSDYDVDHIVPQSFLKDDSIDNKVLTRSDKN
RGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYET
RIDLSQLGGDSRADPKKKRKV
Protein sequence
SpCas9 Variant 5 (MA sequence-NSL-SpCas9 WT-SRAD
linker-6His)
SEQ ID NO: 605
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADHHHHHH
Protein sequence
SpCas9 Variant 6 (MA sequence-6His-linker-SpCas9
WT-SRAD linker-NSL)
SEQ ID NO: 606
MAHHHHHHGGSDKKYSIGLDIGTNSVGWAVITDEYKVPSKKFKVLGNTDRHSIKK
NLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQL
GGDSRADPKKKRKV
Protein sequence
SpCas9 Variant 7 (MGSS leader sequence-6His-TEV
protease cleavage site (ENLYFQGSM)-SpCas9 WT-
GGGS linker-NSL)
SEQ ID NO: 607
MGSSHHHHHHHHENLYFQGSMDKKYSIGLDIGTNSVGWAVITDEYKVPSKKFKVL
GNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGL
YETRIDLSQLGGDGGGSPKKKRKV
Protein sequence
SpCas9 Variant 8 (MA sequence-NSL-SpCas9 WT plus
1 substitution-SRAD linker-NSL-6His)
SEQ ID NO: 608
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLKDDSIDNAVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Protein sequence
SpCas9 Variant 9 (MA sequence-NSL-SpCas9 WT plus
3 substitutions-SRAD linker-NSL-6His)
SEQ ID NO: 609
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEALYLYYLQ
NGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPALESEFVYGDYKVYDVRKMIAKSEQEIGKATAKYF
FYSNIMNFFKTEITLANGEIRKAPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Protein sequence
SpCas9 Variant 10 (MA sequence-NSL-SpCas9 WT plus
3 substitutions-SRAD linker-NSL-6His)
SEQ ID NO: 610
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLADDSIDNKVLTRSDKNRGKSDNVPS
EEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYH
HAHDAYLNAVVGTALIKKYPALESEFVYGDYKVYDVRKMIAKSEQEIGKATAKYF
FYSNIMNFFKTEITLANGEIRKAPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Protein sequence
SpCas9 Variant 11 (MA sequence-NSL-SpCas9 WT plus
4 substitutions-SRAD linker-NSL-6His)
SEQ ID NO: 611
MAPKKKRKVDKKYSIGLDIGTNSVGWAVITDEYKVPSKKFKVLGNTDRHSIKKNL
IGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGAS
AQSFIERMTAFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLK
RRRYTGWGALSRKLINGIRDKQSGKTILDFLKSDGFANRNFMALIHDDSLTFKED
IQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRAI
TKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSRADPKKKRKVHHHHHH
Base editor parts-Cas9-based sequences:
Protein sequence
Dead SpCas9 (dCas9) (SpCas9 D10A and H840A)
SEQ ID NO: 620
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGD
Protein sequence
SpCas9 nickase (nCas9 or Cas9n) (SpCas9 D10A)
SEQ ID NO: 621
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGD
Protein sequence
VQR-SpCas9 nickase
SEQ ID NO: 631
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEKGKSKK
LKSVKELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKQYRSTKEVLDATLIHQSITGLYETRIDLSQLGGD
Protein sequence
EQR-SpCas9 nickase
SEQ ID NO: 632
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESILPKRNSDKLIARKKDWDPKKYGGFESPTVAYSVLVVAKVEKGKSKK
LKSVKELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKQYRSTKEVLDATLIHQSITGLYETRIDLSQLGGD
Protein sequence
VRER-SpCas9 nickase
SEQ ID NO: 633
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEKGKSKK
LKSVKELLGITIMERSSFEKNPIDFLEAKGYKEVKKDLIIKLPKYSLFELENGRK
RMLASARELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKEYRSTKEVLDATLIHQSITGLYETRIDLSQLGGD
Protein sequence
CP1028-SpCas9
SEQ ID NO: 634
EIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYE
TRIDLSQLGGDGGSGGSGGSGGSGGSGGSGGMDKKYSIGLAIGTNSVGWAVITDE
YKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGS
IPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYK
VYDVRKMIAKSEQ
Protein sequence
CP1041-SpCas9
SEQ ID NO: 635
IMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDGGS
GGSGGSGGSGGSGGSGGDKKYSIGLAIGTNSVGWAVITDEYKVPSKKFKVLGNTD
RHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILR
RQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYK
VREINNYHHAHDAYLNAVVGTALIKKYPKLESEFVYGDYKVYDVRKMIAKSEQEI
GKATAKYFFYS
Protein sequence
SpCas9-NG
SEQ ID NO: 636
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQ
TGGFSKESIRPKRNSDKLIARKKDWDPKKYGGFVSPTVAYSVLVVAKVEKGKSKK
LKSVKELLGITIMERSSFEKNPIDFLEAKGYKEVKKDLIIKLPKYSLFELENGRK
RMLASARFLQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
RAFKYFDTTIDRKVYRSTKEVLDATLIHQSITGLYETRIDLSQLGGD
Protein sequence
SaCas9 nickase
SEQ ID NO: 640
GKRNYILGLAIGITSVGYGIIDYETRDVIDAGVRLFKEANVENNEGRRSKRGARR
LKRRRRHRIQRVKKLLFDYNLLTDHSELSGINPYEARVKGLSQKLSEEEFSAALL
HLAKRRGVHNVNEVEEDTGNELSTKEQISRNSKALEEKYVAELQLERLKKDGEVR
GSINRFKTSDYVKEAKQLLKVQKAYHQLDQSFIDTYIDLLETRRTYYEGPGEGSP
FGWKDIKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTNLKVYH
DIKDITARKEIIENAELLDQIAKILTIYQSSEDIQEELTNLNSELTQEEIEQISN
LKGYTGTHNLSLKAINLILDELWHTNDNQIAIFNRLKLVPKKVDLSQQKEIPTTL
VDDFILSPVVKRSFIQSIKVINAIIKKYGLPNDIIIELAREKNSKDAQKMINEMQ
KRNRQTNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKISYETFK
KHILNLAKGKGRISKTKKEYLLEERDINRFSVQKDFINRNLVDTRYATRGLMNLL
RSYFRVNNLDVKVKSINGGFTSFLRRKWKFKKERNKGYKHHAEDALIIANADFIF
KEWKKLDKAKKVMENQMFEEKQAESMPEIETEQEYKEIFITPHQIKHIKDFKDYK
YSHRVDKKPNRELINDTLYSTRKDDKGNTLIVNNLNGLYDKDNDKLKKLINKSPE
KLLMYHHDPQTYQKLKLIMEQYGDEKNPLYKYYEETGNYLTKYSKKDNGPVIKKI
KYYGNKLNAHLDITDDYPNSRNKVVKLSLKPYRFDVYLDNGVYKFVTVKNLDVIK
KENYYEVNSKCYEEAKKLKKISNQAEFIASFYNNDLIKINGELYRVIGVNNDLLN
RIEVNMIDITYREYLENMNDKRPPRIIKTIASKTQSIKKYSTDILGNLYEVKSKK
HPQIIKKG
Protein sequence
SaCas9-KKH
SEQ ID NO: 641
GKRNYILGLAIGITSVGYGIIDYETRDVIDAGVRLFKEANVENNEGRRSKRGARR
LKRRRRHRIQRVKKLLFDYNLLTDHSELSGINPYEARVKGLSQKLSEEEFSAALL
HLAKRRGVHNVNEVEEDTGNELSTKEQISRNSKALEEKYVAELQLERLKKDGEVR
GSINRFKTSDYVKEAKQLLKVQKAYHQLDQSFIDTYIDLLETRRTYYEGPGEGSP
FGWKDIKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTNLKVYH
DIKDITARKEIIENAELLDQIAKILTIYQSSEDIQEELTNLNSELTQEEIEQISN
LKGYTGTHNLSLKAINLILDELWHTNDNQIAIFNRLKLVPKKVDLSQQKEIPTTL
VDDFILSPVVKRSFIQSIKVINAIIKKYGLPNDIIIELAREKNSKDAQKMINEMQ
KRNRQTNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKISYETFK
KHILNLAKGKGRISKTKKEYLLEERDINRFSVQKDFINRNLVDTRYATRGLMNLL
RSYFRVNNLDVKVKSINGGFTSFLRRKWKFKKERNKGYKHHAEDALIIANADFIF
KEWKKLDKAKKVMENQMFEEKQAESMPEIETEQEYKEIFITPHQIKHIKDFKDYK
YSHRVDKKPNRKLINDTLYSTRKDDKGNTLIVNNLNGLYDKDNDKLKKLINKSPE
KLLMYHHDPQTYQKLKLIMEQYGDEKNPLYKYYEETGNYLTKYSKKDNGPVIKKI
KYYGNKLNAHLDITDDYPNSRNKVVKLSLKPYRFDVYLDNGVYKFVTVKNLDVIK
KENYYEVNSKCYEEAKKLKKISNQAEFIASFYKNDLIKINGELYRVIGVNNDLLN
RIEVNMIDITYREYLENMNDKRPPHIIKTIASKTQSIKKYSTDILGNLYEVKSKK
HPQIIKKG
Base editor parts-Cas12a-based sequences:
Protein sequence
Catalytically dead LbCas12a (dLbCas12a)
SEQ ID NO: 650
KLEKFTNCYSLSKTLRFKAIPVGKTQENIDNKRLLVEDEKRAEDYKGVKKLLDRY
YLSFINDVLHSIKLKNLNNYISLFRKKTRTEKENKELENLEINLRKEIAKAFKGN
EGYKSLFKKDIIETILPEFLDDKDEIALVNSFNGFTTAFTGFFDNRENMFSEEAK
STSIAFRCINENLTRYISNMDIFEKVDAIFDKHEVQEIKEKILNSDYDVEDFFEG
EFFNFVLTQEGIDVYNAIIGGFVTESGEKIKGLNEYINLYNQKTKQKLPKFKPLY
KQVLSDRESLSFYGEGYTSDEEVLEVFRNTLNKNSEIFSSIKKLEKLFKNFDEYS
SAGIFVKNGPAISTISKDIFGEWNVIRDKWNAEYDDIHLKKKAVVTEKYEDDRRK
SFKKIGSFSLEQLQEYADADLSVVEKLKEIIIQKVDEIYKVYGSSEKLFDADFVL
EKSLKKNDAVVAIMKDLLDSVKSFENYIKAFFGEGKETNRDESFYGDFVLAYDIL
LKVDHIYDAIRNYVTQKPYSKDKFKLYFQNPQFMGGWDKDKETDYRATILRYGSK
YYLAIMDKKYAKCLQKIDKDDVNGNYEKINYKLLPGPNKMLPKVFFSKKWMAYYN
PSEDIQKIYKNGTFKKGDMFNLNDCHKLIDFFKDSISRYPKWSNAYDFNFSETEK
YKDIAGFYREVEEQGYKVSFESASKKEVDKLVEEGKLYMFQIYNKDFSDKSHGTP
NLHTMYFKLLFDENNHGQIRLSGGAELFMRRASLKKEELVVHPANSPIANKNPDN
PKKTTTLSYDVYKDKRFSEDQYELHIPIAINKCPKNIFKINTEVRVLLKHDDNPY
VIGIARGERNLLYIVVVDGKGNIVEQYSLNEIINNFNGIRIKTDYHSLLDKKEKE
RFEARQNWTSIENIKELKAGYISQVVHKICELVEKYDAVIALEDLNSGFKNSRVK
VEKQVYQKFEKMLIDKLNYMVDKKSNPCATGGALKGYQITNKFESFKSMSTQNGF
IFYIPAWLTSKIDPSTGFVNLLKTKYTSIADSKKFISSFDRIMYVPEEDLFEFAL
DYKNFSRTDADYIKKWKLYSYGNRIRIFRNPKKNNVFDWEEVCLTSAYKELFNKY
GINYQQGDIRALLCEQSDKAFYSSFMALMSLMLQMRNSITGRTDVDFLISPVKNS
DGIFYDSRNYEAQENAILPKNADANGAYNIARKVLWAIGQFKKAEDEKLDKVKIA
ISNKEWLEYAQTSVK
Protein sequence
Engineered AsCas12a (enAsCas12a)
SEQ ID NO: 651
MTQFEGFTNLYQVSKTLRFELIPQGKTLKHIQEQGFIEEDKARNDHYKELKPIID
RIYKTYADQCLQLVQLDWENLSAAIDSYRKEKTEETRNALIEEQATYRNAIHDYF
IGRTDNLTDAINKRHAEIYKGLFKAELFNGKVLKQLGTVTTTEHENALLRSFDKF
TTYFSGFYRNRKNVFSAEDISTAIPHRIVQDNFPKFKENCHIFTRLITAVPSLRE
HFENVKKAIGIFVSTSIEEVFSFPFYNQLLTQTQIDLYNQLLGGISREAGTEKIK
GLNEVLNLAIQKNDETAHIIASLPHRFIPLFKQILSDRNTLSFILEEFKSDEEVI
QSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTS
EILSHAHAALDQPLPTTLKKQEEKEILKSQLDSLLGLYHLLDWFAVDESNEVDPE
FSARLTGIKLEMEPSLSFYNKARNYATKKPYSVEKFKLNFQMPTLARGWDVNREK
NNGAILFVKNGLYYLGIMPKQKGRYKALSFEPTEKTSEGFDKMYYDYFPDAAKMI
PKCSTQLKAVTAHFQTHTTPILLSNNFIEPLEITKEIYDLNNPEKEPKKFQTAYA
KKTGDQKGYREALCKWIDFTRDFLSKYTKTTSIDLSSLRPSSQYKDLGEYYAELN
PLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQE
LYDYVNHRLSHDLSDEARALLPNVITKEVSHEIIKDRRFTSDKFFFHVPITLNYQ
AANSPSKFNQRVNAYLKEHPETPIIGIARGERNLIYITVIDSTGKILEQRSLNTI
QQFDYQKKLDNREKERVAARQAWSVVGTIKDLKQGYLSQVIHEIVDLMIHYQAVV
VLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSH
AIDTMVALIRSVLQMRNSNAATGEDYINSPVRDLNGVCFDSRFQNPEWPMDADAN
GAYHIALKGQLLLNHLKESKDLKLQNGISNQDWLAYIQELRN
Base editor parts-linker sequences:
Protein sequence
XTEN linker
SEQ ID NO: 681
SGSETPGTSESATPES
Protein sequence
SGGS linker unit
SEQ ID NO: 682
SGGS
Protein sequence
(SGGS)2-XTEN-(SGGS)2 linker
SEQ ID NO: 683
SGGSSGGSSGSETPGTSESATPESSGGSSGGS
Protein sequence
(SGG)3S linker
SEQ ID NO: 684
SGGSGGSGGS
Protein sequence
GGGS linker unit
SEQ ID NO: 685
GGGS
Protein sequence
GGGGS linker unit
SEQ ID NO: 686
GGGGS
Base editor parts-Nuclear localization signal (NSL) sequences:
Protein sequence
NLS1 (SV40 NLS)
SEQ ID NO: 691
PKKKRKV
Protein sequence
NLS2 (bpNLS)
SEQ ID NO: 692
KRTADGSEFEPKKKRKV
Protein sequence
NLS3 (M + bpNLS)
SEQ ID NO: 693
MKRTADGSEFESPKKKRKV
Cytosine base editors (CBEs):
Protein sequence
BE3 (rAPOBEC1-XTEN-Cas9n-SGGS-UGI-SGGS-NLS1)
SEQ ID NO: 700
MSSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQS
ITGLYETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLSGGSPK
KKRKV
Protein sequence
YE1-BE3 (BE3 W90Y + R126E)
SEQ ID NO: 701
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPENRQGLRDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQS
ITGLYETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLSGGSPK
KKRKV
Protein sequence
YE2-BE3 (BE3 W90Y + R132E)
SEQ ID NO: 702
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPRNRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQS
ITGLYETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLSGGSPK
KKRKV
Protein sequence
EE-BE3 (BE3 R126E + R132E)
SEQ ID NO: 703
MSSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPHV
TLFIYIARLYHHADPENRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQS
ITGLYETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLSGGSPK
KKRKV
Protein sequence
YEE-BE3 (BE3 W90Y + R126E + R132E)
SEQ ID NO: 704
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPENRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQS
ITGLYETRIDLSQLGGDSGGSTNLSDIIEKETGKQLVIQESILMLPEEVEEVIGN
KPESDILVHTAYDESTDENVMLLTSDAPEYKPWALVIQDSNGENKIKMLSGGSPK
KKRKV
Protein sequence
CDA1-BE3 (BE3 but rAPOBEC1 is replaced
with CDA1; CDA1-XTEN-Cas9n-SGGS-UGI-SGGS-NLS1)
SEQ ID NO: 705
MTDAEYVRIHEKLDIYTFKKQFFNNKKSVSHRCYVLFELKRRGERRACFWGYAVN
KPQSGTERGIHAEIFSIRKVEEYLRDNPGQFTINWYSSWSPCADCAEKILEWYNQ
ELRGNGHTLKIWACKLYYEKNARNQIGLWNLRDNGVGLNVMVSEHYQCCRKIFIQ
SSHNQLNENRWLEKTLKRAEKRRSELSIMIQVKILHTTKSPAVSGSETPGTSESA
TPESDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGS
TNLSDIIEKETGKQLVIQESILMLPEEVEEVIGNKPESDILVHTAYDESTDENVM
LLTSDAPEYKPWALVIQDSNGENKIKMLSGGSPKKKRKV
Protein sequence
AID-BE3 (BE3 but rAPOBEC1 is replaced with
AID; AID-XTEN-Cas9n-SGGS-UGI-SGGS-NLS1)
SEQ ID NO: 706
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFT
ARLYFCEDRKAEPEGLRRLHRAGVQIAIMTFKDYFYCWNTFVENHERTFKAWEGL
HENSVRLSRQLRRILLPLYEVDDLRDAFRTLGLSGSETPGTSESATPESDKKYSI
GLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFR
IPYYVGPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNS
RERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTAL
IKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYF
DTTIDRKRYTSTKEVLDATLIHQSITGLYETRIDLSQLGGDSGGSTNLSDIIEKE
TGKQLVIQESILMLPEEVEEVIGNKPESDILVHTAYDESTDENVMLLTSDAPEYK
PWALVIQDSNGENKIKMLSGGSPKKKRKV
Protein sequence
BE3-Gam (Gam-XTEN-rAPOBEC1-XTEN-Cas9n-SGGS-UGI-SGGS-NLS1)
SEQ ID NO: 707
MAKPAKRIKSAAAAYVPQNRDAVITDIKRIGDLQREASRLETEMNDAIAEITEKF
AARIAPIKTDIETLSKGVQGWCEANRDELTNGGKVKTANLVTGDVSWRVRPPSVS
IRGMDAVMETLERLGLQRFIRTKQEINKEAILLEPKAVAGVAGITVKSGIEDFSI
IPFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSITW
FLSWSPCGECSRAITEFLSRYPHVTLFIYIARLYHHADPRNRQGLRDLISSGVTI
QIMTEQESGYCWRNFVNYSPSNEAHWPRYPHLWVRLYVLELYCIILGLPPCLNIL
RRKQPQLTFFTIALQSCHYQRLPPHILWATGLKSGSETPGTSESATPESDKKYSI
GLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFR
IPYYVGPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNS
RERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTAL
IKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYF
DTTIDRKRYTSTKEVLDATLIHQSITGLYETRIDLSQLGGDSGGSTNLSDIIEKE
TGKQLVIQESILMLPEEVEEVIGNKPESDILVHTAYDESTDENVMLLTSDAPEYK
PWALVIQDSNGENKIKMLSGGSPKKKRKV
Protein sequence
BE4 (rAPOBEC1-(SGGS)₂XTEN(SGGS)₂-Cas9n-(SGG)₃S-UGI-
(SGG)₃S-UGI-SGGS-NLS1)
SEQ ID NO: 710
MSSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKR
YTSTKEVLDATLIHQSITGLYETRIDLSQLGGDSGGSGGSGGSTNLSDIIEKETG
KQLVIQESILMLPEEVEEVIGNKPESDILVHTAYDESTDENVMLLTSDAPEYKPW
ALVIQDSNGENKIKMLSGGSGGSGGSTNLSDIIEKETGKQLVIQESILMLPEEVE
EVIGNKPESDILVHTAYDESTDENVMLLTSDAPEYKPWALVIQDSNGENKIKMLS
GGSPKKKRK
Protein sequence
BE4-Gam (Gam-XTEN-rAPOBEC1-(SGGS)₂XTEN(SGGS)₂-Cas9n-
(SGG)₃S-UGI-(SGG)₃S-UGI-SGGS-NLS1)
SEQ ID NO: 711
MAKPAKRIKSAAAAYVPQNRDAVITDIKRIGDLQREASRLETEMNDAIAEITEKF
AARIAPIKTDIETLSKGVQGWCEANRDELTNGGKVKTANLVTGDVSWRVRPPSVS
IRGMDAVMETLERLGLQRFIRTKQEINKEAILLEPKAVAGVAGITVKSGIEDFSI
IPFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSITW
FLSWSPCGECSRAITEFLSRYPHVTLFIYIARLYHHADPRNRQGLRDLISSGVTI
QIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATP
ESSGGSSGGSDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFI
KPILEKMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVP
SEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQ
VNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLG
GDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSD
IIEKETGKQLVIQESILMLPEEVEEVIGNKPESDILVHTAYDESTDENVMLLTSD
APEYKPWALVIQDSNGENKIKMLSGGSPKKKRK
Protein sequence
BE4max (NLS3-rAPOBEC1-(SGGS)₂XTEN(SGGS)₂-Cas9n-
(SGG)₃S-UGI-(SGG)₃S-UGI-SGGS-NLS2)
SEQ ID NO: 712
MKRTADGSEFESPKKKRKVSSETGPVAVDPTLRRRIEPHEFEVFFDPRELRKETC
LLYEINWGGRHSIWRHTSQNTNKHVEVNFIEKFTTERYFCPNTRCSITWFLSWSP
CGECSRAITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNILRRKQPQ
LTFFTIALQSCHYQRLPPHILWATGLKSGGSSGGSSGSETPGTSESATPESSGGS
SGGSDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGS
GGSGGSTNLSDIIEKETGKQLVIQESILMLPEEVEEVIGNKPESDILVHTAYDES
TDENVMLLTSDAPEYKPWALVIQDSNGENKIKMLSGGSGGSGGSTNLSDIIEKET
GKQLVIQESILMLPEEVEEVIGNKPESDILVHTAYDESTDENVMLLTSDAPEYKP
WALVIQDSNGENKIKMLSGGSKRTADGSEFEPKKKRKV
Protein sequence
AncBE4max (NLS3-Anc689APOBEC-(SGGS)₂XTEN(SGGS)₂-
Cas9n-(SGG)₃S-UGI-(SGG)₃S-UGI-SGGS-
NLS2)
SEQ ID NO: 713
MKRTADGSEFESPKKKRKVSSETGPVAVDPTLRRRIEPHEFEVFFDPRELRKETC
LLYEIKWGTSHKIWRHSSKNTTKHVEVNFIEKFTSERHFCPSTSCSITWFLSWSP
CGECSKAITEFLSQHPNVTLVIYVARLYHHMDQQNRQGLRDLVNSGVTIQIMTAP
EYDYCWRNFVNYPPGKEAHWPRYPPLWMKLYALELHAGILGLPPCLNILRRKQPQ
LTFFTIALQSCHYQRLPPHILWATGLKSGGSSGGSSGSETPGTSESATPESSGGS
SGGSDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGS
GGSGGSTNLSDIIEKETGKQLVIQESILMLPEEVEEVIGNKPESDILVHTAYDES
TDENVMLLTSDAPEYKPWALVIQDSNGENKIKMLSGGSGGSGGSTNLSDIIEKET
GKQLVIQESILMLPEEVEEVIGNKPESDILVHTAYDESTDENVMLLTSDAPEYKP
WALVIQDSNGENKIKMLSGGSKRTADGSEFEPKKKRKV
CBE parts-cytidine deaminases:
*the first M may be removed, e.g.,
when not at the N-terminus
Protein sequence
rAPOBEC1
SEQ ID NO: 720
MSSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLK
Protein sequence
YE1-rAPOBEC1 (rAPOBEC1 W90Y + R126E)
SEQ ID NO: 721
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPENRQGLRDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLK
Protein sequence
YE2-rAPOBEC1 (rAPOBEC1 W90Y + R132E)
SEQ ID NO: 722
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPRNRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLK
Protein sequence
EE-rAPOBEC1 (rAPOBEC1 R126E + R132E)
SEQ ID NO: 723
MSSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPHV
TLFIYIARLYHHADPENRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLK
Protein sequence
YEE-rAPOBEC1 (rAPOBEC1 W90Y + R126E + R132E)
SEQ ID NO: 724
MSSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPENRQGLEDLISSGVTIQIMTEQESGYCWRNFVNYSPSNEA
HWPRYPHLWVRLYVLELYCIILGLPPCLNILRRKQPQLTFFTIALQSCHYQRLPP
HILWATGLK
Protein sequence
CDA1
SEQ ID NO: 725
MTDAEYVRIHEKLDIYTFKKQFFNNKKSVSHRCYVLFELKRRGERRACFWGYAVN
KPQSGTERGIHAEIFSIRKVEEYLRDNPGQFTINWYSSWSPCADCAEKILEWYNQ
ELRGNGHTLKIWACKLYYEKNARNQIGLWNLRDNGVGLNVMVSEHYQCCRKIFIQ
SSHNQLNENRWLEKTLKRAEKRRSELSIMIQVKILHTTKSPAV
Protein sequence
AID
SEQ ID NO: 726
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFT
ARLYFCEDRKAEPEGLRRLHRAGVQIAIMTFKDYFYCWNTFVENHERTFKAWEGL
HENSVRLSRQLRRILLPLYEVDDLRDAFRTLGL
Protein sequence
Anc689 APOBEC
SEQ ID NO: 731
SSETGPVAVDPTLRRRIEPHEFEVFFDPRELRKETCLLYEIKWGTSHKIWRHSSK
NTTKHVEVNFIEKFTSERHFCPSTSCSITWFLSWSPCGECSKAITEFLSQHPNVT
LVIYVARLYHHMDQQNRQGLRDLVNSGVTIQIMTAPEYDYCWRNFVNYPPGKEAH
WPRYPPLWMKLYALELHAGILGLPPCLNILRRKQPQLTFFTIALQSCHYQRLPPH
ILWATGLK
Protein sequence
Anc687 APOBEC
SEQ ID NO: 732
SSETGPVAVDPTLRRRIEPHEFEVFFDPRELRKEACLLYEIKWGTSHKIWRNSGK
NTTKHVEVNFIEKFTSERHFCPSISCSITWFLSWSPCWECSKAIREFLSQHPNVT
LVIYVARLFQHMDQQNRQGLRDLVNSGVTIQIMTASEYDHCWRNFVNYPPGKEAH
WPRYPPLWMKLYALELHAGILGLPPCLNILRRKQPQLTFFTIALQSCHYQRLPPH
ILWATGLK
Protein sequence
TadA-CDa
SEQ ID NO: 741
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGVRNSK
RGAAGSLMNVLNYPGMNHRVEITEGILADECAALLCDFYRMPRQVFNSQKKAQSS
IN
Protein sequence
TadA-CDb
SEQ ID NO: 742
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGVRNSK
RGAAGSLMNVLNYPGMNHRVEITEGILADECAALLCDFYRMPRRVFNSQKKAQSS
IN
Protein sequence
TadA-CDc
SEQ ID NO: 743
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGVRNSK
RGAAGSLMNVLNYPGMNHRVEITEGILADECAALLCDFYRIPRQVFNSQKKAQSS
IN
Protein sequence
TadA-CDd
SEQ ID NO: 744
MSEVEFSHEYWMRHALTLAKRARDERKAPVGAVLVLNNRVIGEGWNRAIGLHDPT
AHAEIIALRQGGLVMQNYRLIDATLYVTFEPCVMCAGAMINSRIGRVVFGVRNSK
RGAAGSLMNVLNYPGMNHRVEITEGILADECAALLCDFYRMPRQVFNAQKKAQSS
IN
Protein sequence
TadA-CDe
SEQ ID NO: 745
MSEVEFSHEYWMRHALTLAKRARDERAGPVGAVLVLNNRVIGEGWNRAIGLHDPT
AHAEIMALRQGGLVMQNHRLIDATLYVTFEPCVMCAGAMINSRIGRVVFGVRNSK
RGAAGSLMNVLNYPGMNHRVEITEGILADECAALLCDFYRMPRHVFNSQKKAQSS
IN
Protein sequence
TadA-CDa-V106W
SEQ ID NO: 751
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGWRNSK
RGAAGSLMNVLNYPGMNHRVEITEGILADECAALLCDFYRMPRQVFNSQKKAQSS
IN
Protein sequence
TadA-CDb-V106W
SEQ ID NO: 752
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGWRNSK
RGAAGSLMNVLNYPGMNHRVEITEGILADECAALLCDFYRMPRRVFNSQKKAQSS
IN
Protein sequence
TadA-CDc-V106W
SEQ ID NO: 753
MSEVEFSHEYWMRHALTLAKRARDEGAGPVGAVLVLNNRVIGEGWNRAIGLHDPT
AHAEIMALRQGGLVMQNYRLFDATLYVTFEPCVMCAGAMINSRIGRVVFGWRNSK
RGAAGSLMNVLNYPGMNHRVEITEGILADECAALLCDFYRIPRQVFNSQKKAQSS
IN
Protein sequence
TadA-CDd-V106W
SEQ ID NO: 754
MSEVEFSHEYWMRHALTLAKRARDERKAPVGAVLVLNNRVIGEGWNRAIGLHDPT
AHAEIIALRQGGLVMQNYRLIDATLYVTFEPCVMCAGAMINSRIGRVVFGWRNSK
RGAAGSLMNVLNYPGMNHRVEITEGILADECAALLCDFYRMPRQVFNAQKKAQSS
IN
Protein sequence
TadA-CDe-V106W
SEQ ID NO: 755
MSEVEFSHEYWMRHALTLAKRARDERAGPVGAVLVLNNRVIGEGWNRAIGLHDPT
AHAEIMALRQGGLVMQNHRLIDATLYVTFEPCVMCAGAMINSRIGRVVFGWRNSK
RGAAGSLMNVLNYPGMNHRVEITEGILADECAALLCDFYRMPRHVFNSQKKAQSS
IN
CBE parts-UGI and Gam:
*the first M may be removed, e.g.,
when not at the N-terminus
Protein sequence
Uracil DNA glycosylase (UDG) inhibitor (UGI)
SEQ ID NO: 760
TNLSDIIEKETGKQLVIQESILMLPEEVEEVIGNKPESDILVHTAYDESTDENVM
LLTSDAPEYKPWALVIQDSNGENKIKML
Protein sequence
Gam (bacteriophage Mu protein)
SEQ ID NO: 761
MAKPAKRIKSAAAAYVPQNRDAVITDIKRIGDLQREASRLETEMNDAIAEITEKF
AARIAPIKTDIETLSKGVQGWCEANRDELTNGGKVKTANLVTGDVSWRVRPPSVS
IRGMDAVMETLERLGLQRFIRTKQEINKEAILLEPKAVAGVAGITVKSGIEDFSI
IPFEQEAGI
Adenine base editors (ABEs):
Protein sequence
ABE7.10 (ecTadA-(SGGS)₂XTEN(SGGS)₂-ecTadA*7.10-
(SGGS)₂XTEN(SGGS)₂-Cas9n-SGGS-NLS1)
SEQ ID NO: 800
MSEVEFSHEYWMRHALTLAKRAWDEREVPVGAVLVHNNRVIGEGWNRPIGRHDPT
AHAEIMALRQGGLVMQNYRLIDATLYVTLEPCVMCAGAMIHSRIGRVVFGARDAK
TGAAGSLMDVLHHPGMNHRVEITEGILADECAALLSDFFRMRRQEIKAQKKAQSS
TDSGGSSGGSSGSETPGTSESATPESSGGSSGGSSEVEFSHEYWMRHALTLAKRA
RDEREVPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMNHRVEI
TEGILADECAALLCYFFRMPRQVFNAQKKAQSSTDSGGSSGGSSGSETPGTSESA
TPESSGGSSGGSDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQ
LGGDSGGSPKKKRKV
Protein sequence
ABEmax (NLS3-ecTadA-(SGGS)₂XTEN(SGGS)₂-ecTadA*7.10-
(SGGS)₂XTEN(SGGS)₂-Cas9n-NLS2)
SEQ ID NO: 801
MKRTADGSEFESPKKKRKVMSEVEFSHEYWMRHALTLAKRAWDEREVPVGAVLVH
NNRVIGEGWNRPIGRHDPTAHAEIMALRQGGLVMQNYRLIDATLYVTLEPCVMCA
GAMIHSRIGRVVFGARDAKTGAAGSLMDVLHHPGMNHRVEITEGILADECAALLS
DFFRMRRQEIKAQKKAQSSTDSGGSSGGSSGSETPGTSESATPESSGGSSGGSSE
VEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGVRNAKTGA
AGSLMDVLHYPGMNHRVEITEGILADECAALLCYFFRMPRQVFNAQKKAQSSTDS
GGSSGGSSGSETPGTSESATPESSGGSSGGSDKKYSIGLAIGTNSVGWAVITDEY
KVPSKKFKVLGNTDRHSIKKNLIGALLFDSGETAEATRLKRTARRRYTRRKNRIC
YLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDA
TLIHQSITGLYETRIDLSQLGGDKRTADGSEFEPKKKRKV
Protein sequence
ABE8e (NLS3-ecTadA*8e-(SGGS)₂XTEN(SGGS)₂-Cas9n-SGGS-NLS2)
SEQ ID NO: 810
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYVTFEPCVMCAG
AMIHSRIGRVVFGVRNSKRGAAGSLMNVLNYPGMNHRVEITEGILADECAALLCD
FYRMPRQVFNAQKKAQSSINSGGSSGGSSGSETPGTSESATPESSGGSSGGSDKK
YSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLGAPAAF
KYFDTTIDRKRYTSTKEVLDATLIHQSITGLYETRIDLSQLGGDSGGSKRTADGS
EFEPKKKRKV
Protein sequence
ABE8e dimer (NLS3-ecTadA-(SGGS)₂XTEN(SGGS)₂-
ecTadA*8e-(SGGS)₂XTEN(SGGS)₂-Cas9n-SGGS-
NLS2)
SEQ ID NO: 811
MKRTADGSEFESPKKKRKVSEVEFSHEYWMRHALTLAKRAWDEREVPVGAVLVHN
NRVIGEGWNRPIGRHDPTAHAEIMALRQGGLVMQNYRLIDATLYVTLEPCVMCAG
AMIHSRIGRVVFGARDAKTGAAGSLMDVLHHPGMNHRVEITEGILADECAALLSD
FFRMRRQEIKAQKKAQSSTDSGGSSGGSSGSETPGTSESATPESSGGSSGGSSEV
EFSHEYWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNSKRGAA
GSLMNVLNYPGMNHRVEITEGILADECAALLCDFYRMPRQVFNAQKKAQSSINSG
GSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFL
YLASHYEKLKGSPEDNEQKQLFVEQHKHYLDEIIEQISEFSKRVILADANLDKVL
SAYNKHRDKPIREQAENIIHLFTLTNLGAPAAFKYFDTTIDRKRYTSTKEVLDAT
LIHQSITGLYETRIDLSQLGGDSGGSKRTADGSEFEPKKKRKV
ABE parts-adenine deaminases:
*the first M may be removed, e.g., when
not at the N-terminus
Protein sequence
Wild-type E. coli TadA (ecTadA)
SEQ ID NO: 820
MSEVEFSHEYWMRHALTLAKRAWDEREVPVGAVLVHNNRVIGEGWNRPIGRHDPT
AHAEIMALRQGGLVMQNYRLIDATLYVTLEPCVMCAGAMIHSRIGRVVFGARDAK
TGAAGSLMDVLHHPGMNHRVEITEGILADECAALLSDFFRMRRQEIKAQKKAQSS
TD
Protein sequence
ecTadA*6.3 (ecTadA containing A106V, D108N, D147Y,
and E155V; L84F, H123Y, and I157F; H36L,
R51L, S146C, and K157N; and P48S)
SEQ ID NO: 821
SEVEFSHEYWMRHALTLAKRAWDEREVPVGAVLVLNNRVIGEGWNRSIGLHDPTAH
AEIMALRQGGLVMQNYRLIDATLYVTFEPCVMCAGAMIHSRIGRVVFGVRNAKTG
AAGSLMDVLHYPGMNHRVEITEGILADECAALLCYFFRMRRQVFNAQKKAQSSTD
Protein sequence
ecTadA*6.4 (ecTadA containing A106V, D108N, D147Y,
and E155V; L84F, H123Y, and 1157F; H36L,
R51L, S146C, and K157N; and P48S and A142N)
SEQ ID NO: 822
SEVEFSHEYWMRHALTLAKRAWDEREVPVGAVLVLNNRVIGEGWNRSIGLHDPTA
HAEIMALRQGGLVMQNYRLIDATLYVTFEPCVMCAGAMIHSRIGRVVFGVRNAKT
GAAGSLMDVLHYPGMINHRVEITEGILADECNALLCYFFRMRRQVFNAQKKAQSS
TD
Protein sequence
ecTadA*7.8 (ecTadA containing A106V, D108N, D147Y,
and E155V; L84F, H123Y, and I157F; H36L,
R51L, S146C, and K157N; A142N; and W23L
and P48A)
SEQ ID NO: 823
SEVEFSHEYWMRHALTLAKRALDEREVPVGAVLVLNNRVIGEGWNRAIGLHDPTA
HAEIMALRQGGLVMQNYRLIDATLYVTFEPCVMCAGAMIHSRIGRVVFGVRNAKT
GAAGSLMDVLHYPGMNHRVEITEGILADECNALLCYFFRMRRQVFNAQKKAQSST
D
Protein sequence
ecTadA*7.9 (ecTadA containing A106V, D108N, D147Y,
and E155V; L84F, H123Y, and 1157F; H36L,
R51L, S146C, and K157N; A142N; and W23L,
P48A, and R152P)
SEQ ID NO: 824
SEVEFSHEYWMRHALTLAKRALDEREVPVGAVLVLNNRVIGEGWNRAIGLHDPTA
HAEIMALRQGGLVMQNYRLIDATLYVTFEPCVMCAGAMIHSRIGRVVFGVRNAKT
GAAGSLMDVLHYPGMNHRVEITEGILADECNALLCYFFRMPRQVFNAQKKAQSST
D
Protein sequence
ecTadA*7.10 (ecTadA containing A106V, D108N, D147Y,
and E155V; L84F, H123Y, and 1157F; H36L,
R51L, S146C, and K157N; and W23R, P48A,
and R152P)
SEQ ID NO: 825
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNAKT
GAAGSLMDVLHYPGMNHRVEITEGILADECAALLCYFFRMPRQVFNAQKKAQSST
D
Protein sequence
ecTadA*8e (ecTadA*7.10 further containing A109S, T111R,
D119N, H122N, Y147D, F149Y, T1661, and
D167N)
SEQ ID NO: 826
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKR
GAAGSLMNVLNYPGMNHRVEITEGILADECAALLCDFYRMPRQVFNAQKKAQSSI
N
Protein sequence
ecTadA*8e-V106W
SEQ ID NO: 827
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGWRNSKRGAAGSLMNVLNYPGMNHRVEITEGI
LADECAALLCDFYRMPRQVFNAQKKAQSSIN
Protein sequence
ecTadA*8e-V82G
SEQ ID NO: 828
SEVEFSHEYWMRHALTLAKRARDEREVPVGAVLVLNNRVIGEGWN
RAIGLHDPTAHAEIMALRQGGLVMQNYRLIDATLYGTFEPCVMCA
GAMIHSRIGRVVFGVRNSKRGAAGSLMNVLNYPGMNHRVEITEGI
LADECAALLCDFYRMPRQVFNAQKKAQSSIN
Protein sequence
ecTadA*8e-K20A-R21A
SEQ ID NO: 829
SEVEFSHEYWMRHALTLAAAARDEREVPVGAVLVLNNRVIGEGWN
RAIGLHDPTAHAEIMALRQGGLVMQNYRLIDATLYVTFEPCVMCA
GAMIHSRIGRVVFGVRNSKRGAAGSLMNVLNYPGMNHRVEITEGI
LADECAALLCDFYRMPRQVFNAQKKAQSSIN
Dual base editors (DEs):
Protein sequence
TadDE (NLS3-TadA*Dual-(SGGS)₂XTEN(SGGS)₂-Cas9n-
(SGG)₃S-UGI-(SGG)₃S-UGI-SGGS-NLS2)
SEQ ID NO: 900
MKRTADGSEFESPKKKRKVSEVEFSHEYWMRHALTLAKRARDEGE
APVGAVLVLNNRVIGEGWNRRIGLHDPTAHAEIMALRQGGLVMQN
SRLIDATLYVTFEPCVMCAGAMINSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSGGSGGSTNLSDIIEKETGKQLVIQESILMLPE
EVEEVIGNKPESDILVHTAYDESTDENVMLLTSDAPEYKPWALVI
QDSNGENKIKMLSGGSGGSGGSTNLSDIIEKETGKQLVIQESILM
LPEEVEEVIGNKPESDILVHTAYDESTDENVMLLTSDAPEYKPWA
LVIQDSNGENKIKMLSGGSKRTADGSEFEPKKKRKV
Protein sequence
TadDE-V106W (NLS3-TadA*DualV106W-(SGGS)₂XTEN(SGGS)₂-
Cas9n-(SGG)₃S-UGI-(SGG)₃S-UGI-SGGS-
NLS2)
SEQ ID NO: 901
MKRTADGSEFESPKKKRKVSEVEFSHEYWMRHALTLAKRARDEGE
APVGAVLVLNNRVIGEGWNRRIGLHDPTAHAEIMALRQGGLVMQN
SRLIDATLYVTFEPCVMCAGAMINSRIGRVVFGWRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSGGSGGSTNLSDIIEKETGKQLVIQESILMLPE
EVEEVIGNKPESDILVHTAYDESTDENVMLLTSDAPEYKPWALVI
QDSNGENKIKMLSGGSGGSGGSTNLSDIIEKETGKQLVIQESILM
LPEEVEEVIGNKPESDILVHTAYDESTDENVMLLTSDAPEYKPWA
LVIQDSNGENKIKMLSGGSKRTADGSEFEPKKKRKV
DE parts-dual deaminases:
Protein sequence
TadA*Dual
SEQ ID NO: 920
MSEVEFSHEYWMRHALTLAKRARDEGEAPVGAVLVLNNRVIGEGW
NRRIGLHDPTAHAEIMALRQGGLVMQNSRLIDATLYVTFEPCVMC
AGAMINSRIGRVVFGVRNSKRGAAGSLMNVLNYPGMNHRVEITEG
ILADECAALLCDFYRMPRQVFNAQKKAQSSIN
Protein sequence
TadA*Dual-V106W
SEQ ID NO: 921
MSEVEFSHEYWMRHALTLAKRARDEGEAPVGAVLVLNNRVIGEGW
NRRIGLHDPTAHAEIMALRQGGLVMQNSRLIDATLYVTFEPCVMC
AGAMINSRIGRVVFGWRNSKRGAAGSLMNVLNYPGMNHRVEITEG
ILADECAALLCDFYRMPRQVFNAQKKAQSSIN

What is claimed is:

1. An isolated guide RNA (gRNA) for Clustered Regularly Interspaced Short Palindromic Repeats (CRISPR)-mediated gene editing, wherein the gRNA comprises a CRISPR RNA (crRNA) sequence comprising a targeting sequence comprising at least or consisting of 17 nucleic acids, optionally 17, 18, 19, 20, 21, 22, 23, 24, 25, 26, 27, 28, 29, or 30 nucleic acids, the targeting sequence comprising:
(i) a sequence of at least 17 consecutive nucleic acids, optionally 17, 18, 19, 20, 21, 22, 23, 24, 25, 26, 27, 28, 29, or 30 consequent nucleic acids, contained in SEQ ID NO: 80 or 81; or
(ii) a sequence of at least 17 nucleic acids comprising one or more mutations, optionally one, two, three, four, or five mutations, said mutations relative to the sequence of at least 17 consecutive nucleic acids of (i), optionally 17, 18, 19, 20, 21, 22, 23, 24, 25, 26, 27, 28, 29, or 30 consequent nucleic acids, contained in SEQ ID NO: 80 or 81, optionally wherein the one or more mutations are at any nucleic acid position(s) or are at position(s) other than the 4th to the 7th nucleic acid positions from the 3′-end of the at least 17 consecutive nucleic acids, or
(iii) a sequence of at least 17 nucleic acids which comprises at least 85, 90, 95, 96, 97, 98 or 99% sequence identity to the sequence of at least 17 consecutive nucleic acids of (i), optionally 17, 18, 19, 20, 21, 22, 23, 24, 25, 26, 27, 28, 29, or 30 consequent nucleic acids, contained in SEQ ID NO: 80 or 81;
optionally wherein the gRNA comprises one or more of the following features:
(a) the at least 17 consecutive nucleic acids are immediately upstream of a protospacer adjacent motif (PAM) or protospacer flanking site (PFS) of a CRISPR-associated (Cas) endonuclease in SEQ ID NO: 80 or 81, optionally wherein the Cas endonuclease is Cas9, further optionally wherein the Cas endonuclease is Streptococcus pyogenes Cas9 (SpCas9) and/or the PAM sequence is 5′-NGG-3′ wherein N represents any nucleotide;
(b) the at least 17 consecutive nucleic acids are immediately downstream of the PAM or PFS of a Cas endonuclease in SEQ ID NO: 80 or 81, optionally wherein the Cas endonuclease is Cpf1 and/or the PAM sequence is 5′-TTTN-3′ wherein N represents any nucleotide;
(c) the sequence of said at least 17 consecutive nucleic acids comprises or consists of (c-1) SEQ ID NO: 120, 121, 122, or 123 or (c-2) SEQ ID NO: 130, 131, 132, or 133;
(d) the targeting sequence is 17, 18, 19, 20, 21, 22, 23, 24, 25, 26, 27, 28, 29, or 30 nucleotides in length;
(e) the targeting sequence comprises or consists of (e-1) SEQ ID NO: 120, 121, 122, or 123 or (e-2) SEQ ID NO: 130, 131, 132, or 133;
(f):
(f-1) the gRNA is a single guide RNA (sgRNA) comprising (i) a crRNA sequence comprising the targeting sequence and a crRNA backbone sequence and (ii) a trans-activating CRISPR RNA (tracrRNA) sequence in a single strand, optionally wherein the crRNA sequence and the tracrRNA sequence are linked via a linker optionally comprising the nucleic acid sequence of GAAA, further optionally wherein the gRNA comprises the targeting sequence followed by a sgRNA backbone sequence of any of SEQ ID NOS: 111-114, optionally wherein the sgRNA backbone sequence is followed by one or more uracils, further optionally 1-10 uracils, or
(f-2) the gRNA is a dual guide RNA (dgRNA) formed by hybridization between (i) a crRNA sequence comprising the targeting sequence and a crRNA backbone sequence and (ii) a tracrRNA, optionally wherein the crRNA backbone sequence and the tracrRNA comprise SEQ ID NOS: 115 and 116, respectively, or SEQ ID NOS: 117 and 118, respectively;
(g) the sequence of the gRNA comprises or consists of (g-1) any of SEQ ID NOS: 125-128, 225-228, 325-328, and 425-428 or (g-1) SEQ ID NOS: 135-138, 235-238, 335-338, and 435-438;
(h) the gRNA is synthetic or recombinant; and/or
(i) the gRNA is a synthetic sgRNA and comprises at least one chemical modification, optionally (i-1) 2′-O-methylation further optionally at first three and last three bases and/or (i-2) one or more 3′ phosphorothioate bonds, further optionally between first three and last two bases.

2. A polynucleotide or polynucleotides encoding the isolated gRNA of claim 1, or a vector comprising the polynucleotide or polynucleotides operably linked to one or more regulatory sequences.

3. (canceled)

4. A ribonucleoprotein (RNP), which comprises:
(a) one or more isolated gRNAs of claim 1; which is complexed with
(b) a Cas endonuclease,
optionally wherein: the Cas endonuclease is:
(i) selected from the group consisting of Cas9, Cas3, Cas8a2, Cas8b, Cas8c, Cas10, Cas11, Cas12, Cas 12a or Cpf1, Cas13, Cas13a, C2c1, C2c3, and C2c2;
(ii) a class 2 Cas endonuclease, optionally a type II, type V, or type VI Cas nuclease;
(iii) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9); and/or
(iv) Cas9, optionally comprising any one of SEQ ID NOS: 600-611,
and optionally wherein the RNP is formed by mixing at an approximately equimolar ratio (I) a solution comprising the one or more isolated gRNAs, optionally wherein the pH of the solution is about 6 to 8, about 6.5 to 7.5, further optionally about 7, and (II) a solution comprising the Cas endonuclease, optionally wherein the pH of the solution is about 6 to 8, about 6.5 to 7.5, further optionally about 7, further optionally wherein the mixing is for about 5 minutes.

5. A composition comprising:
(A) a pharmaceutically acceptable carrier; and
(B) one or more RNPs according to claim 4; and
(C) optionally a template DNA,
optionally wherein the one or more RNP comprises:
(I) a first RNP comprising a first isolated gRNA and a first Cas endonuclease, wherein the first isolated gRNA comprises a first crRNA sequence comprising a first targeting sequence, optionally wherein the first targeting sequence comprises or consists of:
(i) SEQ ID NO: 120, 121, 122, or 123; or
(ii) a sequence of at least 17 nucleic acids comprising or consisting of a sequence of at least 17 nucleic acids comprising one or more mutations, optionally one, two, three, four, or five mutations, relative to SEQ ID NO: 120, 121, 122, or 123, optionally wherein the one or more mutations are at any nucleic acid position(s) or are at position(s) other than the 4th to the 7th nucleic acid positions from the 3′-end of SEQ ID NO: 120, 121, 122, or 123,
further optionally wherein the sequence of the first isolated gRNA comprises or consists of any of SEQ ID NOS: 125-128, 225-228, 325-328 and 425-428; and/or
(II) a second RNP comprising a second isolated gRNA and a second Cas endonuclease, wherein the second isolated gRNA comprises a second crRNA sequence comprising a second targeting sequence, optionally wherein the second targeting sequence comprises or consists of:
(i) SEQ ID NO: 130, 131, 132, or 133; or
(ii) a sequence of at least 17 nucleic acids comprising or consisting of a sequence of at least 17 nucleic acids comprising one or more mutations, optionally one, two, three, four, or five mutations, relative to SEQ ID NO: 130, 131, 132, or 133, optionally wherein the one or more mutations are at any nucleic acid position(s) or are at position(s) other than the 4th to the 7th nucleic acid positions from the 3′-end of SEQ ID NO: 130, 131, 132, or 133,
further optionally wherein the sequence of the second isolated gRNA comprises or consists of any of SEQ ID NOS: 135-138, 235-238, 335-338, and 435-438,
optionally wherein the composition further comprises:
(III) a third RNP comprising a third isolated gRNA and a third Cas endonuclease, wherein the third isolated gRNA comprises a third crRNA sequence comprising a third targeting sequence, optionally wherein the third targeting sequence comprises or consists of:
(i) SEQ ID NO: 140, 141, 142, or 143; or
(ii) a sequence of at least 17 nucleic acids comprising or consisting of a sequence of at least 17 nucleic acids comprising one or more mutations, optionally one, two, three, four, or five mutations, relative to SEQ ID NO: 140, 141, 142, or 143, optionally wherein the one or more mutations are at any nucleic acid position(s) or are at position(s) other than the 4th to the 7th nucleic acid positions from the 3′-end of SEQ ID NO: 140, 141, 142, or 143,
further optionally wherein the sequence of the third isolated gRNA comprises or consists of any of SEQ ID NOS: 145-148, 245-248, 345-348, and 445-448; and/or
(IV) a fourth RNP comprising a fourth isolated gRNA and a fourth Cas endonuclease, wherein the fourth isolated gRNA comprises a fourth crRNA sequence comprising a fourth targeting sequence, optionally wherein the fourth targeting sequence comprises or consists of:
(i) SEQ ID NO: 150, 151, 152, or 153; or
(ii) a sequence of at least 17 nucleic acids comprising or consisting of a sequence of at least 17 nucleic acids comprising one or more mutations, optionally one, two, three, four, or five mutations, relative to SEQ ID NO: 150, 151, 152, or 153, optionally wherein the one or more mutations are at any nucleic acid position(s) or are at position(s) other than the 4th to the 7th nucleic acid positions from the 3′-end of SEQ ID NO: 150, 151, 152, or 153,
further optionally wherein the sequence of the fourth isolated gRNA comprises or consists of any of SEQ ID NOS: 155-158, 255-258, 355-358, and 455-458,
further optionally wherein the first, second, third, and/or fourth gRNA(s) independently comprise(s) one or more of the following features:
(a) the targeting sequence is 17, 18, 19, 20, 21, 22, 23, 24, 25, 26, 27, 28, 29, or 30 nucleotides in length;
(b)
(b-1) the gRNA is a sgRNA comprising (i) a crRNA sequence comprising the targeting sequence and a crRNA backbone sequence and (ii) a tracrRNA sequence in a single strand, optionally wherein the crRNA sequence and the tracrRNA sequence are linked via a linker optionally comprising the nucleic acid sequence of GAAA, further optionally wherein the gRNA comprises the targeting sequence followed by a sgRNA backbone sequence of any of SEQ ID NOS: 111-114, optionally wherein the sgRNA backbone sequence is followed by one or more uracils, further optionally 1-10 uracils, or
(b-2) the gRNA is a dgRNA formed by hybridization between (i) a crRNA comprising the targeting sequence and a crRNA backbone sequence and (ii) a tracrRNA, optionally wherein the crRNA backbone sequence and the tracrRNA comprise SEQ ID NOS: 115 and 116, respectively, or SEQ ID NOS: 117 and 118;
(c) the gRNA is synthetic or recombinant; and/or
(d) the gRNA comprises at least one chemical modification, optionally (d-1) 2′-O-methylation optionally at first three and last three bases and/or (d-2) one or more 3′ phosphorothioate bonds, optionally between first three and last two bases,
and optionally wherein the pharmaceutically acceptable carrier comprises a lipid-based transfection competent vesicle (TCV),
yet further optionally wherein:
(a) the pharmaceutically acceptable carrier comprises a lipid-based TCV, and
(b) the template DNA, if present, and/or the one or more RNPs are encapsulated in the TCV,
optionally wherein the template DNA, if present, is co-encapsulated with or separately encapsulated from one or more of the one or more RNPs.

6. (canceled)

7. A composition comprising:
(A) a pharmaceutically acceptable carrier; and
(B) (a) one or more isolated gRNAs according to claim 1 or one or more polynucleotides encoding the one or more isolated gRNAs, and
(b) a Cas endonuclease or a polynucleotide encoding a Cas endonuclease; and
(C) optionally a template DNA or a polynucleotide encoding a template DNA,
optionally wherein the one or more isolated gRNAs comprises:
(I) a first isolated gRNA which comprises a first crRNA sequence comprising a first targeting sequence, optionally wherein the first targeting sequence comprises or consists of:
(i) SEQ ID NO: 120, 121, 122, or 123; or
(ii) a sequence of at least 17 nucleic acids comprising or consisting of a sequence of at least 17 consecutive nucleic acids comprising one or more mutations, optionally one, two, three, four, or five mutations, relative to SEQ ID NO: 120, 121, 122, or 123, optionally wherein the one or more mutations are at any nucleic acid position(s) or are at position(s) other than the 4th to the 7th nucleic acid positions from the 3′-end of SEQ ID NO: 120, 121, 122, or 123,
further optionally wherein the sequence of the first isolated gRNA comprises or consists of any of SEQ ID NOS: 125-128, 225-228, 325-328, and 425-428; and/or
(II) a second isolated gRNA which comprises a second crRNA sequence comprising a second targeting sequence, optionally wherein the second targeting sequence comprises or consists of:
(i) SEQ ID NO: 130, 131, 132, or 133; or
(ii) a sequence of at least 17 nucleic acids comprising or consisting of a sequence of at least 17 consecutive nucleic acids comprising one or more mutations, optionally one, two, three, four, or five mutations, relative to SEQ ID NO: 130, 131, 132, or 133, optionally wherein the one or more mutations are at any nucleic acid position(s) or are at position(s) other than the 4th to the 7th nucleic acid positions from the 3′-end of SEQ ID NO: 130, 131, 132, or 133,
further optionally wherein the sequence of the second isolated gRNA comprises or consists of SEQ ID NOS: 135-138, 235-238, 335-338, and 435-438,
optionally wherein the composition further comprises:
(III) a third isolated gRNA which comprises a third crRNA sequence comprising a third targeting sequence, optionally wherein the third targeting sequence comprises or consists of:
(i) SEQ ID NO: 140, 141, 142, or 143; or
(ii) a sequence of at least 17 nucleic acids comprising or consisting of a sequence of at least 17 consecutive nucleic acids comprising one or more mutations, optionally one, two, three, four, or five mutations, relative to SEQ ID NO: 140, 141, 142, or 143, optionally wherein the one or more mutations are at any nucleic acid position(s) or are at position(s) other than the 4th to the 7th nucleic acid positions from the 3′-end of SEQ ID NO: 140, 141, 142, or 143,
further optionally wherein the sequence of the third isolated gRNA comprises or consists of any of SEQ ID NOS: 145-148, 245-248, 345-348, and 445-448; and/or
(IV) a fourth isolated gRNA which comprises a fourth crRNA sequence comprising a fourth targeting sequence, optionally wherein the fourth targeting sequence comprises or consists of:
(i) SEQ ID NO: 150, 151, 152, or 153; or
(ii) a sequence of at least 17 nucleic acids comprising or consisting of a sequence of at least 17 consecutive nucleic acids comprising one or more mutations, optionally one, two, three, four, or five mutations, relative to SEQ ID NO: 150, 151, 152, or 153, optionally wherein the one or more mutations are at any nucleic acid position(s) or are at position(s) other than the 4th to the 7th nucleic acid positions from the 3′-end of SEQ ID NO: 150, 151, 152, or 153,
further optionally wherein the sequence of the fourth isolated gRNA comprises or consists of any of SEQ ID NOS: 155-158, 255-258, 355-358, and 455-458,
further optionally wherein the first, second, third, and/or fourth gRNA(s) independently comprise(s) one or more of the following features:
(a) the targeting sequence is 17, 18, 19, 20, 21, 22, 23, 24, 25, 26, 27, 28, 29, or 30 nucleotides in length;
(b) the gRNA comprises a tracrRNA sequence, wherein:
(b-1) the gRNA is a sgRNA comprising (i) a crRNA sequence comprising the targeting sequence and a crRNA backbone sequence and (ii) a tracrRNA sequence in a single strand, optionally wherein the crRNA sequence and the tracrRNA sequence are linked via a linker optionally comprising the nucleic acid sequence of GAAA, further optionally wherein the gRNA comprises the targeting sequence followed by a sgRNA backbone sequence of any of SEQ ID NOS: 111-114, optionally wherein the sgRNA backbone sequence is followed by one or more uracils, further optionally 1-10 uracils, or
(b-2) the gRNA is a dgRNA formed by hybridization between (i) a crRNA comprising the targeting sequence and a crRNA backbone sequence and (ii) a tracrRNA, optionally wherein the crRNA backbone sequence and the tracrRNA comprise SEQ ID NOS: 115 and 116, respectively, or SEQ ID NOS: 117 and 118;
(c) the gRNA is synthetic or recombinant, and/or
(d) the gRNA is a synthetic sgRNA and comprises at least one chemical modification, optionally (d-1) 2′-O-methylation optionally at first three and last three bases and/or (d-2) one or more 3′ phosphorothioate bonds, optionally between first three and last two bases,
optionally wherein the Cas endonuclease is:
(i) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2;
(ii) a class 2 Cas endonuclease, optionally a type II, type V, or type VI Cas nuclease;
(iii) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of SpCas9 (e.g., CP1012-SpCas9, CP1028-SpCas9, CP1041-SpCaS9, CP1249-SpCas9, and CP1300-SpCas9), eSpCas9, HypaCas9, FokI-Fused dCas9, xCas9, SpRY (variant of SpCas9), and SpG (variant of SpCas9),
(iv) Cas9, optionally comprising any one of SEQ ID NOS: 600-611, and/or
(v) Cas12a of Lachnospiraceae bacterium ND2006 (LbCas12a), Acidaminococcus sp. BV3L6 (AsCas12a), or Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a;
and further optionally wherein the pharmaceutically acceptable carrier comprises a lipid-based TCV,
yet further optionally wherein, in (B), the composition comprises at least one of the following:
(I) (a) said one or more isolated gRNAs. and
(b) a vector comprising said polynucleotide encoding a Cas endonuclease;
(II) (a) one or more vectors comprising said one or more polynucleotides encoding said one or more isolated gRNAs, and
(b) said Cas endonuclease:
(III) (a) one or more vectors comprising said one or more polynucleotides encoding the one or more isolated gRNAs, and
(b) a vector comprising said polynucleotide encoding a Cas endonuclease; or
(IV) (a) said one or more isolated gRNAs, and
(b) said Cas endonuclease.
optionally wherein the composition comprises one or more of the following features:
(i) said e one or more vectors of (II) (a) and (III) (a) and said vector of (I) (b) and (III) (b) are individually selected from plasmids. RNA replicons, virus-like particles (VLPs), and viral vectors, optionally retroviral, lentiviral, or adenoviral vectors;
(ii) in (II) (a) and/or (III) (a), when said one or more isolated gRNAs are more than one isolated gRNAs, said more than one gRNAs are encoded by one or more nucleic acids comprised in a single vector or comprised in separate vectors; and/or
(iii) in (III), said one or more isolated gRNAs and said Cas endonuclease are encoded by one or more nucleic acids comprised in a single vector or comprised in separate vectors.

8. (canceled)

9. The composition of claim 5, wherein the template DNA comprises:
(I) a single-strand oligo DNA nucleotide molecule (ssODN) comprising or consisting of a 5′ homology arm, an optional central region, and a 3′ homology arm, wherein:
(a) (i) said 5′ homology arm comprises or consists of (i-1) a sequence corresponding to the first nucleotide to at least the 10th nucleotide counting from the 3′-end of SEQ ID NO: 581, (i-2) a sequence selected from any of SEQ ID NOS: 511, 521, 531, 541, 551, 561, 571, and 581, or (i-3) a sequence comprising at least one (such as one, two, three, four, five, six, seven, eight, nine, or ten) mutation(s) relative to the sequence of (i-1) or (i-2),
(ii) said optional central region is 1-100 nucleotides (nt) in length; and
(iii) said 3′ homology arm comprises or consists of (iii-1) a sequence corresponding to the first nucleotide to at least the 10th nucleotide counting from the 5′-end of SEQ ID NO: 582, (iii-2) a sequence selected from any of SEQ ID NOS: 512, 522, 532, 542, 552, 562, 572, and 582, or (iii-3) a sequence comprising at least one (such as one, two, three, four, five, six, seven, eight, nine, or ten) mutation(s) relative to the sequence of (iii-1) or (iii-2),
optionally wherein the ssODN comprises or consists of a sequence selected from any of SEQ ID NOs: 510, 520, 530, 540, 550, 560, 570, and 580;
(b) the sequence of the ssODN is fully complementary to the sequence any of any of the ssODNs of (a); and/or
(II) a double-strand DNA molecule, which comprises a first strand comprising or consisting of any of the ssODN sequences of (I) and a second strand complementary to the first strand.

10-12. (canceled)

13. A CRISPR-mediated gene editing reporter system comprising:
(A) (I) a cell comprising a DNA molecule comprising:
(a) a DNA span comprising:
(i) at least one first segment comprising or consisting of SEQ ID NO: 20, 21, 22, or 23 or comprising or consisting of a sequence comprising one or more mutations, optionally one, two, three, four, or five mutations, relative to SEQ ID NO: 20, 21, 22, or 23; and/or
(ii) at least one second segment comprising or consisting of SEQ ID NO: 30, 31, 32, or 33 or comprising or consisting of a sequence comprising one or more mutations, optionally one, two, three, four, or five mutations, relative to SEQ ID NO: 30, 31, 32, or 33,
wherein the DNA span is/are flanked by:
(iii) a third segment comprising or consisting of SEQ ID NO: 40, 41, 42, or 43 or comprising or consisting of a sequence comprising one or more mutations, optionally one, two, three, four, or five mutations, relative to SEQ ID NO: 40, 41, 42, or 43; and
(iv) a fourth segment comprising or consisting of SEQ ID NO: 50, 51, 52, or 53 or comprising or consisting of a sequence comprising one or more mutations, optionally one, two, three, four, or five mutations, relative to SEQ ID NO: 50, 51, 52, or 53, wherein:
(a-1) the third and fourth segments may be upstream and downstream, respectively, of the DNA span, or the third and fourth segments may be downstream and upstream, respectively, of the DNA span, and
(a-2) at least one terminator sequence is contained within said DNA span; and
(b) a reporter gene sequence located downstream of the third and fourth segments, optionally wherein the DNA molecule comprises any of SEQ ID NOS: 1, 60, 70, 80, 81, and 90;
(II) a tissue comprising said cell, or
(III) a transgenic animal comprising said cell and/or said tissue; and
(B) CRISPR-mediated gene editing agents, comprising:
(1) multiple isolated gRNAs at least one of which is according to claim 1, or one or more polynucleotides encoding the multiple isolated gRNAs, wherein the multiple isolated gRNAs comprise:
(I) a first isolated gRNA which comprises a first crRNA sequence comprising or consisting of a first targeting sequence, optionally wherein the first targeting sequence comprises or consists of:
(i) SEQ ID NO: 120, 121, 122, or 123; or
(ii) a sequence of at least 17 nucleic acids comprising or consisting of a sequence of at least 17 nucleic acids comprising one or more mutations, optionally one, two, three, four, or five mutations, relative to SEQ ID NO: 120, 121, 122, or 123, optionally wherein the one or more mutations are at any nucleic acid position(s) or are at position(s) other than the 4th to the 7th nucleic acid positions from the 3′-end of SEQ ID NO: 120, 121, 122, or 123,
further optionally wherein the sequence of the first isolated gRNA comprises or consists of any of SEQ ID NOS: 125-128, 225-228, 325-328, and 425-428; and/or
(II) a second isolated gRNA which comprises a second crRNA sequence comprising or consisting of a second targeting sequence, optionally wherein the second targeting sequence comprises or consists of:
(i) SEQ ID NO: 130, 131, 132, or 133; or
(ii) a sequence of at least 17 nucleic acids comprising or consisting of a sequence of at least 17 nucleic acids comprising one or more mutations, optionally one, two, three, four, or five mutations, relative to SEQ ID NO: 130, 131, 132, or 133, optionally wherein the one or more mutations are at any nucleic acid position(s) or are at position(s) other than the 4th to the 7th nucleic acid positions from the 3′-end of SEQ ID NO: 130, 131, 132, or 133,
further optionally wherein the sequence of the second isolated gRNA comprises or consists of any of SEQ ID NOS: 135-138, 235-238, 335-338, and 435-438,
and further comprises:
(III) a third isolated gRNA which comprises a third crRNA sequence comprising or consisting of a third targeting sequence, optionally wherein the third targeting sequence comprises or consists of:
(i) SEQ ID NO: 140, 141, 142, or 143; or
(ii) a sequence of at least 17 nucleic acids comprising or consisting of a sequence of at least 17 nucleic acids comprising one or more mutations, optionally one, two, three, four, or five mutations, relative to SEQ ID NO: 140, 141, 142, or 143, optionally wherein the one or more mutations are at any nucleic acid position(s) or are at position(s) other than the 4th to the 7th nucleic acid positions from the 3′-end of SEQ ID NO: 140, 141, 142, or 143,
further optionally wherein the sequence of the third isolated gRNA comprises or consists of any of SEQ ID NOS: 145-148, 245-248, 345-348, and 445-448; and
(IV) a fourth isolated gRNA which comprises a fourth crRNA sequence comprising a fourth targeting sequence, optionally wherein the fourth targeting sequence comprises or consists of:
(i) SEQ ID NO: 150, 151, 152, or 153; or
(ii) a sequence of at least 17 nucleic acids comprising or consisting of a sequence of at least 17 nucleic acids comprising one or more mutations, optionally one, two, three, four, or five mutations, relative to SEQ ID NO: 150, 151, 152, or 153, optionally wherein the one or more mutations are at any nucleic acid position(s) or are at position(s) other than the 4th to the 7th nucleic acid positions from the 3′-end of SEQ ID NO: 150, 151, 152, or 153,
further optionally wherein the sequence of the fourth isolated gRNA comprises or consists of any of SEQ ID NOS: 155-158, 255-258, 355-358, and 455-458; and
(2) a Cas endonuclease or a polynucleotide encoding a Cas endonuclease; and
(3) optionally a template DNA or a polynucleotide encoding a template DNA,
optionally wherein (1) the multiple isolated gRNAs or the one or more polynucleotides encoding the multiple isolated gRNAs and (2) the Cas endonuclease or the polynucleotide encoding a Cas endonuclease, and (3) optionally the template DNA or the polynucleotide encoding a template DNA, are comprised in a composition
optionally wherein:
in (A) (I), the cell is a cell line or a primary cell;
in (A) (I), the cell is a cell of a tissue or organ of interest;
in (A) (I) (b). the reporter gene encodes a fluorescent marker, optionally monomeric cherry (mCherry). tandem dimer Tomato (tdTomato), red fluorescent protein (RFP), DsRed1. DsRed S197Y, green fluorescent protein (GFP), enhanced FP (EGFP), yellow fluorescent protein (YFP), enhanced yellow fluorescent protein (EYFP), cyan fluorescent protein (CFP), or enhanced cyan (ECFP);
in (A) (III), the transgenic animal is a rodent, further optionally a mouse or a rat.
in (A) (III), the transgenic animal comprises said DNA span flanked by said third and fourth segments at the Rosa26 locus; and/or
in (A) (III), the transgenic animal is the Ai9 or Ai14 mouse.

14. (canceled)

15. A method of testing in vitro the level of CRISPR-mediated gene editing events in a cell, the method comprising:
(a) applying in vitro to one or more cells CRISPR-mediated gene editing agents, wherein said CRISPR-mediated gene editing agents comprise a CRISPR-mediated gene editing reporter system according to claim 13 and said one or more cells are according to claim 13- or 14; and
(b) analyzing the level of CRISPR-mediated gene editing events in said one or more cells, optionally by
(i) quantifying the reporter gene expression in said one or more cells, optionally via flow cytometry, fluorescent microscopy, or qPCR; and/or
(ii) determining the presence or absence or level of (ii-1) the DNA sequence flanked by the sites cleavable by the third and fourth isolated gRNAs or (ii-2) the transcript thereof in said one or more cells, optionally via PCR or qPCR, respectively,
further optionally wherein:
(i) said CRISPR-mediated gene editing agents are contained in a composition comprising a pharmaceutically acceptable carrier of interest and the method is for testing the effect of the carrier of interest on CRISPR-mediated gene editing events;
(ii) said cell is of a cell type of interest, and the method is for testing whether the cell type of interest is compatible with CRISPR-mediated gene editing; and/or
(iii) said cell is of a cell type of interest, the CRISPR-mediated gene editing agents are contained in a composition comprising a pharmaceutically acceptable carrier of interest, and the method is for testing whether the carrier of interest is compatible with the cell type of interest.

16. A method of testing ex vivo the level of CRISPR-mediated gene editing events in a tissue, the method comprising:
(a) applying ex vivo to one or more tissues CRISPR-mediated gene editing agents, wherein said CRISPR-mediated gene editing agents comprise a CRISPR-mediated gene editing reporter system according to claim 13, and said one or more tissues are according to claim 13- or 14; and
(b) analyzing the level of CRISPR-mediated gene editing events in said one or more tissues, optionally by
(i) quantifying the reporter gene expression in the one or more tissues or cells contained therein, optionally via flow cytometry, fluorescent microscopy, or qPCR; and/or
(ii) determining the presence or absence or level of (ii-1) the DNA sequence flanked by the sites cleavable by the third and fourth isolated gRNAs or (ii-2) the transcript thereof in the one or more tissues or cells contained therein, optionally via PCR or qPCR, respectively,
optionally wherein:
(i) the CRISPR-mediated gene editing agents are contained in a composition comprising a pharmaceutically acceptable carrier of interest and the method is for testing the effect of the carrier of interest on CRISPR-mediated gene editing events;
(ii) the tissue is of a tissue type of interest, and the method is for testing whether the tissue type of interest is compatible with CRISPR-mediated gene editing; and/or
(iii) the tissue is of a tissue type of interest, the CRISPR-mediated gene editing agents are contained in a composition comprising a pharmaceutically acceptable carrier of interest, and the method is for testing whether the carrier of interest is compatible with the tissue type of interest.

17. A method of testing in vivo the level of CRISPR-mediated gene editing events in an animal, the method comprising:
(a) applying to one or more transgenic animals CRISPR-mediated gene editing agents, wherein said CRISPR-mediated gene editing agents comprise a CRISPR-mediated gene editing reporter system according to claim 13 and said one or more transgenic animals are further according to claim 13; and
(b) analyzing the level of CRISPR-mediated gene editing events in said one or more transgenic animals, optionally by
(i) quantifying the reporter gene expression in the one or more transgenic animals or tissues or cells derived therefrom, optionally via flow cytometry, fluorescent microscopy, or qPCR; and/or
(ii) determining the presence or absence or level of (ii-1) the DNA sequence flanked by the sites cleavable by the third and fourth isolated gRNAs or (ii-2) the transcript thereof in the one or more transgenic animals or tissues or cells derived therefrom, optionally via PCR or qPCR, respectively,
optionally wherein:
(i) the CRISPR-mediated gene editing agents are contained in a composition comprising a pharmaceutically acceptable carrier of interest and the method is for testing the effect of the carrier of interest on CRISPR-mediated gene editing events in vivo;
(ii) the transgenic animal is of a species of interest, and the method is for testing whether the species of interest is compatible with CRISPR-mediated gene editing;
(iii) the transgenic animal is of a species of interest, the CRISPR-mediated gene editing agents are contained in a composition comprising a pharmaceutically acceptable carrier of interest, and the method is for testing whether the carrier of interest is compatible with the transgenic animal is of a species of interest;
(iv) the method is for evaluating the level of side effects and/or adverse events;
(v) the applying of step (a) comprises administrating via an administration route of interest, and the method is for testing whether the administration route of interest is suited for effecting CRISPR-mediated gene editing, optionally based on the level of gene editing evens and/or the level of side effects and/or adverse events; and/or
(vi) the applying of step (a) comprises administrating the CRISPR-mediated gene editing agents at a dose or a dose range of interest, and the method is for determining an approximate dose or dose rage or a dosing regimen suited for effecting CRISPR-mediated gene editing, optionally based on the level of gene editing evens and/or the level of side effects and/or adverse events.

18. A method of knocking out a DNA segment of interest in a cell, tissue, or subject, wherein:
(i) said DNA segment of interest is comprised in an intervening sequence flanked by a 5′ first site cleavable by CRISPR-mediated gene editing via a first gRNA and a 3′ second site cleavable by CRISPR-mediated gene editing via a second gRNA; and
(ii) said intervening sequence comprises at least one third site cleavable by CRISPR-mediated gene editing via a third gRNA,
the method comprising applying CRISPR-mediated gene editing agents, which comprise:
(a) said first gRNA, said second gRNA, and said third gRNA (which are optionally comprised at equimolar ratios) or one or more polynucleotides encoding said first gRNA, said second gRNA, and said third gRNA;
(b) a Cas endonuclease or a polynucleotide encoding a Cas endonuclease; and
(c) optionally a template DNA or a polynucleotide encoding a template DNA, further optionally wherein the template DNA comprises (c-1) a 5′ homology arm homologous or complementary to the DNA sequence immediately upstream of the first site and (c-2) a 3′ homology arm homologous or complementary to the DNA sequence immediately downstream of the second site,
to the cell, tissue, or subject,
optionally wherein the subject is a human or a non-human subject, further optionally a non-human primate or selected from a rodent (mouse, rat, guinea pig, hamster), rabbit, cat, dog, pig, goat, sheep, horse, or monkey, and still further optionally a mouse or a rat,
and further optionally wherein said intervening sequence: (i) comprises two or more third sites cleavable by CRISPR-mediated gene editing via the third gRNA and/or (ii) is about 10-10000 nucleotides in length, about 20-5000 nucleotides in length, about 50-2500 nucleotides in length, about 100-2000 nucleotides in length, about 500-2000 nucleotides in length, or about 500-1500 nucleotides in length,
yet further optionally wherein said intervening sequence further comprises at least one fourth site cleavable by CRISPR-mediated gene editing via a fourth gRNA which comprises a different target specificity relative to the third gRNA, and wherein the CRISPR-mediated gene editing agents further comprise the fourth gRNA, optionally wherein said intervening sequence comprises two or more fourth sites cleavable by CRISPR-mediated gene editing via the fourth gRNA.

19. (canceled)

20. A composition for knocking out a DNA segment of interest in a cell, tissue, or subject via the method of claim 18, wherein the composition comprises:
(a) said first gRNA, said second gRNA, and said third gRNA (which are optionally comprised at equimolar ratios) or one or more polynucleotides encoding said first gRNA, said second gRNA, and said third gRNA (optionally at an equimolar ratio);
(b) the Cas endonuclease or a polynucleotide encoding the Cas endonuclease; and
(c) optionally the template DNA or a polynucleotide encoding the template DNA wherein the presence of said third gRNA increases the efficiency or probability of knocking out the DNA segment of interest,
optionally wherein the composition further comprise the fourth gRNA, wherein the presence of the fourth gRNA increases the efficiency or probability of knocking out the DNA segment of interest.

21. (canceled)

22. A method of effecting CRISPR-mediated gene editing in the eye, optionally in the cornea, iris, retina, or subretinal tissue, the method comprising administering CRISPR-mediated gene editing agents directly into the eye of a subject, optionally into the cornea, retina, or subretinal tissue,
wherein the CRISPR-mediated gene editing agents comprise:
(a) one or more gRNAs or one or more polynucleotides encoding the one or more gRNAs;
(b) a Cas endonuclease or a polynucleotide encoding a Cas endonuclease; and
(c) optionally a template DNA or a polynucleotide encoding a template DNA,
and any one or more of (a)-(c) are encapsulated in a TCV,
optionally wherein the subject is a human, non-human, non-human primate, a rodent (mouse, rat, guinea pig, hamster), rabbit, cat, dog, pig, goat, sheep, horse, or monkey, and/or wherein the method is for treating a genetic disease or disorder of the eye, optionally a disease affecting one or more of the cornea. iris or retina,
further optionally wherein the method is for knocking out a DNA segment of interest in the eye or a cell thereof of the subject, wherein:
(i) said DNA segment of interest is comprised in an intervening sequence flanked by a 5′ first site cleavable by CRISPR-mediated gene editing via a first gRNA and a 3′ second site cleavable by CRISPR-mediated gene editing via a second gRNA; and
(ii) said intervening sequence comprises at least one third site cleavable by CRISPR-mediated gene editing via a third gRNA,
and wherein:
in (a), said one or more gRNAs comprise or consist of the first gRNA, the second gRNA, and the third gRNA (optionally at an equimolar ratio), or said one or more polynucleotides encoding the one or more gRNAs comprise or consist of one or more polynucleotides encoding the first gRNA, the second gRNA, and the third gRNA; and
in (c), if said template DNA is present, the template DNA optionally comprises (c-1) a 5′ homology arm homologous or complementary to the DNA sequence immediately upstream of the first site and (c-2) a 3′ homology arm homologous or complementary to the DNA sequence immediately downstream of the second site, or if said polynucleotide encoding a template DNA is present, the polynucleotide optionally encodes (c-1) a 5′ homology arm homologous or complementary to the DNA sequence immediately upstream of the first site and (c-2) a 3′ homology arm homologous or complementary to the DNA sequence immediately downstream of the second site,
optionally wherein said intervening sequence: (i) comprises two or more third sites cleavable by CRISPR-mediated gene editing via the third gRNA and/or (ii) is about 10-10000 nucleotides in length, about 20-5000 nucleotides in length, about 50-2500 nucleotides in length, about 100-2000 nucleotides in length, about 500-2000 nucleotides in length, or about 500-1500 nucleotides in length.
further optionally wherein said intervening sequence further comprises at least one fourth site cleavable by CRISPR-mediated gene editing via a fourth gRNA which comprises a different target specificity relative to the third gRNA, and wherein the CRISPR-mediated gene editing agents further comprise the fourth gRNA, optionally wherein said intervening sequence comprises two or more fourth sites cleavable by CRISPR-mediated gene editing via the fourth gRNA.

23-24. (canceled)

25. The method of claim 22, wherein the TCV comprises at least one cationic or ionizable cationic lipid, optionally wherein the TCV comprises one or more of the following features:
(i) said at least one cationic or ionizable cationic lipid comprises, essentially consists of, or consists of a lipid selected from the group consisting of N,N-dimethyl-2,3-dioleyloxy) propylamine (DODMA), 1,2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino) butanoate (MC3), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxyl) propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAR.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl) propyl-N,N-N-triethylammonium chloride (“DOTMA”); 1,2-Dioleyloxy-3-trimethylaminopropane chloride salt (“DOTAP.C1”); 3.beta.-(N-(N′,N′-dimethylaminoethane)-carbamoyl) cholesterol (“DC-Chol”), N-(1-(2,3-dioleyloxyl) propyl)-N-2-(sperminecarboxamido)ethyl)-N,N-dimethyl-ammonium trifluoracetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof; and/or
the amount of the at least one cationic or ionizable cationic lipid relative to the total components of the TCV is: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %;
(ii) said TCV further comprises at least one helper lipid, optionally wherein the at least one helper lipid comprises, essentially consists of, or consists of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof,
optionally wherein the amount of the at least one helper lipid relative to the total components of the TCV is about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %;
(iii) said TCV further comprises at least one phospholipid, optionally wherein the at least one phospholipid comprises, essentially consists of, or consists of a lipid selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof,
optionally wherein the amount of the at least one phospholipid relative to the total components of the TCV is about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %;
(iv) said TCV further comprises at least one cholesterol or cholesterol derivative, optionally wherein the at least one cholesterol or cholesterol derivative comprises, essentially consists of, or consists of a cholesterol or cholesterol derivative selected from the group consisting of cholesterol, N,N-dimethyl-N-ethylcarboxamidocholesterol (DC-Chol), 1,4-bis(3-N-oleylamino-propyl) piperazine, imidazole cholesterol ester (ICE), and any combinations thereof,
optionally wherein the amount of the at least one cholesterol or cholesterol derivative relative to the total components of the TCV is about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%;
(v) said TCV further comprises at least one PEG or PEG-lipid, optionally wherein the at least one PEG-lipid comprises, essentially consists of, or consists of a PEG-lipid selected from the group consisting of PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combinations thereof, optionally wherein the total amount of said at least one PEG or PEG-lipid in said TCV is at most 2 mol %, 1.5 mol %, 1.0 mol %, 0.5 mol %, or 0.1 to 0.5 mol %,
optionally wherein the amount of the at least one PEG or PEG-lipid relative to the total components of the TCV is about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %;
(vi) said TCV further does not comprise any PEG or PEG-lipid;
(vii) said TCV is substantially, essentially, or entirely free of destabilizing agents, and/or
(viii) said TCV is substantially, essentially, or entirely free of any permanently cationic lipids and/or any permanently anionic lipids
or any combination of (i) to (viii),
further optionally wherein:
(I) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC; and
(iv) at least one cholesterol or cholesterol derivative,
optionally wherein the amounts of the at least one ionizable cationic lipid, the at least one helper lipid, the at least one phospholipid, and the at least one cholesterol or cholesterol derivative, relative to the total components of the TCV, is about 20 mol %, about 30 mol %, about 10 mol %, and about 40 mol %, respectively; or
(II) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC:
(iv) at least one cholesterol or cholesterol derivative; and
(v) at least one PEG or PEG-lipid, which is optionally PEG-DMG, optionally wherein the amounts of the at least one ionizable cationic lipid, the at least one helper lipid, the at least one phospholipid, the at least one cholesterol or cholesterol derivative, and the at least one PEG or PEG-lipid, relative to the total components of the TCV, is about 20 mol %, about 30 mol %, about 10 mol %, about 39 mol %, and about 1 mol %, respectively,
further optionally wherein:
(a) the size of the TCV before encapsulation is in a range of about 9 nm to about 80 nm, optionally about 10-40 nm, further optionally about 20-35 nm, at pH of about 3.5 to 4 or at pH of about 4;
(b) the size of the TCV after encapsulation of the at least one cargo is in a range of about 80 nm to about 1500 nm, optionally about 800 nm to about 1400 or about 1000 nm to about 1200 nm or about 80 nm to about 300 nm or about 100 nm to about 250 nm;
(c) the TCV after encapsulation is further comprised in a matrix vesicle, which is optionally for gradual release of the TCV; and/or
(d) the final ethanol concentration of the composition is 5% (v/v) or below, preferably 0.5% (v/v) or below,
yet further optionally wherein the TCV further comprises, is contained in a composition which comprises, and/or is stored in the presence of at least one cryoprotectant, optionally wherein:
(a) the cryoprotectant comprises a sugar-based molecule, which is optionally sucrose, trehalose, or a combination thereof:
(b) the concentration of the cryoprotectant is about 1% to about 40%, about 3% to about 30%, about 5% to about 30%, about 10% to about 20%, or about 15%;
(c) the TCV is stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months; or
(d) any combination of (a)-(c).

26-28. (canceled)

29. A composition comprising:
(A) a pharmaceutically acceptable carrier, which is or comprises a lipid-based TCV; and
(B) a RNP, which is or comprises a base editor complexed with a gRNA,
wherein the RNP is encapsulated in the lipid based TCV,
optionally wherein the base editor:
(I) is an adenine base editor (ABE), a cytidine base editor (CBE), or a dual editor (DE); and/or
(II) comprises a Cas-derived platform protein linked to a deaminase, optionally wherein:
(a) the Cas-derived platform protein is, comprises, or is derived from a Cas-derived nickase (nCas) or a catalytically dead Cas (dCas);
(b) the deaminase is:
(i) an adenine deaminase;
(ii) a cytidine deaminase; or
(ii) an adenine cytidine deaminase (dual deaminase); and/or
(c) the gRNA is designed to effect base editing in the presence of a target DNA and the Cas-derived platform protein, optionally wherein the base editing comprises:
(i) transversion of a target adenine to a guanine;
(ii) transversion of a target cytidine to a thymidine; or
(iii) transversion of a target adenine to a guanine and transversion of a target cytidine to a thymidine,
and optionally wherein said gRNA comprises one or more of the following features:
(a) said gRNA comprises a targeting sequence of 17, 18, 19, 20, 21, 22, 23, 24, 25, 26, 27, 28, 29, or 30 nucleotides in length;
(b) (b-1) said gRNA is a sgRNA comprising (i) a crRNA sequence comprising the targeting sequence and a crRNA backbone sequence and (ii) a tracrRNA sequence in a single strand, optionally wherein the crRNA sequence and the tracrRNA sequence are linked via a linker optionally comprising the nucleic acid sequence of GAAA, further optionally wherein said gRNA comprises the targeting sequence followed by a sgRNA backbone sequence of any of SEQ ID NOS: 111-114, optionally wherein said sgRNA backbone sequence is followed by one or more uracils, further optionally 1-10 uracils, or
(b-2) said gRNA is a dgRNA formed by hybridization between (i) a crRNA comprising the targeting sequence and a crRNA backbone sequence and (ii) a tracrRNA, optionally wherein the crRNA backbone sequence and the tracrRNA comprise SEQ ID NOS: 115 and 116, respectively, or SEQ ID NOS: 117 and 118;
(c) said gRNA is synthetic or recombinant; and/or
(d) said gRNA comprises at least one chemical modification, optionally (d-1) 2′-O-methylation optionally at first three and last three bases and/or (d-2) one or more 3′ phosphorothioate bonds, optionally between first three and last two bases.

30. The composition of claim 29, wherein:
(1) the base editor comprises a Cas-derived platform protein linked to a deaminase, optionally wherein the Cas-derived platform protein:
(i) is or comprises a Cas-derived nickase (nCas) or a catalytically dead Cas (dCas);
(ii) is derived from a Cas endonuclease which is:
(i-1) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2;
(i-2) a class 2 Cas endonuclease, optionally a type II, type V, or type VI Cas nuclease;
(i-3) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, circular permutants of SpCas9 (e.g., CP1012-SpCas9, CP1028-SpCas9, CP1041-SpCaS9, CP1249-SpCas9, and CP1300-SpCas9), eSpCas9, HypaCas9, FokI-Fused dCas9, xCas9, SpRY (variant of SpCas9), and SpG (variant of SpCas9);
(i-4) Cas9, optionally comprising any one of SEQ ID NOS: 600-611; and/or
(i-5) Cas12a of Lachnospiraceae bacterium ND2006 (LbCas12a), Acidaminococcus sp. BV3L6 (AsCas12a), or Francisella tularensis subsp. novicidain U112 (FnCas12a), or BpCas12a, CMtCas12a, EeCas12a, Lb2Cas12a, Lb3Cas12a, LiCas12a, MbCas12a, PbCas12a, PcCas12a, PeCas12a, PdCas12a, PmCas12a, or SsCas12a;
(iii) is or comprises any of the following or a variant thereof:
(iii-1) a SpCas9 nickase, optionally comprising the sequence of SEQ ID NO: 621 or a Cas9 variant (optionally SpCas9 variant) comprising the D10A substitution of SpCas9;
(iii-2) a dead SpCas9, (dCas9) optionally comprising the sequence of SEQ ID NO: 620 or a Cas9 variant, optionally SpCas9 variant further optionally one comprising the D10A and H840A substitutions of dCas9;
(iii-3) a VQR-SpCas9 nickase, optionally comprising the sequence of SEQ ID NO: 631;
(iii-4) a EQR-SpCas9 nickase, optionally comprising the sequence of SEQ ID NO: 632;
(iii-5) a VRER-SpCas9 nickase, optionally comprising the sequence of SEQ ID NO: 633;
(iii-6) a CP1028-SpCas9, optionally comprising the sequence of SEQ ID NO: 634;
(iii-7) a CP1041-SpCas9, optionally comprising the sequence of SEQ ID NO: 635;
(iii-8) a SpCas9-NG, optionally comprising the sequence of SEQ ID NO: 636;
(iii-9) SaCas9 nickase (SEQ ID NO: 640);
(iii-10) SaCas9-KKH (SEQ ID NO: 641);
(iii-11) a catalytically dead LbCas12a (dLbCas12a), optionally comprising the sequence of SEQ ID NO: 650; or
(iii-12) an engineered AsCas12a (enAsCas12a), optionally comprising the sequence of SEQ ID NO: 660;
(2) the base editor comprises a Cas-derived platform protein linked to a deaminase, and wherein the deaminase is:
(a) an adenine deaminase which:
(i) is derived from a TadA, optionally from TadA of E. coli (ecTadA), optionally comprising the sequence of SEQ ID NO: 820; and/or
(ii) is or comprises any of the following:
(ii-1) ecTadA*8e (SEQ ID NO: 826) or a TadA variant (optionally ecTadA variant or ecTadA*7.10 variant) comprising at least 90%, at least 95%, at least 96%, at least 97%, at least 98%, at least 99% sequence identity to SEQ ID NO: 826 and/or comprising one or more of the substitutions A109S, T111R, D119N, H122N, Y147D, F149Y, T166I, and D167N of ecTadA*8e;
(ii-2) ecTadA*8e-V106W (SEQ ID NO: 827) or a TadA variant (optionally ecTadA variant or ecTadA*8e variant) comprising at least 90%, at least 95%, at least 96%, at least 97%, at least 98%, at least 99% sequence identity to SEQ ID NO: 827 and/or 106W of ecTadA*8e-V106W;
(ii-3) ecTadA*8e-V82G (SEQ ID NO: 828) or a TadA variant (optionally ecTadA variant or ecTadA*8e variant) comprising at least 90%, at least 95%, at least 96%, at least 97%, at least 98%, at least 99% sequence identity to SEQ ID NO: 828 and/or the V82G substitution of ecTadA*8e-V82G:
(ii-4) ecTadA*8e-K20A-R21A (SEQ ID NO: 829) or a TadA variant (optionally ecTadA variant or ecTadA*8e variant) comprising at least 90%, at least 95%, at least 96%, at least 97%, at least 98%, at least 99% sequence identity to SEQ ID NO: 829 and/or comprising the K20A and/or R21A substitutions of ecTadA*8e-K20A-R21A;
(ii-5) ecTadA*6.3 (SEQ ID NO: 821) or a TadA variant (optionally ecTadA variant) comprising at least 90%, at least 95%, at least 96%, at least 97%, at least 98%, at least 99% sequence identity to SEQ ID NO: 821 and/or comprising one or more of the substitutions A106V, D108N, D147Y, and E155V; L84F. H123Y, and 1157F; H36L, R51L, S146C. and K157N; and P48S of ecTadA*6.3;
(ii-6) ecTadA*6.4 (SEQ ID NO: 822) or a TadA variant (optionally ecTadA variant) comprising at least 90%, at least 95%, at least 96%, at least 97%, at least 98%, at least 99% sequence identity to SEQ ID NO: 822 and/or comprising one or more of the substitutions A106V, D108N, D147Y, and E155V; L84F, H123Y, and I157F; H36L, R51L, S146C, and K157N; and P48S and A142N of ecTadA*6.4;
(ii-7) ecTadA*7.8 (SEQ ID NO: 823) or a TadA variant (optionally ecTadA variant) comprising at least 90%, at least 95%, at least 96%, at least 97%, at least 98%, at least 99% sequence identity to SEQ ID NO: 823 and/or comprising one or more of the substitutions A106V. D108N, D147Y, and E155V; L84F. H123Y, and I157F; H36L, R51L, S146C, and K157N; A142N; and W23L and P48A of ecTadA*7.8;
(ii-8) ecTadA*7.9 (SEQ ID NO: 824) or a TadA variant (optionally ecTadA variant) comprising at least 90%, at least 95%, at least 96%, at least 97%, at least 98%, at least 99% sequence identity to SEQ ID NO: 824 and/or comprising one or more of the substitutions A106V. D108N, D147Y, and E155V; L84F. H123Y, and 1157F; H36L, R51L. S146C, and K157N; A142N; and W23L, P48A, and R152P of ecTadA*7.9; or
(ii-9) ecTadA*7.10 (SEQ ID NO: 825) or a TadA variant (optionally ecTadA variant) comprising at least 90%, at least 95%, at least 96%, at least 97%, at least 98%, at least 99% sequence identity to SEQ ID NO: 825 and/or comprising one or more of the substitutions A106V. D108N, D147Y, and E155V; L84F. H123Y, and 1157F; H36L. R51L. S146C, and K157N; and W23R, P48A, and R152P of ecTadA*7.10;
(b) a cytidine deaminase which:
(i) is derived from APOBEC, optionally from APOBEC1, further optionally from APOBECI of rat (rAPOBEC1) (SEQ ID NO: 720) or of human;
(ii) is or comprises any of the following:
(ii-1) rAOPBEC1 (SEQ ID NO: 720) or a AOPBEC1 variant (optionally rAOPBEC1 variant) comprising at least 90%, at least 95%, at least 96%, at least 97%, at least 98%, at least 99% sequence identity to SEQ ID NO: 720;
(ii-2) YE1-rAPOBEC1 (SEQ ID NO: 721) or a AOPBEC1 variant (optionally rAOPBEC1 variant or YE1-rAPOBEC1 variant) comprising at least 90%, at least 95%, at least 96%, at least 97%, at least 98%, at least 99% sequence identity to SEQ ID NO: 721 and/or comprising the W90Y and/or R126E substitutions of YE1-rAPOBEC1;
(ii-3) YE2-rAPOBEC1 (SEQ ID NO: 722) or a AOPBEC1 variant (optionally rAOPBEC1 variant or YE2-rAPOBEC1 variant) comprising at least 90%, at least 95%, at least 96%, at least 97%, at least 98%, at least 99% sequence identity to SEQ ID NO: 722 and/or comprising the W90Y and/or R132E substitutions of YE2-rAPOBEC1;
(ii-4) EE-rAPOBEC1 (SEQ ID NO: 723) or a AOPBEC1 variant (optionally rAOPBEC1 variant or EE-rAPOBEC1 variant) comprising at least 90%, at least 95%, at least 96%, at least 97%, at least 98%, at least 99% sequence identity to SEQ ID NO: 723 and/or comprising the R126E and/or R132E substitutions of EE-rAPOBEC1;
(ii-5) YEE-rAPOBEC1 (SEQ ID NO: 724) or a AOPBEC1 variant (optionally rAOPBEC1 variant or YEE-rAPOBEC1 variant) comprising at least 90%, at least 95%, at least 96%, at least 97%, at least 98%, at least 99% sequence identity to SEQ ID NO: 724 and/or comprising one or more of the substitutions W90Y, R126E, and R132E of YEE-rAPOBEC1;
(ii-6) Anc689 APOBEC (SEQ ID NO: 731) or a AOPBEC variant (optionally Anc689 APOBEC variant) comprising at least 90%, at least 95%, at least 96%, at least 97%, at least 98%, at least 99% sequence identity to SEQ ID NO: 731; or
(ii-7) Anc687 APOBEC (SEQ ID NO: 732) or a AOPBEC variant (optionally Anc687 APOBEC variant) comprising at least 90%, at least 95%, at least 96%, at least 97%, at least 98%, at least 99% sequence identity to SEQ ID NO: 732;
(iii) is derived from CDA1 or from AID;
(iv) is or comprises any of the following or a variant thereof comprising at least 90%, at least 95%, at least 96%, at least 97%, at least 98%, at least 99% sequence identity to thereto:
(iv-1) CDA1 (SEQ ID NO: 725); or
(iv-2) AID (SEQ ID NO: 726);
(v) is derived from a TadA deaminase, optionally from TadA of E. coli (ecTadA), optionally comprising the sequence of SEQ ID NO: 820; and/or
(vi) is or comprises any of the following or a variant thereof comprising at least 90%, at least 95%, at least 96%, at least 97%, at least 98%, at least 99% sequence identity to thereto:
(iv-1) TadA-CDa (SEQ ID NO: 741);
(iv-2) TadA-CDb (SEQ ID NO: 742);
(iv-3) TadA-CDc (SEQ ID NO: 743);
(iv-4) TadA-CDd (SEQ ID NO: 744);
(iv-5) TadA-CDe (SEQ ID NO: 745);
(iv-6) TadA-CDa-V106W (SEQ ID NO: 751);
(iv-7) TadA-CDb-V106W (SEQ ID NO: 752);
(iv-8) TadA-CDc-V106W (SEQ ID NO: 753);
(iv-9) TadA-CDd-V106W (SEQ ID NO: 754); or
(iv-10) TadA-CDe-V106W (SEQ ID NO: 755); or
(c) an adenine and cytidine deaminase (dual deaminase) which;
(i) is derived from TadA, optionally from TadA of E. coli (ecTadA) (SEQ ID NO: 820); and/or
(ii) is or comprises any of the following or a variant thereof comprising at least 90%, at least 95%, at least 96%, at least 97%, at least 98%, at least 99% sequence identity to thereto:
(ii-1) TadA*Dual (SEQ ID NO: 920); or
(ii-2) TadA*Dual-V106W (SEQ ID NO: 921);
(3) the base editor is:
(I) an ABE, which comprises:
(a) optionally a nuclear localization signal (NLS);
(b) an/the adenine deaminase;
(c) optionally a linker:
(d) an/the adenine deaminase;
(e) optionally a linker;
(f) a/the Cas-derived platform protein; and
(g) optionally a NLS,
wherein each of (a) to (g) if present, are optionally comprised in the ABE in the recited order in the direction from the N-terminus to the C-terminus;
(II) a CBE, which comprises:
(a) optionally a NLS;
(b) optionally Gam;
(c) optionally a linker;
(d) an/the cytidine deaminase:
(e) optionally a linker;
(f) a/the Cas-derived platform protein; and
(g) optionally a linker;
(h) optionally a uracil DNA glycosylase inhibitor (UGI);
(i) optionally a linker;
(j) optionally a UGI;
(k) optionally a linker; and
(l) optionally a NLS,
wherein each of (a) to (l) if present, are optionally comprised in the CBE in the recited order in the direction from the N-terminus to the C-terminus; or
(III) a DE, which comprises:
(a) optionally a NLS;
(b) optionally Gam;
(c) optionally a linker;
(d) an/the dual deaminase;
(e) optionally a linker;
(f) a/the Cas-derived platform protein; and
(g) optionally a linker;
(h) optionally a UGI;
(i) optionally a linker;
(i) optionally a UGI;
(k) optionally a linker; and
(l) optionally a NLS,
wherein each of (a) to (l) if present, are optionally comprised in the DE in the recited order in the direction from the N-terminus to the C-terminus,
optionally wherein:
(i) the NLS is or comprises any of the following or a variant thereof comprising at least 90%, at least 95%, at least 96%, at least 97%, at least 98%, at least 99% sequence identity to thereto;
(i-1) NLSI (SEQ ID NO: 691);
(i-2) NLS2 (SEQ ID NO: 692); and/or
(i-3) NLS3 (SEQ ID NO: 693);
(ii) the linker(s) individually:
(ii-1) comprise(s) one or more amino acids, optionally one, two, three, four, five, six, seven, eight, nine, ten, eleven, or twelve amino acids;
(ii-2) comprise(s) or consist(s) of G, S, and/or A;
(ii-3) comprise(s) or consist(s) of or comprise(s) or consist(s) of multiple repeats of an amino acid sequence selected from the group consisting of G. GG, GGG, GS, SG, GGS, GSG, SGG, GSS, SGS, SSG, SEQ ID NO: 682, SEQ ID NO: 685, and SEQ ID NO: 686; and/or
(ii-4) comprise(s) or consist(s) of the amino acid sequence of SEQ ID NO: 681, 683, or 684;
(iii) the UGI comprises the amino acid sequence of SEQ ID NO: 760 or an amino acid sequence comprising at least 90%, at least 95%, at least 96%, at least 97%, at least 98%, at least 99% sequence identity to thereto:
(iv) the Gam comprises the amino acid sequence of SEQ ID NO: 770 or an amino acid sequence comprising at least 90%, at least 95%, at least 96%, at least 97%, at least 98%, at least 99% sequence identity to thereto;
(4) the base editor is:
(a) an ABE which is or comprises any of the following or a variant thereof comprising at least 90%, at least 95%, at least 96%, at least 97%, at least 98%, at least 99% sequence identity to thereto:
(i) ABE8e (SEQ ID NO: 810);
(ii) ABE8e dimer (SEQ ID NO: 811);
(iii) ABEmax (SEQ ID NO: 801); and/or
(iv) ABE7.10 (SEQ ID NO: 800);
(b) a CBE which is or comprises any of the following or a variant thereof comprising at least 90%, at least 95%, at least 96%, at least 97%, at least 98%, at least 99% sequence identity to thereto:
(i) BE4max (SEQ ID NO: 712);
(ii) AncBE4max (SEQ ID NO: 713);
(iii) BE4 (SEQ ID NO: 710);
(iv) BE4-Gam (SEQ ID NO: 711);
(v) BE3 (SEQ ID NO: 700);
(vi) YE1-BE3 (SEQ ID NO: 701);
(vii) YE2-BE3 (SEQ ID NO: 702);
(viii) EE-BE3 (SEQ ID NO: 703);
(ix) YEE-BE3 (SEQ ID NO: 704);
(x) CDA1-BE3 (SEQ ID NO: 705);
(xi) AID-BE3 (SEQ ID NO: 706); and/or
(xii) BE3-Gam (SEQ ID NO: 707);
(c) a DE which is or comprises any of the following or a variant thereof comprising at least 90%, at least 95%, at least 96%, at least 97%, at least 98%, at least 99% sequence identity to thereto:
(i) TadDE (SEQ ID NO: 900); and/or
(ii) TadDE-V106W (SEQ ID NO: 901).

31-33. (canceled)

34. The composition of claim 29, wherein:
(1) the RNP is encapsulated in the TCV by:
(i) providing an aqueous solution comprising the TCV, optionally wherein the aqueous solution:
(i-1) has the pH of about 3 to about 8, about 4 to about 7.5, about 3.5 to 4.5, or about 4, optionally wherein said aqueous solution comprises an acetate buffer; and/or
(i-2) is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents; and
(ii) mixing the RNP with the aqueous solution, optionally wherein:
(ii-1) the mixing comprises gentle mixing, optionally by one or more of repeated manual reciprocation of the TCV-generating fluid in a pipette, micromixing, mixing using a staggered herringbone micromixer (SHM), T-junction mixing, or extrusion, and optionally wherein the mixing time is about 0.1 second to about 20 minutes;
(ii-2) the mixing is performed substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents, further optionally substantially, essentially, or entirely free of destabilizing agents;
(iii-3) when more than one RNPs are encapsulated in the TCV, the mixing comprises mixing an equimolar ratio of the more than one RNPs with the aqueous solution;
(2) the TCV comprises at least one cationic or ionizable cationic lipid, optionally wherein the TCV comprises one or more of the following features:
(i) said at least one cationic or ionizable cationic lipid comprises, essentially consists of, or consists of a lipid selected from the group consisting of N,N-dimethyl-2.3-dioleyloxy) propylamine (DODMA), 1.2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino) butanoate (MC3), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxyl) propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAR.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl) propyl-N,N-N-triethylammonium chloride (“DOTMA”); 1.2-Dioleyloxy-3-trimethylaminopropane chloride salt (“DOTAP.C1”); 3.beta.-(N-(N′,N′-dimethylaminoethane)-carbamoyl) cholesterol (“DC-Chol”), N-(1-(2,3-dioleyloxyl) propyl)-N-2-(sperminecarboxamido)ethyl)-N,N-dimethyl-ammonium trifluoracetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof;
(ii) saidTCV further comprises at least one helper lipid, optionally wherein the at least one helper lipid comprises, essentially consists of, or consists of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof;
(iii) said TCV further comprises at least one phospholipid, optionally wherein the at least one phospholipid comprises, essentially consists of, or consists of a lipid selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof;
(iv) saidTCV further comprises at least one cholesterol or cholesterol derivative, optionally wherein the at least one cholesterol or cholesterol derivative comprises, essentially consists of, or consists of a cholesterol or cholesterol derivative selected from the group consisting of cholesterol, N,N-dimethyl-N-ethylcarboxamidocholesterol (DC-Chol), 1,4-bis(3-N-oleylamino-propyl) piperazine, imidazole cholesterol ester (ICE), and any combinations thereof;
(v) said TCV further comprises at least one PEG or PEG-lipid, optionally wherein the at least one PEG-lipid comprises, essentially consists of, or consists of a PEG-lipid selected from the group consisting of PEG-myristoyl diglyceride (PEG-DMG) (e.g., 1.2-dimyristoyl-rac-glycero-3-methoxypolyethylene glycol-2000 (Avanti® Polar Lipids (Birmingham, AL)), which is a mixture of 1.2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1.2-diacyloxypropan-3-amines, and any combinations thereof, optionally wherein the total amount of said at least one PEG or PEG-lipid in said TCV is at most 2 mol %, 1.5 mol %, 1.0 mol %, 0.5 mol %, or 0.1 to 0.5 mol %;
(vi) said TCV further does not comprise any PEG or PEG-lipid;
(vii) the TCV is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents, and/or
(viii) the TCV is substantially, essentially, or entirely free of any permanently cationic lipids and/or any permanently anionic lipids;
or any combination of (i) to (viii),
optionally wherein:
(a) the amount of the at least one cationic or ionizable cationic lipid relative to the total components of the TCV is: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %;
(b) in (ii), the amount of the at least one helper lipid relative to the total components of the TCV is about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %;
(c) in (iii), the amount of the at least one phospholipid relative to the total components of the TCV is about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %;
(d) in (iv), the amount of the at least one cholesterol or cholesterol derivative relative to the total components of the TCV is about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%; and/or
(e) in (v), the amount of the at least one PEG or PEG-lipid relative to the total components of the TCV is about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %; and/or
(3) (I) said TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC; and
(iv) at least one cholesterol or cholesterol derivative,
optionally wherein the amounts of the at least one ionizable cationic lipid, the at least one helper lipid, the at least one phospholipid, and the at least one cholesterol or cholesterol derivative, relative to the total components of the TCV, is about 20 mol %, about 30 mol %, about 10 mol %, and about 40 mol %, respectively; or
(II) the said TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC;
(iv) at least one cholesterol or cholesterol derivative; and
(v) at least one PEG or PEG-lipid, which is optionally PEG-DMG, optionally wherein the amounts of the at least one ionizable cationic lipid, the at least one helper lipid, the at least one phospholipid, the at least one cholesterol or cholesterol derivative, and the at least one PEG or PEG-lipid, relative to the total components of the TCV, is about 20 mol %, about 30 mol %, about 10 mol %, about 39 mol %, and about 1 mol %, respectively,
and wherein said TCV is substantially, essentially, or entirely free of ethanol, methanol, isopropanol, tetrahydrofuran (THF), dimethyl sulfoxide (DMSO), dimethyl formamide (DMF), and acetonitrile (ACN),
optionally wherein said TCV is:
(A) substantially, essentially, or entirely free of organic solvents and detergents;
(B) substantially, essentially, or entirely free of destabilizing agents;
(C) substantially, essentially, or entirely free of any permanently cationic lipids and/or any permanently anionic lipids; and/or
(D) stable for prolonged periods of time at about 1 to about 40° C., about 5 to about 35° C., about 10 to about 30° C., or about 15 to about 25° C.,
further optionally wherein the TCV or the composition further comprises and/or is stored in the presence of at least one cryoprotectant, optionally wherein:
(a) the cryoprotectant comprises a sugar-based molecule, which is optionally sucrose, trehalose, or a combination thereof;
(b) the concentration of the cryoprotectant is about 1% to about 40%, about 3% to about 30%, about 5% to about 30%, about 10% to about 20%, or about 15%;
(c) the TCV is stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months; or
(d) any combination of (a)-(c).

35-37. (canceled)

38. A method of effecting base editing in one or more target cells, comprising applying an effective amount of the composition of claim 29 to the one or more target cells, optionally wherein the applying occurs in vitro, ex vivo, or in vivo.

39. A method of (1) treating a subject and/or (2) treating a disease, a disorder, or a condition in a subject, the method comprising administering an effective amount of the composition of claim 29 to the subject,
optionally wherein:
(a) the administering is:
(i) to one or more of the subject's eyes, optionally
(i-1) intravitreally or via ocular drops;
(i-2) into the cornea, optionally the epithelial, stromal, and/or endothelial cells of the cornea;
(i-3) into the iris,
(i-4) into the retina; or
(i-5) into the subretinal tissue;
(ii) locally administering, optionally to the eye, ear, nose (optionally intranasally), skin (optionally transdermally or epicutaneously), mucosa, skin, or vagina, or by inhalation;
(iii) parenterally administering, optionally by injection (optionally intravenous, intramuscular, subcutaneous, intradermal, intrathecal, intra-arterial, intraarticular, intraosseous, or intraperitoneal administration) or by inhalation; or
(iv) enterally administering, optionally orally, sublingually, buccally, or rectally;
(b) the method is for effecting base editing in:
(i) the cornea, optionally an epithelial, stromal, and/or endothelial cell of the cornea;
(ii) the iris;
(iii) the retina, optionally a photoreceptor cell, a bipolar cell, a retinal ganglion cell, a muller glial cell, a horizontal cell, and/or a amacrine cell of the retina; or
(iv) the subretinal tissue; and/or
(c) the subject is a human, non-human, non-human primate, a rodent (mouse, rat, guinea pig, hamster), rabbit, cat, dog, pig, goat, sheep, horse, or monkey,
further optionally wherein:
in (1), the subject has or has a risk of contracting with a genetic disease associated with one or more genetic mutations and the composition is designed to reverse or alter the genetic mutation(s); and/or
in (2), the disease a disorder, or a condition is associated with one or more genetic mutations and the composition is designed to reverse or alter the genetic mutation(s).

40-41. (canceled)

42. A method of preparing the composition of claim 29, comprising:
(i) providing an aqueous solution comprising the TCV, optionally wherein the aqueous solution:
(i-1) has the pH of about 3 to about 8, about 4 to about 7.5, about 3.5 to 4.5, or about 4, optionally wherein said aqueous solution comprises an acetate buffer; and/or
(i-2) is: substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents; further optionally substantially, essentially, or entirely free of destabilizing agents; and
(ii) mixing the RNP with the aqueous solution, optionally wherein:
(ii-1) the mixing comprises gentle mixing (optionally repeated manual reciprocation of the TCV-generating fluid in a pipette), micromixing, mixing using a staggered herringbone micromixer (SHM), T-junction mixing, or extrusion, and optionally wherein the mixing time is about 0.1 second to about 20 minutes;
(ii-2) the mixing is performed substantially, essentially, or entirely free of ethanol, methanol, isopropanol, tetrahydrofuran (THF), dimethyl sulfoxide (DMSO), dimethyl formamide (DMF), and acetonitrile (ACN); and/or substantially, essentially, or entirely free of sodium dodecyl sulfate (SDS); optionally substantially, essentially, or entirely free of organic solvents and/or detergents, further optionally substantially, essentially, or entirely free of destabilizing agents;
(iii-3) when more than one RNPs are encapsulated in the TCV, the mixing comprises mixing an equimolar ratio of the more than one RNPs with the aqueous solution.
